(19) United States
(12) Patent Application Publication
(10) Pub. No.: US 2023/0193271 A1
(43) Pub. Date: Jun. 22, 2023

(54) Treatment Of Kidney Diseases With Angiopoietin Like 3 (ANGPTL3) Inhibitors

(71) Applicant: Regeneron Pharmaceuticals, Inc., Tarrytown, NY (US)

(72) Inventors: Mary E. Haas, Tarrytown, NY (US); Luca Andrea Lotta, Tarrytown, NY (US); Aris Baras, Tarrytown, NY (US); Manuel Allen Revez Ferreira, Tarrytown, NY (US); Kishor Devalaraja-Narashimha, Tarrytown, NY (US); Lori Morton, Tarrytown, NY (US)

(21) Appl. No.: 18/069,503

(22) Filed: Dec. 21, 2022

Related U.S. Application Data
(60) Provisional application No. 63/292,581, filed on Dec. 22, 2021.
(60) Provisional application No. 63/340,254, filed on May 10, 2022.

(51) Int. Cl. C12N 15/113; C07K 16/28; A61P 13/12; C12N 9/22

(52) U.S. Cl. CPC C12N 15/113; C07K 16/2839; A61P 13/12; C12N 9/22; C12N 2310/11

(57) ABSTRACT

The present disclosure provides methods of treating a subject having a kidney disease or at risk of developing a kidney disease by administering an Angiopoietin Like 3 (ANGPTL3) inhibitor, and methods of identifying subjects having an increased risk of developing a kidney disease.

### REFERENCE TO SEQUENCE LISTING

This application includes a Sequence Listing filed electronically as an XML file named 381203628SEQ, created on Nov. 28, 2022, with a size of 1,673 kilobytes. The Sequence Listing is incorporated herein by reference.

### FIELD

The present disclosure relates generally to the treatment of subjects having a kidney disease or at risk of developing a kidney disease with a Angiopoietin Like 3 (ANGPTL3) inhibitor, and methods of identifying subjects having an increased risk of developing a kidney disease.

### BACKGROUND

In the United States, based on data from the 1999-2006 National Health and Nutrition Examination Survey (NHANES) study, an estimated 11.1 percent (22.4 million) of adults aged 20 or older have chronic kidney disease (CKD) stages 1-3. An additional 0.8 million U.S. adults aged 20 or older have CKD stage 4, and more than 0.3 million have stage 5 CKD and receive hemodialysis. Analyses of NHANES data between 1988-1994 and 1999-2004 suggest that the prevalence of CKD is rising for every CKD stage, but with a particular increase in the prevalence of individuals classified with CKD stage 3. The number of patients with stage 5 CKD requiring dialysis also has increased. It has been estimated that more than 700,000 individuals will have End Stage Renal Disease (ESRD) by 2015. Although CKD can be caused by primary kidney disease (e.g., glomerular diseases, tubulointerstitial diseases, obstruction, and polycystic kidney disease), in the vast majority of patients with CKD, the kidney damage is associated with other medical conditions such as diabetes and hypertension. In 2008, excluding those with ESRD, 48 percent of Medicare patients with CKD had diabetes, 91 percent had hypertension, and 46 percent had atherosclerotic heart disease. Other risk factors for CKD include age, obesity, family history, and ethnicity. CKD has been associated with numerous adverse health outcomes.

A Glomerular Filtration Rate (GFR) of 90 mL/min or higher (Stage 1) is normal in most healthy people. Usually few symptoms are present at this stage of CKD. A GFR of 60-89 mL/min (Stage 2) may for some patients, such as the elderly or infants, be normal if no kidney damage is present. A GFR between 60-89 mL/min for three months or longer along with kidney damage is a sign of early CKD. Usually few symptoms are present at this stage. A GFR between 30-59 mL/min (Stage 3) for a patient is indicative of moderate CKD, and are more likely to develop anemia, early bone disease or high blood pressure, and may desire to see a nephrologist. A GFR between 15-29 mL/min (Stage 4) indicates that the patient has severe CKD, and will likely need dialysis or a kidney transplant in the future. A GFR of 15 mL/min or less (Stage 5) indicates that the patient has chronic CKD, and have ESRD. The kidneys have lost almost all ability to function effectively at this stage. They will need dialysis or a kidney transplant to live.

The ANGPTL3 gene encodes a member of a family of secreted proteins that function in angiogenesis. The encoded protein, which is expressed predominantly in the liver, is further processed into an N-terminal coiled-coil domain-containing chain and a C-terminal fibrinogen chain. The N-terminal chain is important for lipid metabolism, while the C-terminal chain may be involved in angiogenesis. Mutations in this gene cause familial hypobetalipoproteinemia type 2.

### SUMMARY

The present disclosure provides methods of treating a subject having a kidney disease or at risk of developing a kidney disease, the methods comprising administering an ANGPTL3 inhibitor to the subject, wherein the kidney disease is not nephrotic syndrome.

The present disclosure also provides methods of treating a subject having a kidney disease or at risk of developing a kidney disease by administering a kidney disease therapeutic agent, the methods comprising: determining whether the subject has an ANGPTL3 predicted loss-of-function or missense variant nucleic acid molecule by: obtaining or having obtained a biological sample from the subject; and performing or having performed a sequence analysis on the biological sample to determine if the subject has a genotype comprising the ANGPTL3 predicted loss-of-function or missense variant nucleic acid molecule; and administering or continuing to administer the kidney disease therapeutic agent in a standard dosage amount to a subject that is ANGPTL3 reference, and/or administering an ANGPTL3 inhibitor to the subject; administering or continuing to administer the kidney disease therapeutic agent in an amount that is the same as or less than a standard dosage amount to a subject that is heterozygous for the ANGPTL3 predicted loss-of-function or missense variant nucleic acid molecule, and/or administering an ANGPTL3 inhibitor to the subject; or administering or continuing to administer the kidney disease therapeutic agent in an amount that is the same as or less than a standard dosage amount to a subject that is homozygous for the ANGPTL3 predicted loss-of-function or missense variant nucleic acid molecule; wherein the presence of a genotype having the ANGPTL3 predicted loss-of-function or missense variant nucleic acid molecule indicates the subject has a decreased risk of developing a kidney disease; and wherein the kidney disease is not nephrotic syndrome.

The present disclosure also provides methods of identifying a subject having an increased risk of developing a kidney disease, the methods comprising: determining or having determined the presence or absence of an ANGPTL3 predicted loss-of-function or missense variant nucleic acid molecule in a biological sample obtained from the subject; wherein: when the subject is ANGPTL3 reference, then the subject has an increased risk of developing a kidney disease; and when the subject is heterozygous or homozygous for the ANGPTL3 predicted loss-of-function or missense variant nucleic acid molecule, then the subject has a decreased risk of developing a kidney disease; and wherein the kidney disease is not nephrotic syndrome.

The present disclosure also provides kidney disease therapeutic agent for use in the treatment and/or prevention of a kidney disease in a subject having an ANGPTL3 predicted loss-of-function or missense variant nucleic acid molecule, wherein the kidney disease is not nephrotic syndrome.

The present disclosure also provides ANGPTL3 inhibitors for use in the treatment and/or prevention of a kidney disease in a subject that is ANGPTL3 reference, or is heterozygous for ANGPTL3 predicted loss-of-function or missense variant nucleic acid molecule, wherein the kidney disease is not nephrotic syndrome.

### BRIEF DESCRIPTION OF THE DRAWINGS

The accompanying figures, which are incorporated in and constitute a part of this specification, illustrate several features of the present disclosure.

FIG. 1 shows associations of circulating levels of ANGPTL3 measured at baseline samples with risk of newly-onset chronic kidney disease (n=977/38,043) and the observed risk of newly-onset chronic kidney disease among the quartiles of plasma ANGPTL3 measured via Olink® Explore 1536 assay; analyses were performed among individuals without prior history of chronic kidney disease at baseline; the adjusted hazard ratios and corresponding confidence intervals were estimated using Cox regression, stratified by sex and study center, adjusted for age-at-baseline, systolic blood pressure, high-density lipoprotein cholesterol, low-density lipoprotein cholesterol, smoking status, alcohol consumption, history of diabetes, and eGFR_{creatinine}; abbreviations: HR, hazard ratios, CI, confidence interval, SD, standard deviation, Q1-Q5: the 1^{st} quartile, . . . , the fifth quartile.

FIG. 2 (Panel A) is a graph depicting the results of urinary albumin normalized to urinary creatinine in wildtype lean (Lean) and uninephrectomized db/db C57BLKS (db/db) mice; FIG. 2 (Panel B) is a graph depicting the results of an ELISA analysis of serum ANGPTL3 in wildtype lean (Lean) and uninephrectomized db/db C57BLKS (db/db) mice. **, unpaired t-test p<0.01.

FIG. 3 (Panel A) is a graph depicting the results of urinary albumin normalized to urinary creatinine in sham-operated (Sham) and ⅚^{th} nephrectomy remnant kidney model (RKM) mice; FIG. 3 (Panel B) is a graph depicting the results of an ELISA analysis of serum ANGPTL3 in in sham-operated (Sham) and ⅚^{th} nephrectomy remnant kidney model (RKM) mice. *, unpaired t-test p<0.05; *** unpaired t-test p<0.0005

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or Alternately phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates. In some embodiments, the subject is a human. In some embodiments, the human is a patient under the care of a physician.

It has been observed in accordance with the present disclosure that ANGPTL3 predicted loss-of-function or missense variant nucleic acid molecules (whether these variants are homozygous or heterozygous in a particular subject) associate with a decreased risk of developing a kidney disease. It is believed that ANGPTL3 predicted loss-of-function coding variant nucleic acid molecules have not been associated with kidney disease. Therefore, subjects that are ANGPTL3 reference or heterozygous for an ANGPTL3 predicted loss-of-function or missense variant nucleic acid molecule may be treated with an ANGPTL3 inhibitor such that a kidney disease is inhibited or prevented, the symptoms thereof are reduced or prevented, and/or development of symptoms is repressed or prevented. It is also believed that such subjects having a kidney disease may further be treated with a kidney disease therapeutic agent.

For purposes of the present disclosure, any particular subject, such as a human, can be categorized as having one of three ANGPTL3 genotypes: i) ANGPTL3 reference; ii) heterozygous for an ANGPTL3 predicted loss-of-function or missense variant nucleic acid molecule; or iii) homozygous for an ANGPTL3 predicted loss-of-function or missense variant nucleic acid molecule. A subject is ANGPTL3 reference when the subject does not have a copy of an ANGPTL3 predicted loss-of-function or missense variant nucleic acid molecule. A subject is heterozygous for an ANGPTL3 predicted loss-of-function or missense variant nucleic acid molecule when the subject has a single copy of an ANGPTL3 predicted loss-of-function or missense variant nucleic acid molecule. An ANGPTL3 predicted loss-of-function or missense variant nucleic acid molecule is any nucleic acid molecule (such as, a genomic nucleic acid molecule, an mRNA molecule, or a cDNA molecule) encoding a variant ANGPTL3 polypeptide having a partial loss-of-function, a complete loss-of-function, a predicted partial loss-of-function, or a predicted complete loss-of-function. In some embodiments, the ANGPTL3 predicted loss-of-function or missense variant nucleic acid molecule comprises a variation in a coding region. In some embodiments, the ANGPTL3 predicted loss-of-function or missense variant nucleic acid molecule does not comprise a variation in a non-coding region, except for splice acceptor regions (two bases before the start of any exon except the first). A subject who has an ANGPTL3 polypeptide having a partial loss-of-function (or predicted partial loss-of-function) is hypomorphic for ANGPTL3. A subject is homozygous for an ANGPTL3 predicted loss-of-function or missense variant nucleic acid molecule when the subject has two copies (same or different) of an ANGPTL3 predicted loss-of-function or missense variant nucleic acid molecule.

For subjects that are genotyped or determined to be ANGPTL3 reference, such subjects have an increased risk of developing a kidney disease. For subjects that are genotyped or determined to be either ANGPTL3 reference or heterozygous for an ANGPTL3 predicted loss-of-function or missense variant nucleic acid molecule, such subjects or subjects can be treated with an ANGPTL3 inhibitor.

In any of the embodiments described herein, the subject in whom a kidney disease is treated or prevented by administering the ANGPTL3 inhibitor can be anyone at risk for developing a kidney disease including, but not limited to, subjects with a genetic predisposition for developing a kidney disease. Additional risk factors include, but are not limited to, diabetes, hypertension, obesity, excessive salt intake, age, smoking, excessive alcohol consumption, heavy metal exposure, hyperlipidemia, and the presence of autoimmune diseases. In addition, in some embodiments, any subject can be at risk of developing a kidney disease. In some embodiments, administering an ANGPTL3 inhibitor may be carried out to prevent development of an additional kidney disease in a subject who has already had a kidney disease.

In any of the embodiments described herein, the ANGPTL3 predicted loss-of-function or missense variant nucleic acid molecule can be any nucleic acid molecule (such as, for example, genomic nucleic acid molecule, mRNA molecule, or cDNA molecule) encoding an ANGPTL3 variant polypeptide having a partial loss-of-function, a complete loss-of-function, a predicted partial loss-of-function, or a predicted complete loss-of-function. In some embodiments, the ANGPTL3 predicted loss-of-function or missense variant nucleic acid molecule is any nucleic acid molecule (such as, a genomic nucleic acid molecule, an mRNA molecule, or a cDNA molecule) resulting in decreased or aberrant expression of ANGPTL3 mRNA or polypeptide. In some embodiments, the ANGPTL3 predicted loss-of-function or missense variant nucleic acid molecule is associated with a reduced in vitro response to ANGPTL3 ligands compared with reference ANGPTL3. In some embodiments, the ANGPTL3 predicted loss-of-function or missense variant nucleic acid molecule is an ANGPTL3 variant nucleic acid molecule that results or is predicted to result in a premature truncation of an ANGPTL3 polypeptide compared to the human reference genome sequence. In some embodiments, the ANGPTL3 predicted loss-of-function or missense variant nucleic acid molecule is a variant that is predicted to be damaging to the protein function (and hence, in this case, protective to the human) by in vitro prediction algorithms such as Polyphen, SIFT, or similar algorithms. In some embodiments, the ANGPTL3 predicted loss-of-function or missense variant nucleic acid molecule is a variant that causes or is predicted to cause a nonsynonymous amino-acid substitution in an ANGPTL3 nucleic acid molecule and whose allele frequency is less than 1/100 alleles in the population from which the subject is selected. In some embodiments, the ANGPTL3 predicted loss-of-function or missense variant nucleic acid molecule is any rare missense variant (allele frequency <0.1%; or 1 in 1,000 alleles), or any splice-site, stop-gain, start-loss, stop-loss, frameshift, or in-frame indel, or other frameshift ANGPTL3 variant.

In any of the embodiments described herein, the ANGPTL3 predicted loss-of-function polypeptide can be any ANGPTL3 polypeptide having a partial loss-of-function, a complete loss-of-function, a predicted partial loss-of-function, or a predicted complete loss-of-function.

In any of the embodiments described herein, the ANGPTL3 predicted loss-of-function or missense variant nucleic acid molecule can include variations at positions of chromosome 1 using the nucleotide sequence of the ANGPTL3 reference genomic nucleic acid molecule (see, ENST00000371129 annotated in the in the Ensembl database (URL: world wide web at “//useast.ensembl.org/index.html”)) as a reference sequence. The sequence provided in ENST00000371129 for the ANGPTL3 genomic nucleic acid molecule is only an exemplary sequence. Other sequences for the ANGPTL3 genomic nucleic acid molecule are also possible. Exemplary ANGPTL3 predicted loss-of-function or missense variant nucleic acid molecules include, but are not limited to those recited in Table 4.

Any one or more (i.e., any combination) of the ANGPTL3 predicted loss-of-function or missense variant nucleic acid molecules described herein can be used within any of the methods described herein to determine whether a subject has an increased or decreased risk of developing a kidney disease. The combinations of particular variants can form a mask used for statistical analysis of the particular correlation of ANGPTL3 and an increased or decreased risk of developing a kidney disease. In some embodiments, the mask used for statistical analysis of the particular correlation of ANGPTL3 and an increased or decreased risk of developing a kidney disease can exclude any one or more of these ANGPTL3 predicted loss-of-function or missense variant nucleic acid molecules described herein.

In any of the embodiments described herein, the subject can have a kidney disease. In any of the embodiments described herein, the subject can be at risk of developing a kidney disease. In any of the embodiments described herein, the kidney disease is chronic kidney disease, a kidney stone, chronic glomerulonephritis, nephronophthisis, chronic interstitial nephritis, and/or nephrosclerosis. In some embodiments, the kidney disease is chronic kidney disease. In some embodiments, the kidney disease is a kidney stone. In some embodiments, the kidney disease is chronic glomerulonephritis. In some embodiments, the kidney disease is nephronophthisis. In some embodiments, the kidney disease is chronic interstitial nephritis. In some embodiments, the kidney disease is nephrosclerosis. In any of the embodiments described herein, the kidney disease is not nephrotic syndrome.

Other kidney disease include, but are not limited to, acquired cystic disease, acute (postinfectious) glomerulonephritis, acute infectious interstitial nephritis, acute interstitial nephritis, acute pyelonephritis, acute renal failure, acute transplant failure, acute tubular necrosis, adult polycystic kidney disease, AL amyloid, analgesic nephrosis, ANCA-associated vasculitis, anti-glomerular basement membrane disease (Goodpasture's Syndrome), antibody-mediated kidney graft rejection, asymptomatic hematuria, asymptomatic proteinuria, atypical hemolytic uremic syndrome, autosomal dominant polycystic kidney disease, autosomal recessive polycystic kidney disease, BK virus-associated nephropathy, Bence Jones cast nephrosis, benign familial hematuria, benign nephrosclerosis and atheromatous embolization, bilateral cortical necrosis, C3 glomerulonephritis, cardiac surgery associated acute kidney injury, chronic allograft nephropathy, chronic glomerulonephritis, chronic interstitial nephritis, chronic pyelonephritis, chronic renal failure, chronic transplant failure, circulating immune complex nephritis, contrast-induced nephropathy, crescentic glomerulonephritis, cryoglobulinemia, cystic renal dysplasia, delayed graft function, dense deposit disease, diabetic glomerulosclerosis, diabetic nephropathy, dialysis cystic disease, drug induced (allergic) acute interstitial nephritis, ectopic kidney, eosinophilic granulomatosis with polyangiitis, Fabry's disease, familial juvenile nephronophthisis-medullary cystic disease complex, focal segmental glomerulosclerosis (segmental hyalinosis), glomerulocystic disease, glomerulonephritis, glomerulonephritis associated with bacterial endocarditis, glomerulosclerosis, granulomatosis with polyangiitis, hemolytic-uremic syndrome, Henoch-Schonlein purpura, hepatitis-associated glomerulonephritis, hereditary nephritis (Alport syndrome), Human Immunodeficiency Virus-associated nephropathy, horseshoe kidney, hydronephrosis, hyperoxaluria, hypertensive nephropathy, IgA nephropathy, infantile polycystic kidney disease, ischemic acute tubular necrosis, light-chain deposit disease, lupus nephritis, malignant nephrosclerosis, medullary cystic disease, membranoproliferative (mesangiocapillary) glomerulonephritis, membranous glomerulonephritis, membranous nephropathy, mesangial proliferative glomerulonephritis (includes Berger's Disease), microscopic polyangiitis, minimal change glomerular disease, nephritic syndrome, nephroblastoma (Wilms tumor), nephronophthisis (medullary cystic disease complex), pigment nephropathy, plasma cell dyscrasias (monoclonal immunoglobulin-induced renal damage), polyarteritis nodosa, polycystic kidney disease, proteinuria, pyelonephritis, rapidly progressive (crescentic) glomerulonephritis, renal agenesis, renal amyloidosis, renal cell carcinoma, renal dysgenesis, renal dysplasia, renal hypoplasia, renal infection, renal osteodystrophy, renal stones (urolithiasis), renal tubular acidosis, renal vasculitis, renovascular hypertension, scleroderma (progressive systemic sclerosis), secondary acquired glomerulonephritis, sepsis-associated acute kidney injury, simple renal cysts, systemic lupus erythematosus, T-cell-mediated kidney graft rejection, thin basement membrane nephropathy, thrombotic microangiopathy, thrombotic thrombocytopenic purpura, toxic acute tubular necrosis, tubular defects, tubulointerstitial disease in multiple myeloma, urate nephropathy, urinary obstruction, and vasculitis.

Symptoms of chronic kidney disease include, but are not limited to, nausea, vomiting, loss of appetite, fatigue and weakness, sleep problems, changes urination volume, decreased mental sharpness, muscle twitches and cramps, swelling of feet and ankles, persistent itching, chest pain, fluid build-up around the lining of the heart, shortness of breath, fluid build-up in the lungs, and high blood pressure (hypertension) that's difficult to control.

Symptoms of a kidney stone include, but are not limited to, severe, sharp pain in the side and back, below the ribs, pain that radiates to the lower abdomen and groin, pain that comes in waves and fluctuates in intensity, pain or burning sensation while urinating, pink, red or brown urine, cloudy or foul-smelling urine, a persistent need to urinate, urinating more often than usual or urinating in small amounts, nausea and vomiting, and fever and chills if an infection is present.

Symptoms of chronic glomerulonephritis include, but are not limited to, pink or cola-colored urine from red blood cells in your urine (hematuria), foamy urine due to excess protein (proteinuria), high blood pressure (hypertension), and fluid retention (edema) with swelling evident in the face, hands, feet and abdomen.

Symptoms of nephronophthisis include, but are not limited to, increased urine production (polyuria), excessive thirst (polydipsia), general weakness, and extreme tiredness (fatigue).

Symptoms of chronic interstitial nephritis include, but are not limited to, blood in the urine, fever, increased or decreased urine output, mental status changes (drowsiness, confusion, coma), nausea, vomiting, rash, swelling of any area of body, and weight gain (from retaining fluid).

Symptoms of nephrosclerosis include, but are not limited to, impaired vision, blood in the urine, loss of weight, and the accumulation of urea and other nitrogenous waste products in the blood, a condition known as uremia.

The present disclosure provides methods of treating a subject having a kidney disease or at risk of developing a kidney disease, the methods comprising administering an ANGPTL3 inhibitor to the subject, wherein the kidney disease is not nephrotic syndrome.

In some embodiments, the ANGPTL3 inhibitor comprises an inhibitory nucleic acid molecule. Examples of inhibitory nucleic acid molecules include, but are not limited to, antisense nucleic acid molecules, small interfering RNAs (siRNAs), and short hairpin RNAs (shRNAs). Such inhibitory nucleic acid molecules can be designed to target any region of an ANGPTL3 nucleic acid molecule. In some embodiments, the antisense RNA, siRNA, or shRNA hybridizes to a sequence within an ANGPTL3 genomic nucleic acid molecule or mRNA molecule and decreases expression of the ANGPTL3 polypeptide in a cell in the subject. In some embodiments, the ANGPTL3 inhibitor comprises an antisense molecule that hybridizes to an ANGPTL3 genomic nucleic acid molecule or mRNA molecule and decreases expression of the ANGPTL3 polypeptide in a cell in the subject. In some embodiments, the ANGPTL3 inhibitor comprises an siRNA that hybridizes to an ANGPTL3 genomic nucleic acid molecule or mRNA molecule and decreases expression of the ANGPTL3 polypeptide in a cell in the subject. In some embodiments, the ANGPTL3 inhibitor comprises an shRNA that hybridizes to an ANGPTL3 genomic nucleic acid molecule or mRNA molecule and decreases expression of the ANGPTL3 polypeptide in a cell in the subject.

In some embodiments, the antisense nucleic acid molecules comprise or consist of any of the nucleotide sequences represented by SEQ ID NOs: 179-503. In some embodiments, the siRNA molecules comprise or consist of any of the nucleotide sequences (sense and antisense strands presented one after the other) represented by SEQ ID NOs: 504-1367 (e.g., the sense strand is, for example, SEQ ID NO: 504 and the corresponding antisense strand is SEQ ID NO: 505; the sense strand is, for example, SEQ ID NO: 506 and the corresponding antisense strand is SEQ ID NO: 507; etc.).

The inhibitory nucleic acid molecules can comprise RNA, DNA, or both RNA and DNA. The inhibitory nucleic acid molecules can also be linked or fused to a heterologous nucleic acid sequence, such as in a vector, or a heterologous label. For example, the inhibitory nucleic acid molecules can be within a vector or as an exogenous donor sequence comprising the inhibitory nucleic acid molecule and a heterologous nucleic acid sequence. The inhibitory nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The inhibitory nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The inhibitory nucleic acid molecules can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₙCH₃, —O(CH₂)ₙOCH₃, —O(CH₂)ₙNH₂, —O(CH₂)ₙCH₃, —O(CH₂)ₙ—ONH₂, and —O(CH₂)ₙ—ON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl (2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used.

In some embodiments, the siRNA molecules have backbone modifications. In some embodiments, the modified phosphodiester groups that link consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo- and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N₆-methyladenosine, inosine, and N₇-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative siRNA has the following formula:

Sense: mN*mN*/i2FN/mN/i2FN/mN/i2FN/mN/i2FN/mN/i2FN/mN/i2FN/mN/i2FN/mN/i2FN/*mN*/32FN/

### Antisense: /52FN/*/i2FN/*mN/i2FN/mN/i2FN/mN/i2FN/mN/i2FN/mN/i2FN/mN/i2FN/mN/i2FN/mN/i2FN/mN*N*N

wherein: “N” is the base; “2F” is a 2′-F modification; “m” is a 2′-O-methyl modification, “I” is an internal base; and “*” is a phosphorothioate backbone linkage.

In any of the embodiments described herein for antisense molecules and siRNA molecules, the molecules can comprise 1, 2, or 3 additional nucleotides at the 5′ end, 3′ end, or both the 5′ end and 3′ end. In some embodiments the antisense molecules and siRNA molecules comprise 1, 2, or 3 additional nucleotides at the 5′ end. In some embodiments the antisense molecules and siRNA molecules comprise 1, 2, or 3 additional nucleotides at the 3′ end. In some embodiments the antisense molecules and siRNA molecules comprise 1, 2, or 3 additional nucleotides at both the 5′ end and 3′ end.

In any of the embodiments described herein for antisense molecules and siRNA molecules, the molecules can comprise a substantially identical sequence having at least 80%, at least 85%, at least 90%, at least 95%, at least 98%, or at least 99% homology to the nucleotide sequences disclosed herein. In some embodiments, the antisense molecules and siRNA molecules have at least 80% homology to the nucleotide sequences disclosed herein. In some embodiments, the antisense molecules and siRNA molecules have at least 85% homology to the nucleotide sequences disclosed herein. In some embodiments, the antisense molecules and siRNA molecules have at least 90% homology to the nucleotide sequences disclosed herein. In some embodiments, the antisense molecules and siRNA molecules have at least 95% homology to the nucleotide sequences disclosed herein. In some embodiments, the antisense molecules and siRNA molecules have at least 98% homology to the nucleotide sequences disclosed herein. In some embodiments, the antisense molecules and siRNA molecules have at least 99% homology to the nucleotide sequences disclosed herein.

In any of the embodiments described herein, the inhibitory nucleic acid molecules may be administered, for example, as one to two hour i.v. infusions or s.c. injections. In any of the embodiments described herein, the inhibitory nucleic acid molecules may be administered at dose levels that range from about 50 mg to about 900 mg, from about 100 mg to about 800 mg, from about 150 mg to about 700 mg, or from about 175 to about 640 mg (2.5 to 9.14 mg/kg; 92.5 to 338 mg/m²— based on an assumption of a body weight of 70 kg and a conversion of mg/kg to mg/m² dose levels based on a mg/kg dose multiplier value of 37 for humans).

In some embodiments, the siRNA molecules comprise or consist of the nucleotide sequences (sense and antisense strands) recited in U.S. Pat. No. 10,995,335 and PCT Publication No. WO 2019/055633, which are incorporated herein by reference in their entirety.

In some embodiments, the siRNA molecules comprise or consist of the nucleotide sequences (sense and antisense strands) recited in U.S. Pat. No. 10,875,884 and PCT Publication Nos. WO 2015/168589, WO 2015/100394, and WO 2011/085271, which are incorporated herein by reference in their entirety.

In some embodiments, the siRNA molecules comprise or consist of the nucleotide sequences (sense and antisense strands) recited in U.S. Pat. Nos. 10,570,393 and 10,337,010, and PCT Publication Nos. WO 2016/168286 and WO 2012/177784, which are incorporated herein by reference in their entirety.

The present disclosure also provides vectors comprising any one or more of the inhibitory nucleic acid molecules. In some embodiments, the vectors comprise any one or more of the inhibitory nucleic acid molecules and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

The present disclosure also provides compositions comprising any one or more of the inhibitory nucleic acid molecules. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In some embodiments, the ANGPTL3 inhibitor comprises a nuclease agent that induces one or more nicks or double-strand breaks at a recognition sequence(s) or a DNA-binding protein that binds to a recognition sequence within an ANGPTL3 genomic nucleic acid molecule. The recognition sequence can be located within a coding region of the ANGPTL3 gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the ANGPTL3 gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify an ANGPTL3 genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of ANGPTL3 nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in an ANGPTL3 genomic nucleic acid molecule or it can be a nickase that creates a single-strand break in an ANGPTL3 genomic nucleic acid molecule. Additional examples of Cas proteins include, but are not limited to, Cas1, Cas1B, Cast, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (Cas6), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. In some embodiments, a Cas system, such as Cas12a, can have multiple gRNAs encoded into a single crRNA. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of ANGPTL3 genomic nucleic acid molecules can be generated by contacting a cell with a Cas protein and one or more gRNAs that hybridize to one or more gRNA recognition sequences within a target genomic locus in the ANGPTL3 genomic nucleic acid molecule. The gRNA recognition sequence can include or be proximate to the start codon of an ANGPTL3 genomic nucleic acid molecule or the stop codon of an ANGPTL3 genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in an ANGPTL3 genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2-6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10, about 2 to about 5 base pairs, or three base pairs upstream or downstream of the PAM sequence. In some embodiments (such as when Cas9 from S. pyogenes or a closely related Cas9 is used), the PAM sequence of the non-complementary strand can be 5′-NGG-3′, where N is any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within an ANGPTL3 genomic nucleic acid molecule. An exemplary gRNA is a gRNA effective to direct a Cas enzyme to bind to or cleave an ANGPTL3 genomic nucleic acid molecule, wherein the gRNA comprises a DNA-targeting segment that hybridizes to a gRNA recognition sequence within the ANGPTL3 genomic nucleic acid molecule. Exemplary gRNAs comprise a DNA-targeting segment that hybridizes to a gRNA recognition sequence present within an ANGPTL3 genomic nucleic acid molecule that includes or is proximate to the start codon or the stop codon. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the start codon or located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

The Cas protein and the gRNA form a complex, and the Cas protein cleaves the target ANGPTL3 genomic nucleic acid molecule. The Cas protein can cleave the nucleic acid molecule at a site within or outside of the nucleic acid sequence present in the target ANGPTL3 genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the ANGPTL3 genomic nucleic acid molecule to which a DNA-targeting segment of a gRNA will bind.

Such methods can result, for example, in an ANGPTL3 genomic nucleic acid molecule in which a region of the ANGPTL3 genomic nucleic acid molecule is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the ANGPTL3 genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In some embodiments, the methods of treatment and/or prevention further comprise detecting the presence or absence of an ANGPTL3 predicted loss-of-function or missense variant nucleic acid molecule in a biological sample from the subject. In some embodiments, the ANGPTL3 predicted loss-of-function or missense variant nucleic acid molecule can be any of the ANGPTL3 predicted loss-of-function or missense variant nucleic acid molecules disclosed herein.

The present disclosure also provides methods of treating a subject with a kidney disease therapeutic agent, wherein the subject has a kidney disease or is at risk of developing a kidney disease, wherein the kidney disease is not nephrotic syndrome. In some embodiments, the methods comprise determining whether the subject has an ANGPTL3 predicted loss-of-function or missense variant nucleic acid molecule by obtaining or having obtained a biological sample from the subject, and performing or having performed a sequence analysis on the biological sample to determine if the subject has a genotype comprising the ANGPTL3 predicted loss-of-function or missense variant nucleic acid molecule. In some embodiments, the methods further comprise administering or continuing to administer the kidney disease therapeutic agent in a standard dosage amount to a subject that is ANGPTL3 reference, and/or administering an ANGPTL3 inhibitor to the subject. In some embodiments, the methods further comprise administering or continuing to administer the kidney disease therapeutic agent in an amount that is the same as or less than a standard dosage amount to a subject that is heterozygous for the ANGPTL3 predicted loss-of-function or missense variant nucleic acid molecule, and/or administering an ANGPTL3 inhibitor to the subject. In some embodiments, the methods further comprise administering or continuing to administer the kidney disease therapeutic agent in an amount that is the same as or less than a standard dosage amount to a subject that is homozygous for the ANGPTL3 predicted loss-of-function or missense variant nucleic acid molecule. The presence of a genotype having the ANGPTL3 predicted loss-of-function or missense variant nucleic acid molecule indicates the subject has a decreased risk of developing a kidney disease. In some embodiments, the subject is ANGPTL3 reference. In some embodiments, the subject is heterozygous for an ANGPTL3 predicted loss-of-function or missense variant nucleic acid molecule.

For subjects that are genotyped or determined to be either ANGPTL3 reference or heterozygous for an ANGPTL3 predicted loss-of-function or missense variant nucleic acid molecule, such subjects can be administered an ANGPTL3 inhibitor, as described herein.

Detecting the presence or absence of an ANGPTL3 predicted loss-of-function or missense variant nucleic acid molecule in a biological sample from a subject and/or determining whether a subject has an ANGPTL3 predicted loss-of-function or missense variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when the subject is ANGPTL3 reference, the subject is administered a kidney disease therapeutic agent in a standard dosage amount. In some embodiments, when the subject is heterozygous for an ANGPTL3 predicted loss-of-function or missense variant nucleic acid molecule, the subject is administered a kidney disease therapeutic agent in a dosage amount that is the same as or less than a standard dosage amount.

In some embodiments, the treatment and/or prevention methods comprise detecting the presence or absence of an ANGPTL3 predicted loss-of-function polypeptide in a biological sample from the subject. In some embodiments, when the subject does not have an ANGPTL3 predicted loss-of-function polypeptide, the subject is administered a kidney disease therapeutic agent in a standard dosage amount. In some embodiments, when the subject has an ANGPTL3 predicted loss-of-function polypeptide, the subject is administered a kidney disease therapeutic agent in a dosage amount that is the same as or less than a standard dosage amount.

The present disclosure also provides methods of treating a subject with a kidney disease therapeutic agent, wherein the subject has a kidney disease or is at risk of developing a kidney disease, wherein the kidney disease is not nephrotic syndrome. In some embodiments, the method comprises determining whether the subject has an ANGPTL3 predicted loss-of-function polypeptide by obtaining or having obtained a biological sample from the subject, and performing or having performed an assay on the biological sample to determine if the subject has an ANGPTL3 predicted loss-of-function polypeptide. When the subject does not have an ANGPTL3 predicted loss-of-function polypeptide, the kidney disease therapeutic agent is administered or continued to be administered to the subject in a standard dosage amount, and/or an ANGPTL3 inhibitor is administered to the subject. When the subject has an ANGPTL3 predicted loss-of-function polypeptide, the kidney disease therapeutic agent is administered or continued to be administered to the subject in an amount that is the same as or less than a standard dosage amount, and/or an ANGPTL3 inhibitor is administered to the subject. The presence of an ANGPTL3 predicted loss-of-function polypeptide indicates the subject has a decreased risk of developing a kidney disease. In some embodiments, the subject has an ANGPTL3 predicted loss-of-function polypeptide. In some embodiments, the subject does not have an ANGPTL3 predicted loss-of-function polypeptide.

The present disclosure also provides methods of preventing a subject from developing a kidney disease by administering a kidney disease therapeutic agent, wherein the kidney disease is not nephrotic syndrome. In some embodiments, the method comprises determining whether the subject has an ANGPTL3 predicted loss-of-function polypeptide by obtaining or having obtained a biological sample from the subject, and performing or having performed an assay on the biological sample to determine if the subject has an ANGPTL3 predicted loss-of-function polypeptide. When the subject does not have an ANGPTL3 predicted loss-of-function polypeptide, the kidney disease therapeutic agent is administered or continued to be administered to the subject in a standard dosage amount, and/or an ANGPTL3 inhibitor is administered to the subject. When the subject has an ANGPTL3 predicted loss-of-function polypeptide, the kidney disease therapeutic agent is administered or continued to be administered to the subject in an amount that is the same as or less than a standard dosage amount, and/or an ANGPTL3 inhibitor is administered to the subject. The presence of an ANGPTL3 predicted loss-of-function polypeptide indicates the subject has a decreased risk of developing a kidney disease. In some embodiments, the subject has an ANGPTL3 predicted loss-of-function polypeptide. In some embodiments, the subject does not have an ANGPTL3 predicted loss-of-function polypeptide.

Detecting the presence or absence of an ANGPTL3 predicted loss-of-function polypeptide in a biological sample from a subject and/or determining whether a subject has an ANGPTL3 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

In some embodiments, the ANGPTL3 inhibitor is a small molecule. In some embodiments, the ANGPTL3 inhibitor is (12mer-)heparin (Gunn et al., J. Biol. Chem., 2021, 296, 1-12) or CAT-2003 (Liu et al., Arteriosclerosis, Thrombosis, and Vascular Biology, 2014, 34, A237). In some embodiments, the ANGPTL3 inhibitor is a vaccine. In some embodiments, the vaccine comprises a peptide corresponding to the LPL inhibitory domain of ANGPTL3. In some embodiments, the vaccine comprises a peptide having an amino acid sequence comprising amino acids 32 to 41 of ANGPTL3 (i.e., EPKSRFAMLD; SEQ ID NO:9) (see, Fukami et al., Cell Reports Med., 2021, 100446).

In some embodiments, the ANGPTL3 inhibitor is an antibody, or antigen-binding fragment thereof. In some embodiments, the antibody, or antigen-binding fragment thereof, binds specifically to human ANGPTL3. Exemplary antibodies, and fragments thereof, are disclosed in PCT Publication WO 2020/243031, which is incorporated herein by reference in its entirety.

In some embodiments, the antibody is a fully human monoclonal antibody (mAb), or antigen-binding fragment thereof, that specifically binds and neutralizes, inhibits, blocks, abrogates, reduces, or interferes with, at least one activity of ANGTPL3, in particular, human ANGPTL3 (SEQ ID NO:161). The activity of ANGPTL3 that can be neutralized, inhibited, blocked, abrogated, reduced or interfered with, by the antibodies or fragments thereof of the present disclosure, includes, but is not limited to, inhibition of LPL activity, induction of angiogenesis, and the like. In some embodiments, an antibody or fragment thereof can neutralize, inhibit, block, abrogate, reduce, or interfere with, an activity of ANGPTL3 by binding to an epitope of ANGPTL3 that is directly involved in the targeted activity of ANGPTL3. In some embodiments, an antibody or fragment thereof can neutralize, inhibit, block, abrogate, reduce, or interfere with, an activity of ANGPTL3 by binding to an epitope of ANGPTL3 that is not directly involved in the targeted activity of ANGPTL3, but the antibody or fragment binding thereto sterically or conformationally inhibits, blocks, abrogates, reduces, or interferes with, the targeted activity of ANGPTL3. In some embodiments, an antibody or fragment thereof binds to an epitope of ANGPTL3 that is not directly involved in the targeted activity (e.g., inhibiting LPL activity, inducing angiogenesis, and the like) of ANGPTL3 (i.e., a non-blocking antibody), but the antibody or fragment binding thereto results in the enhancement of the clearance of ANGPTL3 from the circulation, compared to the clearance of ANGPTL3 in the absence of the antibody or fragment thereof, thereby indirectly inhibiting, blocking, abrogating, reducing, or interfering with, an activity of ANGPTL3. Clearance of ANGPTL3 from the circulation can be particularly enhanced by combining two or more different non-blocking antibodies that do not compete with one another for specific binding to ANGPTL3.

The antibodies (Abs) can be full-length (for example, an IgG1 or IgG4 antibody) or may comprise only an antigen-binding portion (for example, a Fab, F(ab′)₂ or scFv fragment), and may be modified to affect functionality, e.g., to eliminate residual effector functions (Reddy et al., J. Immunol., 2000, 164, 1925-1933).

In some embodiments, the antibody or antigen-binding fragment of an antibody comprises a heavy chain variable region (HCVR) selected from the group consisting of SEQ ID NO:2, 18, 34, 50, 66, 82, 98, 114, 130, 146 and 164, or a substantially similar sequence thereof having at least 90%, at least 95%, at least 98% or at least 99% sequence identity. In some embodiments, the antibody or antigen-binding fragment thereof comprises a HCVR having an amino acid sequence selected from the group consisting of SEQ ID NO:2, 18, 34, 66, 82, 114, and 164. In some embodiments, the antibody or an antigen-binding fragment thereof comprises a HCVR having an amino acid sequence of SEQ ID NO:66.

In some embodiments, an antibody or antigen-binding fragment of an antibody comprises a light chain variable region (LCVR) selected from the group consisting of SEQ ID NO:10, 26, 42, 58, 74, 90, 106, 122, 138, 154 and 172, or a substantially similar sequence thereof having at least 90%, at least 95%, at least 98% or at least 99% sequence identity. In another embodiment, the antibody or antigen-binding portion of an antibody comprises a LCVR having an amino acid sequence selected from the group consisting of SEQ ID NO:10, 26, 42, 74, 90, 122 and 172. In some embodiments, the antibody or antigen-binding portion of an antibody comprises a LCVR having an amino acid sequence of SEQ ID NO:74.

In some embodiments, the antibody or fragment thereof comprises a HCVR and LCVR sequence pair (HCVR/LCVR) selected from the group consisting of SEQ ID NO:2/10, 18/26, 34/42, 50/58, 66/74, 82/90, 98/106, 114/122, 130/138, 146/154 and 164/172. In some embodiments, the antibody or fragment thereof comprises a HCVR and LCVR sequence pair selected from the group consisting of SEQ ID NO:2/10, 18/26, 34/42, 66/74, 82/90, 114/122 and 164/172. In some embodiments, the antibody or fragment thereof comprises a HCVR and LCVR sequence pair of SEQ ID NO:66/74.

In some embodiments, the antibody or antigen-binding fragment thereof comprises a heavy chain complementarity determining region 3 (HCDR3) amino acid sequence selected from the group consisting of SEQ ID NO:8, 24, 40, 56, 72, 88, 104, 120, 136, 152 and 170, or a substantially similar sequence thereof having at least 90%, at least 95%, at least 98% or at least 99% sequence identity; and a light chain CDR3 (LCDR3) amino acid sequence selected from the group consisting of SEQ ID NO:16, 32, 48, 64, 80, 96, 112, 128, 144, 160 and 178, or substantially similar sequences thereof having at least 90%, at least 95%, at least 98% or at least 99% sequence identity. In some embodiments, the antibody or fragment thereof comprises a HCDR3/LCDR3 amino acid sequence pair comprising SEQ ID NO:8/16, 24/32, 40/48, 56/64, 72/80, 88/96, 104/112, 120/128, 136/144, 152/160 or 170/178. In some embodiments, the antibody or fragment thereof comprises a HCDR3/LCDR3 amino acid sequence pair comprising SEQ ID NO:8/16, 24/32, 40/48, 72/80, 88/96, 120/128 or 170/178. In some embodiments, the antibody or fragment thereof comprises a HCDR3/LCDR3 amino acid sequence pair comprising SEQ ID NO:72/80.

In some embodiments, the antibody or fragment thereof further comprises a heavy chain CDR1 (HCDR1) amino acid sequence selected from the group consisting of SEQ ID NO:4, 20, 36, 52, 68, 84, 100, 116, 132, 148 and 166, or a substantially similar sequence thereof having at least 90%, at least 95%, at least 98% or at least 99% sequence identity; and a heavy chain CDR2 (HCDR2) amino acid sequence selected from the group consisting of SEQ ID NO:6, 22, 38, 54, 70, 86, 102, 118, 134, 150 and 168, or a substantially similar sequence thereof having at least 90%, at least 95%, at least 98% or at least 99% sequence identity; and optionally further comprises a light chain CDR1 (LCDR1) amino acid sequence selected from the group consisting of SEQ ID NO:12, 28, 44, 60, 76, 92, 108, 124, 140, 156 and 174, or a substantially similar sequence thereof having at least 90%, at least 95%, at least 98% or at least 99% sequence identity; and/or a light chain CDR2 (LCDR2) amino acid sequence selected from the group consisting of SEQ ID NO:14, 30, 46, 62, 78, 94, 110, 126, 142, 158 and 176, or a substantially similar sequence thereof having at least 90%, at least 95%, at least 98% or at least 99% sequence identity.

Amino acid sequences not depicted in the Sequence Listing are shown below.

| Sequence | SEQ ID NO: |
|---|---|
| Ala Ala Ser | 14 |
| Lys Ala Ser | 30 |
| Ala Ala Ser | 46 |
| Lys Ala Ser | 62 |
| Lys Ala Ser | 78 |
| Thr Thr Ser | 94 |
| Pro Ala Ser | 110 |
| Thr Ala Ser | 126 |
| Lys Val Ser | 142 |
| Val Ala Ala | 158 |
| Lys Ala Ser | 176 |

In some embodiments, the antibody or antigen-binding fragment thereof comprises a HCDR1/HCDR2/HCDR3 combination selected from the group consisting of SEQ ID NO:4/6/8, 20/22/24, 36/38/40, 52/54/56, 68/70/72, 84/86/88, 100/102/104, 116/118/120, 132/134/136, 148/150/152 and 166/168/170; and/or a LCDR1/LCDR2/LCDR3 combination selected from the group consisting of SEQ ID NO:12/14/16, 28/30/32, 44/46/48, 60/62/64, 76/78/80, 92/94/96, 108/110/112, 124/126/128, 140/142/144, 156/158/160 and 174/176/178. In some embodiments, the heavy and light chain CDR amino acid sequences comprise a CDR sequence combination selected from the group consisting of SEQ ID NO:4/6/8/12/14/16, 20/22/24/28/30/32, 36/38/40/44/46/48, 52/54/56/60/62/64, 68/70/72/76/78/80, 84/86/88/92/94/96, 100/102/104/108/110/112, 116/118/120/124/126/128, 132/134/136/140/142/144, 148/150/152/156/158/160 and 166/168/170/174/176/178. In some embodiments, the heavy and light chain CDR amino acid sequences comprise a CDR sequence combination of SEQ ID NO: 4/6/8/12/14/16, 20/22/24/28/30/32, 36/38/40/44/46/48, 68/70/72/76/78/80, 84/86/88/92/94/96, 116/118/120/124/126/128 or 166/168/170/174/176/178. In some embodiments, the heavy and light chain CDR amino acid sequences comprise a CDR sequence combination of SEQ ID NO:68/70/72/76/78/80.

In some embodiments, the antibody or antigen-binding fragment thereof, which specifically binds ANGPTL3, comprises heavy and light chain CDR domains contained within HCVR/LCVR pairs selected from the group consisting of SEQ ID NO:2/10, 18/26, 34/42, 50/58, 66/74, 82/90, 98/106, 114/122, 130/138, 146/154 and 164/172. Methods and techniques for identifying CDRs within HCVR and LCVR amino acid sequences are known in the art and can be applied to identify CDRs within the specified HCVR and/or LCVR amino acid sequences disclosed herein. Conventional definitions that can be applied to identify the boundaries of CDRs include the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol., 1997, 273, 927-948; and Martin et al., Proc. Natl. Acad. Sci. USA, 1989, 86, 9268-9272. Public databases are also available for identifying CDR sequences within an antibody. In some embodiments, the antibody or fragment thereof comprises CDR sequences contained within a HCVR and LCVR pair of SEQ ID NO: 2/10, 18/26, 34/42, 66/74, 82/90, 114/122 or 164/172. In some embodiments, the antibody or fragment thereof comprises CDR sequences contained within a HCVR and LCVR pair of SEQ ID NO:66/74.

In some embodiments, the antibody or antigen-binding fragment thereof competes for specific binding to ANGPTL3 with an antibody or antigen-binding fragment comprising heavy and light chain CDR sequences contained in a HCVR/LCVR sequence pair of SEQ ID NO:2/10, 18/26, 34/42, 50/58, 66/74, 82/90, 98/106, 114/122, 130/138, 146/154 or 164/172. In some embodiments, the antibody or antigen-binding fragment thereof competes for specific binding to ANGPTL3 with an antibody or fragment thereof comprising a HCVR/LCVR sequence pair of SEQ ID NO:66/74. In some embodiments, the antibody or antigen-binding fragment thereof competes for specific binding to ANGPTL3 with an antibody or fragment thereof comprising a heavy and light chain CDR sequence combination selected from the group consisting of 4/6/8/12/14/16, 20/22/24/28/30/32, 36/38/40/44/46/48, 52/54/56/60/62/64, 68/70/72/76/78/80, 84/86/88/92/94/96, 100/102/104/108/110/112, 116/118/120/124/126/128, 132/134/136/140/142/144, 148/150/152/156/158/160 and 166/168/170/174/176/178. In some embodiments, the antibody or antigen-binding fragment thereof competes for specific binding to ANGPTL3 with an antibody or fragment thereof comprising a heavy and light chain CDR sequence combination of SEQ ID NOS:68/70/72/76/78/80.

In some embodiments, the antibody or antigen-binding fragment thereof binds the same epitope on ANGPTL3 that is recognized by an antibody or fragment thereof comprising heavy and light chain CDR sequences from a HCVR/LCVR sequence pair of SEQ ID NO:2/10, 18/26, 34/42, 50/58, 66/74, 82/90, 98/106, 114/122, 130/138, 146/154 or 164/172. In some embodiments, the antibody or antigen-biding fragment thereof binds the same epitope on ANGPTL3 as that recognized by the antibody or fragment thereof comprising a HCVR/LCVR sequence pair of SEQ ID NO:66/74. In some embodiments, the antibody or fragment thereof binds the same epitope on ANGPTL3 that is recognized by an antibody or fragment thereof comprising a heavy and light chain CDR sequence combination selected from the group consisting of 4/6/8/12/14/16, 20/22/24/28/30/32, 36/38/40/44/46/48, 52/54/56/60/62/64, 68/70/72/76/78/80, 84/86/88/92/94/96, 100/102/104/108/110/112, 116/118/120/124/126/128, 132/134/136/140/142/144, 148/150/152/156/158/160 and 166/168/170/174/176/178. In some embodiments, such an epitope is recognized by an antibody or fragment thereof comprising a heavy and light chain CDR sequence combination of SEQ ID NO:68/70/72/76/78/80.

In some embodiments, an isolated anti-ANGPTL3 antibody or antigen-binding fragment thereof that binds to an epitope situated within the N-terminal coiled-coil region at residues 17 to 209 of SEQ ID NO:161 and neutralizes, inhibits, abrogates, reduces or interferes with, at least one activity of ANGPTL3. In some embodiments, the isolated antibody or antigen-binding fragment thereof specifically binds to an epitope situated within the N-terminal coiled-coil region of ANGPTL3 (SEQ ID NO:161) and neutralizes, inhibits, abrogates, reduces or interferes with, at least one activity of ANGPTL3, with the proviso that the antibody or fragment thereof does not bind to the ANGPTL3 peptide of SEQ ID NO:162 (corresponds to residues Glu32 to Leu57 of ANGPTL3 of SEQ ID NO:161). In some embodiments, the antibody or fragment thereof specifically binds to an epitope within residues 17 to 200, 17 to 100, 17 to 70, 17 to 65, 17 to 60, 17 to 57, or 17 to 50, of ANGPTL3 (SEQ ID NO:161), optionally with the proviso that the antibody or fragment thereof does not bind to the ANGPTL3 peptide of SEQ ID NO:162. In some embodiments, the antibody or fragment thereof specifically binds to an epitope within residues 40 to 200, 40 to 100, 40 to 70, 50 to 200, 50 to 100, 50 to 70, 58 to 200, 58 to 100, 58 to 70, 58 to 68, or 61 to 66, of ANGPTL3 (SEQ ID NO:161), optionally with the proviso that the antibody or fragment thereof does not bind to the ANGPTL3 peptide of SEQ ID NO:162. In some embodiments, the antibody or antibody fragment binds an epitope which may involve more than one of the enumerated epitopes or residues within the N-terminal coiled-coil region of ANGPTL3, optionally with the proviso that the antibody or fragment thereof does not bind to the ANGPTL3 peptide of SEQ ID NO:162.

The present disclosure also provides nucleic acid molecules that encode anti-ANGPTL3 antibodies or fragments thereof, in particular, any one of those described above. Recombinant expression vectors carrying these nucleic acids, and host cells, e.g., bacterial cells, such as E. coli, or mammalian cells, such as CHO cells, into which such vectors have been introduced, are also encompassed herein, as are methods of producing the antibodies by culturing the host cells under conditions permitting production of the antibodies, and recovering the antibodies produced.

In some embodiments, the antibody or fragment thereof comprises a HCVR encoded by a nucleic acid sequence selected from the group consisting of SEQ ID NO: 1, 17, 33, 49, 65, 81, 97, 113, 129, 145 and 163, or a substantially identical sequence having at least 90%, at least 95%, at least 98%, or at least 99% homology thereof. In some embodiments, the antibody or fragment thereof comprises a HCVR encoded by a nucleic acid sequence of SEQ ID NO:1, 17, 33, 65, 81, 113 or 163. In some embodiments, the antibody or fragment thereof comprises a HCVR encoded by a nucleic acid sequence of SEQ ID NO:65.

In some embodiments, an antibody or antigen-binding fragment thereof comprises a LCVR encoded by a nucleic acid sequence selected from the group consisting of SEQ ID NO: 9, 25, 41, 57, 73, 89, 105, 121, 137, 153 and 171, or a substantially identical sequence having at least 90%, at least 95%, at least 98%, or at least 99% homology thereof. In some embodiments, the antibody or fragment thereof comprises a LCVR encoded by a nucleic acid sequence of SEQ ID NO:9, 25, 41, 73, 89, 121 or 171. In some embodiments, the antibody or fragment thereof comprises a LCVR encoded by a nucleic acid sequence of SEQ ID NO:73.

In some embodiments, the antibody or fragment thereof comprises a HCVR and LCVR (HCVR/LCVR) sequence pair encoded by a nucleic acid sequence pair selected from the group consisting of SEQ ID NO:1/9, 17/25, 33/41, 49/57, 65/73, 81/89, 97/105, 113/121, 129/137, 145/153 and 163/171. In some embodiments, the antibody or fragment thereof comprises a HCVR/LCVR sequence pair encoded by a nucleic acid sequence pair of SEQ ID NO:1/9, 17/25, 33/41, 65/73, 81/89, 113/121 or 163/171. In some embodiments, the antibody or fragment thereof comprises a HCVR/LCVR sequence pair encoded by a nucleic acid sequence pair of SEQ ID NO:65/73.

In some embodiments, the antibody or antigen-binding fragment thereof comprises a HCDR3 domain encoded by a nucleotide sequence selected from the group consisting of SEQ ID NO:7, 23, 39, 55, 71, 87, 103, 119, 135, 151 and 169, or a substantially identical sequence having at least 90%, at least 95%, at least 98%, or at least 99% homology thereof; and a LCDR3 domain encoded by a nucleotide sequence selected from the group consisting of SEQ ID NO:15, 31, 47, 63, 79, 95, 111, 127, 143, 159 and 177, or a substantially identical sequence having at least 90%, at least 95%, at least 98%, or at least 99% homology thereof. In some embodiments, the antibody or fragment thereof comprises a HCDR3 and LCDR3 sequence pair encoded by the nucleic acid sequence pair selected from the group consisting of SEQ ID NO:7/15, 23/31, 39/47, 55/63, 71/79, 87/95, 103/111, 119/127, 135/143, 151/159 and 169/177. In some embodiments, the antibody or fragment thereof comprises a HCDR3 and LCDR3 sequence pair encoded by the nucleic acid sequence pair of SEQ ID NO:7/15, 23/31, 39/47, 71/79, 87/95, 119/127 or 169/177. In some embodiments, the antibody or fragment thereof comprises a HCDR3 and LCDR3 sequence pair encoded by the nucleic acid sequence pair of SEQ ID NO:71/79.

In some embodiments, the antibody or fragment thereof further comprises a HCDR1 domain encoded by a nucleotide sequence selected from the group consisting of SEQ ID NO: 3, 19, 35, 51, 67, 83, 99, 115, 131, 147 and 165, or a substantially identical sequence having at least 90%, at least 95%, at least 98%, or at least 99% homology thereof; and a HCDR2 domain encoded by a nucleotide sequence selected from the group consisting of SEQ ID NO:5, 21, 37, 53, 69, 85, 101, 117, 133, 149 and 167, or a substantially identical sequence having at least 90%, at least 95%, at least 98%, or at least 99% homology thereof; and optionally further comprises a LCDR1 domain encoded by a nucleotide sequence selected from the group consisting of SEQ ID NO:11, 27, 43, 59, 75, 91, 107, 123, 139, 155 and 173, or a substantially identical sequence having at least 90%, at least 95%, at least 98%, or at least 99% homology thereof; and/or a LCDR2 domain encoded by a nucleotide sequence selected from the group consisting of SEQ ID NO:13, 29, 45, 61, 77, 93, 109, 125, 141, 157 and 175, or a substantially identical sequence having at least 90%, at least 95%, at least 98%, or at least 99% homology thereof.

Nucleotide sequences not depicted in the Sequence Listing are shown below.

| Sequence | SEQ ID NO: |
|---|---|
| gctgcatcc | 13 |
| aaggcgtct | 29 |
| gctgcatcc | 45 |
| aaggcgtct | 61 |
| aaggcgtct | 77 |
| actacttcc | 93 |
| cctgcatcc | 109 |
| actgcatcc | 125 |
| aaggtttct | 141 |
| gttgcagcc | 157 |
| aaggcgtct | 175 |

In some embodiments, the antibody or antigen-binding fragment thereof comprises a HCDR1/HCDR2/HCDR3 combination encoded by a nucleotide sequence combination selected from the group consisting of SEQ ID NO:3/5/7, 19/21/23, 35/37/39, 51/53/55, 67/69/71, 83/85/87, 99/101/103, 115/117/119, 131/133/135, 147/149/151 and 165/167/169; and/or a LCDR1/LCDR2/LCDR3 combination encoded by a nucleotide sequence combination selected from the group consisting of SEQ ID NO:11/13/15, 27/29/31, 43/45/47, 59/61/63, 75/77/79, 91/93/95, 107/109/111, 123/125/127, 139/141/143, 155/157/159 and 173/175/177. In some embodiments, the antibody or fragment thereof comprises heavy and light chain CDR sequences encoded by a nucleotide sequence combination of SEQ ID NO:67/69/71/75/77/79.

In some embodiments, the anti-ANGPTL3 antibody or antigen-binding fragment thereof comprises a heavy chain variable region (HCVR) encoded by nucleotide sequence segments derived from V_{H}, D_{H} and J_{H} germline sequences, and a light chain variable region (LCVR) encoded by nucleotide sequence segments derived from V_{K} and J_{K} germline sequences, wherein the HCVR and the LCVR are encoded by nucleotide sequence segments derived from a germline gene combination selected from the group consisting of: (i) V_{H}3-43, D_{H}3-3, 43, V_{K}1-5 and J_{K}2; (ii) V_{H}3-11, D_{H}1-1, J_{H}4, V_{K}1-39 and J_{K}4; (iii) V_{H}3-30, D_{H}1-7, J_{H}6, V_{K}1-5 and J_{K}1; (iv) V_{H}3-30, D_{H}1-26, J_{H}6, V_{K}1-12 and J_{K}3; (v) V_{H}3-30, D_{H}3-10, J_{H}6, V_{K}1-12 and J_{K}3; and (vi) V_{H}3-23, D_{H}3-10, J_{H}4, V_{K}1-5 and J_{K}1.

In some embodiments, the antibody or antigen-binding fragment thereof specifically binds to ANGPTL3 with an equilibrium dissociation constant (K_{D}) of about 7 nM or less, about 6 nM or less, about 5 nM or less, about 4 nM or less, about 3 nM or less, about 2 nM or less, or about 1 nM or less, as measured by surface plasmon resonance assay (for example, BIACORE™). In some embodiments, the antibody exhibits a K_{D} of about 800 pM or less, about 700 pM or less; about 600 pM or less; about 500 pM or less; about 400 pM or less; about 300 pM or less; about 200 pM or less; about 100 pM or less; or about 50 pM or less.

In some embodiments, the anti-ANGPTL3 antibodies have a modified glycosylation pattern. In some applications, modification to remove undesirable glycosylation sites may be useful, or e.g., removal of a fucose moiety to increase antibody dependent cellular cytotoxicity (ADCC) function (see, Shield et al., J. Biol. Chem., 2002, 277, 26733). In other applications, removal of N-glycosylation site may reduce undesirable immune reactions against the therapeutic antibodies, or increase affinities of the antibodies. In yet other applications, modification of galactosylation can be made in order to modify complement dependent cytotoxicity (CDC).

The present disclosure also provides compositions comprising a combination of an antibody or antigen-binding fragment thereof and a second therapeutic agent. The second therapeutic agent may be one or more of any agent such as (1) 3-hydroxy-3-methylglutaryl-coenzyme A (HMG-CoA) reductase inhibitors, such as cerivastatin, atorvastatin, simvastatin, pitavastatin, rosuvastatin, fluvastatin, lovastatin, pravastatin, and the like; (2) inhibitors of cholesterol uptake and/or bile acid re-absorption; (3) niacin, which increases lipoprotein catabolism; (4) fibrates or amphipathic carboxylic acids, which reduce low-density lipoprotein (LDL) level, improve high-density lipoprotein (HDL) and TG levels, and reduce the number of non-fatal heart attacks; and (5) activators of the LXR transcription factor that plays a role in cholesterol elimination such as 22-hydroxycholesterol, or fixed combinations such as ezetimibe plus simvastatin; a statin with a bile resin (e.g., cholestyramine, colestipol, colesevelam), a fixed combination of niacin plus a statin (e.g., niacin with lovastatin); or with other lipid lowering agents such as omega-3-fatty acid ethyl esters (for example, omacor). Furthermore, the second therapeutic agent can be one or more other inhibitors of ANGPTL3 as well as inhibitors of other molecules, such as ANGPTL4, ANGPTL5, ANGPTL6 and proprotein convertase subtilisin/kexin type 9 (PCSK9), which are involved in lipid metabolism, in particular, cholesterol and/or triglyceride homeostasis. Inhibitors of these molecules include small molecules and antibodies that specifically bind to these molecules and block their activity.

In some embodiments, the second therapeutic agent may be one or more anti-cancer agents, such as chemotherapeutic agents, anti-angiogenic agents, growth inhibitory agents, cytotoxic agents, apoptotic agents, and other agents well known in the art to treat cancer or other proliferative diseases or disorders, as well as other therapeutic agents, such as analgesics, anti-inflammatory agents, including non-steroidal anti-inflammatory drugs (NSAIDS), such as Cox-2 inhibitors, and the like, so as to ameliorate and/or reduce the symptoms accompanying the underlying cancer/tumor.

The present disclosure also provides methods of concomitantly treating a cardiovascular disease (e.g., such as atherosclerosis, aneurysm, hypertension, angina, stroke, cerebrovascular diseases, congestive heart failure, coronary artery diseases, myocardial infarction, peripheral vascular diseases, and the like) and any of the kidney diseases disclosed herein.

Examples of kidney disease therapeutic agents that treat or inhibit chronic kidney disease include, but are not limited to, erythropoietin, a diuretic (such as, for example, furosemide, bumetanide, ethacrynic acid, metolazone, and hydrochlorothiazide), a blood pressure medication, a phosphate binder, sodium bicarbonate, a cholesterol medication, and a gliflozin, or any combination thereof. In some embodiments, the therapeutic agent that treats or inhibits chronic kidney disease comprises an SGLT2 inhibitor such as, for example, canagliflozin, dapagliflozin, empagliflozin, ipragliflozin, luseogliflozin, or tofogliflozin, or any combination thereof.

Examples of kidney disease therapeutic agents that treat or inhibit a kidney stone include, but are not limited to, potassium citrate, a diuretic (such as, for example, furosemide, bumetanide, ethacrynic acid, metolazone, and hydrochlorothiazide), allopurinol, acetohydroxamic acid, tamsulosin, nifedipine, d-penicillamine, tiopronin, and mercaptopropionyl glycine, or any combination thereof.

Examples of kidney disease therapeutic agents that treat or inhibit chronic glomerulonephritis include, but are not limited to, an angiotensin-converting enzyme (ACE) inhibitor (such as, for example, lisinopril, enalapril, captopril, benazepril, fosinopril, and quinapril), a diuretic (such as, for example, furosemide, bumetanide, ethacrynic acid, metolazone, and hydrochlorothiazide), a calcium channel blocker (such as, for example, amlodipine, nifedipine, felodipine, isradipine, verapamil, and diltiazem), a beta-adrenergic blocker (such as, for example, metoprolol, bisoprolol, esmolol, atenolol, propranolol, sotalol, labetalol, pindolol, and penbutolol), an alpha-adrenergic agonist (such as, for example, clonidine, tizanidine, and dexmedetomidine), a corticosteroid (such as, for example, prednisone), and an immunosuppressant (such as, for example, cyclosphosphamide), or any combination thereof.

Examples of kidney disease therapeutic agents that treat or inhibit nephronophthisis include, but are not limited to, erythropoietin and a blood pressure medication, or any combination thereof.

Examples of kidney disease therapeutic agents that treat or inhibit chronic interstitial nephritis include, but are not limited to, a corticosteroid, erythropoietin, a blood pressure medication, a statin, and a chelating agent (such as, for example, succimer and edetate calcium disodium), or a combination thereof.

Examples of kidney disease therapeutic agents that treat or inhibit nephrosclerosis include, but are not limited to, a diuretic (such as, for example, furosemide, bumetanide, ethacrynic acid, metolazone, and hydrochlorothiazide), an ACE inhibitor (such as, for example, lisinopril, enalapril, captopril, benazepril, fosinopril, and quinapril), an ARB (such as, for example, losartan, and valsartan), a calcium channel blocker (such as, for example, amlodipine, nifedipine, felodipine, isradipine, verapamil, and diltiazem), a beta-adrenergic blocker (such as, for example, metoprolol, bisoprolol, esmolol, atenolol, propranolol, sotalol, labetalol, pindolol, and penbutolol), an alpha-adrenergic agonist (such as, for example, clonidine, tizanidine, and dexmedetomidine), a renin inhibitor (such as, for example, aliskiren), a vasodilator (such as, for example, minoxidil and hydralazine), and an alpha-1 blocker (such as, for example, doxazosin), or any combination thereof.

In some embodiments, the dose of the kidney disease therapeutic agent can be decreased by about 10%, by about 20%, by about 30%, by about 40%, by about 50%, by about 60%, by about 70%, by about 80%, or by about 90% for subjects that are heterozygous for an ANGPTL3 predicted loss-of-function or missense variant nucleic acid molecule (i.e., a less than the standard dosage amount) compared to subjects that are ANGPTL3 reference (who may receive a standard dosage amount). In some embodiments, the dose of the kidney disease therapeutic agent can be decreased by about 10%, by about 20%, by about 30%, by about 40%, or by about 50%. In addition, the subjects that are heterozygous for an ANGPTL3 predicted loss-of-function or missense variant nucleic acid molecule can be administered less frequently compared to subjects that are ANGPTL3 reference.

In some embodiments, the dose of the kidney disease therapeutic agent can be decreased by about 10%, by about 20%, by about 30%, by about 40%, by about 50%, for subjects that are homozygous for an ANGPTL3 predicted loss-of-function or missense variant nucleic acid molecule compared to subjects that are heterozygous for an ANGPTL3 predicted loss-of-function or missense variant nucleic acid molecule. In some embodiments, the dose of the kidney disease therapeutic agent can be decreased by about 10%, by about 20%, by about 30%, by about 40%, or by about 50%. In addition, the dose of kidney disease therapeutic agent in subjects that are homozygous for an ANGPTL3 predicted loss-of-function or missense variant nucleic acid molecule can be administered less frequently compared to subjects that are heterozygous for an ANGPTL3 predicted loss-of-function or missense variant nucleic acid molecule.

Administration of the kidney disease therapeutic agent and/or ANGPTL3 inhibitors can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the kidney disease therapeutic agent and/or ANGPTL3 inhibitors can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in a kidney disease, a decrease/reduction in the severity of a kidney disease (such as, for example, a reduction or inhibition of development of a kidney disease), a decrease/reduction in symptoms and kidney disease-related effects, delaying the onset of symptoms and kidney disease-related effects, reducing the severity of symptoms of kidney disease-related effects, reducing the number of symptoms and kidney disease-related effects, reducing the latency of symptoms and kidney disease-related effects, an amelioration of symptoms and kidney disease-related effects, reducing secondary symptoms, reducing secondary infections, preventing relapse to a kidney disease, decreasing the number or frequency of relapse episodes, increasing latency between symptomatic episodes, increasing time to sustained progression, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of a kidney disease development/progression (such as, for example, a complete or partial avoidance/inhibition or a delay), and an increased survival time of the affected host animal, following administration of a therapeutic protocol. Treatment of a kidney disease encompasses the treatment of a subject already diagnosed as having any form of a kidney disease at any clinical stage or manifestation, the delay of the onset or evolution or aggravation or deterioration of the symptoms or signs of a kidney disease, and/or preventing and/or reducing the severity of a kidney disease.

The present disclosure also provides methods of identifying a subject having an increased risk of developing a kidney disease, wherein the kidney disease is not nephrotic syndrome. In some embodiments, the method comprises determining or having determined in a biological sample obtained from the subject the presence or absence of an ANGPTL3 predicted loss-of-function or missense variant nucleic acid molecule (such as a genomic nucleic acid molecule, mRNA molecule, and/or cDNA molecule). When the subject lacks an ANGPTL3 predicted loss-of-function or missense variant nucleic acid molecule (i.e., the subject is genotypically categorized as ANGPTL3 reference), then the subject has an increased risk of developing a kidney disease. When the subject has an ANGPTL3 predicted loss-of-function or missense variant nucleic acid molecule (i.e., the subject is heterozygous or homozygous for an ANGPTL3 predicted loss-of-function or missense variant nucleic acid molecule), then the subject has a decreased risk of developing a kidney disease.

Having a single copy of ANGPTL3 predicted loss-of-function or missense variant nucleic acid molecule is more protective of a subject from developing a kidney disease than having no copies of an ANGPTL3 predicted loss-of-function or missense variant nucleic acid molecule. Without intending to be limited to any particular theory or mechanism of action, it is believed that a single copy of an ANGPTL3 predicted loss-of-function or missense variant nucleic acid molecule (i.e., heterozygous for an ANGPTL3 predicted loss-of-function or missense variant nucleic acid molecule) is protective of a subject from developing a kidney disease, and it is also believed that having two copies of an ANGPTL3 predicted loss-of-function or missense variant nucleic acid molecule (i.e., homozygous for an ANGPTL3 predicted loss-of-function or missense variant nucleic acid molecule) may be more protective of a subject from developing a kidney disease, relative to a subject with a single copy. Thus, in some embodiments, a single copy of an ANGPTL3 predicted loss-of-function or missense variant nucleic acid molecule may not be completely protective, but instead, may be partially or incompletely protective of a subject from developing a kidney disease. While not desiring to be bound by any particular theory, there may be additional factors or molecules involved in the development of a kidney disease that are still present in a subject having a single copy of an ANGPTL3 predicted loss-of-function or missense variant nucleic acid molecule, thus resulting in less than complete protection from the development of a kidney disease.

Determining whether a subject has an ANGPTL3 predicted loss-of-function or missense variant nucleic acid molecule in a biological sample from a subject and/or determining whether a subject has an ANGPTL3 predicted loss-of-function or missense variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when a subject is identified as having an increased risk of developing a kidney disease, the subject is administered a kidney disease therapeutic agent, and/or an ANGPTL3 inhibitor, as described herein. For example, when the subject is ANGPTL3 reference, and therefore has an increased risk of developing a kidney disease, the subject is administered an ANGPTL3 inhibitor. In some embodiments, such a subject is also administered a kidney disease therapeutic agent. In some embodiments, when the subject is heterozygous for an ANGPTL3 predicted loss-of-function or missense variant nucleic acid molecule, the subject is administered the kidney disease therapeutic agent in a dosage amount that is the same as or less than a standard dosage amount, and is also administered an ANGPTL3 inhibitor. In some embodiments, such a subject is also administered a kidney disease therapeutic agent. In some embodiments, when the subject is homozygous for an ANGPTL3 predicted loss-of-function or missense variant nucleic acid molecule, the subject is administered the kidney disease therapeutic agent in a dosage amount that is the same as or less than a standard dosage amount. In some embodiments, the subject is ANGPTL3 reference. In some embodiments, the subject is heterozygous for an ANGPTL3 predicted loss-of-function or missense variant nucleic acid molecule. In some embodiments, the subject is homozygous for an ANGPTL3 predicted loss-of-function or missense variant nucleic acid molecule.

In some embodiments, any of the methods described herein can further comprise determining the subject's aggregate burden of having an ANGPTL3 predicted loss-of-function or missense variant nucleic acid molecule, and/or an ANGPTL3 predicted loss-of-function variant polypeptide associated with a decreased risk of developing a kidney disease. The aggregate burden is the sum of all variants in the ANGPTL3 gene, which can be carried out in an association analysis with a kidney disease. In some embodiments, the subject is homozygous for one or more ANGPTL3 predicted loss-of-function or missense variant nucleic acid molecules associated with a decreased risk of developing a kidney disease. In some embodiments, the subject is heterozygous for one or more ANGPTL3 predicted loss-of-function or missense variant nucleic acid molecules associated with a decreased risk of developing a kidney disease. The result of the association analysis suggests that ANGPTL3 predicted loss-of-function or missense variant nucleic acid molecules are associated with decreased risk of developing a kidney disease. When the subject has a lower aggregate burden, the subject is at a higher risk of developing a kidney disease and the subject is administered or continued to be administered the therapeutic agent that treats, prevents, or inhibits a kidney disease in a standard dosage amount, and/or an ANGPTL3 inhibitor. When the subject has a greater aggregate burden, the subject is at a lower risk of developing a kidney disease and the subject is administered or continued to be administered the kidney disease therapeutic agent in an amount that is the same as or less than the standard dosage amount. The greater the aggregate burden, the lower the risk of developing a kidney disease. The gene burden analysis can comprise any of the ANGPTL3 predicted loss-of-function or missense variant nucleic acid molecules disclosed herein, such as those listed in Table 4.

In some embodiments, the subject's aggregate burden of having any one or more ANGPTL3 predicted loss-of-function or missense variant nucleic acid molecules represents a weighted sum of a plurality of any of the ANGPTL3 predicted loss-of-function or missense variant nucleic acid molecules. In some embodiments, the aggregate burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the ANGPTL3 gene where the genetic burden is the number of alleles multiplied by the association estimate with a kidney disease or related outcome for each allele (e.g., a weighted polygenic burden score). In some embodiments, when the subject has an aggregate burden above a desired threshold score, the subject has a decreased risk of developing a kidney disease. In some embodiments, when the subject has an aggregate burden below a desired threshold score, the subject has an increased risk of developing a kidney disease.

In some embodiments, the aggregate burden may be divided into quintiles, e.g., top quintile, intermediate quintile, and bottom quintile, wherein the top quintile of aggregate burden corresponds to the lowest risk group and the bottom quintile of aggregate burden corresponds to the highest risk group. In some embodiments, a subject having a greater aggregate burden comprises the highest weighted aggregate burdens, including, but not limited to the top 10%, top 20%, top 30%, top 40%, or top 50% of aggregate burdens from a subject population. In some embodiments, the genetic variants comprise the genetic variants having association with a kidney disease in the top 10%, top 20%, top 30%, top 40%, or top 50% of p-value range for the association. In some embodiments, each of the identified genetic variants comprise the genetic variants having association with a kidney disease with p-value of no more than about 10⁻², about 10⁻³, about 10⁻⁴, about 10⁻⁵, about 10′, about 10⁻², about 10′, about 10⁻⁹, about 10⁻¹⁰, about 10⁻¹¹, about 10⁻¹², about 10⁻¹³, about 10⁻¹⁴, about or 10⁻¹⁵. In some embodiments, the identified genetic variants comprise the genetic variants having association with a kidney disease with p-value of less than 5×10⁻⁸. In some embodiments, the identified genetic variants comprise genetic variants having association with a kidney disease in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, from about 6.5 to about 7.0, or greater than 7.0. In some embodiments, high-risk subjects comprise subjects having aggregate burdens in the bottom decile, quintile, or tertile in a reference population. The threshold of the aggregate burden is determined on the basis of the nature of the intended practical application and the risk difference that would be considered meaningful for that practical application.

In some embodiments, when a subject is identified as having an increased risk of developing a kidney disease, the subject is further administered a kidney disease therapeutic agent, and/or an ANGPTL3 inhibitor, as described herein. For example, when the subject is ANGPTL3 reference, and therefore has an increased risk of developing a kidney disease, the subject is administered an ANGPTL3 inhibitor. In some embodiments, such a subject is also administered a kidney disease therapeutic agent. In some embodiments, when the subject is heterozygous for an ANGPTL3 predicted loss-of-function or missense variant nucleic acid molecule, the subject is administered the kidney disease therapeutic agent in a dosage amount that is the same as or less than a standard dosage amount, and is also administered an ANGPTL3 inhibitor. In some embodiments, the subject is ANGPTL3 reference. In some embodiments, the subject is heterozygous for an ANGPTL3 predicted loss-of-function or missense variant nucleic acid molecule. Furthermore, when the subject has a lower aggregate burden for having an ANGPTL3 predicted loss-of-function or missense variant nucleic acid molecule, and therefore has an increased risk of developing a kidney disease, the subject is administered a kidney disease therapeutic agent. In some embodiments, when the subject has a lower aggregate burden for having an ANGPTL3 predicted loss-of-function or missense variant nucleic acid molecule, the subject is administered the kidney disease therapeutic agent in a dosage amount that is the same as or greater than the standard dosage amount administered to a subject who has a greater aggregate burden for having an ANGPTL3 predicted loss-of-function or missense variant nucleic acid molecule.

The present disclosure also provides methods of detecting the presence or absence of an ANGPTL3 predicted loss-of-function or missense variant nucleic acid molecule (i.e., a genomic nucleic acid molecule, an mRNA molecule, or a cDNA molecule produced from an mRNA molecule) in a biological sample from a subject. It is understood that gene sequences within a population and mRNA molecules encoded by such genes can vary due to polymorphisms such as single-nucleotide polymorphisms.

The biological sample can be derived from any cell, tissue, or biological fluid from the subject. The biological sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any an ANGPTL3 predicted loss-of-function or missense variant nucleic acid molecule, preliminary processing designed to isolate or enrich the biological sample for the genomic DNA can be employed. A variety of techniques may be used for this purpose. When detecting the level of any an ANGPTL3 predicted loss-of-function or missense variant nucleic acid molecule, different techniques can be used enrich the biological sample with mRNA molecules. Various methods to detect the presence or level of an mRNA molecule or the presence of a particular variant genomic DNA locus can be used.

In some embodiments, detecting an ANGPTL3 predicted loss-of-function or missense variant nucleic acid molecule in a subject comprises performing a sequence analysis on a biological sample obtained from the subject to determine whether ANGPTL3 genomic nucleic acid molecule in the biological sample, and/or an ANGPTL3 mRNA molecule in the biological sample, and/or an ANGPTL3 cDNA molecule produced from an mRNA molecule in the biological sample, comprises one or more variations that cause a loss-of-function (partial or complete) or are predicted to cause a loss-of-function (partial or complete).

In some embodiments, the methods of detecting the presence or absence of an ANGPTL3 predicted loss-of-function or missense variant nucleic acid molecule (such as, for example, a genomic nucleic acid molecule, an mRNA molecule, and/or a cDNA molecule produced from an mRNA molecule) in a subject, comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising an ANGPTL3 genomic nucleic acid molecule or mRNA molecule, and if mRNA, optionally reverse transcribing the mRNA into cDNA. Such assays can comprise, for example determining the identity of these positions of the particular ANGPTL3 nucleic acid molecule. In some embodiments, the method is an in vitro method.

In some embodiments, the determining step, detecting step, or sequence analysis comprises sequencing at least a portion of the nucleotide sequence of the ANGPTL3 genomic nucleic acid molecule, the ANGPTL3 mRNA molecule, or the ANGPTL3 cDNA molecule in the biological sample, wherein the sequenced portion comprises one or more variations that cause a loss-of-function (partial or complete) or are predicted to cause a loss-of-function (partial or complete).

In some embodiments, the assay comprises sequencing the entire nucleic acid molecule. In some embodiments, only an ANGPTL3 genomic nucleic acid molecule is analyzed. In some embodiments, only an ANGPTL3 mRNA is analyzed. In some embodiments, only an ANGPTL3 cDNA obtained from ANGPTL3 mRNA is analyzed.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject.

In some embodiments, the assay comprises contacting the biological sample with a primer or probe, such as an alteration-specific primer or alteration-specific probe, that specifically hybridizes to an ANGPTL3 variant genomic sequence, variant mRNA sequence, or variant cDNA sequence and not the corresponding ANGPTL3 reference sequence under stringent conditions, and determining whether hybridization has occurred.

In some embodiments, the determining step, detecting step, or sequence analysis comprises: a) amplifying at least a portion of the ANGPTL3 nucleic acid molecule that encodes the ANGPTL3 polypeptide; b) labeling the amplified nucleic acid molecule with a detectable label; c) contacting the labeled nucleic acid molecule with a support comprising an alteration-specific probe; and d) detecting the detectable label.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising an ANGPTL3 variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to ANGPTL3 predicted loss-of-function or missense variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein, and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to ANGPTL3 predicted loss-of-function or missense variant nucleic acid molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the ANGPTL3 predicted loss-of-function or missense variant nucleic acid molecules disclosed herein. The primers described herein can be used to amplify any ANGPTL3 predicted loss-of-function or missense variant nucleic acid molecule, or a fragment thereof.

In the context of the disclosure “specifically hybridizes” means that the probe or primer (such as, for example, the alteration-specific probe or alteration-specific primer) does not hybridize to a nucleic acid sequence encoding an ANGPTL3 reference genomic nucleic acid molecule, an ANGPTL3 reference mRNA molecule, and/or an ANGPTL3 reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×his or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides kidney disease therapeutic agents for use in the treatment and/or prevention of a kidney disease in a subject having an ANGPTL3 predicted loss-of-function or missense variant nucleic acid molecule, wherein the kidney disease is not nephrotic syndrome. Any of the kidney disease therapeutic agents described herein can be used herein. Any of the ANGPTL3 predicted loss-of-function or missense variant nucleic acid molecules disclosed herein can be used herein.

The present disclosure also provides uses of kidney disease therapeutic agents for use in the preparation of a medicament for treating and/or preventing a kidney disease in a subject having an ANGPTL3 predicted loss-of-function or missense variant nucleic acid molecule, wherein the kidney disease is not nephrotic syndrome. Any of the kidney disease therapeutic agents described herein can be used herein. Any of the ANGPTL3 predicted loss-of-function or missense variant nucleic acid molecules disclosed herein can be used herein.

The present disclosure also provides ANGPTL3 inhibitors for use in the treatment and/or prevention of a kidney disease in a subject that is ANGPTL3 reference, or is heterozygous for an ANGPTL3 predicted loss-of-function or missense variant nucleic acid molecule, wherein the kidney disease is not nephrotic syndrome. Any of the ANGPTL3 inhibitors described herein can be used herein. Any of the ANGPTL3 predicted loss-of-function or missense variant nucleic acid molecules disclosed herein can be used herein.

The present disclosure also provides ANGPTL3 inhibitors in the preparation of a medicament for treating and/or preventing a kidney disease in a subject that is ANGPTL3 reference, or is heterozygous for an ANGPTL3 predicted loss-of-function or missense variant nucleic acid molecule, wherein the kidney disease is not nephrotic syndrome. Any of the ANGPTL3 inhibitors described herein can be used herein. Any of the ANGPTL3 predicted loss-of-function or missense variant nucleic acid molecules disclosed herein can be used herein.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: Loss-of-Function of ANGPTL3 is Associated with Improved Kidney Function and Decreased Risk of Kidney Diseases

To identify genetic factors contributing to predisposition for or protection against chronic kidney disease, exome sequencing was performed in 546,327 participants from the UK Biobank cohort (UKB) and Geisinger Health System DiscovEHR study (GHS). Associations with estimated glomerular filtration rate (eGFR), a widely used biomarker of kidney function in which higher levels indicate better function, were estimated for the burden of rare loss-of-function and missense variants in ANGPTL3 identified by exome sequencing. The rare loss-of-function and missense variants in ANGPTL3 were subsequently evaluated for association with clinical diagnoses of kidney disease in UKB and GHS.

In this analysis, the burden of rare (alternative allele frequency [AAF]<0.1%) predicted loss-of-function (pLOF) or missense genetic variants in ANGPTL3 was strongly associated with increased eGFR (see, Table 1). Importantly, the burden of pLOF or missense genetic variants was associated with increased eGFR regardless of whether eGFR was calculated using serum creatinine (eGFR_{creatinine}) or serum cystatin (eGFR_{cystatin}), indicating an effect of ANGPTL3 rare variants on kidney function and not creatinine or cystatin metabolism per se.

TABLE 1
Associations with increased eGFR levels for the burden of rare (AAF < 0.1%)
pLOF or missense variants in the ANGPTL3 gene

| | | Effect per allele | | Genotype counts, | |
|---|---|---|---|---|---|
| Genetic exposure | Outcome | [95% CI] | Pval | Ref\|Het\|Hom | AAF |
| ANGPTL3 pLOF or any | eGFR_{creatinine} | 0.04 [0.02, 0.07] SD, | 1.7 × 10⁻⁴ | 541,571\|4,750\|6 | 0.0044 |
| missense variants | | 0.75 [0.36, 1.14] mL/min | | | |
| ANGPTL3 pLOF or 5/5 | eGFR_{creatinine} | 0.05 [0.02, 0.08] SD, | 0.002 | 543,886\|2,437\|4 | 0.0022 |
| missense variants | | 0.86 [0.32, 1.40] mL/min | | | |
| ANGPTL3 pLOF or any | eGFR_{cystatin} | 0.03 [0.01, 0.06] SD, | 0.013 | 429,440\|3,684\|5 | 0.0043 |
| missense variants | | 0.52 [0.11, 0.92] mL/min | | | |
| ANGPTL3 pLOF or 5/5 | eGFR_{cystatin} | 0.05 [0.01, 0.08] SD, | 0.012 | 431,279\|1,847\|3 | 0.0021 |
| missense variants | | 0.73 [0.16, 1.3] mL/min | | | |

Abbreviations: CI, confidence interval; SD, standard deviation; mL/min, milliliters per minute; AAF, alternative allele frequency; Ref, homozygous reference genotype; Het, heterozygous carrier of rare coding variant (defined in ‘Genetic exposure’) in ANGPTL3; Hom, homozygous carrier of rare coding variant (defined in ‘Genetic exposure’) in ANGPTL3; pLOF, predicted loss of function; missense (5/5), missense variant predicted to be deleterious by 5 out of 5 in silico prediction algorithms; eGFR_{creatinine}, estimated glomerular filtration rate calculated using serum creatinine; eGFR_{cystatin}, estimated glomerular filtration rate calculated using serum cystatin.

An association for rare ANGPTL3 pLOF variants alone (AAF<1%, excluding missense variants) with increased eGFR was also observed (see, Table 2), indicating that the association for rare pLOF plus missense variants reflects a loss-of-function in ANGPTL3.

TABLE 2
Associations with increased eGFR levels for the burden of rare (AAF < 1%)
pLOF variants in the ANGPTL3 gene

| | | | | Genotype counts, | |
|---|---|---|---|---|---|
| Genetic exposure | Outcome | Effect per allele [95% CI] | Pval | Ref\|Het\|Hom | AAF |
| ANGPTL3 pLOF | eGFR_{creatinine} | 0.06 [0.02, 0.11] SD, | 0.008 | 545,101\|1,225\|1 | 0.00112 |
| variants | | 1.03 [0.26, 1.79] mL/min | | | |

Abbreviations: CI, confidence interval; SD, standard deviation; mL/min, milliliters per minute; AAF, alternative allele frequency; Ref, homozygous reference genotype; Het, heterozygous carrier of rare pLOF variant in ANGPTL3; Hom, homozygous carrier of rare pLOF variant in ANGPTL3; pLOF, predicted loss of function; eGFR_{creatinine}, estimated glomerular filtration rate calculated using serum creatinine.

### Example 2: Loss-of-Function in ANGPTL3 is Associated with Protection from Chronic Kidney Disease

The association of rare coding variants in ANGPTL3 was next estimated with kidney disease outcomes. The burdens of ANGPTL3 rare coding variants were associated with protection against reduced eGFR, microalbuminuria (increased excretion of albumin protein in the urine, an orthogonal marker of kidney damage) any kidney disease (a broadly-defined composite outcome including kidney diseases of different types and etiology), any chronic kidney disease including or excluding microalbuminuria, and severe chronic kidney disease such as stage 5+ or end-stage renal disease (see, Table 3). Heterozygous carriers of these genetic variants had 7% to 45% lower odds of kidney disease compared to non-carriers. Therefore, loss-of-function or deleterious missense variation in ANGPTL3 is associated with protection against various types and severities of chronic kidney disease in humans.

TABLE 3
Association of any missense, predicted deleterious missense variants or
predicted loss of function variants in ANGPTL3 with protection from reduced
eGFR, microalbuminuria, any kidney disease, chronic kidney disease, or
end-stage renal disease in UK Biobank and Geisinger Health System

| | | | | | Genotype | |
|---|---|---|---|---|---|---|
| | | | | Genotype | counts in | |
| Genetic | | | | counts in cases, | controls, | |
| exposure | Outcome | OR [95% Cl] | Pval | Ref\|Het\|Hom | Ref\|Het\|Hom | AAF |
| ANGPTL3 | 1 | 0.66 | 0.030 | 22,530\|41\|0 | 310,675\|709\|1 | 0.00113 |
| pLOF | | [0.45, 0.96] | | | | |
| variants, | 2 | 0.73 | 0.026 | 31,569\|58\|0 | 489,971\|1,097\|1 | 0.00111 |
| AAF <1% | | [0.56, 0.96] | | | | |
| | 3 | 0.75 | 0.047 | 40,821\|75\|0 | 217,259\|516\|0 | 0.00114 |
| | | [0.56, 1.00] | | | | |
| ANGPTL3 | 4 | 0.93 | 0.028 | 230,784\|1,906\|1 | 310,209\|2,835\|5 | 0.00435 |
| pLOF or | | [0.87, 0.99] | | | | |
| any | 5 | 0.83 | 0.016 | 22,553\|176\|2 | 165,744\|1,509\|1 | 0.00445 |
| missense | | [0.71, 0.97] | | | | |
| variants | 6 | 0.90 | 0.033 | 83,825\|706\|2 | 216,704\|2,012\|1 | 0.00449 |
| (AAF <0.1%) | | [0.82, 0.99] | | | | |
| | 7 | 0.88 | 0.026 | 59,169\|479\|2 | 216,704\|2,012\|1 | 0.00449 |
| | | [0.78, 0.98] | | | | |
| | 8 | 0.55 | 0.008 | 3,120\|13\|0 | 216,864\|1,979\|0 | 0.00449 |
| | | [0.35, 0.85] | | | | |
| | 9 | 0.59 | 0.022 | 2,853\|12\|0 | 216,864\|1,979\|0 | 0.0044 |
| | | [0.37, 0.93] | | | | |
| ANGPTL3 | 10 | 0.91 | 0.045 | 231,715\|976\|0 | 311,589\|1,456\|4 | 0.00224 |
| pLOF or | | [0.83, 1.00] | | | | |
| 5/5 | 11 | 0.79 | 0.029 | 22,645\|85\|1 | 166,501\|752\|1 | 0.00221 |
| missense | | [0.64, 0.98] | | | | |
| variants | 12 | 0.85 | 0.021 | 84,179\|353\|1 | 217,673\|1,0431\| | 0.00231 |
| (AAF <0.1%) | | [0.74, 0.98] | | | | |
| | 13 | 0.80 | 0.024 | 31,535\|124\|0 | 490,856\|2,187\|3 | 0.00221 |
| | | [0.66, 0.97] | | | | |
| | 14 | 0.80 | 0.028 | 40,736\|160\|0 | 216,756\|1,019\|0 | 0.00228 |
| | | [0.65, 0.98] | | | | |
| | 15 | 0.81 | 0.009 | 59,415\|234\|1 | 217,673\|1,043\|1 | 0.0023 |
| | | [0.69, 0.95] | | | | |

Outcomes:
1 = CKD (eGFR diagnosis);
2 = CKD (EHR diagnosis);
3 = CKD, any;
4 = Reduced eGFR;
5 = Microalbuminuria;
6 = Any kidney disease;
7 = CKD, any including microalbuminuria;
8 = CKD, stage 5+;
9 = End stage kidney disease;
10 = Reduced eGFR;
11 = Microalbuminuria;
12 = Any kidney disease;
13 = CKD (EHR diagnosis);
14 = CKD, any;
15 = CKD, any including microalbuminuria
Abbreviations:
Cl, confidence interval;
AAF, alternative allele frequency;
Ref, homozygous reference genotype;
Het, heterozygous carrier of rare coding variant (defined in ‘Genetic exposure’) in ANGPTL3;
Hom, homozygous carrier of rare coding variant (defined in ‘Genetic exposure’) in ANGPTL3;
pLOF, predicted loss of function;
missense (5/5), missense variant predicted to be deleterious by 5 out of 5 in silico prediction algorithms;
CKD, chronic kidney disease;
eGFR, estimated glomerular filtration rate;
EHR, electronic health record;
Reduced eGFR, eGFR < 90 mL/min;
CKD (eGFR diagnosis), eGFR <60 mL/min (cases) vs eGFR >90 mL/min (controls);
all other diseases defined in Table 5.

The associations between ANGPTL3 and kidney phenotypes were driven by multiple rare pLOF or missense variants in the ANGPTL3 gene (see, Table 4).

TABLE 4
Missense or pLOF variants in ANGPTL3 that were identified by exome
sequencing and included in the gene burden association analyses

| | | | | | Variant | Variant | |
|---|---|---|---|---|---|---|---|
| Genomic | | | | | classified | classified | |
| coordinate for | Predicted | | | Variant | as pLOF or | as pLOF or | |
| the genetic | conseq. | Coding | Protein amino | classified | 5/5 | any | |
| variant, C:P:R:A | of variant | DNA change | acid change | as pLOF | missense | missense | AAF |
| 1:62597582:C:T | MS (0/5) | c.16C>T | p.Leu6Phe | No | No | Yes | 1.2E−06 |
| 1:62597595:T:C | MS (3/5) | c.29T>C | p.Ile10Thr | No | No | Yes | 2.4E−06 |
| 1:62597600:C:T | MS (1/5) | c.34C>T | p.Pro12Ser | No | No | Yes | 4.4E−06 |
| 1:62597606:G:C | MS (3/5) | c.40G>C | p.Val14Leu | No | No | Yes | 2.4E−04 |
| 1:62597607:T:A | MS (4/5) | c.41T>A | p.Val14Asp | No | No | Yes | 1.2E−06 |
| 1:62597607:T:C | MS (2/5) | c.41T>C | p.Val14Ala | No | No | Yes | 2.4E−05 |
| 1:62597612:T:C | MS (1/5) | c.46T>C | p.Ser16Pro | No | No | Yes | 4.9E−06 |
| 1:62597616:C:G | MS (4/5) | c.50C>G | p.Ser17Cys | No | No | Yes | 6.5E−05 |
| 1:62597619:G:A | MS (0/5) | c.53G>A | p.Arg18Lys | No | No | Yes | 1.2E−06 |
| 1:62597622:T:C | MS (0/5) | c.56T>C | p.Ile19Thr | No | No | Yes | 4.9E−06 |
| 1:62597627:C:A | MS (1/5) | c.61C>A | p.Gln21Lys | No | No | Yes | 3.8E−06 |
| 1:62597628:A:G | MS (1/5) | c.62A>G | p.Gln21Arg | No | No | Yes | 2.4E−06 |
| 1:62597629:A:C | MS (1/5) | c.63A>C | p.Gln21His | No | No | Yes | 1.5E−04 |
| 1:62597632:C:G | MS (1/5) | c.66C>G | p.Asp22Glu | No | No | Yes | 2.4E−04 |
| 1:62597636:T:C | MS (0/5) | c.70T>C | p.Ser24Pro | No | No | Yes | 9.5E−06 |
| 1:62597637:C:T | MS (0/5) | c.71C>T | p.Ser24Leu | No | No | Yes | 4.4E−06 |
| 1:62597639:T:C | MS (0/5) | c.73T>C | p.Ser25Pro | No | No | Yes | 1.3E−05 |
| 1:62597642:T:C | MS (0/5) | c.76T>C | p.Phe26Leu | No | No | Yes | 2.7E−04 |
| 1:62597649:C:G | MS (4/5) | c.83C>G | p.Ser28Cys | No | No | Yes | 1.2E−06 |
| 1:62597654:T:C | MS (0/5) | c.88T>C | p.Ser30Pro | No | No | Yes | 1.2E−06 |
| 1:62597660:G:A | MS (4/5) | c.94G>A | p.Glu32Lys | No | No | Yes | 5.9E−05 |
| 1:62597660:G:C | MS (3/5) | c.94G>C | p.Glu32Gln | No | No | Yes | 1.9E−06 |
| 1:62597663:C:A | MS (1/5) | c.97C>A | p.Pro33Thr | No | No | Yes | 1.2E−06 |
| 1:62597664:C:T | MS (2/5) | c.98C>T | p.Pro33Leu | No | No | Yes | 1.2E−06 |
| 1:62597668:A:T | MS (3/5) | c.102A>T | p.Lys34Asn | No | No | Yes | 5.1E−05 |
| 1:62597669:T:C | MS (1/5) | c.103T>C | p.Ser35Pro | No | No | Yes | 3.6E−06 |
| 1:62597681:A:G | MS (3/5) | c.115A>G | p.Met39Val | No | No | Yes | 1.2E−06 |
| 1:62597682:T:C | MS (4/5) | c.116T>C | p.Met39Thr | No | No | Yes | 4.9E−06 |
| 1:62597696:A:C | MS (2/5) | c.130A>C | p.Lys44Gln | No | No | Yes | 1.2E−06 |
| 1:62597702:T:G | MS (4/5) | c.136T>G | p.Leu46Val | No | No | Yes | 1.2E−06 |
| 1:62597730:A:G | MS (3/5) | c.164A>G | p.His55Arg | No | No | Yes | 2.4E−04 |
| 1:62597739:A:G | MS (1/5) | c.173A>G | p.Lys58Arg | No | No | Yes | 1.2E−06 |
| 1:62597751:A:C | MS (4/5) | c.185A>C | p.His62Pro | No | No | Yes | 8.8E−06 |
| 1:62597772:A:G | MS (3/5) | c.206A>G | p.Asn69Ser | No | No | Yes | 1.2E−06 |
| 1:62597780:T:C | MS (3/5) | c.214T>C | p.Phe72Leu | No | No | Yes | 1.2E−06 |
| 1:62597782:T:G | MS (3/5) | c.216T>G | p.Phe72Leu | No | No | Yes | 4.4E−06 |
| 1:62597783:C:A | MS (1/5) | c.217C>A | p.Gln73Lys | No | No | Yes | 1.2E−04 |
| 1:62597792:A:T | MS (2/5) | c.226A>T | p.Asn76Tyr | No | No | Yes | 5.1E−05 |
| 1:62597798:T:C | MS (4/5) | c.232T>C | p.Phe78Leu | No | No | Yes | 1.9E−06 |
| 1:62597801:G:A | MS (4/5) | c.235G>A | p.Asp79Asn | No | No | Yes | 7.6E−06 |
| 1:62597804:C:G | MS (1/5) | c.238C>G | p.Gln80Glu | No | No | Yes | 1.2E−05 |
| 1:62597805:A:G | MS (1/5) | c.239A>G | p.Gln80Arg | No | No | Yes | 1.2E−06 |
| 1:62597806:G:C | MS (2/5) | c.240G>C | p.Gln80His | No | No | Yes | 1.2E−06 |
| 1:62597808:C:A | MS (4/5) | c.242C>A | p.Ser81Tyr | No | No | Yes | 1.2E−06 |
| 1:62597817:A:T | MS (4/5) | c.251A>T | p.Asp84Val | No | No | Yes | 1.9E−05 |
| 1:62597823:C:T | MS (4/5) | c.257C>T | p.Ser86Leu | No | No | Yes | 9.5E−06 |
| 1:62597826:T:C | MS (3/5) | c.260T>C | p.Leu87Pro | No | No | Yes | 3.6E−06 |
| 1:62597837:G:A | MS (2/5) | c.271G>A | p.Glu91Lys | No | No | Yes | 1.9E−06 |
| 1:62597838:A:G | MS (4/5) | c.272A>G | p.Glu91Gly | No | No | Yes | 4.9E−04 |
| 1:62597848:A:C | MS (4/5) | c.282A>C | p.Glu94Asp | No | No | Yes | 1.2E−06 |
| 1:62597849:G:C | MS (3/5) | c.283G>C | p.Glu95Gln | No | No | Yes | 1.9E−06 |
| 1:62597852:G:A | MS (3/5) | c.286G>A | p.Glu96Lys | No | No | Yes | 1.2E−06 |
| 1:62597852:G:C | MS (4/5) | c.286G>C | p.Glu96Gln | No | No | Yes | 1.2E−06 |
| 1:62597857:G:C | MS (3/5) | c.291G>C | p.Lys97Asn | No | No | Yes | 1.2E−06 |
| 1:62597868:G:C | MS (1/5) | c.302G>C | p.Arg101Thr | No | No | Yes | 1.1E−05 |
| 1:62597869:A:T | MS (1/5) | c.303A>T | p.Arg101Ser | No | No | Yes | 5.9E−05 |
| 1:62597876:T:C | MS (0/5) | c.310T>C | p.Tyr104His | No | No | Yes | 2.4E−04 |
| 1:62597894:A:G | MS (4/5) | c.328A>G | p.Asn110Asp | No | No | Yes | 4.0E−05 |
| 1:62597904:T:A | MS (3/5) | c.338T>A | p.Val113Glu | No | No | Yes | 4.4E−06 |
| 1:62597907:A:C | MS (4/5) | c.341A>C | p.Lys114Thr | No | No | Yes | 1.2E−06 |
| 1:62597913:T:C | MS (2/5) | c.347T>C | p.Met116Thr | No | No | Yes | 1.2E−06 |
| 1:62597918:C:T | MS (2/5) | c.352C>T | p.Leu118Phe | No | No | Yes | 2.4E−05 |
| 1:62597919:T:C | MS (3/5) | c.353T>C | p.Leu118Pro | No | No | Yes | 4.4E−06 |
| 1:62597927:A:C | MS (3/5) | c.361A>C | p.Asn121His | No | No | Yes | 1.2E−06 |
| 1:62597927:A:G | MS (1/5) | c.361A>G | p.Asn121Asp | No | No | Yes | 4.9E−06 |
| 1:62597931:C:T | MS (1/5) | c.365C>T | p.Ser122Leu | No | No | Yes | 3.6E−06 |
| 1:62597943:G:A | MS (0/5) | c.377G>A | p.Ser126Asn | No | No | Yes | 2.4E−06 |
| 1:62597951:G:A | MS (3/5) | c.385G>A | p.Glu129Lys | No | No | Yes | 2.0E−04 |
| 1:62597952:A:G | MS (4/5) | c.386A>G | p.Glu129Gly | No | No | Yes | 2.4E−06 |
| 1:62597954:G:C | MS (4/5) | c.388G>C | p.Glu130Gln | No | No | Yes | 1.2E−06 |
| 1:62597958:A:C | MS (4/5) | c.392A>C | p.Lys131Thr | No | No | Yes | 1.2E−06 |
| 1:62597959:A:C | MS (3/5) | c.393A>C | p.Lys131Asn | No | No | Yes | 5.9E−05 |
| 1:62597963:C:A | MS (1/5) | c.397C>A | p.Leu133Ile | No | No | Yes | 1.0E−05 |
| 1:62597967:T:C | MS (4/5) | c.401T>C | p.Leu134Pro | No | No | Yes | 2.4E−06 |
| 1:62597978:G:C | MS (3/5) | c.412G>C | p.Val138Leu | No | No | Yes | 7.6E−06 |
| 1:62597985:A:C | MS (0/5) | c.419A>C | p.Tyr140Ser | No | No | Yes | 1.2E−06 |
| 1:62597993:G:A | MS (1/5) | c.427G>A | p.Glu143Lys | No | No | Yes | 4.8E−06 |
| 1:62598003:C:T | MS (1/5) | c.437C>T | p.Thr146Ile | No | No | Yes | 1.2E−06 |
| 1:62598005:A:G | MS (0/5) | c.439A>G | p.Asn147Asp | No | No | Yes | 1.2E−06 |
| 1:62598006:A:C | MS (0/5) | c.440A>C | p.Asn147Thr | No | No | Yes | 2.5E−05 |
| 1:62598012:T:C | MS (1/5) | c.446T>C | p.Ile149Thr | No | No | Yes | 1.2E−06 |
| 1:62598017:A:G | MS (1/5) | c.451A>G | p.Asn151Asp | No | No | Yes | 2.7E−04 |
| 1:62598033:C:A | MS (1/5) | c.467C>A | p.Pro156Gln | No | No | Yes | 2.5E−04 |
| 1:62598038:C:T | MS (1/5) | c.472C>T | p.His158Tyr | No | No | Yes | 1.2E−06 |
| 1:62598041:C:A | MS (1/5) | c.475C>A | p.Pro159Thr | No | No | Yes | 2.4E−04 |
| 1:62598045:A:C | MS (3/5) | c.479A>C | p.Glu160Ala | No | No | Yes | 5.1E−05 |
| 1:62598046:A:C | MS (2/5) | c.480A>C | p.Glu160Asp | No | No | Yes | 1.2E−06 |
| 1:62598048:T:C | MS (1/5) | c.482T>C | p.Val161Ala | No | No | Yes | 1.2E−05 |
| 1:62598056:C:A | MS (4/5) | c.490C>A | p.Leu164Ile | No | No | Yes | 2.4E−06 |
| 1:62598056:C:T | MS (4/5) | c.490C>T | p.Leu164Phe | No | No | Yes | 8.2E−05 |
| 1:62598705:G:A | MS (2/5) | c.505G>A | p.Glu169Lys | No | No | Yes | 2.4E−06 |
| 1:62598707:A:T | MS (3/5) | c.507A>T | p.Glu169Asp | No | No | Yes | 5.1E−05 |
| 1:62598708:A:G | MS (1/5) | c.508A>G | p.Lys170Glu | No | No | Yes | 2.4E−06 |
| 1:62598719:T:A | MS (0/5) | c.519T>A | p.Asn173Lys | No | No | Yes | 1.2E−06 |
| 1:62598722:C:G | MS (1/5) | c.522C>G | p.Ser174Arg | No | No | Yes | 1.2E−06 |
| 1:62598728:A:T | MS (1/5) | c.528A>T | p.Lys176Asn | No | No | Yes | 2.4E−04 |
| 1:62598732:C:T | MS (4/5) | c.532C>T | p.Leu178Phe | No | No | Yes | 1.2E−06 |
| 1:62598735:C:T | MS (1/5) | c.535C>T | p.Leu179Phe | No | No | Yes | 1.2E−06 |
| 1:62598744:G:A | MS (3/5) | c.544G>A | p.Val182Met | No | No | Yes | 2.4E−05 |
| 1:62598745:T:C | MS (4/5) | c.545T>C | p.Val182Ala | No | No | Yes | 2.4E−06 |
| 1:62598747:G:A | MS (1/5) | c.547G>A | p.Glu183Lys | No | No | Yes | 1.2E−06 |
| 1:62598750:G:A | MS (1/5) | c.550G>A | p.Aspl84Asn | No | No | Yes | 2.4E−04 |
| 1:62598751:A:C | MS (1/5) | c.551A>C | p.Aspl84Ala | No | No | Yes | 8.8E−06 |
| 1:62598757:A:G | MS (3/5) | c.557A>G | p.Tyr186Cys | No | No | Yes | 5.7E−06 |
| 1:62598769:A:T | MS (3/5) | c.569A>T | p.Asn190Ile | No | No | Yes | 9.7E−06 |
| 1:62598770:C:A | MS (2/5) | c.570C>A | p.Asn190Lys | No | No | Yes | 1.2E−06 |
| 1:62598777:C:T | MS (3/5) | c.577C>T | p.His193Tyr | No | No | Yes | 1.2E−06 |
| 1:62598779:T:G | MS (0/5) | c.579T>G | p.His193Gln | No | No | Yes | 8.8E−06 |
| 1:62598780:A:T | MS (2/5) | c.580A>T | p.Ser194Cys | No | No | Yes | 1.2E−06 |
| 1:62598782:T:A | MS (1/5) | c.582T>A | p.Ser194Arg | No | No | Yes | 1.2E−06 |
| 1:62598796:T:C | MS (3/5) | c.596T>C | p.Ile199Thr | No | No | Yes | 1.1E−05 |
| 1:62598802:A:G | MS (1/5) | c.602A>G | p.Asn201Ser | No | No | Yes | 4.4E−06 |
| 1:62601082:C:A | MS (2/5) | c.607C>A | p.Leu203Ile | No | No | Yes | 2.4E−04 |
| 1:62601090:G:T | MS (1/5) | c.615G>T | p.Arg205Ser | No | No | Yes | 1.2E−06 |
| 1:62601094:A:G | MS (0/5) | c.619A>G | p.Ser207Gly | No | No | Yes | 1.2E−06 |
| 1:62601098:T:G | MS (1/5) | c.623T>G | p.Ile208Ser | No | No | Yes | 1.2E−06 |
| 1:62601106:C:T | MS (1/5) | c.631C>T | p.Pro211Ser | No | No | Yes | 5.1E−05 |
| 1:62601107:C:T | MS (0/5) | c.632C>T | p.Pro211Leu | No | No | Yes | 1.2E−06 |
| 1:62601113:A:G | MS (2/5) | c.638A>G | p.Glu213Gly | No | No | Yes | 1.2E−06 |
| 1:62601115:A:T | MS (1/5) | c.640A>T | p.Ile214Phe | No | No | Yes | 2.0E−04 |
| 1:62601116:T:C | MS (1/5) | c.641T>C | p.Ile214Thr | No | No | Yes | 1.2E−06 |
| 1:62601122:T:C | MS (1/5) | c.647T>C | p.Leu216Pro | No | No | Yes | 8.5E−06 |
| 1:62601132:G:C | MS (1/5) | c.657G>C | p.Lys219Asn | No | No | Yes | 1.2E−06 |
| 1:62601133:C:A | MS (1/5) | c.658C>A | p.Pro220Thr | No | No | Yes | 2.4E−06 |
| 1:62601140:C:T | MS (1/5) | c.665C>T | p.Ala222Val | No | No | Yes | 2.4E−04 |
| 1:62601142:C:T | MS (2/5) | c.667C>T | p.Pro223Ser | No | No | Yes | 5.1E−05 |
| 1:62601143:C:A | MS (3/5) | c.668C>A | p.Pro223Gln | No | No | Yes | 4.4E−06 |
| 1:62601152:C:A | MS (2/5) | c.677C>A | p.Thr226Asn | No | No | Yes | 1.2E−06 |
| 1:62601154:C:G | MS (1/5) | c.679C>G | p.Pro227Ala | No | No | Yes | 4.4E−06 |
| 1:62601154:C:T | MS (1/5) | c.679C>T | p.Pro227Ser | No | No | Yes | 3.6E−06 |
| 1:62601166:T:A | MS (1/5) | c.691T>A | p.Leu231Met | No | No | Yes | 2.2E−05 |
| 1:62601175:A:C | MS (0/5) | c.700A>C | p.Ile234Leu | No | No | Yes | 4.4E−06 |
| 1:62601176:T:C | MS (0/5) | c.701T>C | p.Ile234Thr | No | No | Yes | 1.2E−06 |
| 1:62601196:G:A | MS (1/5) | c.721G>A | p.Gly241Ser | No | No | Yes | 9.7E−06 |
| 1:62601778:C:T | MS (1/5) | c.731C>T | p.Ala244Val | No | No | Yes | 6.7E−06 |
| 1:62601803:A:T | MS (1/5) | c.756A>T | p.Arg252Ser | No | No | Yes | 1.2E−06 |
| 1:62601813:A:G | MS (0/5) | c.766A>G | p.Thr256Ala | No | No | Yes | 8.7E−05 |
| 1:62601823:T:C | MS (1/5) | c.776T>C | p.Met259Thr | No | No | Yes | 9.1E−04 |
| 1:62601823:T:G | MS (2/5) | c.776T>G | p.Met259Arg | No | No | Yes | 9.5E−06 |
| 1:62601825:T:C | MS (4/5) | c.778T>C | p.Tyr260His | No | No | Yes | 3.6E−06 |
| 1:62601836:A:C | MS (2/5) | c.789A>C | p.Arg263Ser | No | No | Yes | 1.8E−06 |
| 1:62601836:A:T | MS (2/5) | c.789A>T | p.Arg263Ser | No | No | Yes | 1.8E−05 |
| 1:62601837:C:T | MS (4/5) | c.790C>T | p.Pro264Ser | No | No | Yes | 2.4E−06 |
| 1:62601850:A:C | MS (3/5) | c.803A>C | p.Gln268Pro | No | No | Yes | 3.7E−05 |
| 1:62601864:T:A | MS (4/5) | c.817T>A | p.Tyr273Asn | No | No | Yes | 2.4E−06 |
| 1:62601867:T:C | MS (4/5) | c.820T>C | p.Cys274Arg | No | No | Yes | 5.9E−05 |
| 1:62601876:A:C | MS (0/5) | c.829A>C | p.Ile277Leu | No | No | Yes | 1.9E−06 |
| 1:62601876:A:G | MS (0/5) | c.829A>G | p.Ile277Val | No | No | Yes | 1.3E−05 |
| 1:62601877:T:C | MS (0/5) | c.830T>C | p.Ile277Thr | No | No | Yes | 2.8E−06 |
| 1:62601882:G:C | MS (3/5) | c.835G>C | p.Gly279Arg | No | No | Yes | 2.4E−05 |
| 1:62602285:G:A | MS (1/5) | c.836G>A | p.Gly279Asp | No | No | Yes | 1.2E−06 |
| 1:62602287:A:T | MS (3/5) | c.838A>T | p.Ser280Cys | No | No | Yes | 1.2E−06 |
| 1:62602288:G:T | MS (3/5) | c.839G>T | p.Ser280Ile | No | No | Yes | 1.2E−06 |
| 1:62602289:T:A | MS (0/5) | c.840T>A | p.Ser280Arg | No | No | Yes | 4.9E−06 |
| 1:62602291:C:A | MS (2/5) | c.842C>A | p.Pro281Gln | No | No | Yes | 1.2E−06 |
| 1:62602293:T:C | MS (1/5) | c.844T>C | p.Trp282Arg | No | No | Yes | 1.5E−04 |
| 1:62602297:C:T | MS (3/5) | c.848C>T | p.Thr283Ile | No | No | Yes | 4.4E−06 |
| 1:62602299:T:A | MS (3/5) | c.850T>A | p.Leu284Ile | No | No | Yes | 4.4E−06 |
| 1:62602309:A:T | MS (2/5) | c.860A>T | p.His287Leu | No | No | Yes | 8.8E−06 |
| 1:62602312:G:A | MS (4/5) | c.863G>A | p.Arg288Gln | No | No | Yes | 2.3E−05 |
| 1:62602314:A:T | MS (0/5) | c.865A>T | p.Ile289Leu | No | No | Yes | 4.4E−06 |
| 1:62602317:G:A | MS (2/5) | c.868G>A | p.Asp290Asn | No | No | Yes | 4.9E−06 |
| 1:62602335:A:G | MS (3/5) | c.886A>G | p.Asn296Asp | No | No | Yes | 2.4E−04 |
| 1:62602336:A:G | MS (4/5) | c.887A>G | p.Asn296Ser | No | No | Yes | 6.3E−05 |
| 1:62602341:A:C | MS (4/5) | c.892A>C | p.Thr298Pro | No | No | Yes | 1.2E−06 |
| 1:62602349:G:T | MS (1/5) | c.900G>T | p.Glu300Asp | No | No | Yes | 1.2E−06 |
| 1:62602357:A:C | MS (0/5) | c.908A>C | p.Lys303Thr | No | No | Yes | 8.5E−06 |
| 1:62602360:A:G | MS (4/5) | c.911A>G | p.Tyr304Cys | No | No | Yes | 1.2E−06 |
| 1:62602374:C:T | MS (2/5) | c.925C>T | p.Leu309Phe | No | No | Yes | 5.9E−05 |
| 1:62602375:T:C | MS (4/5) | c.926T>C | p.Leu309Pro | No | No | Yes | 1.2E−06 |
| 1:62602377:G:A | MS (1/5) | c.928G>A | p.Asp310Asn | No | No | Yes | 8.8E−06 |
| 1:62602380:G:A | MS (4/5) | c.931G>A | p.Gly311Arg | No | No | Yes | 4.4E−06 |
| 1:62603998:T:C | MS (1/5) | c.961T>C | p.Tyr321His | No | No | Yes | 4.7E−04 |
| 1:62604004:A:G | MS (4/5) | c.967A>G | p.Ile323Val | No | No | Yes | 9.5E−06 |
| 1:62604010:A:C | MS (1/5) | c.973A>C | p.Lys325Gln | No | No | Yes | 1.2E−06 |
| 1:62604013:C:A | MS (3/5) | c.976C>A | p.Gln326Lys | No | No | Yes | 1.0E−05 |
| 1:62604019:A:G | MS (0/5) | c.982A>G | p.Asn328Asp | No | No | Yes | 1.2E−06 |
| 1:62604022:T:C | MS (4/5) | c.985T>C | p.Tyr329His | No | No | Yes | 4.4E−06 |
| 1:62604032:G:A | MS (3/5) | c.995G>A | p.Arg332Gln | No | No | Yes | 4.2E−05 |
| 1:62604037:G:A | MS (4/5) | c.1000G>A | p.Glu334Lys | No | No | Yes | 2.4E−06 |
| 1:62604039:G:T | MS (3/5) | c.1002G>T | p.Glu334Asp | No | No | Yes | 1.4E−05 |
| 1:62604040:T:A | MS (4/5) | c.1003T>A | p.Leu335Met | No | No | Yes | 1.3E−05 |
| 1:62604055:G:A | MS (2/5) | c.1018G>A | p.Asp340Asn | No | No | Yes | 1.2E−06 |
| 1:62604058:A:G | MS (2/5) | c.1021A>G | p.Asn341Asp | No | No | Yes | 1.2E−06 |
| 1:62604065:A:G | MS (0/5) | c.1028A>G | p.His343Arg | No | No | Yes | 9.3E−05 |
| 1:62604067:T:C | MS (0/5) | c.1030T>C | p.Tyr344His | No | No | Yes | 1.2E−06 |
| 1:62604071:T:C | MS (0/5) | c.1034T>C | p.Ile345Thr | No | No | Yes | 2.9E−06 |
| 1:62604082:T:C | MS (2/5) | c.1045T>C | p.Phe349Leu | No | No | Yes | 3.6E−06 |
| 1:62604085:T:C | MS (0/5) | c.1048T>C | p.Tyr350His | No | No | Yes | 1.2E−06 |
| 1:62604091:G:A | MS (4/5) | c.1054G>A | p.Gly352Arg | No | No | Yes | 4.4E−06 |
| 1:62604097:C:T | MS (3/5) | c.1060C>T | p.His354Tyr | No | No | Yes | 1.2E−06 |
| 1:62604100:G:A | MS (4/5) | c.1063G>A | p.Glu355Lys | No | No | Yes | 6.5E−06 |
| 1:62604113:C:T | MS (3/5) | c.1076C>T | p.Thr359Met | No | No | Yes | 1.2E−06 |
| 1:62604118:C:T | MS (4/5) | c.1081C>T | p.His361Tyr | No | No | Yes | 6.0E−06 |
| 1:62604128:C:G | MS (0/5) | c.1091C>G | p.Ala364Gly | No | No | Yes | 2.4E−06 |
| 1:62604128:C:T | MS (0/5) | c.1091C>T | p.Ala364Val | No | No | Yes | 3.4E−05 |
| 1:62604130:A:G | MS (1/5) | c.1093A>G | p.Ile365Val | No | No | Yes | 2.4E−06 |
| 1:62604131:T:C | MS (1/5) | c.1094T>C | p.Ile365Thr | No | No | Yes | 1.2E−06 |
| 1:62604141:T:A | MS (3/5) | c.1104T>A | p.Asn368Lys | No | No | Yes | 4.4E−06 |
| 1:62604142:G:A | MS (0/5) | c.1105G>A | p.Val369Ile | No | No | Yes | 1.0E−04 |
| 1:62604149:A:C | MS (0/5) | c.1112A>C | p.Asn371Thr | No | No | Yes | 2.1E−05 |
| 1:62604149:A:G | MS (0/5) | c.1112A>G | p.Asn371Ser | No | No | Yes | 5.0E−05 |
| 1:62604149:A:T | MS (2/5) | c.1112A>T | p.Asn371Ile | No | No | Yes | 2.5E−04 |
| 1:62604158:C:T | MS (2/5) | c.1121C>T | p.Pro374Leu | No | No | Yes | 1.1E−05 |
| 1:62604160:G:A | MS (3/5) | c.1123G>A | p.Glu375Lys | No | No | Yes | 2.0E−04 |
| 1:62604160:G:C | MS (4/5) | c.1123G>C | p.Glu375Gln | No | No | Yes | 1.2E−06 |
| 1:62604161:A:C | MS (3/5) | c.1124A>C | p.Glu375Ala | No | No | Yes | 4.4E−06 |
| 1:62604172:T:G | MS (4/5) | c.1135T>G | p.Leu379Val | No | No | Yes | 1.2E−06 |
| 1:62604175:G:A | MS (0/5) | c.1138G>A | p.Val380Met | No | No | Yes | 9.5E−06 |
| 1:62604187:T:C | MS (1/5) | c.1150T>C | p.Trp384Arg | No | No | Yes | 3.8E−05 |
| 1:62604197:A:G | MS (0/5) | c.1160A>G | p.Lys387Arg | No | No | Yes | 4.4E−06 |
| 1:62604198:A:C | MS (0/5) | c.1161A>C | p.Lys387Asn | No | No | Yes | 4.4E−06 |
| 1:62604202:A:C | MS (2/5) | c.1165A>C | p.Lys389Gln | No | No | Yes | 5.9E−05 |
| 1:62604202:A:G | MS (1/5) | c.1165A>G | p.Lys389Glu | No | No | Yes | 1.2E−06 |
| 1:62604203:A:G | MS (2/5) | c.1166A>G | p.Lys389Arg | No | No | Yes | 6.1E−06 |
| 1:62604206:G:A | MS (0/5) | c.1169G>A | p.Gly390Glu | No | No | Yes | 1.2E−06 |
| 1:62604213:C:G | MS (0/5) | c.1176C>G | p.Phe392Leu | No | No | Yes | 1.2E−06 |
| 1:62604214:A:G | MS (0/5) | c.1177A>G | p.Asn393Asp | No | No | Yes | 4.1E−05 |
| 1:62604215:A:G | MS (0/5) | c.1178A>G | p.Asn393Ser | No | No | Yes | 1.2E−06 |
| 1:62604216:C:G | MS (1/5) | c.1179C>G | p.Asn393Lys | No | No | Yes | 1.2E−06 |
| 1:62604223:G:C | MS (2/5) | c.1186G>C | p.Glu396Gln | No | No | Yes | 1.2E−06 |
| 1:62604224:A:G | MS (4/5) | c.1187A>G | p.Glu396Gly | No | No | Yes | 8.6E−06 |
| 1:62604225:G:C | MS (4/5) | c.1188G>C | p.Glu396Asp | No | No | Yes | 4.4E−06 |
| 1:62604230:A:C | MS (2/5) | c.1193A>C | p.Tyr398Ser | No | No | Yes | 4.9E−06 |
| 1:62604644:T:C | MS (2/5) | c.1210T>C | p.Trp404Arg | No | No | Yes | 4.4E−06 |
| 1:62604647:C:T | MS (0/5) | c.1213C>T | p.His405Tyr | No | No | Yes | 4.4E−06 |
| 1:62604648:A:G | MS (0/5) | c.1214A>G | p.His405Arg | No | No | Yes | 1.2E−06 |
| 1:62604649:T:G | MS (0/5) | c.1215T>G | p.His405Gln | No | No | Yes | 2.4E−04 |
| 1:62604652:T:G | MS (0/5) | c.1218T>G | p.Asp406Glu | No | No | Yes | 1.8E−05 |
| 1:62604653:G:A | MS (0/5) | c.1219G>A | p.Glu407Lys | No | No | Yes | 4.4E−06 |
| 1:62604653:G:C | MS (0/5) | c.1219G>C | p.Glu407Gln | No | No | Yes | 1.2E−06 |
| 1:62604654:A:T | MS (0/5) | c.1220A>T | p.Glu407Val | No | No | Yes | 4.4E−06 |
| 1:62604659:G:A | MS (2/5) | c.1225G>A | p.Gly409Arg | No | No | Yes | 1.2E−06 |
| 1:62604663:A:C | MS (2/5) | c.1229A>C | p.Glu410Ala | No | No | Yes | 6.7E−06 |
| 1:62604663:A:G | MS (1/5) | c.1229A>G | p.Glu410Gly | No | No | Yes | 5.1E−05 |
| 1:62604666:A:G | MS (1/5) | c.1232A>G | p.Asn411Ser | No | No | Yes | 1.3E−05 |
| 1:62604668:A:C | MS (3/5) | c.1234A>C | p.Asn412His | No | No | Yes | 4.9E−06 |
| 1:62604705:C:T | MS (0/5) | c.1271C>T | p.Ser424Phe | No | No | Yes | 2.4E−06 |
| 1:62604707:A:G | MS (3/5) | c.1273A>G | p.Lys425Glu | No | No | Yes | 5.9E−05 |
| 1:62604713:G:C | MS (1/5) | c.1279G>C | p.Glu427Gln | No | No | Yes | 1.5E−05 |
| 1:62604716:A:G | MS (3/5) | c.1282A>G | p.Arg428Gly | No | No | Yes | 1.2E−06 |
| 1:62604723:G:A | MS (1/5) | c.1289G>A | p.Arg430Lys | No | No | Yes | 1.2E−04 |
| 1:62604723:G:C | MS (4/5) | c.1289G>C | p.Arg430Thr | No | No | Yes | 2.8E−05 |
| 1:62604725:G:A | MS (4/5) | c.1291G>A | p.Gly431Arg | No | No | Yes | 4.4E−06 |
| 1:62604725:G:C | MS (4/5) | c.1291G>C | p.Gly431Arg | No | No | Yes | 1.2E−06 |
| 1:62604728:T:A | MS (1/5) | c.1294T>A | p.Leu432Ile | No | No | Yes | 8.8E−06 |
| 1:62604731:T:A | MS (2/5) | c.1297T>A | p.Ser433Thr | No | No | Yes | 1.2E−06 |
| 1:62604732:C:A | MS (0/5) | c.1298C>A | p.Ser433Tyr | No | No | Yes | 4.8E−06 |
| 1:62604732:C:T | MS (0/5) | c.1298C>T | p.Ser433Phe | No | No | Yes | 1.5E−05 |
| 1:62604743:C:A | MS (1/5) | c.1309C>A | p.Gln437Lys | No | No | Yes | 4.4E−06 |
| 1:62604744:A:C | MS (1/5) | c.1310A>C | p.Gln437Pro | No | No | Yes | 1.8E−05 |
| 1:62604749:G:A | MS (2/5) | c.1315G>A | p.Gly439Arg | No | No | Yes | 9.7E−06 |
| 1:62604750:G:T | MS (4/5) | c.1316G>T | p.Gly439Val | No | No | Yes | 2.8E−06 |
| 1:62604754:G:T | MS (1/5) | c.1320G>T | p.Arg440Ser | No | No | Yes | 5.1E−05 |
| 1:62604761:T:C | MS (1/5) | c.1327T>C | p.Ser443Pro | No | No | Yes | 1.2E−06 |
| 1:62604764:A:T | MS (1/5) | c.1330A>T | p.Ile444Leu | No | No | Yes | 9.5E−06 |
| 1:62604765:T:C | MS (4/5) | c.1331T>C | p.Ile444Thr | No | No | Yes | 1.0E−05 |
| 1:62604770:T:C | MS (4/5) | c.1336T>C | p.Ser446Pro | No | No | Yes | 1.8E−05 |
| 1:62604776:A:G | MS (4/5) | c.1342A>G | p.Lys448Glu | No | No | Yes | 4.9E−06 |
| 1:62604785:A:G | MS (2/5) | c.1351A>G | p.Ile451Val | No | No | Yes | 2.4E−06 |
| 1:62604799:T:G | MS (0/5) | c.1365T>G | p.Asp455Glu | No | No | Yes | 4.2E−05 |
| 1:62604810:T:C | MS (0/5) | c.1376T>C | p.Phe459Ser | No | No | Yes | 5.4E−06 |
| 1:62604811:T:G | MS (1/5) | c.1377T>G | p.Phe459Leu | No | No | Yes | 4.4E−06 |
| 1:62604812:G:C | MS (2/5) | c.1378G>C | p.Glu460Gln | No | No | Yes | 1.2E−06 |
| 1:62604813:A:C | MS (2/5) | c.1379A>C | p.Glu460Ala | No | No | Yes | 1.2E−06 |
| 1:62597562:T:TA | FS | c.1dupA | p.Met1fs | No | Yes | Yes | 2.4E−06 |
| 1:62597568:T:G | start_lost | c.2T>G | p.Met1? | No | Yes | Yes | 5.1E−05 |
| 1:62597608:T:TA | FS | c.43dupA | p.Ile15fs | No | Yes | Yes | 2.7E−05 |
| 1:62597633:A:AG | FS | c.67_68insG | p.Asn23fs | No | Yes | Yes | 1.2E−06 |
| 1:62597634:A:AT | FS | c.70dupT | p.Ser24fs | No | Yes | Yes | 1.2E−06 |
| 1:62597670:C:G | SG | c.104C>G | p.Ser35* | No | Yes | Yes | 1.2E−06 |
| 1:62597673:G:C | MS (5/5) | c.107G>C | p.Arg36Thr | No | Yes | Yes | 1.2E−06 |
| 1:62597674:A:T | MS (5/5) | c.108A>T | p.Arg36Ser | No | Yes | Yes | 4.4E−06 |
| 1:62597676:T:C | MS (5/5) | c.110T>C | p.Phe37Ser | No | Yes | Yes | 2.4E−06 |
| 1:62597676:T:G | MS (5/5) | c.110T>G | p.Phe37Cys | No | Yes | Yes | 1.2E−06 |
| 1:62597679:C:A | MS (5/5) | c.113C>A | p.Ala38Asp | No | Yes | Yes | 2.4E−06 |
| 1:62597685:T:C | MS (5/5) | c.119T>C | p.Leu40Ser | No | Yes | Yes | 4.4E−06 |
| 1:62597690:G:A | MS (5/5) | c.124G>A | p.Asp42Asn | No | Yes | Yes | 2.2E−05 |
| 1:62597691:A:T | MS (5/5) | c.125A>T | p.Asp42Val | No | Yes | Yes | 4.4E−06 |
| 1:62597693:G:A | MS (5/5) | c.127G>A | p.Val43Ile | No | Yes | Yes | 1.2E−06 |
| 1:62597694:T:C | MS (5/5) | c.128T>C | p.Val43Ala | No | Yes | Yes | 4.0E−05 |
| 1:62597715:T:A | MS (5/5) | c.149T>A | p.Leu50His | No | Yes | Yes | 4.4E−06 |
| 1:62597717:C:G | MS (5/5) | c.151C>G | p.Leu51Val | No | Yes | Yes | 7.4E−05 |
| 1:62597723:T:G | MS (5/5) | c.157T>G | p.Leu53Val | No | Yes | Yes | 1.2E−06 |
| 1:62597727:G:A | MS (5/5) | c.161G>A | p.Gly54Glu | No | Yes | Yes | 1.2E−06 |
| 1:62597731:TG:T | FS | c.167delG | p.Gly56fs | No | Yes | Yes | 2.4E−06 |
| 1:62597736:T:G | MS (5/5) | c.170T>G | p.Leu57Arg | No | Yes | Yes | 4.4E−06 |
| 1:62597742:AC:A | FS | c.177delC | p.Phe60fs | No | Yes | Yes | 1.1E−06 |
| 1:62597743:CTT:C | FS | c.179_180delTT | p.Phe60fs | No | Yes | Yes | 5.1E−05 |
| 1:62597751:A:G | MS (5/5) | c.185A>G | p.His62Arg | No | Yes | Yes | 3.8E−06 |
| 1:62597754:A:C | MS (5/5) | c.188A>C | p.Lys63Thr | No | Yes | Yes | 1.8E−04 |
| 1:62597754:A:T | MS (5/5) | c.188A>T | p.Lys63Met | No | Yes | Yes | 8.8E−06 |
| 1:62597757:C:T | MS (5/5) | c.191C>T | p.Thr64Met | No | Yes | Yes | 1.9E−06 |
| 1:62597759:A:T | SG | c.193A>T | p.Lys65* | No | Yes | Yes | 1.2E−06 |
| 1:62597774:G:A | MS (5/5) | c.208G>A | p.Asp70Asn | No | Yes | Yes | 8.7E−05 |
| 1:62597787:A:C | MS (5/5) | c.221A>C | p.Lys74Thr | No | Yes | Yes | 1.2E−06 |
| 1:62597789:C:T | MS (5/5) | c.223C>T | p.Leu75Phe | No | Yes | Yes | 5.9E−05 |
| 1:62597801:G:T | MS (5/5) | c.235G>T | p.Asp79Tyr | No | Yes | Yes | 4.4E−06 |
| 1:62597815:T:G | SG | c.249T>G | p.Tyr83* | No | Yes | Yes | 1.2E−06 |
| 1:62597823:C:G | MS (5/5) | c.257C>G | p.Ser86Trp | No | Yes | Yes | 1.2E−06 |
| 1:62597846:G:T | SG | c.280G>T | p.Glu94* | No | Yes | Yes | 1.2E−06 |
| 1:62597862:T:C | MS (5/5) | c.296T>C | p.Leu99Pro | No | Yes | Yes | 4.4E−06 |
| 1:62597885:C:T | SG | c.319C>T | p.Gln107* | No | Yes | Yes | 4.4E−06 |
| 1:62597896:T:G | MS (5/5) | c.330T>G | p.Asn110Lys | No | Yes | Yes | 1.2E−06 |
| 1:62597901:A:C | MS (5/5) | c.335A>C | p.Glu112Ala | No | Yes | Yes | 1.2E−06 |
| 1:62597915:T:G | MS (5/5) | c.349T>G | p.Ser117Ala | No | Yes | Yes | 4.4E−06 |
| 1:62597946:T:C | MS (5/5) | c.380T>C | p.Leu127Pro | No | Yes | Yes | 1.2E−06 |
| 1:62597954:G:GA | FS | c.394dupA | p.Ile132fs | No | Yes | Yes | 2.4E−06 |
| 1:62597967:T:A | MS (5/5) | c.401T>A | p.Leu134His | No | Yes | Yes | 1.2E−06 |
| 1:62597972:CAA:C | FS | c.410_411delAA | p.Lys137fs | No | Yes | Yes | 1.2E−06 |
| 1:62598020:C:T | SG | c.454C>T | p.Gln152* | No | Yes | Yes | 1.2E−06 |
| 1:62598048:TA:T | FS | c.484delA | p.Thr162fs | No | Yes | Yes | 1.2E−06 |
| 1:62598059:A:G | MS (5/5) | c.493A>G | p.Lys165Glu | No | Yes | Yes | 2.4E−04 |
| 1:62598059:AAAGT:A | FS | c.494_495+2delAAGT | p.Lys165fs | No | Yes | Yes | 2.4E−05 |
| 1:62598700:T:C | MS (5/5) | c.500T>C | p.Phe167Ser | No | Yes | Yes | 4.4E−06 |
| 1:62598711:C:T | SG | c.511C>T | p.Gln171* | No | Yes | Yes | 1.2E−06 |
| 1:62598714:G:C | MS (5/5) | c.514G>C | p.Asp172His | No | Yes | Yes | 2.7E−05 |
| 1:62598714:G:T | MS (5/5) | c.514G>T | p.Asp172Tyr | No | Yes | Yes | 1.0E−04 |
| 1:62598715:A:G | MS (5/5) | c.515A>G | p.Asp172Gly | No | Yes | Yes | 1.2E−06 |
| 1:62598724:T:C | MS (5/5) | c.524T>C | p.Ile175Thr | No | Yes | Yes | 1.2E−05 |
| 1:62598762:CAATT:C | FS | c.565_568delTTAA | p.Leu189fs | No | Yes | Yes | 1.2E−06 |
| 1:62598787:T:C | MS (5/5) | c.587T>C | p.Ile196Thr | No | Yes | Yes | 7.1E−04 |
| 1:62598798:GA:G | FS | c.602delA | p.Asn201fs | No | Yes | Yes | 5.9E−05 |
| 1:62598799:A:G | MS (5/5) | c.599A>G | p.Glu200Gly | No | Yes | Yes | 1.2E−06 |
| 1:62598807:G:A | SD | c.606+1G>A | | No | Yes | Yes | 5.1E−05 |
| 1:62598807:G:C | SD | c.606+1G>C | | No | Yes | Yes | 1.2E−06 |
| 1:62601080:A:G | SA | c.607−2A>G | | No | Yes | Yes | 2.4E−06 |
| 1:62601198:T:C | SD | c.721+2T>C | | No | Yes | Yes | 4.4E−06 |
| 1:62601783:T:C | MS (5/5) | c.736T>C | p.Cys246Arg | No | Yes | Yes | 4.4E−06 |
| 1:62601783:T:G | MS (5/5) | c.736T>G | p.Cys246Gly | No | Yes | Yes | 1.2E−06 |
| 1:62601796:A:G | MS (5/5) | c.749A>G | p.Tyr250Cys | No | Yes | Yes | 5.6E−06 |
| 1:62601804:G:T | MS (5/5) | c.757G>T | p.Gly253Cys | No | Yes | Yes | 2.1E−04 |
| 1:62601855:T:C | MS (5/5) | c.808T>C | p.Phe270Leu | No | Yes | Yes | 1.2E−06 |
| 1:62601861:G:T | MS (5/5) | c.814G>T | p.Val272Phe | No | Yes | Yes | 1.2E−06 |
| 1:62601868:G:A | MS (5/5) | c.821G>A | p.Cys274Tyr | No | Yes | Yes | 7.6E−06 |
| 1:62601870:GATGTT:G | FS | c.826_830delGTTAT | p.Val276fs | No | Yes | Yes | 1.2E−06 |
| 1:62602284:G:C | SA | c.836−1G>C | | No | Yes | Yes | 5.9E−05 |
| 1:62602297:C:A | MS (5/5) | c.848C>A | p.Thr283Lys | No | Yes | Yes | 3.8E−06 |
| 1:62602300:T:C | MS (5/5) | c.851T>C | p.Leu284Ser | No | Yes | Yes | 4.8E−06 |
| 1:62602305:C:A | MS (5/5) | c.856C>A | p.Gln286Lys | No | Yes | Yes | 8.8E−06 |
| 1:62602311:C:T | SG | c.862C>T | p.Arg288* | No | Yes | Yes | 7.3E−06 |
| 1:62602316:AGATG:A | FS | c.871_874delGGAT | p.Gly291fs | No | Yes | Yes | 1.2E−06 |
| 1:62602317:G:C | MS (5/5) | c.868G>C | p.Asp290His | No | Yes | Yes | 2.7E−05 |
| 1:62602323:T:C | MS (5/5) | c.874T>C | p.Ser292Pro | No | Yes | Yes | 1.1E−05 |
| 1:62602331:C:A | MS (5/5) | c.882C>A | p.Asn294Lys | No | Yes | Yes | 5.1E−05 |
| 1:62602332:T:C | MS (5/5) | c.883T>C | p.Phe295Leu | No | Yes | Yes | 4.4E−06 |
| 1:62602332:T:G | MS (5/5) | c.883T>G | p.Phe295Val | No | Yes | Yes | 5.1E−05 |
| 1:62602337:T:A | MS (5/5) | c.888T>A | p.Asn296Lys | No | Yes | Yes | 9.3E−04 |
| 1:62602338:GA:G | FS | c.892delA | p.Thr298fs | No | Yes | Yes | 1.2E−06 |
| 1:62602342:C:T | MS (5/5) | c.893C>T | p.Thr298Met | No | Yes | Yes | 1.2E−05 |
| 1:62602345:G:A | SG | c.896G>A | p.Trp299* | No | Yes | Yes | 5.9E−05 |
| 1:62602346:G:A | SG | c.897G>A | p.Trp299* | No | Yes | Yes | 1.2E−06 |
| 1:62602353:T:C | MS (5/5) | c.904T>C | p.Tyr302His | No | Yes | Yes | 4.4E−06 |
| 1:62602355:C:A | SG | c.906C>A | p.Tyr302* | No | Yes | Yes | 1.2E−06 |
| 1:62602373:GC:G | FS | c.925delC | p.Asp310fs | No | Yes | Yes | 2.4E−06 |
| 1:62602381:G:A | SD | c.931+1G>A | | No | Yes | Yes | 2.4E−06 |
| 1:62603986:C:CT | FS | c.950dupT | p.Glu318fs | No | Yes | Yes | 7.1E−04 |
| 1:62603993:A:G | MS (5/5) | c.956A>G | p.Lys319Arg | No | Yes | Yes | 4.6E−05 |
| 1:62603996:T:C | MS (5/5) | c.959T>C | p.Ile320Thr | No | Yes | Yes | 1.2E−06 |
| 1:62604005:T:C | MS (5/5) | c.968T>C | p.Ile323Thr | No | Yes | Yes | 2.4E−06 |
| 1:62604017:CTAAT:C | FS | c.983_986delATTA | p.Asn328fs | No | Yes | Yes | 7.3E−06 |
| 1:62604024:T:A | SG | c.987T>A | p.Tyr329* | No | Yes | Yes | 1.2E−06 |
| 1:62604031:C:T | SG | c.994C>T | p.Arg332* | No | Yes | Yes | 7.3E−06 |
| 1:62604035:T:A | MS (5/5) | c.998T>A | p.Ile333Asn | No | Yes | Yes | 1.2E−06 |
| 1:62604035:T:G | MS (5/5) | c.998T>G | p.Ile333Ser | No | Yes | Yes | 3.1E−04 |
| 1:62604037:G:T | SG | c.1000G>T | p.Glu334* | No | Yes | Yes | 1.2E−06 |
| 1:62604044:A:G | MS (5/5) | c.1007A>G | p.Glu336Gly | No | Yes | Yes | 1.2E−06 |
| 1:62604049:T:C | MS (5/5) | c.1012T>C | p.Trp338Arg | No | Yes | Yes | 1.2E−06 |
| 1:62604051:G:C | MS (5/5) | c.1014G>C | p.Trp338Cys | No | Yes | Yes | 1.2E−06 |
| 1:62604083:T:G | MS (5/5) | c.1046T>G | p.Phe349Cys | No | Yes | Yes | 1.2E−06 |
| 1:62604109:T:C | MS (5/5) | c.1072T>C | p.Tyr358His | No | Yes | Yes | 6.1E−06 |
| 1:62604160:GA:G | FS | c.1127delA | p.Asn376fs | No | Yes | Yes | 1.0E−04 |
| 1:62604170:AT:A | FS | c.1136delT | p.Leu379fs | No | Yes | Yes | 1.2E−06 |
| 1:62604184:A:T | MS (5/5) | c.1147A>T | p.Thr383Ser | No | Yes | Yes | 7.3E−05 |
| 1:62604185:C:G | MS (5/5) | c.1148C>G | p.Thr383Ser | No | Yes | Yes | 9.0E−07 |
| 1:62604185:C:T | MS (5/5) | c.1148C>T | p.Thr383Ile | No | Yes | Yes | 5.7E−06 |
| 1:62604187:TG:T | FS | c.1153delG | p.Asp385fs | No | Yes | Yes | 5.1E−05 |
| 1:62604190:G:C | MS (5/5) | c.1153G>C | p.Asp385His | No | Yes | Yes | 5.9E−05 |
| 1:62604190:G:T | MS (5/5) | c.1153G>T | p.Asp385Tyr | No | Yes | Yes | 1.2E−06 |
| 1:62604218:G:C | MS (5/5) | c.1181G>C | p.Cys394Ser | No | Yes | Yes | 1.2E−06 |
| 1:62604224:A:AGG | FS | c.1189_1190dupGG | p.Tyr398fs | No | Yes | Yes | 1.2E−06 |
| 1:62604224:AG:A | FS | c.1190delG | p.Gly397fs | No | Yes | Yes | 1.3E−05 |
| 1:62604233:C:A | SG | c.1196C>A | p.Ser399* | No | Yes | Yes | 1.0E−04 |
| 1:62604235:G:A | MS (5/5) | c.1198G>A | p.Gly400Arg | No | Yes | Yes | 1.2E−06 |
| 1:62604639:G:A | SG | c.1205G>A | p.Trp402* | No | Yes | Yes | 2.4E−06 |
| 1:62604640:G:GT | FS | c.1207dupT | p.Trp403fs | No | Yes | Yes | 1.2E−06 |
| 1:62604643:G:C | MS (5/5) | c.1209G>C | p.Trp403Cys | No | Yes | Yes | 4.4E−06 |
| 1:62604656:T:C | MS (5/5) | c.1222T>C | p.Cys408Arg | No | Yes | Yes | 1.1E−05 |
| 1:62604669:AC:A | FS | c.1237delC | p.Leu413fs | No | Yes | Yes | 1.2E−06 |
| 1:62604684:A:G | MS (5/5) | c.1250A>G | p.Tyr417Cys | No | Yes | Yes | 1.8E−05 |
| 1:62604701:A:C | MS (5/5) | c.1267A>C | p.Lys423Gln | No | Yes | Yes | 1.2E−06 |
| 1:62604726:G:A | MS (5/5) | c.1292G>A | p.Gly431Glu | No | Yes | Yes | 3.9E−05 |
| 1:62604737:A:G | MS (5/5) | c.1303A>G | p.Lys435Glu | No | Yes | Yes | 4.4E−06 |
| 1:62604780:T:G | MS (5/5) | c.1346T>G | p.Met449Arg | No | Yes | Yes | 1.2E−06 |
| 1:62604803:G:T | SG | c.1369G>T | p.Glu457* | No | Yes | Yes | 5.9E−05 |
| 1:62604806:AG:A | FS | c.1373delG | p.Ser458fs | No | Yes | Yes | 1.2E−06 |
| 1:62604809:T:TGAATCTG | FS | c.1375_1376insGAATCTG | p.Phe459fs | No | Yes | Yes | 1.2E−06 |
| 1:62597855:A:T | SG | c.289A>T | p.Lys97* | Yes | Yes | Yes | 3.8E−06 |
| 1:62597909:AAT:A | FS | c.346_347delAT | p.Met116fs | Yes | Yes | Yes | 8.6E−06 |
| 1:62597921:GAACTC:G | FS | c.363_367delCTCAA | p.Asn121fs | Yes | Yes | Yes | 5.6E−04 |
| 1:62597951:G:T | SG | c.385G>T | p.Glu129* | Yes | Yes | Yes | 1.9E−06 |
| 1:62597996:CAACT:C | FS | c.439_442delAACT | p.Asn147fs | Yes | Yes | Yes | 2.1E−04 |
| 1:62598753:CA:C | FS | c.555delA | p.Gln185fs | Yes | Yes | Yes | 5.7E−06 |
| 1:62598771:CA:C | FS | c.573delA | p.Gln191fs | Yes | Yes | Yes | 7.2E−06 |
| 1:62598774:C:T | SG | c.574C>T | p.Gln192* | Yes | Yes | Yes | 1.9E−06 |
| 1:62598774:CA:C | FS | c.575delA | p.Gln192fs | Yes | Yes | Yes | 2.6E−05 |
| 1:62601115:AT:A | FS | c.643delT | p.Ser215fs | Yes | Yes | Yes | 2.7E−05 |
| 1:62601168:GA:G | FS | c.695delA | p.Asn232fs | Yes | Yes | Yes | 1.4E−05 |
| 1:62601197:G:A | SD | c.721+1G>A | | Yes | Yes | Yes | 1.2E−06 |
| 1:62601197:G:T | SD | c.721+1G>T | | Yes | Yes | Yes | 4.4E−06 |
| 1:62601880:C:A | SG | c.833C>A | p.Ser278* | Yes | Yes | Yes | 1.3E−05 |
| 1:62601880:C:G | SG | c.833C>G | p.Ser278* | Yes | Yes | Yes | 5.7E−06 |
| 1:62602297:C:CA | FS | c.849dupA | p.Leu284fs | Yes | Yes | Yes | 4.4E−06 |
| 1:62602381:G:GT | SD | c.931+1_931+2insT | | Yes | Yes | Yes | 1.6E−05 |
| 1:62602382:T:C | SD | c.931+2T>C | | Yes | Yes | Yes | 4.4E−06 |
| 1:62604236:G:T | SD | c.1198+1G>T | | Yes | Yes | Yes | 5.6E−06 |
| 1:62604645:G:A | SG | c.1211G>A | p.Trp404* | Yes | Yes | Yes | 3.1E−05 |
| 1:62604762:C:CT | FS | c.1329dupT | p.Ile444fs | Yes | Yes | Yes | 9.5E−06 |

MS = missense;
FS = frameshift;
SG = stop_gained;
SD = splice_donor;
SA = splice_acceptor;
C:P:R:A indicates the genomic coordinates of the genetic variant including chromosome (C), physical genomic position in base pairs (P), reference allele (R) and alternative allele (A) relative to build 38 of the Human Genome sequence by the Human Genome Reference Consortium. Coding DNA and protein changes follow the Human Genome Variation Society nomenclature and refer to ANGPTL3 transcript (ENST00000371129 annotated in the in the Ensembl database (URL: world wide web at “//useast.ensembl.org/index.html”)). Missense (0/5), Missense (1/5), Missense (2/5), Missense (3/5), Missense (4/5), Missense (5/5) indicate missense variants predicted to be damaging by 0, 1, 2, 3, 4, or 5 in silico algorithms, respectively. AAF indicates the alternative allele frequency. pLOF indicates predicted loss of function variant. Variant classified as indicates whether the variant was included in gene burden grouping specified.

### Participating Cohorts

Genetic association studies were performed in the United Kingdom (UK) Biobank (UKB) cohort (Sudlow et al., PLoS Med., 2015, 12, e1001779) and the DiscoverEHR cohort from the Geisinger Health System (GHS) MyCode Community Health Initiative (Carey et al., Genet. Med., 2016, 18, 906-13). The UKB is a population-based cohort study of people aged between 40 and 69 years recruited through 22 testing centers in the UK between 2006-2010. Over 430,000 participants of varying ancestries from UKB with available whole-exome sequencing and clinical phenotype data were included. The GHS MyCode study Community Health Initiative is a health system-based cohort of patients from Central and Eastern Pennsylvania (USA) recruited in 2007-2019. Over 130,000 European ancestry participants from GHS with available whole-exome sequencing and clinical phenotype data were included.

### Phenotype Definitions

eGFR was calculated from clinical laboratory measurements for creatinine extracted from electronic health records (EHRs) of participants from GHS. Median values were calculated for all participants with two or more measurements. In UKB, eGFR_{creatinine} was calculated from creatinine measured on a Beckman Coulter AU5800 clinical chemistry analyzer and eGFR_{cystatin} was calculated from cystatin measured by immunoturbidimetric analysis on a Siemens Advia 1800 clinical chemistry analyzer; both creatinine and cystatin were measured at the baseline visit of the study. In UKB, urine albumin:creatinine ratio (UACR) was derived from creatinine measured by enzymatic analysis and albumin measured by immunoturbidimetric analysis on a Beckman Coulter AU5400 clinical chemistry analyzer on spot urine measured at the baseline visit of the study. Prior to genetic association analysis, continuous phenotype values were transformed by the inverse standard normal function, applied within each ancestry group and separately in men and women. Kidney disease outcomes were defined according to the International Classification of Diseases Tenth Revision (ICD-10) and read codes stored in EHRs. Self-reported disease status was used when available. Office of Population Censuses and Surveys Classification of Interventions and Procedures version 4 (OPCS4) codes were used for medical procedures in UKB.

Individuals with kidney diseases were identified as described in Table 5, combining EHR records, self-reports and eGFR and UACR measurements. Participants were excluded from the control population for all kidney disease outcomes if they met any of the following criteria:

1. ICD10: N00, N01, N02, N03, N04, N05, N06, N07, N08, N10, N11, N12, N13, N14, N15, N16, N17, N18, N19, N20, N21, N22, N23, N25, N26, N27, N28, N29, Q60, Q61, Q63, C64, R944, I12, I13, T861, Z940, O904, O084, I824, Y841, Z490, Z491, Z492, Z992

2. eGFR<90 ml/min

3. Self-reported illness code (UKB-specific): 1192, 1193, 1194, 1197, 1519, 1520, 1607, 1608, 1609, 1427, 1405

4. Self-reported operation code (UKB-specific): 1195, 1197, 1476, 1487, 1580, 1581, 1582, 1618

5. OPCS4 operation code (UKB-specific): M01, M02, M03, M04, M05, M061, M068, M069, M07, M08, M09, M10, M111, M112, M131, M132, M133, M137, M14, M164, L746, M172, M174, M178, M179, X401, X402, X403, X404, X405, X406, X408, X409, X411, X412, X418, X419, X421, X428, X429, M012, M013, M014, M015, M018, M019, M084

6. Source of end-stage renal disease report code (UKB-specific): 0, 1, 2

7. Urinary albumin:creatinine ratio >10 mg/g (UKB-specific)

8. EHR code for kidney transplant or dialysis in billing procedures or surgery (GHS-specific).

TABLE 5
Definitions of kidney disease and outcomes in UKB and GHS cohort

| | Case definition |
|---|---|
| Kidney disease | Participants were included as a case if they met any of the criteria |
| outcome | specific to each disease |
| Microalbuminuria | UKB.UACR > 30 mg/g |
| Any kidney | ICD10: |
| disease | N00, N01, N02, N03, N04, N05, N06, N07, N08, N10, N11, N12, N13, N14, N15, |
| | N16, N17, N18, N19, N25, N26, N27, N28, N29, Q60, Q61, Q63, C64, R944, |
| | I12, I13, T861, Z940, O904, O084, T824, Y841, Z490, Z491, Z492, Z992 |
| | UKB.f.20002: 1192, 1193, 1194, 1519, 1520, 1607, 1608, 1609, 1427, 1405 |
| | UKB.f.20004: 1195, 1476, 1487, 1580, 1581, 1582, 1618 |
| | UKB.OPCS4: |
| | M01, M02, M03, M04, M05, M068, M069, M078, M079, M08, M10, M111, |
| | M112, M131, M132, M133, M137, M164, L746, M172, M174, M178, M179, |
| | X401, X402, X403, X404, X405, X406, X408, X409, X411, X412, X418, X419, |
| | X421, X428, X429, M012, M013, M014, M015, M018, M019, M084 |
| | UKB.f.42027: 0, 1, 2 |
| | GHS.EHR: kidney transplant or dialysis in billing procedures or surgery |
| | eGFR < 60 ml/min |
| | UKB.UACR > 30 mg/g |
| Chronic kidney | ICD10: N18, Q611, Q612, Q613, Q615, I12, I13, T861, Z940, Z492 |
| disease, any | UKB.f.20002: 1193, 1607, 1427 |
| | UKB.f.20004: 1195, 1476, 1580, 1581, 1582 |
| | UKB.OPSC4: |
| | L746, M012, M013, M014, M015, M018, M019, M084, M174, M178, M179, |
| | X402, X405, X406, X411, X412 |
| | UKB.f.42027: 0, 1, 2 |
| | GHS.EHR: kidney transplant in billing procedures or surgery |
| | eGFR < 60 ml/min |
| | UKB.UACR > 300 mg/g |
| Chronic kidney | ICD10: N18, Q611, Q612, Q613, Q615, I12, I13, T861, Z940, Z492 |
| disease, any | UKB.f.20002: 1193, 1607, 1427 |
| including | UKB.f.20004: 1195, 1476, 1580, 1581, 1582 |
| microalbuminuria | UKB.OPSC4: |
| | L746, M012, M013, M014, M015, M018, M019, M084, M174, M178, M179, |
| | X402, X405, X406, X411, X412 |
| | UKB.f.42027: 0, 1, 2 |
| | GHS.EHR: kidney transplant in billing procedures or surgery |
| | eGFR < 60 ml/min |
| | UKB.UACR > 30 mg/g |
| CKD stage 3+ | ICD10: N183, N184, N185, N180, N186, T861, Z492, Z940, N165 |
| | UKB.f.20002: 1193 |
| | UKB.f.20004: 1476, 1195, 1580, 1581, 1582 |
| | UKB.OPSC4: |
| | M012, M013, M014, M015, M018, M019, M084, M174, M178, M179, X402, |
| | X405, X406, X411, X412 |
| | UKB.f.42027: 0, 1, 2 |
| | GHS.EHR: kidney transplant in billing procedures or surgery |
| | eGFR < 60 ml/min |
| CKD stage 5+ | ICD10: N185, N180, N186, T861, Z492, Z940, N165 |
| | UKB.f.20002: 1193 |
| | UKB.f.20004: 1476, 1195, 1580, 1581, 1582 |
| | UKB.OPSC4: |
| | M012, M013, M014, M015, M018, M019, M084, M174, M178, M179, X402, |
| | X405, X406, X411, X412 |
| | UKB.f.42027: 0, 1, 2 |
| | GHS.EHR: kidney transplant in billing procedures or surgery |
| | eGFR < 15 ml/min |
| Renal | ICD10: T824, T861, Y841, Z490, Z491, Z492, Z940, Z992 |
| replacement | UKB.f.20002: 1193 |
| therapy | UKB.f.20004: 1195, 1476, 1580, 1581, 1582 |
| | UKB.OPSC4: |
| | L746, M012, M013, M014, M015, M018, M019, M084, M174, M178, M179, |
| | X401, X402, X403, X404, X405, X406, X408, X409, X411, X412, X418, |
| | X419, X421, X428, X429 |
| | UKB.f.42027: 0, 1, 2 |
| | GHS.EHR: kidney transplant or dialysis in billing procedures or |
| | surgery |
| End stage renal | ICD10: N180, N186, T861, 2492, 2940, N165 |
| disease | UKB.f.20002: 1193 |
| | UKB.f.20004: 1476, 1195, 1580, 1581, 1582 |
| | UKB.OPSC4: |
| | M012, M013, M014, M015, M018, M019, M084, M174, M178, M179, X402, |
| | X405, X406, X411, X412 |
| | UKB.f.42027: 0, 1, 2 |
| | GHS.EHR: kidney transplant in billing procedures or surgery |

ICD10 indicates the 10th revision of the International Statistical Classification of Diseases and Related Health Problems; UKB.OPCS4 indicates Office of Population Censuses and Surveys (OPCS) Classification of Interventions and Procedures version 4 as used in the UK Biobank (UKB); UKB.f.20002 indicates self-reported non-cancer illness codes as used in UKB; UKB.f.20004 indicates self-reported medical procedures as used in UKB; UKB.f.42027 indicates source of end-stage renal disease code as defined by central working group for UKB; GHS.EHR indicates billing procedures or surgeries in Geisinger Health System (GHS) relevant to disease definition; UKB.UACR indicates urine albumin:creatinine ratio measured in UKB.

### Genotype Data

High coverage whole exome sequencing was performed as previously described (Science, 2016, 354:aaf6814; and Nature, 2020, 586, 749-756) and as summarized below. NimbleGen probes (VCRome; for part of the GHS cohort) or a modified version of the xGen design available from Integrated DNA Technologies (IDT; for the rest of GHS and other cohorts) were used for target sequence capture of the exome. A unique 6 base pair (bp) barcode (VCRome) or 10 bp barcode (IDT) was added to each DNA fragment during library preparation to facilitate multiplexed exome capture and sequencing. Equal amounts of sample were pooled prior to exome capture. Sequencing was performed using 75 bp paired-end reads on Illumina v4 HiSeq 2500 (for part of the GHS cohort) or NovaSeq (for the rest of GHS and other cohorts) instruments. Sequencing had a coverage depth (i.e., number of sequence-reads covering each nucleotide in the target areas of the genome) sufficient to provide greater than 20× coverage over 85% of targeted bases in 96% of VCRome samples and 20× coverage over 90% of targeted bases in 99% of IDT samples. Data processing steps included sample de-multiplexing using Illumina software, alignment to the GRCh38 Human Genome reference sequence including generation of binary alignment and mapping files (BAM), processing of BAM files (e.g., marking of duplicate reads and other read mapping evaluations). Variant calling was performed using the GLNexus system (DOI: 10.1101/343970). Variant mapping and annotation were based on the GRCh38 Human Genome reference sequence and Ensembl v85 gene definitions using the snpEff software. The snpEff predictions that involve protein-coding transcripts with an annotated start and stop were then combined into a single functional impact prediction by selecting the most deleterious functional effect class for each gene. The hierarchy (from most to least deleterious) for these annotations was frameshift, stop-gain, stop-loss, splice acceptor, splice donor, stop-lost, in-frame indel, missense, other annotations. Predicted LOF genetic variants included: a) insertions or deletions resulting in a frameshift, b) insertions, deletions or single nucleotide variants resulting in the introduction of a premature stop codon or in the loss of the transcription start site or stop site, and c) variants in donor or acceptor splice sites. Missense variants were classified for likely functional impact according to the number of in silico prediction algorithms that predicted deleteriousness using SIFT (Adzhubei et al., Nat. Methods, 2010, 7, 248-9) and Polyphen2_HVAR (Adzhubei et al., Nat. Methods, 2010, 7, 248-9), LRT (Chun et al., Genome Res., 2009, 19, 1553-61) and MutationTaster (Schwarz et al., Nat. Methods, 2010, 7, 575-6). For each gene, the alternative allele frequency (AAF) and functional annotation of each variant determined inclusion into these 7 gene burden exposures: 1) pLOF variants with AAF<1%; 2) pLOF or missense variants predicted deleterious by 5/5 algorithms with AAF<1%; 3) pLOF or missense variants predicted deleterious by 5/5 algorithms with AAF<0.1%; 4) pLOF or missense variants predicted deleterious by at least 1/5 algorithms with AAF<1%; 5) pLOF or missense variants predicted deleterious by at least 1/5 algorithms with AAF<0.1%; 6) pLOF or any missense with AAF<1%; 7) pLOF or any missense variants with AAF<0.1%.

Association Analysis of Gene Burden of Rare pLOF and Missense Variations

The burden of rare predicted loss-of-function or missense variants in a given gene and phenotype were examined for association by fitting a linear (for quantitative traits) or firth bias-corrected logistic (for binary traits) regression model adjusted for a polygenic score that approximates a genomic kinship matrix using REGENIE (Mbatchou et al., Nat. Genetics, 2020, 53, 1097-1103). Analyses were stratified by ancestry and adjusted for age, age², sex, age-by-sex and age²-by-sex interaction terms, experimental batch-related covariates, 10 common variant-derived principal components, and 20 rare variant-derived principal components. Results across cohorts for each variant-phenotype association were combined using fixed effects inverse variance weighted meta-analysis. In gene burden tests, all individuals are labeled as heterozygotes if they carry one or more qualifying rare variant (as described above based on frequency and functional annotation) and as homozygotes if they carry any qualifying variant in the homozygous state. This “composite genotype” is then used to test for association.

### Example 3: Association Between Loss-of-Function of ANGPTL3 and Improved Kidney Function is Unique Among Lipid Genes

Loss-of-function genetic variation in ANGPTL3 is known to associate with decreased circulating LDL cholesterol and triglyceride levels (Romeo et al., J. Clin. Invest., 2009, 119, 70-79; and Musunuru et al., N. Engl. J. Med., 2010, 363, 2220-2227), raising the question of whether the association between loss-of-function genetic variation in ANGPTL3 and improved kidney function is a consequence of lipid lowering. To address this question, we examined whether genetic variation in other known lipid-regulating genes was also associated with improved kidney function.

Low-frequency coding variants in ANGPTL4, PCSK9, and LPL were strongly associated with decreased circulating triglycerides (ANGPTL4 p.Glu40Lys, LPL p.Ser474*) or LDL cholesterol (PCSK9 p.Arg46Leu), (see Table 5). However, no significant increases in eGFR were observed with these genetic variants (Table 6), as would be expected if the increase in eGFR was secondary to decreased lipids.

TABLE 5
Associations with decreased triglyceride or LDL cholesterol levels for low-frequency
coding variants in ANGPTL4, PCSK9, and LPL genes

| Genetic exposure | | | | Genotype | |
|---|---|---|---|---|---|
| (Gene, amino acid | | Effect per allele | | counts, Ref/ | |
| variation) | Outcome | [95% CI] | P | Het/Hom | AAF |
| rs116843064 | Triglycerides | −0.24 [−0.25, −0.22] SD, | 6.3E−277 | 503,591/ | 0.0188 |
| (ANGPTL4, | | −20.68 [−21.82, −19.54] | | 19,287/ | |
| p.Glu40Lys) | | mg/dL | | 196 | |
| rs116843064 | LDL | 0.03 [0.02, 0.05] SD, | 1.6E−06 | 458,414/ | 0.0190 |
| (ANGPTL4, | cholesterol | 1.25 [0.74, 1.76] | | 17,733/ | |
| p.Glu40Lys) | | mg/dL | | 189 | |
| rs11591147 (PCSK9, | Triglycerides | −0.01 [−0.02, 0.01] SD, | 0.33 | 506,140/ | 0.0164 |
| p.Arg46Leu) | | −0.60 [−1.82, 0.62] | | 16,844/ | |
| | | mg/dL | | 148 | |
| rs11591147 (PCSK9, | LDL | −0.37 [−0.39, −0.36] SD, | 3.0E−528 | 461,013/ | 0.0163 |
| p.Arg46Leu) | cholesterol | −13.57 [−14.11, −13.03] | | 15,244/ | |
| | | mg/dL | | 136 | |
| rs328 (LPL, | Triglycerides | −0.20 [−0.20, −0.19] SD, | 8.0E−920 | 425,377/ | 0.100 |
| p.Ser474Ter) | | −17.17 [−17.69, −16.65] | | 94,558/ | |
| | | mg/dL | | 5,298 | |
| rs328 (LPL, | LDL | 0.02 [0.02, 0.03] SD, | 1.1E−14 | 386,450/ | 0.101 |
| p.Ser474Ter) | cholesterol | 0.89 [0.66, 1.12] | | 86,930/ | |
| | | mg/dL | | 4,878 | |

Abbreviations: CI, confidence interval; SD, standard deviation; mg/dL, milligrams per deciliter; AAF, alternative allele frequency; Ref, homozygous reference genotype; Het, heterozygous carrier of genetic exposure variant; Hom, homozygous carriers of genetic exposure variant; LDL, low-density lipoprotein; Glu, glutamate; Lys, lysine; Arg, arginine; Leu, leucine; Ser, serine; Ter, termination codon.

TABLE 6
Lack of associations with increased eGFR levels for low-frequency coding variants in
ANGPTL4, PCSK9, and LPL genes

| Genetic exposure | | | | Genotype | |
|---|---|---|---|---|---|
| (Gene, amino acid | | Effect per allele | | counts, Ref/ | |
| variation) | Outcome | [95% CI] | P | Het/Hom | AAF |
| rs116843064 | eGFR_{creatinine} | −0.00 [−0.01, 0.01] SD, | 0.75 | 523,796/ | 0.01892 |
| (ANGPTL4, | | −0.03 [−0.22, 0.16] | | 20,195/ | |
| p.Glu40Lys) | | mL/min | | 200 | |
| rs11591147 (PCSK9, | eGFR_{creatinine} | −0.03 [−0.04, −0.02] SD, | 8.0E−07 | 526,577/ | 0.01635 |
| p.Arg46Leu) | | −0.52 [−0.72, −0.31] | | 17,489/ | |
| | | mL/min | | 155 | |
| rs328 (LPL, | eGFR_{creatinine} | −0.00 [−0.01, 0.00] SD, | 0.15 | 442,385/ | 0.10018 |
| p.Ser474Ter) | | −0.06 [−0.15, 0.02] | | 98,410/ | |
| | | mL/min | | 5,526 | |

Abbreviations: CI, confidence interval; SD, standard deviation; mL/min, milliliters per minute; AAF, alternative allele frequency; Ref, homozygous reference genotype; Het, heterozygous carrier of genetic exposure variant; Hom, homozygous carriers of genetic exposure variant; eGFR_{creatinine}, estimated glomerular filtration rate calculated using serum creatinine; Glu, glutamate; Lys, lysine; Arg, arginine; Leu, leucine; Ser, serine; Ter, termination codon.

Low-frequency coding variants with significant effects on lipids exist in only a subset of lipid-regulating genes. Therefore, to more comprehensively examine whether coding variation in lipid-regulating genes associated reduced lipids with improved kidney function, we also examined associations between the burden of rare coding genetic variants in additional lipid-regulating genes and kidney function. The burden of rare coding genetic variants, either pLOF alone or pLOF and missense variants combined, in ANGPTL3, ANGPTL4, ANGPTL8, or PCSK9 was associated with decreased triglyceride or LDL cholesterol levels, whereas the burden of rare coding genetic variants in LPL or LDLR was associated with increased triglyceride or LDL cholesterol levels (Table 7), consistent with their known roles in lipid biology.

TABLE 7
Associations with triglyceride or LDL cholesterol levels for the burden of rare pLOF
or missense variants in lipid-regulating genes

| | | | | Genotype | |
|---|---|---|---|---|---|
| | | | | counts, | |
| | | Effect per allele | | Ref/Het/ | |
| Genetic exposure | Outcome | [95% CI] | P | Hom | AAF |
| ANGPTL3 pLOF; AAF < | Triglycerides | −0.64 [−0.69, −0.59] | 1.3E−123 | 524,069/ | 0.00111 |
| 1% | | SD, | | 1,168/1 | |
| | | −55.85 [−60.48, | | | |
| | | −51.22] mg/dL | | | |
| ANGPTL3 pLOF; AAF < | LDL | −0.24 [−0.29, −0.18] | 5.5E−17 | 477,170/ | 0.00114 |
| 1% | cholesterol | SD, | | 1,092/0 | |
| | | −8.66 [−10.68, −6.63] | | | |
| | | mg/dL | | | |
| ANGPTL3 pLOF or | Triglycerides | −0.55 [−0.59, −0.51] | 4.4E−180 | 522,914/ | 0.00222 |
| missense (5/5); AAF < | | SD, | | 2,321/3 | |
| 0.1% | | −48.09 [−51.39, | | | |
| | | −44.80] mg/dL | | | |
| ANGPTL3 pLOF or | LDL | −0.20 [−0.24, −0.16] | 2.1E−22 | 476,101/ | 0.00226 |
| missense (5/5); AAF < | cholesterol | SD, | | 2,160/1 | |
| 0.1% | | −7.17 [−8.62, −5.73] | | | |
| | | mg/dL | | | |
| ANGPTL4 pLOF; AAF < | Triglycerides | −0.39 [−0.45, −0.33] | 1.1E−32 | 523,846/ | 0.00077 |
| 1% | | SD, | | 805/0 | |
| | | −33.95 [−39.54, | | | |
| | | −28.36] mg/dL | | | |
| ANGPTL4 pLOF; AAF < | LDL | 0.05 [−0.02, 0.11] | 0.19 | 476,987/ | 0.0008 |
| 1% | cholesterol | SD, | | 762/0 | |
| | | 1.65 [−0.83, 4.12] | | | |
| | | mg/dL | | | |
| ANGPTL4 pLOF or | Triglycerides | −0.35 [−0.39, −0.30] | 3.4E−52 | 522,979/ | 0.00159 |
| missense (5/5); AAF < | | SD, | | 1,672/0 | |
| 1% | | −30.13 [−34.01, | | | |
| | | −26.24] mg/dL | | | |
| ANGPTL4 pLOF or | LDL | 0.03 [−0.01, 0.08] | 0.17 | 476,190/ | 0.00163 |
| missense (5/5); AAF < | cholesterol | SD, | | 1,559/0 | |
| 1% | | 1.20 [−0.53, 2.93] | | | |
| | | mg/dL | | | |
| ANGPTL8 pLOF; AAF < | Triglycerides | −0.35 [−0.41, −0.29] | 8.8E− | 524,294/ | 0.0009 |
| 1% | | SD, | 31 | 942/2 | |
| | | −30.34 [−35.49, | | | |
| | | −25.18] mg/dL | | | |
| ANGPTL8 pLOF; AAF < | LDL | −0.02 [−0.08, 0.04] | 0.52 | 477,390/ | 0.00091 |
| 1% | cholesterol | SD, | | 871/1 | |
| | | −0.76 [−3.07, 1.55] | | | |
| | | mg/dL | | | |
| ANGPTL8 pLOF or | Triglycerides | −0.38 [−0.44, −0.31] | 9.1E−27 | 524,544/ | 0.00066 |
| missense (5/5); AAF < | | SD, | | 692/2 | |
| 0.1% | | −32.76 [−38.76, | | | |
| | | −26.77] mg/dL | | | |
| ANGPTL8 pLOF or | LDL | −0.02 [−0.09, 0.06] | 0.62 | 477,641/ | 0.00065 |
| missense (5/5); AAF < | cholesterol | SD, | | 620/1 | |
| 0.1% | | −0.69 [−3.42, 2.04] | | | |
| | | mg/dL | | | |
| LPL pLOF; AAF < 1% | Triglycerides | 0.72 [0.57, 0.86] SD, | 2.7E−22 | 524,492/ | 0.00015 |
| | | 62.58 [49.95, 75.21] | | 159/0 | |
| | | mg/dL | | | |
| LPL pLOF; AAF < 1% | LDL | −0.18 [−0.33, −0.03] | 0.021 | 477,606/ | 0.00015 |
| | cholesterol | SD, | | 143/0 | |
| | | −6.56 [−12.12, −1.01] | | | |
| | | mg/dL | | | |
| LPL pLOF or missense | Triglycerides | 0.40 [0.35, 0.46] SD, | 3.6E−43 | 523,634/ | 0.00097 |
| (5/5); AAF < 0.1% | | 35.16 [30.16, 40.16] | | 1,017/0 | |
| | | mg/dL | | | |
| LPL pLOF or missense | LDL | −0.07 [−0.13, −0.01] | 0.02 | 476,841/ | 0.00095 |
| (5/5); AAF < 0.1% | cholesterol | SD, | | 908/0 | |
| | | −2.62 [−4.83, −0.41] | | | |
| | | mg/dL | | | |
| LDLR pLOF; AAF < 1% | Triglycerides | −0.12 [−0.20, −0.03] | 0.0094 | 524,225/ | 0.00041 |
| | | SD, | | 426/0 | |
| | | −10.18 [−17.86, | | | |
| | | −2.50] mg/dL | | | |
| LDLR pLOF; AAF < 1% | LDL | 0.28 [0.19, 0.38] SD, | 5.5E−09 | 477,359/ | 0.00041 |
| | cholesterol | 10.28 [6.82, 13.74] | | 390/0 | |
| | | mg/dL | | | |
| LDLR pLOF or missense | Triglycerides | −0.05 [−0.09, −0.02] | 0.0041 | 522,786/ | 0.00234 |
| (5/5); AAF < 0.1% | | SD, | | 2,451/1 | |
| | | −4.70 [−7.91, −1.49] | | | |
| | | mg/dL | | | |
| LDLR pLOF or missense | LDL | 0.49 [0.45, 0.53] SD, | 1.6E−129 | 476,015/ | 0.00235 |
| (5/5); AAF < 0.1% | cholesterol | 17.84 [16.39, 19.28] | | 2,247/0 | |
| | | mg/dL | | | |
| PCSK9 pLOF; AAF < 1% | Triglycerides | −0.13 [−0.19, −0.07] | 9.7E−06 | 524,266/ | 0.00093 |
| | | SD, | | 971/1 | |
| | | −11.52 [−16.63, | | | |
| | | −6.42] mg/dL | | | |
| PCSK9 pLOF; AAF < 1% | LDL | −0.94 [−1.00, −0.88] | 1.2E−193 | 477,378/ | 0.00093 |
| | cholesterol | SD, | | 883/1 | |
| | | −34.13 [−36.38, | | | |
| | | −31.87] mg/dL | | | |
| PCSK9 pLOF or | Triglycerides | −0.07 [−0.12, −0.03] | 5.3E−04 | 523,346/ | 0.0018 |
| missense (5/5); AAF < | | SD, | | 1,889/3 | |
| 0.1% | | −6.46 [−10.12, −2.81] | | | |
| | | mg/dL | | | |
| PCSK9 pLOF or | LDL | −0.70 [−0.75, −0.66] | 9.9E−212 | 476,530/ | 0.00181 |
| missense (5/5); AAF < | cholesterol | SD, | | 1,729/3 | |
| 0.1% | | −25.53 [−27.15, | | | |
| | | −23.92] mg/dL | | | |

Abbreviations: CI, confidence interval; SD, standard deviation; mg/dL, milligrams per deciliter; AAF, alternative allele frequency; Ref, homozygous reference genotype; Het, heterozygous carrier of rare coding variant (defined in 'Genetic exposure'); Hom, homozygous carrier of rare coding variant (defined in 'Genetic exposure'); pLOF, predicted loss of function; missense (5/5), missense variant predicted to be deleterious by 5 out of 5 in silico prediction algorithms; LDL, low-density lipoprotein

However, when examining effects on kidney function, only the burden of rare coding genetic variants in ANGPTL3 showed a significant effect on kidney function directionally concordant with its effects on lipids (that is, decreased triglycerides or LDL associated with increased eGFR, or increased triglycerides or LDL associated with decreased eGFR, see Table 8). Therefore, the association between loss-of-function genetic variation in ANGPTL3 and improved kidney function reflects a unique role of ANGPTL3 function as opposed to generic lipid-lowering.

TABLE 8
The burden of rare pLOF or missense variants in ANGPTL3, but not ANGPTL4,
ANGPTL8, LPL, PCSK9, or LDLR show significant, inverse direction of effect on lipids (see
Table 7) and eGFR

| | | Expected | | | Genotype | |
|---|---|---|---|---|---|---|
| | | direction | | | counts, | |
| Genetic | | of | Observed | | Ref/Het/ | |
| exposure | Outcome | effect | effect per allele | P | Hom | AAF |
| ANGPTL3 pLOF; | eGFR_{creatinine} | Increase | 0.06 | 0.0084 | 545,101/ | 0.00112 |
| AAF <1% | | | [0.02, 0.11] | | 1,225/1 | |
| | | | SD, 1.03 | | | |
| | | | [0.26, 1.79] | | | |
| | | | mL/min | | | |
| ANGPTL3 pLOF | eGFR_{creatinine} | Increase | 0.05 | 0.0019 | 543,886/ | 0.00224 |
| or deleterious | | | [0.02, 0.08] | | 2,437/4 | |
| missense (5/5); | | | SD, 0.86 | | | |
| AAF <0.1% | | | [0.32, 1.4] | | | |
| | | | mL/min | | | |
| ANGPTL4 pLOF; | eGFR_{creatinine} | Increase | 0.01 | 0.76 | 544,886/ | 0.00078 |
| AAF <1% | | | [−0.05, 0.06] | | 854/0 | |
| | | | SD, 0.14 | | | |
| | | | [−0.76, 1.05] | | | |
| | | | mL/min | | | |
| ANGPTL4 pLOF | eGFR_{creatinine} | Increase | 0.01 | 0.7 | 543,979/ | 0.00161 |
| or deleterious | | | [−0.03, 0.04] | | 1,761/0 | |
| missense (5/5); | | | SD, 0.12 | | | |
| AAF <1% | | | [−0.51, 0.76] | | | |
| | | | mL/min | | | |
| ANGPTL8 pLOF; | eGFR_{creatinine} | Increase | −0.06 | 0.0086 | 545,312/ | 0.00093 |
| AAF <1% | | | [−0.11, −0.02] | | 1,013/2 | |
| | | | SD, −1.1 | | | |
| | | | [−1.91, −0.28] | | | |
| | | | mL/min | | | |
| ANGPTL8 pLOF | eGFR_{creatinine} | Increase | −0.06 | 0.051 | 545,620/ | 0.00065 |
| or deleterious | | | [−0.12, 0.00] | | 705/2 | |
| missense (5/5); | | | SD, −1.03 | | | |
| AAF <0.1% | | | [−2.06, 0] | | | |
| | | | mL/min | | | |
| LPL pLOF; | eGFR_{creatinine} | Decrease | 0.11 | 0.099 | 545,578/ | 0.00015 |
| AAF <1% | | | [−0.02, 0.24] | | 162/0 | |
| | | | SD, 1.82 | | | |
| | | | [−0.34, 3.98] | | | |
| | | | mL/min | | | |
| LPL pLOF or | eGFR_{creatinine} | Decrease | 0.05 | 0.045 | 544,648/ | 0.001 |
| deleterious | | | [0.00, 0.10] | | 1,089/3 | |
| missense (5/5); | | | SD, 0.82 | | | |
| AAF <0.1% | | | [0.02, 1.62] | | | |
| | | | mL/min | | | |
| LDLR pLOF; | eGFR_{creatinine} | Decrease | −0.02 | 0.58 | 545,299/ | 0.0004 |
| AAF <1% | | | [−0.10, 0.05] | | 441/0 | |
| | | | SD,− 0.36 | | | |
| | | | [−1.62, 0.91] | | | |
| | | | mL/min | | | |
| LDLR pLOF or | eGFR_{creatinine} | Decrease | −0.00 | 0.83 | 543,794/ | 0.00232 |
| deleterious | | | [−0.03, 0.03] | | 2,532/1 | |
| missense (5/5); | | | SD, −0.06 | | | |
| AAF <0.1% | | | [−0.59, 0.48] | | | |
| | | | mL/min | | | |
| PCSK9 pLOF; | eGFR_{creatinine} | Increase | −0.05 | 0.066 | 545,344/ | 0.0009 |
| AAF <1% | | | [−0.10, 0.00] | | 982/1 | |
| | | | SD, −0.83 | | | |
| | | | [−1.71, 0.06] | | | |
| | | | mL/min | | | |
| PCSK9 pLOF or | eGFR_{creatinine} | Increase | −0.03 | 0.11 | 544,376/ | 0.00179 |
| deleterious | | | [−0.07, 0.01] | | 1,948/3 | |
| missense (5/5); | | | SD, −0.51 | | | |
| AAF <0.1% | | | [−1.12, 0.11] | | | |
| | | | mL/min | | | |

Abbreviations:
Cl, confidence interval;
SD, standard deviation;
mL/min, milliliters per minute;
AAF, alternative allele frequency;
Ref, homozygous reference genotype;
Het, heterozygous carrier of rare coding variant (defined in ‘Genetic exposure’);
Hom, homozygous carrier of rare coding variant (defined in ‘Genetic exposure’);
pLOF, predicted loss of function;
missense (5/5), missense variant predicted to be deleterious by 5 out of 5 in silico prediction algorithms;
eGFR_{creatinine}, estimated glomerular filtration rate calculated using serum creatinine;
Expected direction of effect, expected direction of genetic exposure effect on eGFR_{creatinine}, given the most significant (lowest p-value) effect of genetic exposure on LDL (low-density lipoprotein) cholesterol or triglycerides from Table 7 (that is, decreased LDL cholesterol or triglycerides expected to increase eGFR_{creatinine}, whereas increased LDL cholesterol or triglycerides expected to decreased eGFR_{creatinine}).

### Example 4: Circulating ANGPTL3 Levels were Positively Associated with Risk of Newly-Onset Chronic Didney Disease

We assessed the relationship between circulating plasma ANGPTL3 levels and kidney function in 38,043 individuals without evidence of kidney disease or dysfunction. At the time of measurement, plasma ANGTPL3 was weakly correlated with LDL cholesterol (Partial correlation coefficient=0.21), high density lipoprotein (HDL) cholesterol (0.16), triglycerides (0.12), cystatin-C (0.19), creatinine (0.03), and eGFR_{creatinine}. (−0.04), after adjusting for age-at-baseline, sex, and body-mass index.

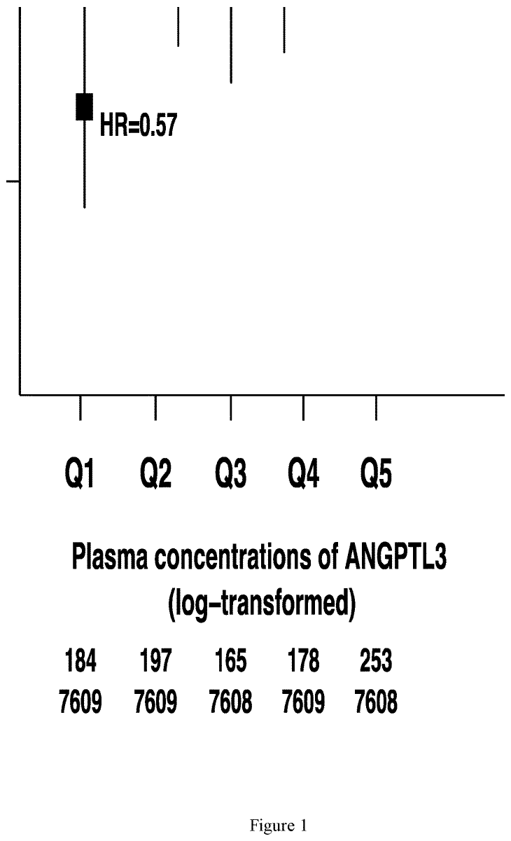

Circulating levels of ANGPTL3 were also positively associated with higher risk of newly-onset CKD (FIG. 1), after stratification for sex and study center, and adjustment for age-at-baseline, smoking (current-smoker, ex-smoker, and never-smoker), alcohol consumption (current-drinker, ex-drinker, and never-drinker), history of diabetes (Yes vs. No), HDL cholesterol, LDL cholesterol, eGFR_{creatinine}. Compared to participants who were in the top quartile of the ANGPTL3 levels, the adjusted hazard ratio (HR) for those who were in the bottom quartile was 0.57 (95% CI: 0.45-0.71; p=1.19E-6). Results remained similar after further adjustment for baseline levels of triglycerides, and hemoglobin A1c.

TABLE 9
adjusted associations of circulating ANGPTL3 levels and risk of newly-onset
chronic kidney disease in UK Biobank

| | No. of | No. of | ANGPTL3 | ANGPTL3, | Hazard ratios |
|---|---|---|---|---|---|
| Quartiles | events | participants | (Iog2-transformed) | NPX units | (95% CI) |
| Q1 | 184 | 7609 | −0.23 | 0.85 | 0.57 (0.48-0.67) |
| Q2 | 197 | 7609 | 0.15 | 1.11 | 0.72 (0.63-0.84) |
| Q3 | 165 | 7608 | 0.37 | 1.29 | 0.69 (0.59-0.80) |
| Q4 | 178 | 7609 | 0.59 | 1.51 | 0.72 (0.62-0.84) |
| Q5 | 253 | 7608 | 0.97 | 1.96 | Reference |

Analyses were conducted among individuals with prior history of physician-diagnosed CKD, or abnormal eGFR_{creatinine} (defined here as: eGFR<60 mL/min/1.73 m² or eGFR>110 mL/min/1.73 m²) at recruitment, and those with missing data on covariates (defined here as: systolic blood pressure, HDL cholesterol, LDL cholesterol, smoking status, alcohol consumption status, history of diabetes at baseline, and eGFR_{creatinine}). Hazard ratios and the corresponding 95% confidence intervals were estimated using Cox regression, stratified by sex and study center, adjusted for age-at-baseline, smoking status, alcohol consumption, history of diabetes, HDL-C, LDL-C, and baseline levels of eGFR_{creatinine}. Abbreviations: HDL, high-density lipoprotein; LDL, low-density lipoprotein; eGFR_{creatinine}, estimated glomerular filtration rate calculated from serum creatinine, NPX, Normalized Protein eXpression relative quantification units.

### Participating Cohorts

Genetic association studies were performed in the United Kingdom (UK) Biobank (UKB) cohort (Sudlow et al., PLoS Med., 2015, 12, e1001779) and the DiscoverEHR cohort from the Geisinger Health System (GHS) MyCode Community Health Initiative (Carey et al., Genet. Med., 2016, 18, 906-13). The UKB is a population-based cohort study of people aged between 40 and 69 years recruited through 22 testing centers in the UK between 2006-2010. Over 430,000 participants of varying ancestries from UKB with available whole-exome sequencing and clinical phenotype data were included. Among a subset of the entire UK Biobank prospective study, proteomic profiling was conducted on blood plasma samples collected from n=54,306 individuals at baseline. The GHS MyCode study Community Health Initiative is a health system-based cohort of patients from Central and Eastern Pennsylvania (USA) recruited in 2007-2019. Over 130,000 European ancestry participants from GHS with available whole-exome sequencing and clinical phenotype data were included.

### Phenotype Definitions

eGFR was calculated from clinical laboratory measurements for creatinine extracted from electronic health records (EHRs) of participants from GHS. Median values were calculated for all participants with two or more measurements. In UKB, eGFR_{creatinine} was calculated from creatinine measured on a Beckman Coulter AU5800 clinical chemistry analyzer and eGFR_{cystatin} was calculated from cystatin measured by immunoturbidimetric analysis on a Siemens Advia 1800 clinical chemistry analyzer; both creatinine and cystatin were measured at the baseline visit of the study. In UKB, urine albumin:creatinine ratio (UACR) was derived from creatinine measured by enzymatic analysis and albumin measured by immunoturbidimetric analysis on a Beckman Coulter AU5400 clinical chemistry analyzer on spot urine measured at the baseline visit of the study. Lipid levels were measured during a visit to the assessment center for UK Biobank participants on a Beckman Coulter analyzer or were extracted from electronic health record datasets for participants from GHS. Direct LDL measurements were available for UK Biobank participants. LDL levels were estimated using the Friedewald equation for the other participating individuals. Individuals known to be on lipid lowering medication had their pre-treatment LDL estimated using a correction factor for individuals known to be on lipid lowering medication. Prior to genetic association analysis, continuous phenotype values were transformed by the inverse standard normal function, applied within each ancestry group and separately in men and women. Proteomic profiling was conducted on blood plasma samples collected at baseline using the Olink® Explore 1536 platform, measuring 1,472 protein analytes, capturing 1,463 unique proteins. Circulating levels of proteins were expressed as Olink's relative quantification unit (i.e., Normalized Protein eXpression) on a log-2 scale (“doi.org/10.1101/2022.06.17.496443”). Kidney disease outcomes were defined according to the International Classification of Diseases Tenth Revision (ICD-10) and read codes stored in EHRs. Self-reported disease status was used when available. Office of Population Censuses and Surveys Classification of Interventions and Procedures version 4 (OPCS4) codes were used for medical procedures in UKB.

Individuals with kidney diseases were identified as described in Table 5, combining EHR records, self-reports and eGFR and UACR measurements. Associations of circulating ANGPTL3 were assessed with incident occurrence of physician-diagnosed CKD in UKB, defined as occurrence of any of the following criteria:

1. ICD10: N18, Y841, Z49, Z992, T861, Z940

2. ICD9: 585

3. Self-reported illness code: 1580, 1581, 1582

For this analysis, individuals were excluded from the study population if they met any of the following criteria: prior history of physician-diagnosed CKD according to the above definition, abnormal eGFR_{creatinine} defined here as <90 ml/min/1.72 m² or >110 ml/min/1.72 m².

For genetic analyses, participants were excluded from the control population for all kidney disease outcomes if they met any of the following criteria:

1. ICD10: N00, N01, N02, N03, N04, N05, N06, N07, N08, N10, N11, N12, N13, N14, N15, N16, N17, N18, N19, N20, N21, N22, N23, N25, N26, N27, N28, N29, Q60, Q61, Q63, C64, R944, I12, I13, T861, Z940, O904, O084, T824, Y841, Z490, Z491, Z492, Z992

2. eGFR<90 ml/min/1.72 m²

3. Self-reported illness code (UKB-specific): 1192, 1193, 1194, 1197, 1519, 1520, 1607, 1608, 1609, 1427, 1405

4. Self-reported operation code (UKB-specific): 1195, 1197, 1476, 1487, 1580, 1581, 1582, 1618

5. OPCS4 operation code (UKB-specific): M01, M02, M03, M04, M05, M061, M068, M069, M07, M08, M09, M10, M111, M112, M131, M132, M133, M137, M14, M164, L746, M172, M 174, M178, M179, X401, X402, X403, X404, X405, X406, X408, X409, X411, X412, X418, X419, X421, X 428, X429, M012, M013, M014, M015, M018, M019, M084

6. Source of end-stage renal disease report code (UKB-specific): 0, 1, 2

7. Urinary albumin:creatinine ratio >10 mg/g (UKB-specific)

8. EHR code for kidney transplant or dialysis in billing procedures or surgery (GHS-specific).

TABLE 10
Definitions of kidney disease and outcomes in UKB and GHS cohort

| | Case definition |
|---|---|
| Kidney disease | Participants were included as a case in genetic analyses if they met any |
| outcome | of the criteria specific to each disease |
| Microalbuminuria | UKB.UACR > 30 mg/g |
| Any kidney | ICD10: |
| disease | N00, N01, N02, N03, N04, N05, N06, N07, N08, N10, N11, N12, N13, N14, N15, N16, |
| | N17, N18, N19, N25, N26, N27, N28, N29, Q60, Q61, Q63, C64, R944, I12, I13, T861, |
| | Z940, O904, O084, T824, Y841, Z490, Z491, Z492, Z992 |
| | UKB.f.20002: 1192, 1193, 1194, 1519, 1520, 1607, 1608, 1609, 1427, 1405 |
| | UKB.f.20004: 1195, 1476, 1487, 1580, 1581, 1582, 1618 |
| | UKB.OPCS4: |
| | M01, M02, M03, M04, M05, M068, M069, M078, M079, M08, M10, M111, M112, |
| | M131, M132, M133, M137, M164, L746, M172, M174, M178, M179, X401, X402, |
| | X403, X404, X405, X406, X408, X409, X411, X412, X418, X419, X421, X428, |
| | X429, M012, M013, M014, M015, M018, M019, M084 |
| | UKB.f.42027: 0, 1, 2 |
| | GHS.EHR: kidney transplant or dialysis in billing procedures or surgery |
| | eGFR < 60 ml/min/1.72 m² |
| | UKB.UACR > 30 mg/g |
| Chronic kidney | ICD10: N18, Q611, Q612, Q613, Q615, I12, I13, T861, Z940, Z492 |
| disease, any | UKB.f.20002: 1193, 1607, 1427 |
| | UKB.f.20004: 1195, 1476, 1580, 1581, 1582 |
| | UKB.OPSC4: |
| | L746, M012, M013, M014, M015, M018, M019, M084, M174, M178, M179, X402, |
| | X405, X406, X411, X412 |
| | UKB.f.42027: 0, 1, 2 |
| | GHS.EHR: kidney transplant in billing procedures or surgery |
| | eGFR < 60 ml/min/1.72 m² |
| | UKB.UACR > 300 mg/g |
| Chronic kidney | ICD10: N18, Q611, Q612, Q613, Q615, I12, I13, T861, Z940, Z492 |
| disease, any | UKB.f.20002: 1193, 1607, 1427 |
| including | UKB.f.20004: 1195, 1476, 1580, 1581, 1582 |
| microalbuminuria | UKB.OPSC4: |
| | L746, M012, M013, M014, M015, M018, M019, M084, M174, M178, M179, X402, |
| | X405, X406, X411, X412 |
| | UKB.f.42027: 0, 1, 2 |
| | GHS.EHR: kidney transplant in billing procedures or surgery |
| | eGFR < 60 ml/min/1.72 m² |
| | UKB.UACR > 30 mg/g |
| CKD stage 3+ | ICD10: N183, N184, N185, N180, N186, T861, Z492, Z940, N165 |
| | UKB.f.20002: 1193 |
| | UKB.f.20004: 1476, 4195, 1580, 1581, 1582 |
| | UKB.OPSC4: |
| | M012, M013, M014, M015, M018, M019, M084, M174, M178, M179, X402, X405, |
| | X406, X411, X412 |
| | UKB.f.42027: 0, 1, 2 |
| | GHS.EHR: kidney transplant in billing procedures or surgery |
| | eGFR < 60 ml/min/1.72 m² |
| CKD stage 5+ | ICD10: N185, N180, N186, T861, Z492, Z940, N165 |
| | UKB.f.20002: 1193 |
| | UKB.f.20004: 1476, 1195, 1580, 1581, 1582 |
| | UKB.OPSC4: |
| | M012, M013, M014, M015, M018, M019, M084, M174, M178, M179, X402, X4 |
| | 05, X406, X411, X412 |
| | UKB.f.42027: 0, 1, 2 |
| | GHS.EHR: kidney transplant in billing procedures or surgery |
| | eGFR < 15 ml/min/1.72 m² |
| Renal | ICD10:T824, T861, Y841, Z490, Z491, Z492, Z940, Z992 |
| replacement | UKB.f.20002: 1193 |
| therapy | UKB.f.20004: 1195, 1476, 1580, 1581, 1582 |
| | UKB.OPSC4: |
| | L746, M012, M013, M014, M015, M018, M019, M084, M174, M178, M179, X401, |
| | X402, X403, X404, X405, X406, X408, X409, X411, X412, X418, X419, X421, |
| | X428, X429 |
| | UKB.f.42027: 0, 1, 2 |
| | GHS.EHR: kidney transplant or dialysis in billing procedures or surgery |
| End stage renal | ICD10: N180, N186, T861, Z492, Z940, N165 |
| disease | UKB.f.20002: 1193 |
| | UKB.f.20004: 1476, 1195, 1580, 1581, 1582 |
| | UKB.OPSC4: |
| | M012, M013, M014, M015, M018, M019, M084, M174, M178, M179, X402, X405, |
| | X406, X411, X412 |
| | UKB.f.42027: 0, 1, 2 |
| | GHS.EHR: kidney transplant in billing procedures or surgery |

ICD10 indicates the 10th revision of the International Statistical Classification of Diseases and Related Health Problems; UKB.OPCS4 indicates Office of Population Censuses and Surveys (OPCS) Classification of Interventions and Procedures version 4 as used in the UK Biobank (UKB); UKB.f.20002 indicates self-reported non-cancer illness codes as used in UKB; UKB.f.20004 indicates self-reported medical procedures as used in UKB; UKB.f.42027 indicates source of end-stage renal disease code as defined by central working group for UKB; GHS.EHR indicates billing procedures or surgeries in Geisinger Health System (GHS) relevant to disease definition; UKB.UACR indicates urine albumin:creatinine ratio measured in UKB.

### Genotype Data

High coverage whole exome sequencing was performed as previously described (Science, 2016, 354:aaf6814; and Nature, 2020, 586, 749-756) and as summarized below. NimbleGen probes (VCRome; for part of the GHS cohort) or a modified version of the xGen design available from Integrated DNA Technologies (IDT; for the rest of GHS and other cohorts) were used for target sequence capture of the exome. A unique 6 base pair (bp) barcode (VCRome) or 10 bp barcode (IDT) was added to each DNA fragment during library preparation to facilitate multiplexed exome capture and sequencing. Equal amounts of sample were pooled prior to exome capture. Sequencing was performed using 75 bp paired-end reads on Illumina v4 HiSeq 2500 (for part of the GHS cohort) or NovaSeq (for the rest of GHS and other cohorts) instruments. Sequencing had a coverage depth (i.e., number of sequence-reads covering each nucleotide in the target areas of the genome) sufficient to provide greater than 20× coverage over 85% of targeted bases in 96% of VCRome samples and 20× coverage over 90% of targeted bases in 99% of IDT samples. Data processing steps included sample de-multiplexing using Illumina software, alignment to the GRCh38 Human Genome reference sequence including generation of binary alignment and mapping files (BAM), processing of BAM files (e.g., marking of duplicate reads and other read mapping evaluations). Variant calling was performed using the GLNexus system (DOI: 10.1101/343970). Variant mapping and annotation were based on the GRCh38 Human Genome reference sequence and Ensembl v85 gene definitions using the snpEff software. The snpEff predictions that involve protein-coding transcripts with an annotated start and stop were then combined into a single functional impact prediction by selecting the most deleterious functional effect class for each gene. The hierarchy (from most to least deleterious) for these annotations was frameshift, stop-gain, stop-loss, splice acceptor, splice donor, stop-lost, in-frame indel, missense, other annotations. Predicted LOF genetic variants included: a) insertions or deletions resulting in a frameshift, b) insertions, deletions or single nucleotide variants resulting in the introduction of a premature stop codon or in the loss of the transcription start site or stop site, and c) variants in donor or acceptor splice sites. Missense variants were classified for likely functional impact according to the number of in silico prediction algorithms that predicted deleteriousness using SIFT (Adzhubei et al., Nat. Methods, 2010, 7, 248-9) and Polyphen2_HVAR (Adzhubei et al., Nat. Methods, 2010, 7, 248-9), LRT (Chun et al., Genome Res., 2009, 19, 1553-61) and MutationTaster (Schwarz et al., Nat. Methods, 2010, 7, 575-6). For each gene, the alternative allele frequency (AAF) and functional annotation of each variant determined inclusion into these 7 gene burden exposures: 1) pLOF variants with AAF<1%; 2) pLOF or missense variants predicted deleterious by 5/5 algorithms with AAF<1%; 3) pLOF or missense variants predicted deleterious by 5/5 algorithms with AAF<0.1%; 4) pLOF or missense variants predicted deleterious by at least 1/5 algorithms with AAF<1%; 5) pLOF or missense variants predicted deleterious by at least 1/5 algorithms with AAF<0.1%; 6) pLOF or any missense with AAF<1%; 7) pLOF or any missense variants with AAF<0.1%.

Association Analysis of Single Nucleotide Polymorphisms or Gene Burden of Rare pLOF and Missense Variations

Associations between phenotypes and either the burden of rare predicted loss-of-function or missense variants in a given gene, or single nucleotide polymorphism low-frequency coding variants, were examined by fitting a linear (for quantitative traits) or firth bias-corrected logistic (for binary traits) regression model adjusted for a polygenic score that approximates a genomic kinship matrix using REGENIE (Mbatchou et al., Nat. Genetics, 2020, 53, 1097-1103). Analyses were stratified by ancestry and adjusted for age, age², sex, age-by-sex and age²-by-sex interaction terms, experimental batch-related covariates, 10 common variant-derived principal components, and 20 rare variant-derived principal components. Results across cohorts for each variant-phenotype association were combined using fixed effects inverse variance weighted meta-analysis. In gene burden tests, all individuals are labeled as heterozygotes if they carry one or more qualifying rare variant (as described above based on frequency and functional annotation) and as homozygotes if they carry any qualifying variant in the homozygous state. This “composite genotype” is then used to test for association. For analyses comparing gene burden effects on LDL or triglycerides to that on eGFR, the gene burden containing missense variants with the strongest effect (largest effect size and most significant p-value) on the relevant lipid for each gene listed was evaluated for its effect on eGFR; the burden of pLOF variants with AAF<1% for each gene was also evaluated.

### Association Analysis of Circulating Levels of ANGPTL3 and Risk of Newly-Onset Chronic Kidney Disease

Participants were included if they: 1) had no prior history of physician-diagnosed CKD, or abnormal baseline eGFR_{creatinine} (defined here as <90 ml/min/1.72 m² or >110 ml/min/1.72 m²; 2) had complete information on covariates, including baseline measurements of systolic blood pressure, HDL cholesterol, LDL cholesterol (directly-measured)), history of diabetes, alcohol consumption, and smoking status; 3) had Olink assay-measured baseline levels of ANGPTL3.

Overall, 38,043 participants were included in the analysis, including 977 newly-onset CKD outcomes. Participants were grouped into quartiles based on their baseline levels of ANGPTL3. Compared to the reference group (defined here as the top quartile), hazard ratios and corresponding 95% confidence interval (Cis) were estimated using Cox proportional regression model with duration (time-on-study) as the underlying timescale, stratified by sex and study center, adjusted for age-at-baseline, systolic blood pressure (continuous), HDL cholesterol (continuous), LDL cholesterol (continuous), history of diabetes (Yes vs. No), alcohol consumption (Current-, Ex- and Never-drinkers), and smoking status (Current-, Ex- and Never-smokers). Sensitivity analyses were performed by further adjusting for baseline triglycerides and hemoglobin A1C in addition to the previous adjustments.

### Example 5: Circulating ANGPTL3 is Increased in Rodent Models of Kidney Disease

Uninephrectomy of db/db mice, which have defective leptin receptor, on the C57BLKS/J stain background, is a common model of the diabetic nephropathy presenting with albuminuria and reduced estimated glomerular filtration rate (Tesch et al., Am. J. Physiol. Renal. Physiol., 2011, 300, F301-F310). Male db/db C57BLKS/J mice were uninephrectomized at 8 weeks of age under ketamine/xylazine anesthesia. Urine was collected for 16-18 hours using diuresis cages (Tecniplast) at 22 weeks of age. Urinary albumin was measured using a commercially available kit (Albuwell M, Ethos Bioscience). The urinary albumin levels were normalized with urinary creatinine levels (Ethos Bioscience). Serum ANGPTL3 levels collected at 22 weeks of age were measured using a commercially available ELISA (BioTechne).

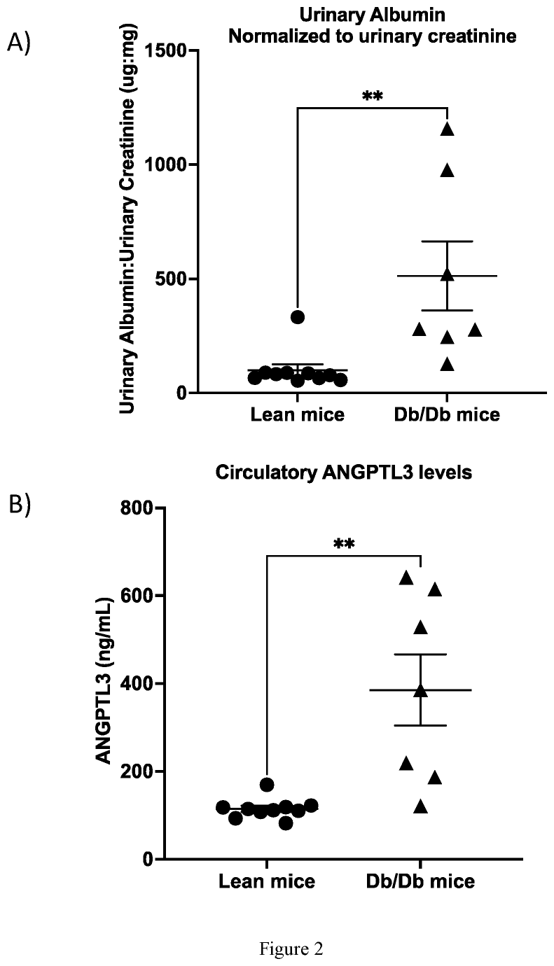

At 22 weeks old, the uninephrectomized db/db mice exhibited signs of kidney disease, including significantly increased urine albumin, compared to wildtype lean controls (p=0.0058, FIG. 2, Panel A). At this timepoint, serum ANGPTL3 was also significantly increased 3.35-fold compared to wildtype controls (p=0.0011, FIG. 2, Panel B).

The remnant kidney model (RKM) or 5/liths nephrectomy is a common model of chronic kidney disease (Leelahavanichkul et al., Kidney Int., 2010, 78(11), 1136-1153). ⅔ of left kidney was resected in 129sv mice (Taconic), followed by uninephrectomy of the right kidney one week later under ketamine/xylazine anesthesia. Urine was collected for 16-18 hours using diuresis cages (Tecniplast) 5 weeks after the second surgery. Urinary albumin was measured using a commercially available kit (Albuwell M, Ethos Bioscience). The urinary albumin levels were normalized with urinary creatinine levels (Ethos Bioscience). Serum ANGPTL3 levels collected 5 weeks after the second surgery were measured using a commercially available ELISA (BioTechne).

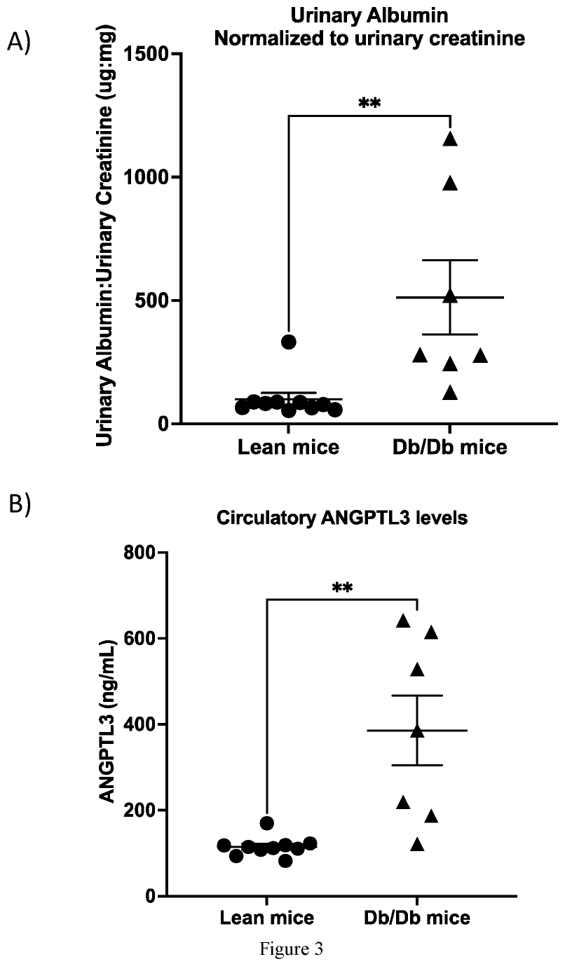

The urinary albumin levels were significantly increased in the RKM mice at 5 weeks post-second surgery compared to sham-operated controls (p=0.0122, FIG. 3, Panel A) indicating kidney injury. At this timepoint, serum ANGPTL3 was also significantly increased ^{˜}1.4-fold in mice with the remnant kidney model compared to sham-operated controls (p<0.0005, FIG. 3, Panel B).

In summary, circulating ANGPTL3 levels are increased in multiple models of chronic kidney disease in rodents.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject having a kidney disease or at risk of developing a kidney disease, the method comprising administering an Angiopoietin Like 3 (ANGPTL3) inhibitor to the subject, wherein the kidney disease is not nephrotic syndrome.

2. The method according to claim 1, wherein the kidney disease is chronic kidney disease, a kidney stone, chronic glomerulonephritis, nephronophthisis, chronic interstitial nephritis, or nephrosclerosis.

3-7. (canceled)

8. The method according to claim 1, wherein the ANGPTL3 inhibitor comprises an inhibitory nucleic acid molecule that hybridizes to an ANGPTL3 nucleic acid molecule.

9. The method according to claim 8, wherein the inhibitory nucleic acid molecule comprises an antisense nucleic acid molecule, a small interfering RNA (siRNA), or a short hairpin RNA (shRNA).

10-12. (canceled)

13. The method according to claim 1, wherein the ANGPTL3 inhibitor comprises a small molecule.

14. The method according to claim 1, wherein the ANGPTL3 inhibitor comprises an antibody.

15. The method according to claim 14, wherein the antibody comprises evinacumab.

16. The method according to claim 1, further comprising detecting the presence or absence of an ANGPTL3 predicted loss-of-function or missense variant nucleic acid molecule in a biological sample from the subject, wherein the ANGPTL3 predicted loss-of-function or missense variant nucleic acid molecule comprises a variation in the coding region.

17. The method according to claim 16, further comprising administering a kidney disease therapeutic agent in a standard dosage amount to a subject wherein the ANGPTL3 predicted loss-of-function or missense variant nucleic acid molecule is absent from the biological sample.

18. The method according to claim 16, further comprising administering a kidney disease therapeutic agent in a dosage amount that is the same as or less than a standard dosage amount to a subject that is heterozygous for the ANGPTL3 predicted loss-of-function or missense variant nucleic acid molecule.

19. The method according to claim 16, wherein the ANGPTL3 predicted loss-of-function variant nucleic acid molecule is a splice-site variant, a stop-gain variant, a start-loss variant, a stop-loss variant, a frameshift variant, an in-frame indel variant, or a variant that encodes a truncated ANGPTL3 predicted loss-of-function polypeptide.

20. A method of treating a subject with a kidney disease therapeutic agent, wherein the subject has a kidney disease or is at risk of developing a kidney disease, by administering a kidney disease therapeutic agent, the method comprising:
determining whether the subject has an Angiopoietin Like 3 (ANGPTL3) predicted loss-of-function or missense variant nucleic acid molecule, wherein the ANGPTL3 predicted loss-of-function or missense variant nucleic acid molecule comprises a variation in the coding region, by:
obtaining or having obtained a biological sample from the subject; and
performing or having performed a sequence analysis on the biological sample to determine if the subject has a genotype comprising the ANGPTL3 predicted loss-of-function or missense variant nucleic acid molecule; and
administering or continuing to administer the kidney disease therapeutic agent in a standard dosage amount to a subject that is ANGPTL3 reference, and/or administering an ANGPTL3 inhibitor to the subject;
administering or continuing to administer the kidney disease therapeutic agent in an amount that is the same as or less than a standard dosage amount to a subject that is heterozygous for the ANGPTL3 predicted loss-of-function or missense variant nucleic acid molecule, and/or administering an ANGPTL3 inhibitor to the subject; or
administering or continuing to administer the kidney disease therapeutic agent in an amount that is the same as or less than a standard dosage amount to a subject that is homozygous for the ANGPTL3 predicted loss-of-function or missense variant nucleic acid molecule;
wherein the presence of a genotype having the ANGPTL3 predicted loss-of-function or missense variant nucleic acid molecule indicates the subject has a decreased risk of developing a kidney disease; and
wherein the kidney disease is not nephrotic syndrome.

21. The method according to claim 20, wherein the kidney disease is chronic kidney disease, a kidney stone, chronic glomerulonephritis, nephronophthisis, and chronic interstitial nephritis, and nephrosclerosis.

22-26. (canceled)

27. The method according to claim 20, wherein the subject is ANGPTL3 reference, and the subject is administered or continued to be administered the kidney disease therapeutic agent in a standard dosage amount, and is administered the ANGPTL3 inhibitor.

28. The method according to claim 20, wherein the subject is heterozygous for ANGPTL3 predicted loss-of-function or missense variant nucleic acid molecule, and the subject is administered or continued to be administered the kidney disease therapeutic agent in an amount that is the same as or less than a standard dosage amount, and is administered the ANGPTL3 inhibitor.

29. The method according to claim 20, wherein the ANGPTL3 predicted loss-of-function variant nucleic acid molecule is a splice-site variant, a stop-gain variant, a start-loss variant, a stop-loss variant, a frameshift variant, an in-frame indel variant, or a variant that encodes a truncated ANGPTL3 predicted loss-of-function polypeptide.

30. The method according to claim 20, wherein the ANGPTL3 inhibitor comprises an inhibitory nucleic acid molecule that hybridizes to an ANGPTL3 nucleic acid molecule.

31. The method according to claim 30, wherein the inhibitory nucleic acid molecule comprises an antisense nucleic acid molecule, a small interfering RNA (siRNA), or a short hairpin RNA (shRNA).

32-34. (canceled)

35. The method according to claim 20, wherein the ANGPTL3 inhibitor comprises a small molecule.

36. The method according to claim 20, wherein the ANGPTL3 inhibitor comprises an antibody.

37. The method according to claim 36, wherein the antibody comprises evinacumab.

38-66. (canceled)


SEQUENCE LISTING (1367 sequences)

SEQ ID NO: 1 - DNA, 369 bp, synthetic construct
  misc_feature 1..369: note = Synthetic
  source 1..369: mol_type = other DNA
caggtgcagctggtggagtctgggggaggcgtggtccagcctgggaggtccctgagactctcctgtgcagcctctggattcaccttcagtagttatggcatgcactgggtccgccaggctccaggcaaggggctggagtgggtggcagttatatcatatgatggaagtaataaatactatgcagactccgtgaagggccgattcaccatttccagagacaattccaagaacacgctgtatctgcaaatgaacagcctgagagctgaggacacggctgtgtattactgtgcgaaagtgggagctactactttctactactactacggtatggacgtctggggccaagggaccacggtcaccgtctcctca

SEQ ID NO: 2 - protein, 123 aa, synthetic construct
  REGION 1..123: note = Synthetic
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKVGATTFYYYYGMDVWGQGTTVTVSS

SEQ ID NO: 3 - DNA, 24 bp, synthetic construct
  misc_feature 1..24: note = Synthetic
  source 1..24: mol_type = other DNA
ggattcaccttcagtagttatggc

SEQ ID NO: 4 - protein, 8 aa, synthetic construct
  REGION 1..8: note = Synthetic
GFTFSSYG

SEQ ID NO: 5 - DNA, 24 bp, synthetic construct
  misc_feature 1..24: note = Synthetic
  source 1..24: mol_type = other DNA
atatcatatgatggaagtaataaa

SEQ ID NO: 6 - protein, 8 aa, synthetic construct
  REGION 1..8: note = Synthetic
ISYDGSNK

SEQ ID NO: 7 - DNA, 48 bp, synthetic construct
  misc_feature 1..48: note = Synthetic
  source 1..48: mol_type = other DNA
gcgaaagtgggagctactactttctactactactacggtatggacgtc

SEQ ID NO: 8 - protein, 16 aa, synthetic construct
  REGION 1..16: note = Synthetic
AKVGATTFYYYYGMDV

SEQ ID NO: 9 - DNA, 321 bp, synthetic construct
  misc_feature 1..321: note = Synthetic
  source 1..321: mol_type = other DNA
gacatccagatgacccagtctccatcttccgtgtctgcatttgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaaaaggctaacagtttcccattcactttcggccctgggaccaaagtggatatcaaa

SEQ ID NO: 10 - protein, 107 aa, synthetic construct
  REGION 1..107: note = Synthetic
DIQMTQSPSSVSAFVGDRVTITCRASQGISSWLAWYQQKPGKAPKLLIYAASSLQSGVPSRFSGSGSGTDFTLTISSLQPEDFATYYCQKANSFPFTFGPGTKVEIK

SEQ ID NO: 11 - DNA, 18 bp, synthetic construct
  misc_feature 1..18: note = Synthetic
  source 1..18: mol_type = other DNA
cagggtattagcagctgg

SEQ ID NO: 12 - protein, 6 aa, synthetic construct
  REGION 1..6: note = Synthetic
QGISSW

SEQ ID NO: 13-14 - skipped (2 entries)

SEQ ID NO: 15 - DNA, 27 bp, synthetic construct
  misc_feature 1..27: note = Synthetic
  source 1..27: mol_type = other DNA
caaaaggctaacagtttcccattcact

SEQ ID NO: 16 - protein, 9 aa, synthetic construct
  REGION 1..9: note = Synthetic
QKANSFPFT

SEQ ID NO: 17 - DNA, 369 bp, synthetic construct
  misc_feature 1..369: note = Synthetic
  source 1..369: mol_type = other DNA
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcaaatgatggaagtaataaatactatgtagattccgtgaagggccgattcaccatgggcagagacaattccaagaacacgctgtatctccaaatgaacagcctgagagttgaggacacggctgtgtattactgtgcgaaaggggctggaactctttactactactactacggtatggacgtctggggccaagggaccacggtcaccgtctcctca

SEQ ID NO: 18 - protein, 123 aa, synthetic construct
  REGION 1..123: note = Synthetic
QVQLVESGGGVVQPGRSLRLSCAASGFTFSSYGMHWVRQAPGKGLEWVAVISNDGSNKYYVDSVKGRFTMGRDNSKNTLYLQMNSLRVEDTAVYYCAKGAGTLYYYYYGMDVWGQGTTVTVSS

SEQ ID NO: 19 - DNA, 24 bp, synthetic construct
  misc_feature 1..24: note = Synthetic
  source 1..24: mol_type = other DNA
ggattcaccttcagtagctatggc

SEQ ID NO: 20 - protein, 8 aa, synthetic construct
  REGION 1..8: note = Synthetic
GFTFSSYG

SEQ ID NO: 21 - DNA, 24 bp, synthetic construct
  misc_feature 1..24: note = Synthetic
  source 1..24: mol_type = other DNA
atatcaaatgatggaagtaataaa

SEQ ID NO: 22 - protein, 8 aa, synthetic construct
  REGION 1..8: note = Synthetic
ISNDGSNK

SEQ ID NO: 23 - DNA, 48 bp, synthetic construct
  misc_feature 1..48: note = Synthetic
  source 1..48: mol_type = other DNA
gcgaaaggggctggaactctttactactactactacggtatggacgtc

SEQ ID NO: 24 - protein, 16 aa, synthetic construct
  REGION 1..16: note = Synthetic
AKGAGTLYYYYYGMDV

SEQ ID NO: 25 - DNA, 321 bp, synthetic construct
  misc_feature 1..321: note = Synthetic
  source 1..321: mol_type = other DNA
gacatccagatgacccagtctccttccaccctgtctgcatctgtaggaaacagagtcaccatcacttgccgggccagtcaaagtattagtagctggttggcctggtatcaacaaaaaccagggaaagcccctaagttcctgatctataaggcgtctagtttagaaagtggggtcccatcaaggttcagcggcagtggatctgggacagaattcactctcaccatcaccagcctgcagcctgatgattttgcaacttattactgccaacagtacaatatttattcgtggacgttcggccaagggaccaaggtggaaatcaaa

SEQ ID NO: 26 - protein, 107 aa, synthetic construct
  REGION 1..107: note = Synthetic
DIQMTQSPSTLSASVGNRVTITCRASQSISSWLAWYQQKPGKAPKFLIYKASSLESGVPSRFSGSGSGTEFTLTITSLQPDDFATYYCQQYNIYSWTFGQGTKVEIK

SEQ ID NO: 27 - DNA, 18 bp, synthetic construct
  misc_feature 1..18: note = Synthetic
  source 1..18: mol_type = other DNA
caaagtattagtagctgg

SEQ ID NO: 28 - protein, 6 aa, synthetic construct
  REGION 1..6: note = Synthetic
QSISSW

SEQ ID NO: 29-30 - skipped (2 entries)

SEQ ID NO: 31 - DNA, 27 bp, synthetic construct
  misc_feature 1..27: note = Synthetic
  source 1..27: mol_type = other DNA
caacagtacaatatttattcgtggacg

SEQ ID NO: 32 - protein, 9 aa, synthetic construct
  REGION 1..9: note = Synthetic
QQYNIYSWT

SEQ ID NO: 33 - DNA, 372 bp, synthetic construct
  misc_feature 1..372: note = Synthetic
  source 1..372: mol_type = other DNA
caggtgcagctggtggagtctgggggaggcgtggtccagcctgggaggtccctgagactctcctgtgcagcctctggattcaccttcagtacctatggcatgcactgggtccgccaggctccaggcaaggggctggagtggatggcagttatatcatttgatagaggtaataaatactatgcagactccgtgaagggccgattcaccatctccagagacaattccaagaacacgctgtatctgcaaatgaacagcctgagagctgaggacacggctgtgtattactgtgcgaaaggggggggttcggggactttctactactactacggtatggacgtctggggccaagggaccacggtcaccgtctcctca

SEQ ID NO: 34 - protein, 124 aa, synthetic construct
  REGION 1..124: note = Synthetic
QVQLVQSGGGVVQPGRSLRLSCAASGFTFSTYGMHWVRQAPGKGLEWMAVISFDRGNKYYADSVKGRFTISRDNSKNTLYLQMNSLRAEDTAVYYCAKGGGSGTFYYYYGMDVWGQGTTVTVSS

SEQ ID NO: 35 - DNA, 24 bp, synthetic construct
  misc_feature 1..24: note = Synthetic
  source 1..24: mol_type = other DNA
ggattcaccttcagtacctatggc

SEQ ID NO: 36 - protein, 8 aa, synthetic construct
  REGION 1..8: note = Synthetic
GFTFSTYG

SEQ ID NO: 37 - DNA, 24 bp, synthetic construct
  misc_feature 1..24: note = Synthetic
  source 1..24: mol_type = other DNA
atatcatttgatagaggtaataaa

SEQ ID NO: 38 - protein, 8 aa, synthetic construct
  REGION 1..8: note = Synthetic
ISFDRGNK

SEQ ID NO: 39 - DNA, 51 bp, synthetic construct
  misc_feature 1..51: note = Synthetic
  source 1..51: mol_type = other DNA
gcgaaaggggggggttcggggactttctactactactacggtatggacgtc

SEQ ID NO: 40 - protein, 17 aa, synthetic construct
  REGION 1..17: note = Synthetic
AKGGGSGTFYYYYGMDV

SEQ ID NO: 41 - DNA, 324 bp, synthetic construct
  misc_feature 1..324: note = Synthetic
  source 1..324: mol_type = other DNA
gacatccagatgacccagtctccatcttccgtgtctgcatctgtaggaaacagagtcaccatcacttgtcgggcgagtcagggtattagcagctggttagcctggtatcaccagaaaccagggaaagtccctaaggtcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttcccattcactttcggccctgggaccaaagtggatatcaaacga

SEQ ID NO: 42 - protein, 108 aa, synthetic construct
  REGION 1..108: note = Synthetic
DIQMTQSPSSVSASVGNRVTITCRASQGISSWLAWYHQKPGKVPKVLIYAASSLQSGVPSRFSGSGSGTDFTLTISSLQPEDFATYYCQQANSFPFTFGPGTKVEIKR

SEQ ID NO: 43 - DNA, 18 bp, synthetic construct
  misc_feature 1..18: note = Synthetic
  source 1..18: mol_type = other DNA
cagggtattagcagctgg

SEQ ID NO: 44 - protein, 6 aa, synthetic construct
  REGION 1..6: note = Synthetic
QGISSW

SEQ ID NO: 45-46 - skipped (2 entries)

SEQ ID NO: 47 - DNA, 27 bp, synthetic construct
  misc_feature 1..27: note = Synthetic
  source 1..27: mol_type = other DNA
caacaggctaacagtttcccattcact

SEQ ID NO: 48 - protein, 9 aa, synthetic construct
  REGION 1..9: note = Synthetic
QQANSFPFT

SEQ ID NO: 49 - DNA, 375 bp, synthetic construct
  misc_feature 1..375: note = Synthetic
  source 1..375: mol_type = other DNA
caggtacagctgcagcagtcaggtccaggactggtgaaaccctcgcagaccctctcactcacctgtgccatctccggggacagtgtctctagcaacagtcctgcttggaactggatcaggcagtccccatcgagaggccttgagtggctgggaaggacatactacaggtccaagtggtataatgattatgcagtgtctgtgagaggtcgaataaccatcaacccagacacatccaataaccagttctccctacatctgaactctgtgactcccgaggacacggcgatgtattactgtgcaagagacaagggtctaacagctcgtccgacctactttgactactggggccagggaaccctggtcaccgtctcctca

SEQ ID NO: 50 - protein, 125 aa, synthetic construct
  REGION 1..125: note = Synthetic
QVQLQQSGPGLVKPSQTLSLTCAISGDSVSSNSPAWNWIRQSPSRGLEWLGRTYYRSKWYNDYAVSVRGRITINPDTSNNQFSLHLNSVTPEDTAMYYCARDKGLTARPTYFDYWGQGTLVTVSS

SEQ ID NO: 51 - DNA, 30 bp, synthetic construct
  misc_feature 1..30: note = Synthetic
  source 1..30: mol_type = other DNA
ggggacagtgtctctagcaacagtcctgct

SEQ ID NO: 52 - protein, 10 aa, synthetic construct
  REGION 1..10: note = Synthetic
GDSVSSNSPA

SEQ ID NO: 53 - DNA, 27 bp, synthetic construct
  misc_feature 1..27: note = Synthetic
  source 1..27: mol_type = other DNA
acatactacaggtccaagtggtataat

SEQ ID NO: 54 - protein, 9 aa, synthetic construct
  REGION 1..9: note = Synthetic
TYYRSKWYN

SEQ ID NO: 55 - DNA, 45 bp, synthetic construct
  misc_feature 1..45: note = Synthetic
  source 1..45: mol_type = other DNA
gcaagagacaagggtctaacagctcgtccgacctactttgactac

SEQ ID NO: 56 - protein, 15 aa, synthetic construct
  REGION 1..15: note = Synthetic
ARDKGLTARPTYFDY

SEQ ID NO: 57 - DNA, 326 bp, synthetic construct
  misc_feature 1..326: note = Synthetic
  source 1..326: mol_type = other DNA
gacatccagatgacccagtctccttccaccctgtctgcatctgtaggagacagagtcaccatcacttgccgggccagtcagagtattaattactggttggcctggtatcagcagaaaccagggaaagcccctaagccccttatctataaggcgtctagtttagaaagtggggtcccatcaaggttcagcggcagtggatctgggacagagttcactctcaccatcagcagcctgcagcctgacgattttacaacttattactgccaacagtataatagttattctccgacgttcggccaagggaccaaggtggaaatcaaacgaac

SEQ ID NO: 58 - protein, 108 aa, synthetic construct
  REGION 1..108: note = Synthetic
DIQMTQSPSTLSASVGDRVTITCRASQSINYWLAWYQQKPGKAPKPLIYKASSLESGVPSRFSGSGSGTEFTLTISSLQPDDFTTYYCQQYNSYSPTFGQGTKLEIKR

SEQ ID NO: 59 - DNA, 18 bp, synthetic construct
  misc_feature 1..18: note = Synthetic
  source 1..18: mol_type = other DNA
cagagtattaattactgg

SEQ ID NO: 60 - protein, 6 aa, synthetic construct
  REGION 1..6: note = Synthetic
QSINYW

SEQ ID NO: 61-62 - skipped (2 entries)

SEQ ID NO: 63 - DNA, 27 bp, synthetic construct
  misc_feature 1..27: note = Synthetic
  source 1..27: mol_type = other DNA
caacagtataatagttattctccgacg

SEQ ID NO: 64 - protein, 9 aa, synthetic construct
  REGION 1..9: note = Synthetic
QQYNSYSPT

SEQ ID NO: 65 - DNA, 378 bp, synthetic construct
  misc_feature 1..378: note = Synthetic
  source 1..378: mol_type = other DNA
gaggtgcagctggtggagtctgggggaggcgtgatacagcctggggggtccctgagactctcctgtgcagcctctggattcaccttcgatgattatgccatgaactgggtccgtcaaggtccagggaagggtctggagtgggtctctgccataagtggtgatggcggtagcacatactatgcagactcggtgaagggccgattcaccatctccagagacaacagcaaaaactccctgtatctgcaaatgaacagtctgagagctgaggacaccgcctttttttactgtgcaaaagatctccgtaatacgatttttggagtggttattcccgatgcttttgatatctggggccaagggacaatggtcaccgtctcttca

SEQ ID NO: 66 - protein, 126 aa, synthetic construct
  REGION 1..126: note = Synthetic
EVQLVESGGGVIQPGGSLRLSCAASGFTFDDYAMNWVRQGPGKGLEWVSAISGDGGSTYYADSVKGRFTISRDNSKNSLYLQMNSLRAEDTAFFYCAKDLRNTIFGVVIPDAFDIWGQGTMVTVSS

SEQ ID NO: 67 - DNA, 24 bp, synthetic construct
  misc_feature 1..24: note = Synthetic
  source 1..24: mol_type = other DNA
ggattcaccttcgatgattatgcc

SEQ ID NO: 68 - protein, 8 aa, synthetic construct
  REGION 1..8: note = Synthetic
GFTFDDYA

SEQ ID NO: 69 - DNA, 24 bp, synthetic construct
  misc_feature 1..24: note = Synthetic
  source 1..24: mol_type = other DNA
ataagtggtgatggcggtagcaca

SEQ ID NO: 70 - protein, 8 aa, synthetic construct
  REGION 1..8: note = Synthetic
ISGDGGST

SEQ ID NO: 71 - DNA, 57 bp, synthetic construct
  misc_feature 1..57: note = Synthetic
  source 1..57: mol_type = other DNA
gcaaaagatctccgtaatacgatttttggagtggttattcccgatgcttttgatatc

SEQ ID NO: 72 - protein, 19 aa, synthetic construct
  REGION 1..19: note = Synthetic
AKDLRNTIFGVVIPDAFDI

SEQ ID NO: 73 - DNA, 324 bp, synthetic construct
  misc_feature 1..324: note = Synthetic
  source 1..324: mol_type = other DNA
gacatccagatgacccagtctccttccaccctgtctgcatctgtaggagacagagtcaccatcacttgccgggccagtcagagcattaggagctggttggcctggtatcagcagaaaccagggaaagcccctaaactcctgatctataaggcgtctagtttagaaagtggggtcccatcaaggttcagcggcagtggatctgggacagaattcactctcaccatcagcagcctgcagcctgatgattttgcaacttattactgccaacaatataatagttattcgtacacttttggccaggggaccaagctggagatcaaacga

SEQ ID NO: 74 - protein, 108 aa, synthetic construct
  REGION 1..108: note = Synthetic
DIQMTQSPSTLSASVGDRVTITCRASQSIRSWLAWYQQKPGKAPKLLIYKASSLESGVPSRFSGSGSGTEFTLTISSLQPDDFATYYCQQYNSYSYTFGQGTKLEIKR

SEQ ID NO: 75 - DNA, 18 bp, synthetic construct
  misc_feature 1..18: note = Synthetic
  source 1..18: mol_type = other DNA
cagagcattaggagctgg

SEQ ID NO: 76 - protein, 6 aa, synthetic construct
  REGION 1..6: note = Synthetic
QSIRSW

SEQ ID NO: 77-78 - skipped (2 entries)

SEQ ID NO: 79 - DNA, 27 bp, synthetic construct
  misc_feature 1..27: note = Synthetic
  source 1..27: mol_type = other DNA
caacaatataatagttattcgtacact

SEQ ID NO: 80 - protein, 9 aa, synthetic construct
  REGION 1..9: note = Synthetic
QQYNSYSYT

SEQ ID NO: 81 - DNA, 354 bp, synthetic construct
  misc_feature 1..354: note = Synthetic
  source 1..354: mol_type = other DNA
caggtgcagctggtggagtcggggggaggcttggtcaagcctggagggtccctgagactctcctgtgcagcctctggattcactttcagtgactactacatgagctggatccgtcaggctccagggaaggggctggagtgggtttcatacattggtagtagtggtgtcaacatgtactacgcagactctgtgaagggccgattcaccatctccagggacaacgccaagaattcattatatctggaaatgaacagcctgagagccgaggacacggctgtttattactgtgcgagagactcttcccaactgggttttgactactggggccagggaaccctggtcaccgtctcctca

SEQ ID NO: 82 - protein, 118 aa, synthetic construct
  REGION 1..118: note = Synthetic
QVQLVESGGGLVKPGGSLRLSCAASGFTFSDYYMSWIRQAPGKGLEWVSYIGSSGVNMYYADSVKGRFTISRDNAKNSLYLEMNSLRAEDTAVYYCARDSSQLGFDYWGQGTLVTVSS

SEQ ID NO: 83 - DNA, 24 bp, synthetic construct
  misc_feature 1..24: note = Synthetic
  source 1..24: mol_type = other DNA
ggattcactttcagtgactactac

SEQ ID NO: 84 - protein, 8 aa, synthetic construct
  REGION 1..8: note = Synthetic
GFTFSDYY

SEQ ID NO: 85 - DNA, 24 bp, synthetic construct
  misc_feature 1..24: note = Synthetic
  source 1..24: mol_type = other DNA
attggtagtagtggtgtcaacatg

SEQ ID NO: 86 - protein, 8 aa, synthetic construct
  REGION 1..8: note = Synthetic
IGSSGVNM

SEQ ID NO: 87 - DNA, 33 bp, synthetic construct
  misc_feature 1..33: note = Synthetic
  source 1..33: mol_type = other DNA
gcgagagactcttcccaactgggttttgactac

SEQ ID NO: 88 - protein, 11 aa, synthetic construct
  REGION 1..11: note = Synthetic
ARDSSQLGFDY

SEQ ID NO: 89 - DNA, 324 bp, synthetic construct
  misc_feature 1..324: note = Synthetic
  source 1..324: mol_type = other DNA
gacatccagatgacccagtctccatcctccctgtctgcatctgtaggagacagagtcaccatcacttgccggacaagtcagaatattatcaactttttaaattggtatcaacagaaacctgggaaggcccctaaactcctgatctatactacttccactttacaaagtggggtcccatcaaggttcagtggcagtggatctgggacagatttcactctctccatcaatagtctacaacctgaagattttgcaacttacttctgtcaacagacttacagtaatccactcactttcggcggagggaccaaggtggagatcaaacga

SEQ ID NO: 90 - protein, 108 aa, synthetic construct
  REGION 1..108: note = Synthetic
DIQMTQSPSSLSASVGDRVTITCRTSQNIINFLNWYQQKPGKAPKLLIYTTSTLQSGVPSRFSGSGSGTDFTLSINSLQPEDFATYFCQQTYSNPLTFGGGTKLEIKR

SEQ ID NO: 91 - DNA, 18 bp, synthetic construct
  misc_feature 1..18: note = Synthetic
  source 1..18: mol_type = other DNA
cagaatattatcaacttt

SEQ ID NO: 92 - protein, 6 aa, synthetic construct
  REGION 1..6: note = Synthetic
QNIINF

SEQ ID NO: 93-94 - skipped (2 entries)

SEQ ID NO: 95 - DNA, 27 bp, synthetic construct
  misc_feature 1..27: note = Synthetic
  source 1..27: mol_type = other DNA
caacagacttacagtaatccactcact

SEQ ID NO: 96 - protein, 9 aa, synthetic construct
  REGION 1..9: note = Synthetic
QQTYSNPLT

SEQ ID NO: 97 - DNA, 360 bp, synthetic construct
  misc_feature 1..360: note = Synthetic
  source 1..360: mol_type = other DNA
gaggtgcagctggtgcagtctgggggaggcgtggtccagcctgggaggtccctgagactctcctgtgcagcgtctggattcaccttcagtaattatggcatgcactgggtccgccaggctccaggcaaggggctggagtgggtggcagttgtttggtatgatggagataataaatactatgcagactccgtgaagggccgattcaccatctccagagacaattccaagaacacgctgtatctgcaaatgaacagcctgagagccgaggacacggctgtgtattactgtgcgagagatattatcacatctcgcccgactttggactactggggccagggaaccctggtcactgtctcctca

SEQ ID NO: 98 - protein, 120 aa, synthetic construct
  REGION 1..120: note = Synthetic
EVQLVQSGGGVVQPGRSLRLSCAASGFTFSNYGMHWVRQAPGKGLEWVAVVWYDGDNKYYADSVKGRFTISRDNSKNTLYLQMNSLRAEDTAVYYCARDIITSRPTLDYWGQGTLVTVSS

SEQ ID NO: 99 - DNA, 24 bp, synthetic construct
  misc_feature 1..24: note = Synthetic
  source 1..24: mol_type = other DNA
ggattcaccttcagtaattatggc

SEQ ID NO: 100 - protein, 8 aa, synthetic construct
  REGION 1..8: note = Synthetic
GFTFSNYG

SEQ ID NO: 101 - DNA, 24 bp, synthetic construct
  misc_feature 1..24: note = Synthetic
  source 1..24: mol_type = other DNA
gtttggtatgatggagataataaa

SEQ ID NO: 102 - protein, 8 aa, synthetic construct
  REGION 1..8: note = Synthetic
VWYDGDNK

SEQ ID NO: 103 - DNA, 39 bp, synthetic construct
  misc_feature 1..39: note = Synthetic
  source 1..39: mol_type = other DNA
gcgagagatattatcacatctcgcccgactttggactac

SEQ ID NO: 104 - protein, 13 aa, synthetic construct
  REGION 1..13: note = Synthetic
ARDIITSRPTLDY

SEQ ID NO: 105 - DNA, 324 bp, synthetic construct
  misc_feature 1..324: note = Synthetic
  source 1..324: mol_type = other DNA
gacatccagttgacccagtctccatccttcctgtctgcatctgtaggagacagagtcaccatcacttgctgggccagtcagggcattaacagttatttagcctggtatcagcaaaaaccagggaaagcccctaagctcctgatctatcctgcatccactttgcaaagtggggtcccatcaaggttcagcggcagtggatctgggacagaattcactctcacaatcagcagcctgcagcctgtagattttgcaacttattactgtcaacagcttaatagttacccgctcactttcggcggagggaccaaggtggagatcaaacga

SEQ ID NO: 106 - protein, 108 aa, synthetic construct
  REGION 1..108: note = Synthetic
DIQLTQSPSFLSASVGDRVTITCWASQGINSYLAWYQQKPGKAPKLLIYPASTLQSGVPSRFSGSGSGTEFTLTISSLQPVDFATYYCQQLNSYPLTFGGGTKVEIKR

SEQ ID NO: 107 - DNA, 18 bp, synthetic construct
  misc_feature 1..18: note = Synthetic
  source 1..18: mol_type = other DNA
cagggcattaacagttat

SEQ ID NO: 108 - protein, 6 aa, synthetic construct
  REGION 1..6: note = Synthetic
QGINSY

SEQ ID NO: 109-110 - skipped (2 entries)

SEQ ID NO: 111 - DNA, 27 bp, synthetic construct
  misc_feature 1..27: note = Synthetic
  source 1..27: mol_type = other DNA
caacagcttaatagttacccgctcact

SEQ ID NO: 112 - protein, 9 aa, synthetic construct
  REGION 1..9: note = Synthetic
QQLNSYPLT

SEQ ID NO: 113 - DNA, 354 bp, synthetic construct
  misc_feature 1..354: note = Synthetic
  source 1..354: mol_type = other DNA
caggtgcagctggtggagtcggggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatatccagggacaacgccaagaactcactgtatctgcaaatgaacagcctgagagccgaggacacggccgtgtattactgtgcgagagattcttcccaactgggttttgactactggggccagggaaccctggtcaccgtctcctca

SEQ ID NO: 114 - protein, 118 aa, synthetic construct
  REGION 1..118: note = Synthetic
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDSSQLGFDYWGQGTLVTVSS

SEQ ID NO: 115 - DNA, 24 bp, synthetic construct
  misc_feature 1..24: note = Synthetic
  source 1..24: mol_type = other DNA
ggattcaccttcagtgactactac

SEQ ID NO: 116 - protein, 8 aa, synthetic construct
  REGION 1..8: note = Synthetic
GFTFSDYY

SEQ ID NO: 117 - DNA, 24 bp, synthetic construct
  misc_feature 1..24: note = Synthetic
  source 1..24: mol_type = other DNA
attagtagtagtggtagtaccata

SEQ ID NO: 118 - protein, 8 aa, synthetic construct
  REGION 1..8: note = Synthetic
ISSSGSTI

SEQ ID NO: 119 - DNA, 33 bp, synthetic construct
  misc_feature 1..33: note = Synthetic
  source 1..33: mol_type = other DNA
gcgagagattcttcccaactgggttttgactac

SEQ ID NO: 120 - protein, 11 aa, synthetic construct
  REGION 1..11: note = Synthetic
ARDSSQLGFDY

SEQ ID NO: 121 - DNA, 324 bp, synthetic construct
  misc_feature 1..324: note = Synthetic
  source 1..324: mol_type = other DNA
gacatccagatgacccagtctccatcctccctgtctgcatctgtaggagacagagtcaccatcacttgccgggcaagtcagagcattatcagctttttaaattggtatcagcagaaaccagggaaggcccctaagctcctgatctatactgcatccagtttgcaaagtggggtcccatcaaggttcagtggcagtggatctgggacagatttcactctcaccatcagcagtctacaacctgaagattttgcaacttactactgtcaacagacttacagtaatccgctcactttcggcggagggaccaaggtggaaatcaaacga

SEQ ID NO: 122 - protein, 108 aa, synthetic construct
  REGION 1..108: note = Synthetic
DIQMTQSPSSLSASVGDRVTITCRASQSIISFLNWYQQKPGKAPKLLIYTASSLQSGVPSRFSGSGSGTDFTLTISSLQPEDFATYYCQQTYSNPLTFGGGTKVEIKR

SEQ ID NO: 123 - DNA, 18 bp, synthetic construct
  misc_feature 1..18: note = Synthetic
  source 1..18: mol_type = other DNA
cagagcattatcagcttt

SEQ ID NO: 124 - protein, 6 aa, synthetic construct
  REGION 1..6: note = Synthetic
QSIISF

SEQ ID NO: 125-126 - skipped (2 entries)

SEQ ID NO: 127 - DNA, 27 bp, synthetic construct
  misc_feature 1..27: note = Synthetic
  source 1..27: mol_type = other DNA
caacagacttacagtaatccgctcact

SEQ ID NO: 128 - protein, 9 aa, synthetic construct
  REGION 1..9: note = Synthetic
QQTYSNPLT

SEQ ID NO: 129 - DNA, 342 bp, synthetic construct
  misc_feature 1..342: note = Synthetic
  source 1..342: mol_type = other DNA
caggtgcagctggtacagtctggacctgaggtgaagaagcctggggcctcagtgagggtctcctgtaaggcttctggttaccttagtgactttattatcacctgggtgcgacaggcccctggacaagggcttgagtggatgggatggatcagcacttacagtggtgacacagactctgcaccgaagttccagggcagagtcaccatgaccacagacacatccacgactacagtcttcttggaactgaggagcctgagatctgacgacacggccgtgtattattgtgcgagagggcggctgtttgactactggggccagggaaccctggtcaccgtctcctca

SEQ ID NO: 130 - protein, 114 aa, synthetic construct
  REGION 1..114: note = Synthetic
QVQLVQSGPEVKKPGASVRVSCKASGYLSDFIITWVRQAPGQGLEWMGWISTYSGDTDSAPKFQGRVTMTTDTSTTTVFLELRSLRSDDTAVYYCARGRLFDYWGQGTLVTVSS

SEQ ID NO: 131 - DNA, 21 bp, synthetic construct
  misc_feature 1..21: note = Synthetic
  source 1..21: mol_type = other DNA
ggttaccttagtgactttatt

SEQ ID NO: 132 - protein, 7 aa, synthetic construct
  REGION 1..7: note = Synthetic
GYLSDFI

SEQ ID NO: 133 - DNA, 24 bp, synthetic construct
  misc_feature 1..24: note = Synthetic
  source 1..24: mol_type = other DNA
atcagcacttacagtggtgacaca

SEQ ID NO: 134 - protein, 8 aa, synthetic construct
  REGION 1..8: note = Synthetic
ISTYSGDT

SEQ ID NO: 135 - DNA, 24 bp, synthetic construct
  misc_feature 1..24: note = Synthetic
  source 1..24: mol_type = other DNA
gcgagagggcggctgtttgactac

SEQ ID NO: 136 - protein, 8 aa, synthetic construct
  REGION 1..8: note = Synthetic
ARGRLFDY

SEQ ID NO: 137 - DNA, 339 bp, synthetic construct
  misc_feature 1..339: note = Synthetic
  source 1..339: mol_type = other DNA
gatgttgtgatgactcagtctccactctccctgcccgtcacccttggacagccggcctccatctcctgcaggtctagtcaaagcctcgtatacagtgatggaaacacctacttgaattggtttcaacagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggccgtacacttttggccaggggaccaagctggagatcaaacga

SEQ ID NO: 138 - protein, 113 aa, synthetic construct
  REGION 1..113: note = Synthetic
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPYTFGQGTKLEIKR

SEQ ID NO: 139 - DNA, 33 bp, synthetic construct
  misc_feature 1..33: note = Synthetic
  source 1..33: mol_type = other DNA
caaagcctcgtatacagtgatggaaacacctac

SEQ ID NO: 140 - protein, 11 aa, synthetic construct
  REGION 1..11: note = Synthetic
QSLVYSDGNTY

SEQ ID NO: 141-142 - skipped (2 entries)

SEQ ID NO: 143 - DNA, 27 bp, synthetic construct
  misc_feature 1..27: note = Synthetic
  source 1..27: mol_type = other DNA
atgcaaggtacacactggccgtacact

SEQ ID NO: 144 - protein, 9 aa, synthetic construct
  REGION 1..9: note = Synthetic
MQGTHWPYT

SEQ ID NO: 145 - DNA, 354 bp, synthetic construct
  misc_feature 1..354: note = Synthetic
  source 1..354: mol_type = other DNA
caggtgcagctggtggagtctgggggagtctcggtcaagcctggagggtccctgcgactctcctgtgcagcctctggattcaccttcagtgactactacatgagctggatccgccaggcgccagggaagggactggagtgggtttcgtacattggtagtagtggtactaatgactactacgcagactctgtgaagggccgattcaccatctccagggacaacgccaagaactcactgtatcttcaaatggacagcctgagagccgaggacacggccgtctattactgtgcgagagattcttcccaaatgggttttgactactggggccagggaaccctggtcactgtctcctca

SEQ ID NO: 146 - protein, 118 aa, synthetic construct
  REGION 1..118: note = Synthetic
QVQLVESGGVSVKPGGSLRLSCAASGFTFSDYYMSWIRQAPGKGLEWVSYIGSSGTNDYYADSVKGRFTISRDNAKNSLYLQMDSLRAEDTAVYYCARDSSQMGFDYWGQGTLVTVSS

SEQ ID NO: 147 - DNA, 24 bp, synthetic construct
  misc_feature 1..24: note = Synthetic
  source 1..24: mol_type = other DNA
ggattcaccttcagtgactactac

SEQ ID NO: 148 - protein, 8 aa, synthetic construct
  REGION 1..8: note = Synthetic
GFTFSDYY

SEQ ID NO: 149 - DNA, 24 bp, synthetic construct
  misc_feature 1..24: note = Synthetic
  source 1..24: mol_type = other DNA
attggtagtagtggtactaatgac

SEQ ID NO: 150 - protein, 8 aa, synthetic construct
  REGION 1..8: note = Synthetic
IGSSGTND

SEQ ID NO: 151 - DNA, 33 bp, synthetic construct
  misc_feature 1..33: note = Synthetic
  source 1..33: mol_type = other DNA
gcgagagattcttcccaaatgggttttgactac

SEQ ID NO: 152 - protein, 11 aa, synthetic construct
  REGION 1..11: note = Synthetic
ARDSSQMGFDY

SEQ ID NO: 153 - DNA, 324 bp, synthetic construct
  misc_feature 1..324: note = Synthetic
  source 1..324: mol_type = other DNA
gacatccagatgacccagtctccatcctccctgtctgcttctgtgggagacagagtcaccatcacttgccgggcaagtcagaacattatcaactttttaaattggtatcagcagagaccagggaaagcccctcagctcctgatctatgttgcagccagcttgcagagtggggtcccatcaaggttcactggcagtggatatgggacagatttcactctcaccatcagcagtctgcaacctgaggatttcgcaacttactactgtcaacagacttacactaacccgctcactttcggcggagggaccaaggtggagatcaaacga

SEQ ID NO: 154 - protein, 108 aa, synthetic construct
  REGION 1..108: note = Synthetic
DIQMTQSPSSLSASVGDRVTITCRASQNIINFLNWYQQRPGKAPQLLIYVAASLQSGVPSRFTGSGYGTDFTLTISSLQPEDFATYYCQQTYTNPLTFGGGTKVEIKR

SEQ ID NO: 155 - DNA, 18 bp, synthetic construct
  misc_feature 1..18: note = Synthetic
  source 1..18: mol_type = other DNA
cagaacattatcaacttt

SEQ ID NO: 156 - protein, 6 aa, synthetic construct
  REGION 1..6: note = Synthetic
QNIINF

SEQ ID NO: 157-158 - skipped (2 entries)

SEQ ID NO: 159 - DNA, 27 bp, synthetic construct
  misc_feature 1..27: note = Synthetic
  source 1..27: mol_type = other DNA
caacagacttacactaacccgctcact

SEQ ID NO: 160 - protein, 9 aa, synthetic construct
  REGION 1..9: note = Synthetic
QQTYTNPLT

SEQ ID NO: 161 - protein, 460 aa, Homo sapiens
MFTIKLLLFIVPLVISSRIDQDNSSFDSLSPEPKSRFAMLDDVKILANGLLQLGHGLKDFVHKTKGQINDIFQKLNIFDQSFYDLSLQTSEIKEEEKELRRTTYKLQVKNEEVKNMSLELNSKLESLLEEKILLQQKVKYLEEQLTNLIQNQPETPEHPEVTSLKTFVEKQDNSIKDLLQTVEDQYKQLNQQHSQIKEIENQLRRTSIQEPTEISLSSKPRAPRTTPFLQLNEIRNVKHDGIPAECTTIYNRGEHTSGMYAIRPSNSQVFHVYCDVISGSPWTLIQHRIDGSQNFNETWENYKYGFGRLDGEFWLGLEKIYSIVKQSNYVLRIELEDWKDNKHYIEYSFYLGNHETNYTLHLVAITGNVPNAIPENKDLVFSTWDHKAKGHFNCPEGYSGGWWWHDECGENNLNGKYNKPRAKSKPERRRGLSWKSQNGRLYSIKSTKMLIHPTDSESFE

SEQ ID NO: 162 - protein, 26 aa, synthetic construct
  REGION 1..26: note = Synthetic
EPKSRFAMLDDVKILANGLLQLGHGL

SEQ ID NO: 163 - DNA, 363 bp, synthetic construct
  misc_feature 1..363: note = Synthetic
  source 1..363: mol_type = other DNA
gaggtgcagcttttggagtctgggggaggcttggtacagcctggggggtccctgagactctcctgtgcagcctctggattcacctttagcacctatgccatgagctgggtccgccaggctccagggaaggggctggagggggtctcaggtattagtggtactggttatagaacatactacgcagactccgtgaagggccgattcaccatctccagagacaattccaagaactcgctgtatctgcaaatgaacagcctgagagccgaggacacggccgtatattactgtgcgaaagatcggggcttactatggttcggggaattaacctactggggccagggaaccctggtcaccgtctcctca

SEQ ID NO: 164 - protein, 121 aa, synthetic construct
  REGION 1..121: note = Synthetic
EVQLLESGGGLVQPGGSLRLSCAASGFTFSTYAMSWVRQAPGKGLEGVSGISGTGYRTYYADSVKGRFTISRDNSKNSLYLQMNSLRAEDTAVYYCAKDRGLLWFGELTYWGQGTLVTVSS

SEQ ID NO: 165 - DNA, 24 bp, synthetic construct
  misc_feature 1..24: note = Synthetic
  source 1..24: mol_type = other DNA
ggattcacctttagcacctatgcc

SEQ ID NO: 166 - protein, 8 aa, synthetic construct
  REGION 1..8: note = Synthetic
GFTFSTYA

SEQ ID NO: 167 - DNA, 24 bp, synthetic construct
  misc_feature 1..24: note = Synthetic
  source 1..24: mol_type = other DNA
attagtggtactggttatagaaca

SEQ ID NO: 168 - protein, 8 aa, synthetic construct
  REGION 1..8: note = Synthetic
ISGTGYRT

SEQ ID NO: 169 - DNA, 42 bp, synthetic construct
  misc_feature 1..42: note = Synthetic
  source 1..42: mol_type = other DNA
gcgaaagatcggggcttactatggttcggggaattaacctac

SEQ ID NO: 170 - protein, 14 aa, synthetic construct
  REGION 1..14: note = Synthetic
AKDRGLLWFGELTY

SEQ ID NO: 171 - DNA, 318 bp, synthetic construct
  misc_feature 1..318: note = Synthetic
  source 1..318: mol_type = other DNA
gacatccagatgacccagtctccttccaccctgtctgcatctgtaggagacagagtcaccatcacttgccgggccagtcagagtattaataactggttggcctggtatcaacagaaaccagggaaggcccctaacctcctgatctataaggcgtctagtttagaaagtggggtcccatcaaggttcagcggcagtggatctgggacagaattcactctcaccatcagcagcctgcagcctgatgattttgcaacttattactgccaacaatataatgattattggacgttcggccaagggaccaaggtggaaatcaaa

SEQ ID NO: 172 - protein, 106 aa, synthetic construct
  REGION 1..106: note = Synthetic
DIQMTQSPSTLSASVGDRVTITCRASQSINNWLAWYQQKPGKAPNLLIYKASSLESGVPSRFSGSGSGTEFTLTISSLQPDDFATYYCQQYNDYWTFGQGTKVEIK

SEQ ID NO: 173 - DNA, 18 bp, synthetic construct
  misc_feature 1..18: note = Synthetic
  source 1..18: mol_type = other DNA
cagagtattaataactgg

SEQ ID NO: 174 - protein, 6 aa, synthetic construct
  REGION 1..6: note = Synthetic
QSINNW

SEQ ID NO: 175-176 - skipped (2 entries)

SEQ ID NO: 177 - DNA, 24 bp, synthetic construct
  misc_feature 1..24: note = Synthetic
  source 1..24: mol_type = other DNA
caacaatataatgattattggacg

SEQ ID NO: 178 - protein, 8 aa, synthetic construct
  REGION 1..8: note = Synthetic
QQYNDYWT

SEQ ID NO: 179 - RNA, 20 nt, synthetic construct
  misc_feature 1..20: note = antisense sequence
  source 1..20: mol_type = other RNA
acgtggaactgttttcttct

SEQ ID NO: 180 - RNA, 20 nt, synthetic construct
  misc_feature 1..20: note = antisense sequence
  source 1..20: mol_type = other RNA
aacgtggaactgttttcttc

SEQ ID NO: 181 - RNA, 20 nt, synthetic construct
  misc_feature 1..20: note = antisense sequence
  source 1..20: mol_type = other RNA
caacgtggaactgttttctt

SEQ ID NO: 182 - RNA, 20 nt, synthetic construct
  misc_feature 1..20: note = antisense sequence
  source 1..20: mol_type = other RNA
gcaacgtggaactgttttct

SEQ ID NO: 183 - RNA, 20 nt, synthetic construct
  misc_feature 1..20: note = antisense sequence
  source 1..20: mol_type = other RNA
aagcaacgtggaactgtttt

SEQ ID NO: 184 - RNA, 20 nt, synthetic construct
  misc_feature 1..20: note = antisense sequence
  source 1..20: mol_type = other RNA
caagcaacgtggaactgttt

SEQ ID NO: 185 - RNA, 20 nt, synthetic construct
  misc_feature 1..20: note = antisense sequence
  source 1..20: mol_type = other RNA
tcaagcaacgtggaactgtt

SEQ ID NO: 186 - RNA, 20 nt, synthetic construct
  misc_feature 1..20: note = antisense sequence
  source 1..20: mol_type = other RNA
tttcaagcaacgtggaactg

SEQ ID NO: 187 - RNA, 20 nt, synthetic construct
  misc_feature 1..20: note = antisense sequence
  source 1..20: mol_type = other RNA
atttcaagcaacgtggaact

SEQ ID NO: 188 - RNA, 20 nt, synthetic construct
  misc_feature 1..20: note = antisense sequence
  source 1..20: mol_type = other RNA
aatttcaagcaacgtggaac

SEQ ID NO: 189 - RNA, 20 nt, synthetic construct
  misc_feature 1..20: note = antisense sequence
  source 1..20: mol_type = other RNA
caatttcaagcaacgtggaa

SEQ ID NO: 190 - RNA, 20 nt, synthetic construct
  misc_feature 1..20: note = antisense sequence
  source 1..20: mol_type = other RNA
tcaatttcaagcaacgtgga

SEQ ID NO: 191 - RNA, 20 nt, synthetic construct
  misc_feature 1..20: note = antisense sequence
  source 1..20: mol_type = other RNA
tctggaggaaataactagag

SEQ ID NO: 192 - RNA, 20 nt, synthetic construct
  misc_feature 1..20: note = antisense sequence
  source 1..20: mol_type = other RNA
gattttggctctggagatag

SEQ ID NO: 193 - RNA, 20 nt, synthetic construct
  misc_feature 1..20: note = antisense sequence
  source 1..20: mol_type = other RNA
tgattttggctctggagata

SEQ ID NO: 194 - RNA, 20 nt, synthetic construct
  misc_feature 1..20: note = antisense sequence
  source 1..20: mol_type = other RNA
ttgattttggctctggagat

SEQ ID NO: 195 - RNA, 20 nt, synthetic construct
  misc_feature 1..20: note = antisense sequence
  source 1..20: mol_type = other RNA
aaggaggccattggctaaaa

SEQ ID NO: 196 - RNA, 20 nt, synthetic construct
  misc_feature 1..20: note = antisense sequence
  source 1..20: mol_type = other RNA
gaaggaggccattggctaaa

SEQ ID NO: 197 - RNA, 20 nt, synthetic construct
  misc_feature 1..20: note = antisense sequence
  source 1..20: mol_type = other RNA
ctgaaggaggccattggcta

SEQ ID NO: 198 - RNA, 20 nt, synthetic construct
  misc_feature 1..20: note = antisense sequence
  source 1..20: mol_type = other RNA
aactgaaggaggccattggc

SEQ ID NO: 199 - RNA, 20 nt, synthetic construct
  misc_feature 1..20: note = antisense sequence
  source 1..20: mol_type = other RNA
gtcccaactgaaggaggcca

SEQ ID NO: 200 - RNA, 20 nt, synthetic construct
  misc_feature 1..20: note = antisense sequence
  source 1..20: mol_type = other RNA
atgtcccaactgaaggaggc

SEQ ID NO: 201 - RNA, 20 nt, synthetic construct
  misc_feature 1..20: note = antisense sequence
  source 1..20: mol_type = other RNA
gaccatgtcccaactgaagg

SEQ ID NO: 202 - RNA, 20 nt, synthetic construct
  misc_feature 1..20: note = antisense sequence
  source 1..20: mol_type = other RNA
agaccatgtcccaactgaag

SEQ ID NO: 203 - RNA, 20 nt, synthetic construct
  misc_feature 1..20: note = antisense sequence
  source 1..20: mol_type = other RNA
aagaccatgtcccaactgaa

SEQ ID NO: 204 - RNA, 20 nt, synthetic construct
  misc_feature 1..20: note = antisense sequence
  source 1..20: mol_type = other RNA
taagaccatgtcccaactga

SEQ ID NO: 205 - RNA, 20 nt, synthetic construct
  misc_feature 1..20: note = antisense sequence
  source 1..20: mol_type = other RNA
ttaagaccatgtcccaactg

SEQ ID NO: 206 - RNA, 20 nt, synthetic construct
  misc_feature 1..20: note = antisense sequence
  source 1..20: mol_type = other RNA
tttaagaccatgtcccaact

SEQ ID NO: 207 - RNA, 20 nt, synthetic construct
  misc_feature 1..20: note = antisense sequence
  source 1..20: mol_type = other RNA
ctttaagaccatgtcccaac

SEQ ID NO: 208 - RNA, 20 nt, synthetic construct
  misc_feature 1..20: note = antisense sequence
  source 1..20: mol_type = other RNA
tctttaagaccatgtcccaa

SEQ ID NO: 209 - RNA, 20 nt, synthetic construct
  misc_feature 1..20: note = antisense sequence
  source 1..20: mol_type = other RNA
cgtcttatggacaaagtctt

SEQ ID NO: 210 - RNA, 20 nt, synthetic construct
  misc_feature 1..20: note = antisense sequence
  source 1..20: mol_type = other RNA
tcgtcttatggacaaagtct

SEQ ID NO: 211 - RNA, 20 nt, synthetic construct
  misc_feature 1..20: note = antisense sequence
  source 1..20: mol_type = other RNA
ttcgtcttatggacaaagtc

SEQ ID NO: 212 - RNA, 20 nt, synthetic construct
  misc_feature 1..20: note = antisense sequence
  source 1..20: mol_type = other RNA
cttcgtcttatggacaaagt

SEQ ID NO: 213 - RNA, 20 nt, synthetic construct
  misc_feature 1..20: note = antisense sequence
  source 1..20: mol_type = other RNA
ccttcgtcttatggacaaag

SEQ ID NO: 214 - RNA, 20 nt, synthetic construct
  misc_feature 1..20: note = antisense sequence
  source 1..20: mol_type = other RNA
cccttcgtcttatggacaaa

SEQ ID NO: 215 - RNA, 20 nt, synthetic construct
  misc_feature 1..20: note = antisense sequence
  source 1..20: mol_type = other RNA
gcccttcgtcttatggacaa

SEQ ID NO: 216 - RNA, 20 nt, synthetic construct
  misc_feature 1..20: note = antisense sequence
  source 1..20: mol_type = other RNA
ggcccttcgtcttatggaca

SEQ ID NO: 217 - RNA, 20 nt, synthetic construct
  misc_feature 1..20: note = antisense sequence
  source 1..20: mol_type = other RNA
tggcccttcgtcttatggac

SEQ ID NO: 218 - RNA, 20 nt, synthetic construct
  misc_feature 1..20: note = antisense sequence
  source 1..20: mol_type = other RNA
ttggcccttcgtcttatgga

SEQ ID NO: 219 - RNA, 20 nt, synthetic construct
  misc_feature 1..20: note = antisense sequence
  source 1..20: mol_type = other RNA
tttggcccttcgtcttatgg

SEQ ID NO: 220 - RNA, 20 nt, synthetic construct
  misc_feature 1..20: note = antisense sequence
  source 1..20: mol_type = other RNA
atttggcccttcgtcttatg

SEQ ID NO: 221 - RNA, 20 nt, synthetic construct
  misc_feature 1..20: note = antisense sequence
  source 1..20: mol_type = other RNA
aatttggcccttcgtcttat

SEQ ID NO: 222 - RNA, 20 nt, synthetic construct
  misc_feature 1..20: note = antisense sequence
  source 1..20: mol_type = other RNA
taatttggcccttcgtctta

SEQ ID NO: 223 - RNA, 20 nt, synthetic construct
  misc_feature 1..20: note = antisense sequence
  source 1..20: mol_type = other RNA
ttaatttggcccttcgtctt

SEQ ID NO: 224 - RNA, 20 nt, synthetic construct
  misc_feature 1..20: note = antisense sequence
  source 1..20: mol_type = other RNA
gtttgcagcgatagatcata

SEQ ID NO: 225 - RNA, 20 nt, synthetic construct
  misc_feature 1..20: note = antisense sequence
  source 1..20: mol_type = other RNA
ggtttgcagcgatagatcat

SEQ ID NO: 226 - RNA, 20 nt, synthetic construct
  misc_feature 1..20: note = antisense sequence
  source 1..20: mol_type = other RNA
tggtttgcagcgatagatca

SEQ ID NO: 227 - RNA, 20 nt, synthetic construct
  misc_feature 1..20: note = antisense sequence
  source 1..20: mol_type = other RNA
ctggtttgcagcgatagatc

SEQ ID NO: 228 - RNA, 20 nt, synthetic construct
  misc_feature 1..20: note = antisense sequence
  source 1..20: mol_type = other RNA
actggtttgcagcgatagat

SEQ ID NO: 229 - RNA, 20 nt, synthetic construct
  misc_feature 1..20: note = antisense sequence
  source 1..20: mol_type = other RNA
cactggtttgcagcgataga

SEQ ID NO: 230 - RNA, 20 nt, synthetic construct
  misc_feature 1..20: note = antisense sequence
  source 1..20: mol_type = other RNA
tcactggtttgcagcgatag

SEQ ID NO: 231 - RNA, 20 nt, synthetic construct
  misc_feature 1..20: note = antisense sequence
  source 1..20: mol_type = other RNA
ttcactggtttgcagcgata

SEQ ID NO: 232 - RNA, 20 nt, synthetic construct
  misc_feature 1..20: note = antisense sequence
  source 1..20: mol_type = other RNA
atttcactggtttgcagcga

SEQ ID NO: 233 - RNA, 20 nt, synthetic construct
  misc_feature 1..20: note = antisense sequence
  source 1..20: mol_type = other RNA
gatttcactggtttgcagcg

SEQ ID NO: 234 - RNA, 20 nt, synthetic construct
  misc_feature 1..20: note = antisense sequence
  source 1..20: mol_type = other RNA
ttgatttcactggtttgcag

SEQ ID NO: 235 - RNA, 20 nt, synthetic construct
  misc_feature 1..20: note = antisense sequence
  source 1..20: mol_type = other RNA
ctcagttccttttcttcttc

SEQ ID NO: 236 - RNA, 20 nt, synthetic construct
  misc_feature 1..20: note = antisense sequence
  source 1..20: mol_type = other RNA
gagttgagttcaagtgacat

SEQ ID NO: 237 - RNA, 20 nt, synthetic construct
  misc_feature 1..20: note = antisense sequence
  source 1..20: mol_type = other RNA
tgagttgagttcaagtgaca

SEQ ID NO: 238 - RNA, 20 nt, synthetic construct
  misc_feature 1..20: note = antisense sequence
  source 1..20: mol_type = other RNA
ctggagtttcaggttgattt

SEQ ID NO: 239 - RNA, 20 nt, synthetic construct
  misc_feature 1..20: note = antisense sequence
  source 1..20: mol_type = other RNA
tctggagtttcaggttgatt

SEQ ID NO: 240 - RNA, 20 nt, synthetic construct
  misc_feature 1..20: note = antisense sequence
  source 1..20: mol_type = other RNA
gttctggagtttcaggttga

SEQ ID NO: 241 - RNA, 20 nt, synthetic construct
  misc_feature 1..20: note = antisense sequence
  source 1..20: mol_type = other RNA
ggtgttctggagtttcaggt

SEQ ID NO: 242 - RNA, 20 nt, synthetic construct
  misc_feature 1..20: note = antisense sequence
  source 1..20: mol_type = other RNA
gggtgttctggagtttcagg

SEQ ID NO: 243 - RNA, 20 nt, synthetic construct
  misc_feature 1..20: note = antisense sequence
  source 1..20: mol_type = other RNA
aagttacttctgggtgttct

SEQ ID NO: 244 - RNA, 20 nt, synthetic construct
  misc_feature 1..20: note = antisense sequence
  source 1..20: mol_type = other RNA
ggagaaggtctttgatgcta

SEQ ID NO: 245 - RNA, 20 nt, synthetic construct
  misc_feature 1..20: note = antisense sequence
  source 1..20: mol_type = other RNA
tggagaaggtctttgatgct

SEQ ID NO: 246 - RNA, 20 nt, synthetic construct
  misc_feature 1..20: note = antisense sequence
  source 1..20: mol_type = other RNA
ctggagaaggtctttgatgc

SEQ ID NO: 247 - RNA, 20 nt, synthetic construct
  misc_feature 1..20: note = antisense sequence
  source 1..20: mol_type = other RNA
ggtctggagaaggtctttga

SEQ ID NO: 248 - RNA, 20 nt, synthetic construct
  misc_feature 1..20: note = antisense sequence
  source 1..20: mol_type = other RNA
cggtctggagaaggtctttg

SEQ ID NO: 249 - RNA, 20 nt, synthetic construct
  misc_feature 1..20: note = antisense sequence
  source 1..20: mol_type = other RNA
acggtctggagaaggtcttt

SEQ ID NO: 250 - RNA, 20 nt, synthetic construct
  misc_feature 1..20: note = antisense sequence
  source 1..20: mol_type = other RNA
ccacggtctggagaaggtct

SEQ ID NO: 251 - RNA, 20 nt, synthetic construct
  misc_feature 1..20: note = antisense sequence
  source 1..20: mol_type = other RNA
attggtcttccacggtctgg

SEQ ID NO: 252 - RNA, 20 nt, synthetic construct
  misc_feature 1..20: note = antisense sequence
  source 1..20: mol_type = other RNA
tattggtcttccacggtctg

SEQ ID NO: 253 - RNA, 20 nt, synthetic construct
  misc_feature 1..20: note = antisense sequence
  source 1..20: mol_type = other RNA
atattggtcttccacggtct

SEQ ID NO: 254 - RNA, 20 nt, synthetic construct
  misc_feature 1..20: note = antisense sequence
  source 1..20: mol_type = other RNA
ttatattggtcttccacggt

SEQ ID NO: 255 - RNA, 20 nt, synthetic construct
  misc_feature 1..20: note = antisense sequence
  source 1..20: mol_type = other RNA
tttatattggtcttccacgg

SEQ ID NO: 256 - RNA, 20 nt, synthetic construct
  misc_feature 1..20: note = antisense sequence
  source 1..20: mol_type = other RNA
gtttatattggtcttccacg

SEQ ID NO: 257 - RNA, 20 nt, synthetic construct
  misc_feature 1..20: note = antisense sequence
  source 1..20: mol_type = other RNA
gtccttctgagctgattttc

SEQ ID NO: 258 - RNA, 20 nt, synthetic construct
  misc_feature 1..20: note = antisense sequence
  source 1..20: mol_type = other RNA
agtccttctgagctgatttt

SEQ ID NO: 259 - RNA, 20 nt, synthetic construct
  misc_feature 1..20: note = antisense sequence
  source 1..20: mol_type = other RNA
ctagtccttctgagctgatt

SEQ ID NO: 260 - RNA, 20 nt, synthetic construct
  misc_feature 1..20: note = antisense sequence
  source 1..20: mol_type = other RNA
tgtgggttcttgaatactag

SEQ ID NO: 261 - RNA, 20 nt, synthetic construct
  misc_feature 1..20: note = antisense sequence
  source 1..20: mol_type = other RNA
ctgtgggttcttgaatacta

SEQ ID NO: 262 - RNA, 20 nt, synthetic construct
  misc_feature 1..20: note = antisense sequence
  source 1..20: mol_type = other RNA
ttctgtgggttcttgaatac

SEQ ID NO: 263 - RNA, 20 nt, synthetic construct
  misc_feature 1..20: note = antisense sequence
  source 1..20: mol_type = other RNA
tggcttggaagatagagaaa

SEQ ID NO: 264 - RNA, 20 nt, synthetic construct
  misc_feature 1..20: note = antisense sequence
  source 1..20: mol_type = other RNA
cttggcttggaagatagaga

SEQ ID NO: 265 - RNA, 20 nt, synthetic construct
  misc_feature 1..20: note = antisense sequence
  source 1..20: mol_type = other RNA
agttcttggtgctcttggct

SEQ ID NO: 266 - RNA, 20 nt, synthetic construct
  misc_feature 1..20: note = antisense sequence
  source 1..20: mol_type = other RNA
tagttcttggtgctcttggc

SEQ ID NO: 267 - RNA, 20 nt, synthetic construct
  misc_feature 1..20: note = antisense sequence
  source 1..20: mol_type = other RNA
gtagttcttggtgctcttgg

SEQ ID NO: 268 - RNA, 20 nt, synthetic construct
  misc_feature 1..20: note = antisense sequence
  source 1..20: mol_type = other RNA
gagtagttcttggtgctctt

SEQ ID NO: 269 - RNA, 20 nt, synthetic construct
  misc_feature 1..20: note = antisense sequence
  source 1..20: mol_type = other RNA
ggagtagttcttggtgctct

SEQ ID NO: 270 - RNA, 20 nt, synthetic construct
  misc_feature 1..20: note = antisense sequence
  source 1..20: mol_type = other RNA
gggagtagttcttggtgctc

SEQ ID NO: 271 - RNA, 20 nt, synthetic construct
  misc_feature 1..20: note = antisense sequence
  source 1..20: mol_type = other RNA
agggagtagttcttggtgct

SEQ ID NO: 272 - RNA, 20 nt, synthetic construct
  misc_feature 1..20: note = antisense sequence
  source 1..20: mol_type = other RNA
aaagggagtagttcttggtg

SEQ ID NO: 273 - RNA, 20 nt, synthetic construct
  misc_feature 1..20: note = antisense sequence
  source 1..20: mol_type = other RNA
gaaagggagtagttcttggt

SEQ ID NO: 274 - RNA, 20 nt, synthetic construct
  misc_feature 1..20: note = antisense sequence
  source 1..20: mol_type = other RNA
caactgaagaaagggagtag

SEQ ID NO: 275 - RNA, 20 nt, synthetic construct
  misc_feature 1..20: note = antisense sequence
  source 1..20: mol_type = other RNA
tcaactgaagaaagggagta

SEQ ID NO: 276 - RNA, 20 nt, synthetic construct
  misc_feature 1..20: note = antisense sequence
  source 1..20: mol_type = other RNA
ttcaactgaagaaagggagt

SEQ ID NO: 277 - RNA, 20 nt, synthetic construct
  misc_feature 1..20: note = antisense sequence
  source 1..20: mol_type = other RNA
agcaggaatgccatcatgtt

SEQ ID NO: 278 - RNA, 20 nt, synthetic construct
  misc_feature 1..20: note = antisense sequence
  source 1..20: mol_type = other RNA
cagcaggaatgccatcatgt

SEQ ID NO: 279 - RNA, 20 nt, synthetic construct
  misc_feature 1..20: note = antisense sequence
  source 1..20: mol_type = other RNA
tcagcaggaatgccatcatg

SEQ ID NO: 280 - RNA, 20 nt, synthetic construct
  misc_feature 1..20: note = antisense sequence
  source 1..20: mol_type = other RNA
ttcagcaggaatgccatcat

SEQ ID NO: 281 - RNA, 20 nt, synthetic construct
  misc_feature 1..20: note = antisense sequence
  source 1..20: mol_type = other RNA
ggtggtacattcagcaggaa

SEQ ID NO: 282 - RNA, 20 nt, synthetic construct
  misc_feature 1..20: note = antisense sequence
  source 1..20: mol_type = other RNA
tggtggtacattcagcagga

SEQ ID NO: 283 - RNA, 20 nt, synthetic construct
  misc_feature 1..20: note = antisense sequence
  source 1..20: mol_type = other RNA
cttgtatgttcacctctgtt

SEQ ID NO: 284 - RNA, 20 nt, synthetic construct
  misc_feature 1..20: note = antisense sequence
  source 1..20: mol_type = other RNA
ccacttgtatgttcacctct

SEQ ID NO: 285 - RNA, 20 nt, synthetic construct
  misc_feature 1..20: note = antisense sequence
  source 1..20: mol_type = other RNA
gccacttgtatgttcacctc

SEQ ID NO: 286 - RNA, 20 nt, synthetic construct
  misc_feature 1..20: note = antisense sequence
  source 1..20: mol_type = other RNA
tgccacttgtatgttcacct

SEQ ID NO: 287 - RNA, 20 nt, synthetic construct
  misc_feature 1..20: note = antisense sequence
  source 1..20: mol_type = other RNA
gttgctgggtctgatggcat

SEQ ID NO: 288 - RNA, 20 nt, synthetic construct
  misc_feature 1..20: note = antisense sequence
  source 1..20: mol_type = other RNA
gagttgctgggtctgatggc

SEQ ID NO: 289 - RNA, 20 nt, synthetic construct
  misc_feature 1..20: note = antisense sequence
  source 1..20: mol_type = other RNA
gagagttgctgggtctgatg

SEQ ID NO: 290 - RNA, 20 nt, synthetic construct
  misc_feature 1..20: note = antisense sequence
  source 1..20: mol_type = other RNA
acttgagagttgctgggtct

SEQ ID NO: 291 - RNA, 20 nt, synthetic construct
  misc_feature 1..20: note = antisense sequence
  source 1..20: mol_type = other RNA
ggactacctgatataacatc

SEQ ID NO: 292 - RNA, 20 nt, synthetic construct
  misc_feature 1..20: note = antisense sequence
  source 1..20: mol_type = other RNA
catggactacctgatataac

SEQ ID NO: 293 - RNA, 20 nt, synthetic construct
  misc_feature 1..20: note = antisense sequence
  source 1..20: mol_type = other RNA
cccacgtttcattgaagttt

SEQ ID NO: 294 - RNA, 20 nt, synthetic construct
  misc_feature 1..20: note = antisense sequence
  source 1..20: mol_type = other RNA
tcccacgtttcattgaagtt

SEQ ID NO: 295 - RNA, 20 nt, synthetic construct
  misc_feature 1..20: note = antisense sequence
  source 1..20: mol_type = other RNA
ctcccacgtttcattgaagt

SEQ ID NO: 296 - RNA, 20 nt, synthetic construct
  misc_feature 1..20: note = antisense sequence
  source 1..20: mol_type = other RNA
tctcccacgtttcattgaag

SEQ ID NO: 297 - RNA, 20 nt, synthetic construct
  misc_feature 1..20: note = antisense sequence
  source 1..20: mol_type = other RNA
ttctcccacgtttcattgaa

SEQ ID NO: 298 - RNA, 20 nt, synthetic construct
  misc_feature 1..20: note = antisense sequence
  source 1..20: mol_type = other RNA
gttctcccacgtttcattga

SEQ ID NO: 299 - RNA, 20 nt, synthetic construct
  misc_feature 1..20: note = antisense sequence
  source 1..20: mol_type = other RNA
agttctcccacgtttcattg

SEQ ID NO: 300 - RNA, 20 nt, synthetic construct
  misc_feature 1..20: note = antisense sequence
  source 1..20: mol_type = other RNA
tagttctcccacgtttcatt

SEQ ID NO: 301 - RNA, 20 nt, synthetic construct
  misc_feature 1..20: note = antisense sequence
  source 1..20: mol_type = other RNA
gtagttctcccacgtttcat

SEQ ID NO: 302 - RNA, 20 nt, synthetic construct
  misc_feature 1..20: note = antisense sequence
  source 1..20: mol_type = other RNA
tgtagttctcccacgtttca

SEQ ID NO: 303 - RNA, 20 nt, synthetic construct
  misc_feature 1..20: note = antisense sequence
  source 1..20: mol_type = other RNA
ttgtagttctcccacgtttc

SEQ ID NO: 304 - RNA, 20 nt, synthetic construct
  misc_feature 1..20: note = antisense sequence
  source 1..20: mol_type = other RNA
tttgtagttctcccacgttt

SEQ ID NO: 305 - RNA, 20 nt, synthetic construct
  misc_feature 1..20: note = antisense sequence
  source 1..20: mol_type = other RNA
atttgtagttctcccacgtt

SEQ ID NO: 306 - RNA, 20 nt, synthetic construct
  misc_feature 1..20: note = antisense sequence
  source 1..20: mol_type = other RNA
tatttgtagttctcccacgt

SEQ ID NO: 307 - RNA, 20 nt, synthetic construct
  misc_feature 1..20: note = antisense sequence
  source 1..20: mol_type = other RNA
atatttgtagttctcccacg

SEQ ID NO: 308 - RNA, 20 nt, synthetic construct
  misc_feature 1..20: note = antisense sequence
  source 1..20: mol_type = other RNA
accatatttgtagttctccc

SEQ ID NO: 309 - RNA, 20 nt, synthetic construct
  misc_feature 1..20: note = antisense sequence
  source 1..20: mol_type = other RNA
agcctcccaaaaccatattt

SEQ ID NO: 310 - RNA, 20 nt, synthetic construct
  misc_feature 1..20: note = antisense sequence
  source 1..20: mol_type = other RNA
aagcctcccaaaaccatatt

SEQ ID NO: 311 - RNA, 20 nt, synthetic construct
  misc_feature 1..20: note = antisense sequence
  source 1..20: mol_type = other RNA
caagcctcccaaaaccatat

SEQ ID NO: 312 - RNA, 20 nt, synthetic construct
  misc_feature 1..20: note = antisense sequence
  source 1..20: mol_type = other RNA
tcaagcctcccaaaaccata

SEQ ID NO: 313 - RNA, 20 nt, synthetic construct
  misc_feature 1..20: note = antisense sequence
  source 1..20: mol_type = other RNA
atcaagcctcccaaaaccat

SEQ ID NO: 314 - RNA, 20 nt, synthetic construct
  misc_feature 1..20: note = antisense sequence
  source 1..20: mol_type = other RNA
catcaagcctcccaaaacca

SEQ ID NO: 315 - RNA, 20 nt, synthetic construct
  misc_feature 1..20: note = antisense sequence
  source 1..20: mol_type = other RNA
ccatcaagcctcccaaaacc

SEQ ID NO: 316 - RNA, 20 nt, synthetic construct
  misc_feature 1..20: note = antisense sequence
  source 1..20: mol_type = other RNA
ctccatcaagcctcccaaaa

SEQ ID NO: 317 - RNA, 20 nt, synthetic construct
  misc_feature 1..20: note = antisense sequence
  source 1..20: mol_type = other RNA
tctccatcaagcctcccaaa

SEQ ID NO: 318 - RNA, 20 nt, synthetic construct
  misc_feature 1..20: note = antisense sequence
  source 1..20: mol_type = other RNA
aattctccatcaagcctccc

SEQ ID NO: 319 - RNA, 20 nt, synthetic construct
  misc_feature 1..20: note = antisense sequence
  source 1..20: mol_type = other RNA
aaattctccatcaagcctcc

SEQ ID NO: 320 - RNA, 20 nt, synthetic construct
  misc_feature 1..20: note = antisense sequence
  source 1..20: mol_type = other RNA
aaaattctccatcaagcctc

SEQ ID NO: 321 - RNA, 20 nt, synthetic construct
  misc_feature 1..20: note = antisense sequence
  source 1..20: mol_type = other RNA
ccaaaattctccatcaagcc

SEQ ID NO: 322 - RNA, 20 nt, synthetic construct
  misc_feature 1..20: note = antisense sequence
  source 1..20: mol_type = other RNA
accaaaattctccatcaagc

SEQ ID NO: 323 - RNA, 20 nt, synthetic construct
  misc_feature 1..20: note = antisense sequence
  source 1..20: mol_type = other RNA
ccaaccaaaattctccatca

SEQ ID NO: 324 - RNA, 20 nt, synthetic construct
  misc_feature 1..20: note = antisense sequence
  source 1..20: mol_type = other RNA
cccaaccaaaattctccatc

SEQ ID NO: 325 - RNA, 20 nt, synthetic construct
  misc_feature 1..20: note = antisense sequence
  source 1..20: mol_type = other RNA
gcccaaccaaaattctccat

SEQ ID NO: 326 - RNA, 20 nt, synthetic construct
  misc_feature 1..20: note = antisense sequence
  source 1..20: mol_type = other RNA
ggcccaaccaaaattctcca

SEQ ID NO: 327 - RNA, 20 nt, synthetic construct
  misc_feature 1..20: note = antisense sequence
  source 1..20: mol_type = other RNA
aggcccaaccaaaattctcc

SEQ ID NO: 328 - RNA, 20 nt, synthetic construct
  misc_feature 1..20: note = antisense sequence
  source 1..20: mol_type = other RNA
taggcccaaccaaaattctc

SEQ ID NO: 329 - RNA, 20 nt, synthetic construct
  misc_feature 1..20: note = antisense sequence
  source 1..20: mol_type = other RNA
tctaggcccaaccaaaattc

SEQ ID NO: 330 - RNA, 20 nt, synthetic construct
  misc_feature 1..20: note = antisense sequence
  source 1..20: mol_type = other RNA
ctctaggcccaaccaaaatt

SEQ ID NO: 331 - RNA, 20 nt, synthetic construct
  misc_feature 1..20: note = antisense sequence
  source 1..20: mol_type = other RNA
tctctaggcccaaccaaaat

SEQ ID NO: 332 - RNA, 20 nt, synthetic construct
  misc_feature 1..20: note = antisense sequence
  source 1..20: mol_type = other RNA
ttctctaggcccaaccaaaa

SEQ ID NO: 333 - RNA, 20 nt, synthetic construct
  misc_feature 1..20: note = antisense sequence
  source 1..20: mol_type = other RNA
cttctctaggcccaaccaaa

SEQ ID NO: 334 - RNA, 20 nt, synthetic construct
  misc_feature 1..20: note = antisense sequence
  source 1..20: mol_type = other RNA
tcttctctaggcccaaccaa

SEQ ID NO: 335 - RNA, 20 nt, synthetic construct
  misc_feature 1..20: note = antisense sequence
  source 1..20: mol_type = other RNA
tatcttctctaggcccaacc

SEQ ID NO: 336 - RNA, 20 nt, synthetic construct
  misc_feature 1..20: note = antisense sequence
  source 1..20: mol_type = other RNA
atatcttctctaggcccaac

SEQ ID NO: 337 - RNA, 20 nt, synthetic construct
  misc_feature 1..20: note = antisense sequence
  source 1..20: mol_type = other RNA
tatatcttctctaggcccaa

SEQ ID NO: 338 - RNA, 20 nt, synthetic construct
  misc_feature 1..20: note = antisense sequence
  source 1..20: mol_type = other RNA
gtatatcttctctaggccca

SEQ ID NO: 339 - RNA, 20 nt, synthetic construct
  misc_feature 1..20: note = antisense sequence
  source 1..20: mol_type = other RNA
agtatatcttctctaggccc

SEQ ID NO: 340 - RNA, 20 nt, synthetic construct
  misc_feature 1..20: note = antisense sequence
  source 1..20: mol_type = other RNA
gagtatatcttctctaggcc

SEQ ID NO: 341 - RNA, 20 nt, synthetic construct
  misc_feature 1..20: note = antisense sequence
  source 1..20: mol_type = other RNA
ggagtatatcttctctaggc

SEQ ID NO: 342 - RNA, 20 nt, synthetic construct
  misc_feature 1..20: note = antisense sequence
  source 1..20: mol_type = other RNA
tggagtatatcttctctagg

SEQ ID NO: 343 - RNA, 20 nt, synthetic construct
  misc_feature 1..20: note = antisense sequence
  source 1..20: mol_type = other RNA
gcttcactatggagtatatc

SEQ ID NO: 344 - RNA, 20 nt, synthetic construct
  misc_feature 1..20: note = antisense sequence
  source 1..20: mol_type = other RNA
agattgcttcactatggagt

SEQ ID NO: 345 - RNA, 20 nt, synthetic construct
  misc_feature 1..20: note = antisense sequence
  source 1..20: mol_type = other RNA
tagattgcttcactatggag

SEQ ID NO: 346 - RNA, 20 nt, synthetic construct
  misc_feature 1..20: note = antisense sequence
  source 1..20: mol_type = other RNA
gttgtctttccagtcttcca

SEQ ID NO: 347 - RNA, 20 nt, synthetic construct
  misc_feature 1..20: note = antisense sequence
  source 1..20: mol_type = other RNA
tgtttgttgtctttccagtc

SEQ ID NO: 348 - RNA, 20 nt, synthetic construct
  misc_feature 1..20: note = antisense sequence
  source 1..20: mol_type = other RNA
gtttcgtgatttcccaagta

SEQ ID NO: 349 - RNA, 20 nt, synthetic construct
  misc_feature 1..20: note = antisense sequence
  source 1..20: mol_type = other RNA
ggtttcgtgatttcccaagt

SEQ ID NO: 350 - RNA, 20 nt, synthetic construct
  misc_feature 1..20: note = antisense sequence
  source 1..20: mol_type = other RNA
gttggtttcgtgatttccca

SEQ ID NO: 351 - RNA, 20 nt, synthetic construct
  misc_feature 1..20: note = antisense sequence
  source 1..20: mol_type = other RNA
agttggtttcgtgatttccc

SEQ ID NO: 352 - RNA, 20 nt, synthetic construct
  misc_feature 1..20: note = antisense sequence
  source 1..20: mol_type = other RNA
tagttggtttcgtgatttcc

SEQ ID NO: 353 - RNA, 20 nt, synthetic construct
  misc_feature 1..20: note = antisense sequence
  source 1..20: mol_type = other RNA
cgtatagttggtttcgtgat

SEQ ID NO: 354 - RNA, 20 nt, synthetic construct
  misc_feature 1..20: note = antisense sequence
  source 1..20: mol_type = other RNA
gcgtatagttggtttcgtga

SEQ ID NO: 355 - RNA, 20 nt, synthetic construct
  misc_feature 1..20: note = antisense sequence
  source 1..20: mol_type = other RNA
agcgtatagttggtttcgtg

SEQ ID NO: 356 - RNA, 20 nt, synthetic construct
  misc_feature 1..20: note = antisense sequence
  source 1..20: mol_type = other RNA
tagcgtatagttggtttcgt

SEQ ID NO: 357 - RNA, 20 nt, synthetic construct
  misc_feature 1..20: note = antisense sequence
  source 1..20: mol_type = other RNA
gtagcgtatagttggtttcg

SEQ ID NO: 358 - RNA, 20 nt, synthetic construct
  misc_feature 1..20: note = antisense sequence
  source 1..20: mol_type = other RNA
tgtagcgtatagttggtttc

SEQ ID NO: 359 - RNA, 20 nt, synthetic construct
  misc_feature 1..20: note = antisense sequence
  source 1..20: mol_type = other RNA
agatgtagcgtatagttggt

SEQ ID NO: 360 - RNA, 20 nt, synthetic construct
  misc_feature 1..20: note = antisense sequence
  source 1..20: mol_type = other RNA
ctagatgtagcgtatagttg

SEQ ID NO: 361 - RNA, 20 nt, synthetic construct
  misc_feature 1..20: note = antisense sequence
  source 1..20: mol_type = other RNA
caactagatgtagcgtatag

SEQ ID NO: 362 - RNA, 20 nt, synthetic construct
  misc_feature 1..20: note = antisense sequence
  source 1..20: mol_type = other RNA
gcaactagatgtagcgtata

SEQ ID NO: 363 - RNA, 20 nt, synthetic construct
  misc_feature 1..20: note = antisense sequence
  source 1..20: mol_type = other RNA
cgcaactagatgtagcgtat

SEQ ID NO: 364 - RNA, 20 nt, synthetic construct
  misc_feature 1..20: note = antisense sequence
  source 1..20: mol_type = other RNA
tcgcaactagatgtagcgta

SEQ ID NO: 365 - RNA, 20 nt, synthetic construct
  misc_feature 1..20: note = antisense sequence
  source 1..20: mol_type = other RNA
atcgcaactagatgtagcgt

SEQ ID NO: 366 - RNA, 20 nt, synthetic construct
  misc_feature 1..20: note = antisense sequence
  source 1..20: mol_type = other RNA
aatcgcaactagatgtagcg

SEQ ID NO: 367 - RNA, 20 nt, synthetic construct
  misc_feature 1..20: note = antisense sequence
  source 1..20: mol_type = other RNA
taatcgcaactagatgtagc

SEQ ID NO: 368 - RNA, 20 nt, synthetic construct
  misc_feature 1..20: note = antisense sequence
  source 1..20: mol_type = other RNA
gtaatcgcaactagatgtag

SEQ ID NO: 369 - RNA, 20 nt, synthetic construct
  misc_feature 1..20: note = antisense sequence
  source 1..20: mol_type = other RNA
cagtaatcgcaactagatgt

SEQ ID NO: 370 - RNA, 20 nt, synthetic construct
  misc_feature 1..20: note = antisense sequence
  source 1..20: mol_type = other RNA
ggacattgccagtaatcgca

SEQ ID NO: 371 - RNA, 20 nt, synthetic construct
  misc_feature 1..20: note = antisense sequence
  source 1..20: mol_type = other RNA
gggacattgccagtaatcgc

SEQ ID NO: 372 - RNA, 20 nt, synthetic construct
  misc_feature 1..20: note = antisense sequence
  source 1..20: mol_type = other RNA
ttttccgggattgcattggg

SEQ ID NO: 373 - RNA, 20 nt, synthetic construct
  misc_feature 1..20: note = antisense sequence
  source 1..20: mol_type = other RNA
gttttccgggattgcattgg

SEQ ID NO: 374 - RNA, 20 nt, synthetic construct
  misc_feature 1..20: note = antisense sequence
  source 1..20: mol_type = other RNA
tgttttccgggattgcattg

SEQ ID NO: 375 - RNA, 20 nt, synthetic construct
  misc_feature 1..20: note = antisense sequence
  source 1..20: mol_type = other RNA
ttgttttccgggattgcatt

SEQ ID NO: 376 - RNA, 20 nt, synthetic construct
  misc_feature 1..20: note = antisense sequence
  source 1..20: mol_type = other RNA
tttgttttccgggattgcat

SEQ ID NO: 377 - RNA, 20 nt, synthetic construct
  misc_feature 1..20: note = antisense sequence
  source 1..20: mol_type = other RNA
ctttgttttccgggattgca

SEQ ID NO: 378 - RNA, 20 nt, synthetic construct
  misc_feature 1..20: note = antisense sequence
  source 1..20: mol_type = other RNA
tctttgttttccgggattgc

SEQ ID NO: 379 - RNA, 20 nt, synthetic construct
  misc_feature 1..20: note = antisense sequence
  source 1..20: mol_type = other RNA
atctttgttttccgggattg

SEQ ID NO: 380 - RNA, 20 nt, synthetic construct
  misc_feature 1..20: note = antisense sequence
  source 1..20: mol_type = other RNA
caaatctttgttttccggga

SEQ ID NO: 381 - RNA, 20 nt, synthetic construct
  misc_feature 1..20: note = antisense sequence
  source 1..20: mol_type = other RNA
caccaaatctttgttttccg

SEQ ID NO: 382 - RNA, 20 nt, synthetic construct
  misc_feature 1..20: note = antisense sequence
  source 1..20: mol_type = other RNA
cccaagtagaaaacaccaaa

SEQ ID NO: 383 - RNA, 20 nt, synthetic construct
  misc_feature 1..20: note = antisense sequence
  source 1..20: mol_type = other RNA
tcccaagtagaaaacaccaa

SEQ ID NO: 384 - RNA, 20 nt, synthetic construct
  misc_feature 1..20: note = antisense sequence
  source 1..20: mol_type = other RNA
ctttgtgatcccaagtagaa

SEQ ID NO: 385 - RNA, 20 nt, synthetic construct
  misc_feature 1..20: note = antisense sequence
  source 1..20: mol_type = other RNA
tgctttgtgatcccaagtag

SEQ ID NO: 386 - RNA, 20 nt, synthetic construct
  misc_feature 1..20: note = antisense sequence
  source 1..20: mol_type = other RNA
tttgctttgtgatcccaagt

SEQ ID NO: 387 - RNA, 20 nt, synthetic construct
  misc_feature 1..20: note = antisense sequence
  source 1..20: mol_type = other RNA
cttttgctttgtgatcccaa

SEQ ID NO: 388 - RNA, 20 nt, synthetic construct
  misc_feature 1..20: note = antisense sequence
  source 1..20: mol_type = other RNA
ccttttgctttgtgatccca

SEQ ID NO: 389 - RNA, 20 nt, synthetic construct
  misc_feature 1..20: note = antisense sequence
  source 1..20: mol_type = other RNA
gtccttttgctttgtgatcc

SEQ ID NO: 390 - RNA, 20 nt, synthetic construct
  misc_feature 1..20: note = antisense sequence
  source 1..20: mol_type = other RNA
tgtccttttgctttgtgatc

SEQ ID NO: 391 - RNA, 20 nt, synthetic construct
  misc_feature 1..20: note = antisense sequence
  source 1..20: mol_type = other RNA
gttgaagtgtccttttgctt

SEQ ID NO: 392 - RNA, 20 nt, synthetic construct
  misc_feature 1..20: note = antisense sequence
  source 1..20: mol_type = other RNA
agttgaagtgtccttttgct

SEQ ID NO: 393 - RNA, 20 nt, synthetic construct
  misc_feature 1..20: note = antisense sequence
  source 1..20: mol_type = other RNA
cagttgaagtgtccttttgc

SEQ ID NO: 394 - RNA, 20 nt, synthetic construct
  misc_feature 1..20: note = antisense sequence
  source 1..20: mol_type = other RNA
cctctggacagttgaagtgt

SEQ ID NO: 395 - RNA, 20 nt, synthetic construct
  misc_feature 1..20: note = antisense sequence
  source 1..20: mol_type = other RNA
aaccctctggacagttgaag

SEQ ID NO: 396 - RNA, 20 nt, synthetic construct
  misc_feature 1..20: note = antisense sequence
  source 1..20: mol_type = other RNA
taaccctctggacagttgaa

SEQ ID NO: 397 - RNA, 20 nt, synthetic construct
  misc_feature 1..20: note = antisense sequence
  source 1..20: mol_type = other RNA
ataaccctctggacagttga

SEQ ID NO: 398 - RNA, 20 nt, synthetic construct
  misc_feature 1..20: note = antisense sequence
  source 1..20: mol_type = other RNA
aataaccctctggacagttg

SEQ ID NO: 399 - RNA, 20 nt, synthetic construct
  misc_feature 1..20: note = antisense sequence
  source 1..20: mol_type = other RNA
gaataaccctctggacagtt

SEQ ID NO: 400 - RNA, 20 nt, synthetic construct
  misc_feature 1..20: note = antisense sequence
  source 1..20: mol_type = other RNA
tgaataaccctctggacagt

SEQ ID NO: 401 - RNA, 20 nt, synthetic construct
  misc_feature 1..20: note = antisense sequence
  source 1..20: mol_type = other RNA
ctcctgaataaccctctgga

SEQ ID NO: 402 - RNA, 20 nt, synthetic construct
  misc_feature 1..20: note = antisense sequence
  source 1..20: mol_type = other RNA
cctcctgaataaccctctgg

SEQ ID NO: 403 - RNA, 20 nt, synthetic construct
  misc_feature 1..20: note = antisense sequence
  source 1..20: mol_type = other RNA
agcctcctgaataaccctct

SEQ ID NO: 404 - RNA, 20 nt, synthetic construct
  misc_feature 1..20: note = antisense sequence
  source 1..20: mol_type = other RNA
cagcctcctgaataaccctc

SEQ ID NO: 405 - RNA, 20 nt, synthetic construct
  misc_feature 1..20: note = antisense sequence
  source 1..20: mol_type = other RNA
ccagcctcctgaataaccct

SEQ ID NO: 406 - RNA, 20 nt, synthetic construct
  misc_feature 1..20: note = antisense sequence
  source 1..20: mol_type = other RNA
accagcctcctgaataaccc

SEQ ID NO: 407 - RNA, 20 nt, synthetic construct
  misc_feature 1..20: note = antisense sequence
  source 1..20: mol_type = other RNA
ccaccagcctcctgaataac

SEQ ID NO: 408 - RNA, 20 nt, synthetic construct
  misc_feature 1..20: note = antisense sequence
  source 1..20: mol_type = other RNA
accaccagcctcctgaataa

SEQ ID NO: 409 - RNA, 20 nt, synthetic construct
  misc_feature 1..20: note = antisense sequence
  source 1..20: mol_type = other RNA
caccaccagcctcctgaata

SEQ ID NO: 410 - RNA, 20 nt, synthetic construct
  misc_feature 1..20: note = antisense sequence
  source 1..20: mol_type = other RNA
actcatcatgccaccaccag

SEQ ID NO: 411 - RNA, 20 nt, synthetic construct
  misc_feature 1..20: note = antisense sequence
  source 1..20: mol_type = other RNA
cactcatcatgccaccacca

SEQ ID NO: 412 - RNA, 20 nt, synthetic construct
  misc_feature 1..20: note = antisense sequence
  source 1..20: mol_type = other RNA
cacactcatcatgccaccac

SEQ ID NO: 413 - RNA, 20 nt, synthetic construct
  misc_feature 1..20: note = antisense sequence
  source 1..20: mol_type = other RNA
tccacactcatcatgccacc

SEQ ID NO: 414 - RNA, 20 nt, synthetic construct
  misc_feature 1..20: note = antisense sequence
  source 1..20: mol_type = other RNA
tctccacactcatcatgcca

SEQ ID NO: 415 - RNA, 20 nt, synthetic construct
  misc_feature 1..20: note = antisense sequence
  source 1..20: mol_type = other RNA
ttaggttgttttctccacac

SEQ ID NO: 416 - RNA, 20 nt, synthetic construct
  misc_feature 1..20: note = antisense sequence
  source 1..20: mol_type = other RNA
gcttagattttgctcttggt

SEQ ID NO: 417 - RNA, 20 nt, synthetic construct
  misc_feature 1..20: note = antisense sequence
  source 1..20: mol_type = other RNA
ggcttagattttgctcttgg

SEQ ID NO: 418 - RNA, 20 nt, synthetic construct
  misc_feature 1..20: note = antisense sequence
  source 1..20: mol_type = other RNA
cctctctggcttagattttg

SEQ ID NO: 419 - RNA, 20 nt, synthetic construct
  misc_feature 1..20: note = antisense sequence
  source 1..20: mol_type = other RNA
tcctctctggcttagatttt

SEQ ID NO: 420 - RNA, 20 nt, synthetic construct
  misc_feature 1..20: note = antisense sequence
  source 1..20: mol_type = other RNA
ctcctctctggcttagattt

SEQ ID NO: 421 - RNA, 20 nt, synthetic construct
  misc_feature 1..20: note = antisense sequence
  source 1..20: mol_type = other RNA
gacttccaagataatcctct

SEQ ID NO: 422 - RNA, 20 nt, synthetic construct
  misc_feature 1..20: note = antisense sequence
  source 1..20: mol_type = other RNA
agacttccaagataatcctc

SEQ ID NO: 423 - RNA, 20 nt, synthetic construct
  misc_feature 1..20: note = antisense sequence
  source 1..20: mol_type = other RNA
gagacttccaagataatcct

SEQ ID NO: 424 - RNA, 20 nt, synthetic construct
  misc_feature 1..20: note = antisense sequence
  source 1..20: mol_type = other RNA
tgagacttccaagataatcc

SEQ ID NO: 425 - RNA, 20 nt, synthetic construct
  misc_feature 1..20: note = antisense sequence
  source 1..20: mol_type = other RNA
gaatctgttggatggatcaa

SEQ ID NO: 426 - RNA, 20 nt, synthetic construct
  misc_feature 1..20: note = antisense sequence
  source 1..20: mol_type = other RNA
tgaatctgttggatggatca

SEQ ID NO: 427 - RNA, 20 nt, synthetic construct
  misc_feature 1..20: note = antisense sequence
  source 1..20: mol_type = other RNA
gcctcagttcattcaaagct

SEQ ID NO: 428 - RNA, 20 nt, synthetic construct
  misc_feature 1..20: note = antisense sequence
  source 1..20: mol_type = other RNA
tgcctcagttcattcaaagc

SEQ ID NO: 429 - RNA, 20 nt, synthetic construct
  misc_feature 1..20: note = antisense sequence
  source 1..20: mol_type = other RNA
gggaaagttgtgatattagc

SEQ ID NO: 430 - RNA, 20 nt, synthetic construct
  misc_feature 1..20: note = antisense sequence
  source 1..20: mol_type = other RNA
cagtcctctgtatttagtca

SEQ ID NO: 431 - RNA, 20 nt, synthetic construct
  misc_feature 1..20: note = antisense sequence
  source 1..20: mol_type = other RNA
ccagtcctctgtatttagtc

SEQ ID NO: 432 - RNA, 20 nt, synthetic construct
  misc_feature 1..20: note = antisense sequence
  source 1..20: mol_type = other RNA
taccagtcctctgtatttag

SEQ ID NO: 433 - RNA, 20 nt, synthetic construct
  misc_feature 1..20: note = antisense sequence
  source 1..20: mol_type = other RNA
caattaccagtcctctgtat

SEQ ID NO: 434 - RNA, 20 nt, synthetic construct
  misc_feature 1..20: note = antisense sequence
  source 1..20: mol_type = other RNA
acaattaccagtcctctgta

SEQ ID NO: 435 - RNA, 20 nt, synthetic construct
  misc_feature 1..20: note = antisense sequence
  source 1..20: mol_type = other RNA
gtacaattaccagtcctctg

SEQ ID NO: 436 - RNA, 20 nt, synthetic construct
  misc_feature 1..20: note = antisense sequence
  source 1..20: mol_type = other RNA
tactctgtgctgacgatttt

SEQ ID NO: 437 - RNA, 20 nt, synthetic construct
  misc_feature 1..20: note = antisense sequence
  source 1..20: mol_type = other RNA
atactctgtgctgacgattt

SEQ ID NO: 438 - RNA, 20 nt, synthetic construct
  misc_feature 1..20: note = antisense sequence
  source 1..20: mol_type = other RNA
catactctgtgctgacgatt

SEQ ID NO: 439 - RNA, 20 nt, synthetic construct
  misc_feature 1..20: note = antisense sequence
  source 1..20: mol_type = other RNA
gaatcacagcatatttaggg

SEQ ID NO: 440 - RNA, 20 nt, synthetic construct
  misc_feature 1..20: note = antisense sequence
  source 1..20: mol_type = other RNA
cttgaaaacctacacgaatg

SEQ ID NO: 441 - RNA, 20 nt, synthetic construct
  misc_feature 1..20: note = antisense sequence
  source 1..20: mol_type = other RNA
ctacttgaaaacctacacga

SEQ ID NO: 442 - RNA, 20 nt, synthetic construct
  misc_feature 1..20: note = antisense sequence
  source 1..20: mol_type = other RNA
tctacttgaaaacctacacg

SEQ ID NO: 443 - RNA, 20 nt, synthetic construct
  misc_feature 1..20: note = antisense sequence
  source 1..20: mol_type = other RNA
atacatgcttgtcaggctgt

SEQ ID NO: 444 - RNA, 20 nt, synthetic construct
  misc_feature 1..20: note = antisense sequence
  source 1..20: mol_type = other RNA
atatacatgcttgtcaggct

SEQ ID NO: 445 - RNA, 20 nt, synthetic construct
  misc_feature 1..20: note = antisense sequence
  source 1..20: mol_type = other RNA
gactgggtctttgtttattg

SEQ ID NO: 446 - RNA, 20 nt, synthetic construct
  misc_feature 1..20: note = antisense sequence
  source 1..20: mol_type = other RNA
agggactgggtctttgttta

SEQ ID NO: 447 - RNA, 20 nt, synthetic construct
  misc_feature 1..20: note = antisense sequence
  source 1..20: mol_type = other RNA
tagggactgggtctttgttt

SEQ ID NO: 448 - RNA, 20 nt, synthetic construct
  misc_feature 1..20: note = antisense sequence
  source 1..20: mol_type = other RNA
ttagggactgggtctttgtt

SEQ ID NO: 449 - RNA, 20 nt, synthetic construct
  misc_feature 1..20: note = antisense sequence
  source 1..20: mol_type = other RNA
atttagggactgggtctttg

SEQ ID NO: 450 - RNA, 20 nt, synthetic construct
  misc_feature 1..20: note = antisense sequence
  source 1..20: mol_type = other RNA
taatttagggactgggtctt

SEQ ID NO: 451 - RNA, 20 nt, synthetic construct
  misc_feature 1..20: note = antisense sequence
  source 1..20: mol_type = other RNA
ctataatttagggactgggt

SEQ ID NO: 452 - RNA, 20 nt, synthetic construct
  misc_feature 1..20: note = antisense sequence
  source 1..20: mol_type = other RNA
tctataatttagggactggg

SEQ ID NO: 453 - RNA, 20 nt, synthetic construct
  misc_feature 1..20: note = antisense sequence
  source 1..20: mol_type = other RNA
ctgttgtgatgtcgataaac

SEQ ID NO: 454 - RNA, 20 nt, synthetic construct
  misc_feature 1..20: note = antisense sequence
  source 1..20: mol_type = other RNA
ggatctgttgtgatgtcgat

SEQ ID NO: 455 - RNA, 20 nt, synthetic construct
  misc_feature 1..20: note = antisense sequence
  source 1..20: mol_type = other RNA
gggatctgttgtgatgtcga

SEQ ID NO: 456 - RNA, 20 nt, synthetic construct
  misc_feature 1..20: note = antisense sequence
  source 1..20: mol_type = other RNA
agggatctgttgtgatgtcg

SEQ ID NO: 457 - RNA, 20 nt, synthetic construct
  misc_feature 1..20: note = antisense sequence
  source 1..20: mol_type = other RNA
tgacaagtgatactgtggta

SEQ ID NO: 458 - RNA, 20 nt, synthetic construct
  misc_feature 1..20: note = antisense sequence
  source 1..20: mol_type = other RNA
ctgacaagtgatactgtggt

SEQ ID NO: 459 - RNA, 20 nt, synthetic construct
  misc_feature 1..20: note = antisense sequence
  source 1..20: mol_type = other RNA
tctgacaagtgatactgtgg

SEQ ID NO: 460 - RNA, 20 nt, synthetic construct
  misc_feature 1..20: note = antisense sequence
  source 1..20: mol_type = other RNA
ttctgacaagtgatactgtg

SEQ ID NO: 461 - RNA, 20 nt, synthetic construct
  misc_feature 1..20: note = antisense sequence
  source 1..20: mol_type = other RNA
gggcactatatcctttaaca

SEQ ID NO: 462 - RNA, 20 nt, synthetic construct
  misc_feature 1..20: note = antisense sequence
  source 1..20: mol_type = other RNA
tgggcactatatcctttaac

SEQ ID NO: 463 - RNA, 20 nt, synthetic construct
  misc_feature 1..20: note = antisense sequence
  source 1..20: mol_type = other RNA
cttgggcactatatccttta

SEQ ID NO: 464 - RNA, 20 nt, synthetic construct
  misc_feature 1..20: note = antisense sequence
  source 1..20: mol_type = other RNA
acttgggcactatatccttt

SEQ ID NO: 465 - RNA, 20 nt, synthetic construct
  misc_feature 1..20: note = antisense sequence
  source 1..20: mol_type = other RNA
aacttgggcactatatcctt

SEQ ID NO: 466 - RNA, 20 nt, synthetic construct
  misc_feature 1..20: note = antisense sequence
  source 1..20: mol_type = other RNA
taacttgggcactatatcct

SEQ ID NO: 467 - RNA, 20 nt, synthetic construct
  misc_feature 1..20: note = antisense sequence
  source 1..20: mol_type = other RNA
ataacttgggcactatatcc

SEQ ID NO: 468 - RNA, 20 nt, synthetic construct
  misc_feature 1..20: note = antisense sequence
  source 1..20: mol_type = other RNA
ccatataacttgggcactat

SEQ ID NO: 469 - RNA, 20 nt, synthetic construct
  misc_feature 1..20: note = antisense sequence
  source 1..20: mol_type = other RNA
accatataacttgggcacta

SEQ ID NO: 470 - RNA, 20 nt, synthetic construct
  misc_feature 1..20: note = antisense sequence
  source 1..20: mol_type = other RNA
caccatataacttgggcact

SEQ ID NO: 471 - RNA, 20 nt, synthetic construct
  misc_feature 1..20: note = antisense sequence
  source 1..20: mol_type = other RNA
tcaccatataacttgggcac

SEQ ID NO: 472 - RNA, 20 nt, synthetic construct
  misc_feature 1..20: note = antisense sequence
  source 1..20: mol_type = other RNA
aggtcaccatataacttggg

SEQ ID NO: 473 - RNA, 20 nt, synthetic construct
  misc_feature 1..20: note = antisense sequence
  source 1..20: mol_type = other RNA
taggtcaccatataacttgg

SEQ ID NO: 474 - RNA, 20 nt, synthetic construct
  misc_feature 1..20: note = antisense sequence
  source 1..20: mol_type = other RNA
gtaggtcaccatataacttg

SEQ ID NO: 475 - RNA, 20 nt, synthetic construct
  misc_feature 1..20: note = antisense sequence
  source 1..20: mol_type = other RNA
ggtaggtcaccatataactt

SEQ ID NO: 476 - RNA, 20 nt, synthetic construct
  misc_feature 1..20: note = antisense sequence
  source 1..20: mol_type = other RNA
aggtaggtcaccatataact

SEQ ID NO: 477 - RNA, 20 nt, synthetic construct
  misc_feature 1..20: note = antisense sequence
  source 1..20: mol_type = other RNA
aaggtaggtcaccatataac

SEQ ID NO: 478 - RNA, 20 nt, synthetic construct
  misc_feature 1..20: note = antisense sequence
  source 1..20: mol_type = other RNA
caaaggtaggtcaccatata

SEQ ID NO: 479 - RNA, 20 nt, synthetic construct
  misc_feature 1..20: note = antisense sequence
  source 1..20: mol_type = other RNA
acaaaggtaggtcaccatat

SEQ ID NO: 480 - RNA, 20 nt, synthetic construct
  misc_feature 1..20: note = antisense sequence
  source 1..20: mol_type = other RNA
gacaaaggtaggtcaccata

SEQ ID NO: 481 - RNA, 20 nt, synthetic construct
  misc_feature 1..20: note = antisense sequence
  source 1..20: mol_type = other RNA
gcttagtctgtggtcctgaa

SEQ ID NO: 482 - RNA, 20 nt, synthetic construct
  misc_feature 1..20: note = antisense sequence
  source 1..20: mol_type = other RNA
agcttagtctgtggtcctga

SEQ ID NO: 483 - RNA, 20 nt, synthetic construct
  misc_feature 1..20: note = antisense sequence
  source 1..20: mol_type = other RNA
cagcttagtctgtggtcctg

SEQ ID NO: 484 - RNA, 20 nt, synthetic construct
  misc_feature 1..20: note = antisense sequence
  source 1..20: mol_type = other RNA
aatttcgacagcttagtctg

SEQ ID NO: 485 - RNA, 20 nt, synthetic construct
  misc_feature 1..20: note = antisense sequence
  source 1..20: mol_type = other RNA
cgttaatttcgacagcttag

SEQ ID NO: 486 - RNA, 20 nt, synthetic construct
  misc_feature 1..20: note = antisense sequence
  source 1..20: mol_type = other RNA
gcgttaatttcgacagctta

SEQ ID NO: 487 - RNA, 20 nt, synthetic construct
  misc_feature 1..20: note = antisense sequence
  source 1..20: mol_type = other RNA
agcgttaatttcgacagctt

SEQ ID NO: 488 - RNA, 20 nt, synthetic construct
  misc_feature 1..20: note = antisense sequence
  source 1..20: mol_type = other RNA
cagcgttaatttcgacagct

SEQ ID NO: 489 - RNA, 20 nt, synthetic construct
  misc_feature 1..20: note = antisense sequence
  source 1..20: mol_type = other RNA
tcagcgttaatttcgacagc

SEQ ID NO: 490 - RNA, 20 nt, synthetic construct
  misc_feature 1..20: note = antisense sequence
  source 1..20: mol_type = other RNA
atcagcgttaatttcgacag

SEQ ID NO: 491 - RNA, 20 nt, synthetic construct
  misc_feature 1..20: note = antisense sequence
  source 1..20: mol_type = other RNA
ttggtattctggccctaaaa

SEQ ID NO: 492 - RNA, 20 nt, synthetic construct
  misc_feature 1..20: note = antisense sequence
  source 1..20: mol_type = other RNA
tttggtattctggccctaaa

SEQ ID NO: 493 - RNA, 20 nt, synthetic construct
  misc_feature 1..20: note = antisense sequence
  source 1..20: mol_type = other RNA
ttttggtattctggccctaa

SEQ ID NO: 494 - RNA, 20 nt, synthetic construct
  misc_feature 1..20: note = antisense sequence
  source 1..20: mol_type = other RNA
attttggtattctggcccta

SEQ ID NO: 495 - RNA, 20 nt, synthetic construct
  misc_feature 1..20: note = antisense sequence
  source 1..20: mol_type = other RNA
cattttggtattctggccct

SEQ ID NO: 496 - RNA, 20 nt, synthetic construct
  misc_feature 1..20: note = antisense sequence
  source 1..20: mol_type = other RNA
ccattttggtattctggccc

SEQ ID NO: 497 - RNA, 20 nt, synthetic construct
  misc_feature 1..20: note = antisense sequence
  source 1..20: mol_type = other RNA
gagccattttggtattctgg

SEQ ID NO: 498 - RNA, 20 nt, synthetic construct
  misc_feature 1..20: note = antisense sequence
  source 1..20: mol_type = other RNA
ggagccattttggtattctg

SEQ ID NO: 499 - RNA, 20 nt, synthetic construct
  misc_feature 1..20: note = antisense sequence
  source 1..20: mol_type = other RNA
aggagccattttggtattct

SEQ ID NO: 500 - RNA, 20 nt, synthetic construct
  misc_feature 1..20: note = antisense sequence
  source 1..20: mol_type = other RNA
gaggagccattttggtattc

SEQ ID NO: 501 - RNA, 20 nt, synthetic construct
  misc_feature 1..20: note = antisense sequence
  source 1..20: mol_type = other RNA
agagaggagccattttggta

SEQ ID NO: 502 - RNA, 20 nt, synthetic construct
  misc_feature 1..20: note = antisense sequence
  source 1..20: mol_type = other RNA
caaatagaggaagcaactca

SEQ ID NO: 503 - RNA, 20 nt, synthetic construct
  misc_feature 1..20: note = antisense sequence
  source 1..20: mol_type = other RNA
aaccaaatagaggaagcaac

SEQ ID NO: 504 - RNA, 19 nt, synthetic construct
  misc_feature 1..19: note = sense sequence
  source 1..19: mol_type = other RNA
cagttccacgttgcttgaa

SEQ ID NO: 505 - RNA, 19 nt, synthetic construct
  misc_feature 1..19: note = antisense sequence
  source 1..19: mol_type = other RNA
ttcaagcaacgtggaactg

SEQ ID NO: 506 - RNA, 19 nt, synthetic construct
  misc_feature 1..19: note = sense sequence
  source 1..19: mol_type = other RNA
ttgttcctctagttatttc

SEQ ID NO: 507 - RNA, 19 nt, synthetic construct
  misc_feature 1..19: note = antisense sequence
  source 1..19: mol_type = other RNA
gaaataactagaggaacaa

SEQ ID NO: 508 - RNA, 19 nt, synthetic construct
  misc_feature 1..19: note = sense sequence
  source 1..19: mol_type = other RNA
tgttcctctagttatttcc

SEQ ID NO: 509 - RNA, 19 nt, synthetic construct
  misc_feature 1..19: note = antisense sequence
  source 1..19: mol_type = other RNA
ggaaataactagaggaaca

SEQ ID NO: 510 - RNA, 19 nt, synthetic construct
  misc_feature 1..19: note = sense sequence
  source 1..19: mol_type = other RNA
ctagttatttcctccagaa

SEQ ID NO: 511 - RNA, 19 nt, synthetic construct
  misc_feature 1..19: note = antisense sequence
  source 1..19: mol_type = other RNA
ttctggaggaaataactag

SEQ ID NO: 512 - RNA, 19 nt, synthetic construct
  misc_feature 1..19: note = sense sequence
  source 1..19: mol_type = other RNA
tagttatttcctccagaat

SEQ ID NO: 513 - RNA, 19 nt, synthetic construct
  misc_feature 1..19: note = antisense sequence
  source 1..19: mol_type = other RNA
attctggaggaaataacta

SEQ ID NO: 514 - RNA, 19 nt, synthetic construct
  misc_feature 1..19: note = sense sequence
  source 1..19: mol_type = other RNA
agttatttcctccagaatt

SEQ ID NO: 515 - RNA, 19 nt, synthetic construct
  misc_feature 1..19: note = antisense sequence
  source 1..19: mol_type = other RNA
aattctggaggaaataact

SEQ ID NO: 516 - RNA, 19 nt, synthetic construct
  misc_feature 1..19: note = sense sequence
  source 1..19: mol_type = other RNA
gttatttcctccagaattg

SEQ ID NO: 517 - RNA, 19 nt, synthetic construct
  misc_feature 1..19: note = antisense sequence
  source 1..19: mol_type = other RNA
caattctggaggaaataac

SEQ ID NO: 518 - RNA, 19 nt, synthetic construct
  misc_feature 1..19: note = sense sequence
  source 1..19: mol_type = other RNA
ttatttcctccagaattga

SEQ ID NO: 519 - RNA, 19 nt, synthetic construct
  misc_feature 1..19: note = antisense sequence
  source 1..19: mol_type = other RNA
tcaattctggaggaaataa

SEQ ID NO: 520 - RNA, 19 nt, synthetic construct
  misc_feature 1..19: note = sense sequence
  source 1..19: mol_type = other RNA
atttcctccagaattgatc

SEQ ID NO: 521 - RNA, 19 nt, synthetic construct
  misc_feature 1..19: note = antisense sequence
  source 1..19: mol_type = other RNA
gatcaattctggaggaaat

SEQ ID NO: 522 - RNA, 19 nt, synthetic construct
  misc_feature 1..19: note = sense sequence
  source 1..19: mol_type = other RNA
ttcctccagaattgatcaa

SEQ ID NO: 523 - RNA, 19 nt, synthetic construct
  misc_feature 1..19: note = antisense sequence
  source 1..19: mol_type = other RNA
ttgatcaattctggaggaa

SEQ ID NO: 524 - RNA, 19 nt, synthetic construct
  misc_feature 1..19: note = sense sequence
  source 1..19: mol_type = other RNA
tcctccagaattgatcaag

SEQ ID NO: 525 - RNA, 19 nt, synthetic construct
  misc_feature 1..19: note = antisense sequence
  source 1..19: mol_type = other RNA
cttgatcaattctggagga

SEQ ID NO: 526 - RNA, 19 nt, synthetic construct
  misc_feature 1..19: note = sense sequence
  source 1..19: mol_type = other RNA
cctccagaattgatcaaga

SEQ ID NO: 527 - RNA, 19 nt, synthetic construct
  misc_feature 1..19: note = antisense sequence
  source 1..19: mol_type = other RNA
tcttgatcaattctggagg

SEQ ID NO: 528 - RNA, 19 nt, synthetic construct
  misc_feature 1..19: note = sense sequence
  source 1..19: mol_type = other RNA
catcatttgattctctatc

SEQ ID NO: 529 - RNA, 19 nt, synthetic construct
  misc_feature 1..19: note = antisense sequence
  source 1..19: mol_type = other RNA
gatagagaatcaaatgatg

SEQ ID NO: 530 - RNA, 19 nt, synthetic construct
  misc_feature 1..19: note = sense sequence
  source 1..19: mol_type = other RNA
tcatttgattctctatctc

SEQ ID NO: 531 - RNA, 19 nt, synthetic construct
  misc_feature 1..19: note = antisense sequence
  source 1..19: mol_type = other RNA
gagatagagaatcaaatga

SEQ ID NO: 532 - RNA, 19 nt, synthetic construct
  misc_feature 1..19: note = sense sequence
  source 1..19: mol_type = other RNA
catttgattctctatctcc

SEQ ID NO: 533 - RNA, 19 nt, synthetic construct
  misc_feature 1..19: note = antisense sequence
  source 1..19: mol_type = other RNA
ggagatagagaatcaaatg

SEQ ID NO: 534 - RNA, 19 nt, synthetic construct
  misc_feature 1..19: note = sense sequence
  source 1..19: mol_type = other RNA
atttgattctctatctcca

SEQ ID NO: 535 - RNA, 19 nt, synthetic construct
  misc_feature 1..19: note = antisense sequence
  source 1..19: mol_type = other RNA
tggagatagagaatcaaat

SEQ ID NO: 536 - RNA, 19 nt, synthetic construct
  misc_feature 1..19: note = sense sequence
  source 1..19: mol_type = other RNA
tttgattctctatctccag

SEQ ID NO: 537 - RNA, 19 nt, synthetic construct
  misc_feature 1..19: note = antisense sequence
  source 1..19: mol_type = other RNA
ctggagatagagaatcaaa

SEQ ID NO: 538 - RNA, 19 nt, synthetic construct
  misc_feature 1..19: note = sense sequence
  source 1..19: mol_type = other RNA
ttgattctctatctccaga

SEQ ID NO: 539 - RNA, 19 nt, synthetic construct
  misc_feature 1..19: note = antisense sequence
  source 1..19: mol_type = other RNA
tctggagatagagaatcaa

SEQ ID NO: 540 - RNA, 19 nt, synthetic construct
  misc_feature 1..19: note = sense sequence
  source 1..19: mol_type = other RNA
ctatctccagagccaaaat

SEQ ID NO: 541 - RNA, 19 nt, synthetic construct
  misc_feature 1..19: note = antisense sequence
  source 1..19: mol_type = other RNA
attttggctctggagatag

SEQ ID NO: 542 - RNA, 19 nt, synthetic construct
  misc_feature 1..19: note = sense sequence
  source 1..19: mol_type = other RNA
tatctccagagccaaaatc

SEQ ID NO: 543 - RNA, 19 nt, synthetic construct
  misc_feature 1..19: note = antisense sequence
  source 1..19: mol_type = other RNA
gattttggctctggagata

SEQ ID NO: 544 - RNA, 19 nt, synthetic construct
  misc_feature 1..19: note = sense sequence
  source 1..19: mol_type = other RNA
atctccagagccaaaatca

SEQ ID NO: 545 - RNA, 19 nt, synthetic construct
  misc_feature 1..19: note = antisense sequence
  source 1..19: mol_type = other RNA
tgattttggctctggagat

SEQ ID NO: 546 - RNA, 19 nt, synthetic construct
  misc_feature 1..19: note = sense sequence
  source 1..19: mol_type = other RNA
tccagagccaaaatcaaga

SEQ ID NO: 547 - RNA, 19 nt, synthetic construct
  misc_feature 1..19: note = antisense sequence
  source 1..19: mol_type = other RNA
tcttgattttggctctgga

SEQ ID NO: 548 - RNA, 19 nt, synthetic construct
  misc_feature 1..19: note = sense sequence
  source 1..19: mol_type = other RNA
ccagagccaaaatcaagat

SEQ ID NO: 549 - RNA, 19 nt, synthetic construct
  misc_feature 1..19: note = antisense sequence
  source 1..19: mol_type = other RNA
atcttgattttggctctgg

SEQ ID NO: 550 - RNA, 19 nt, synthetic construct
  misc_feature 1..19: note = sense sequence
  source 1..19: mol_type = other RNA
cagagccaaaatcaagatt

SEQ ID NO: 551 - RNA, 19 nt, synthetic construct
  misc_feature 1..19: note = antisense sequence
  source 1..19: mol_type = other RNA
aatcttgattttggctctg

SEQ ID NO: 552 - RNA, 19 nt, synthetic construct
  misc_feature 1..19: note = sense sequence
  source 1..19: mol_type = other RNA
caagatttgctatgttaga

SEQ ID NO: 553 - RNA, 19 nt, synthetic construct
  misc_feature 1..19: note = antisense sequence
  source 1..19: mol_type = other RNA
tctaacatagcaaatcttg

SEQ ID NO: 554 - RNA, 19 nt, synthetic construct
  misc_feature 1..19: note = sense sequence
  source 1..19: mol_type = other RNA
aagatttgctatgttagac

SEQ ID NO: 555 - RNA, 19 nt, synthetic construct
  misc_feature 1..19: note = antisense sequence
  source 1..19: mol_type = other RNA
gtctaacatagcaaatctt

SEQ ID NO: 556 - RNA, 19 nt, synthetic construct
  misc_feature 1..19: note = sense sequence
  source 1..19: mol_type = other RNA
agatttgctatgttagacg

SEQ ID NO: 557 - RNA, 19 nt, synthetic construct
  misc_feature 1..19: note = antisense sequence
  source 1..19: mol_type = other RNA
cgtctaacatagcaaatct

SEQ ID NO: 558 - RNA, 19 nt, synthetic construct
  misc_feature 1..19: note = sense sequence
  source 1..19: mol_type = other RNA
gatttgctatgttagacga

SEQ ID NO: 559 - RNA, 19 nt, synthetic construct
  misc_feature 1..19: note = antisense sequence
  source 1..19: mol_type = other RNA
tcgtctaacatagcaaatc

SEQ ID NO: 560 - RNA, 19 nt, synthetic construct
  misc_feature 1..19: note = sense sequence
  source 1..19: mol_type = other RNA
atttgctatgttagacgat

SEQ ID NO: 561 - RNA, 19 nt, synthetic construct
  misc_feature 1..19: note = antisense sequence
  source 1..19: mol_type = other RNA
atcgtctaacatagcaaat

SEQ ID NO: 562 - RNA, 19 nt, synthetic construct
  misc_feature 1..19: note = sense sequence
  source 1..19: mol_type = other RNA
tttgctatgttagacgatg

SEQ ID NO: 563 - RNA, 19 nt, synthetic construct
  misc_feature 1..19: note = antisense sequence
  source 1..19: mol_type = other RNA
catcgtctaacatagcaaa

SEQ ID NO: 564 - RNA, 19 nt, synthetic construct
  misc_feature 1..19: note = sense sequence
  source 1..19: mol_type = other RNA
ttgctatgttagacgatgt

SEQ ID NO: 565 - RNA, 19 nt, synthetic construct
  misc_feature 1..19: note = antisense sequence
  source 1..19: mol_type = other RNA
acatcgtctaacatagcaa

SEQ ID NO: 566 - RNA, 19 nt, synthetic construct
  misc_feature 1..19: note = sense sequence
  source 1..19: mol_type = other RNA
tgctatgttagacgatgta

SEQ ID NO: 567 - RNA, 19 nt, synthetic construct
  misc_feature 1..19: note = antisense sequence
  source 1..19: mol_type = other RNA
tacatcgtctaacatagca

SEQ ID NO: 568 - RNA, 19 nt, synthetic construct
  misc_feature 1..19: note = sense sequence
  source 1..19: mol_type = other RNA
gctatgttagacgatgtaa

SEQ ID NO: 569 - RNA, 19 nt, synthetic construct
  misc_feature 1..19: note = antisense sequence
  source 1..19: mol_type = other RNA
ttacatcgtctaacatagc

SEQ ID NO: 570 - RNA, 19 nt, synthetic construct
  misc_feature 1..19: note = sense sequence
  source 1..19: mol_type = other RNA
ctatgttagacgatgtaaa

SEQ ID NO: 571 - RNA, 19 nt, synthetic construct
  misc_feature 1..19: note = antisense sequence
  source 1..19: mol_type = other RNA
tttacatcgtctaacatag

SEQ ID NO: 572 - RNA, 19 nt, synthetic construct
  misc_feature 1..19: note = sense sequence
  source 1..19: mol_type = other RNA
aaaattttagccaatggcc

SEQ ID NO: 573 - RNA, 19 nt, synthetic construct
  misc_feature 1..19: note = antisense sequence
  source 1..19: mol_type = other RNA
ggccattggctaaaatttt

SEQ ID NO: 574 - RNA, 19 nt, synthetic construct
  misc_feature 1..19: note = sense sequence
  source 1..19: mol_type = other RNA
aaattttagccaatggcct

SEQ ID NO: 575 - RNA, 19 nt, synthetic construct
  misc_feature 1..19: note = antisense sequence
  source 1..19: mol_type = other RNA
aggccattggctaaaattt

SEQ ID NO: 576 - RNA, 19 nt, synthetic construct
  misc_feature 1..19: note = sense sequence
  source 1..19: mol_type = other RNA
attttagccaatggcctcc

SEQ ID NO: 577 - RNA, 19 nt, synthetic construct
  misc_feature 1..19: note = antisense sequence
  source 1..19: mol_type = other RNA
ggaggccattggctaaaat

SEQ ID NO: 578 - RNA, 19 nt, synthetic construct
  misc_feature 1..19: note = sense sequence
  source 1..19: mol_type = other RNA
tagccaatggcctccttca

SEQ ID NO: 579 - RNA, 19 nt, synthetic construct
  misc_feature 1..19: note = antisense sequence
  source 1..19: mol_type = other RNA
tgaaggaggccattggcta

SEQ ID NO: 580 - RNA, 19 nt, synthetic construct
  misc_feature 1..19: note = sense sequence
  source 1..19: mol_type = other RNA
agccaatggcctccttcag

SEQ ID NO: 581 - RNA, 19 nt, synthetic construct
  misc_feature 1..19: note = antisense sequence
  source 1..19: mol_type = other RNA
ctgaaggaggccattggct

SEQ ID NO: 582 - RNA, 19 nt, synthetic construct
  misc_feature 1..19: note = sense sequence
  source 1..19: mol_type = other RNA
gccaatggcctccttcagt

SEQ ID NO: 583 - RNA, 19 nt, synthetic construct
  misc_feature 1..19: note = antisense sequence
  source 1..19: mol_type = other RNA
actgaaggaggccattggc

SEQ ID NO: 584 - RNA, 19 nt, synthetic construct
  misc_feature 1..19: note = sense sequence
  source 1..19: mol_type = other RNA
caatggcctccttcagttg

SEQ ID NO: 585 - RNA, 19 nt, synthetic construct
  misc_feature 1..19: note = antisense sequence
  source 1..19: mol_type = other RNA
caactgaaggaggccattg

SEQ ID NO: 586 - RNA, 19 nt, synthetic construct
  misc_feature 1..19: note = sense sequence
  source 1..19: mol_type = other RNA
aatggcctccttcagttgg

SEQ ID NO: 587 - RNA, 19 nt, synthetic construct
  misc_feature 1..19: note = antisense sequence
  source 1..19: mol_type = other RNA
ccaactgaaggaggccatt

SEQ ID NO: 588 - RNA, 19 nt, synthetic construct
  misc_feature 1..19: note = sense sequence
  source 1..19: mol_type = other RNA
tggcctccttcagttggga

SEQ ID NO: 589 - RNA, 19 nt, synthetic construct
  misc_feature 1..19: note = antisense sequence
  source 1..19: mol_type = other RNA
tcccaactgaaggaggcca

SEQ ID NO: 590 - RNA, 19 nt, synthetic construct
  misc_feature 1..19: note = sense sequence
  source 1..19: mol_type = other RNA
ggcctccttcagttgggac

SEQ ID NO: 591 - RNA, 19 nt, synthetic construct
  misc_feature 1..19: note = antisense sequence
  source 1..19: mol_type = other RNA
gtcccaactgaaggaggcc

SEQ ID NO: 592 - RNA, 19 nt, synthetic construct
  misc_feature 1..19: note = sense sequence
  source 1..19: mol_type = other RNA
gcctccttcagttgggaca

SEQ ID NO: 593 - RNA, 19 nt, synthetic construct
  misc_feature 1..19: note = antisense sequence
  source 1..19: mol_type = other RNA
tgtcccaactgaaggaggc

SEQ ID NO: 594 - RNA, 19 nt, synthetic construct
  misc_feature 1..19: note = sense sequence
  source 1..19: mol_type = other RNA
cctccttcagttgggacat

SEQ ID NO: 595 - RNA, 19 nt, synthetic construct
  misc_feature 1..19: note = antisense sequence
  source 1..19: mol_type = other RNA
atgtcccaactgaaggagg

SEQ ID NO: 596 - RNA, 19 nt, synthetic construct
  misc_feature 1..19: note = sense sequence
  source 1..19: mol_type = other RNA
ctccttcagttgggacatg

SEQ ID NO: 597 - RNA, 19 nt, synthetic construct
  misc_feature 1..19: note = antisense sequence
  source 1..19: mol_type = other RNA
catgtcccaactgaaggag

SEQ ID NO: 598 - RNA, 19 nt, synthetic construct
  misc_feature 1..19: note = sense sequence
  source 1..19: mol_type = other RNA
cttcagttgggacatggtc

SEQ ID NO: 599 - RNA, 19 nt, synthetic construct
  misc_feature 1..19: note = antisense sequence
  source 1..19: mol_type = other RNA
gaccatgtcccaactgaag

SEQ ID NO: 600 - RNA, 19 nt, synthetic construct
  misc_feature 1..19: note = sense sequence
  source 1..19: mol_type = other RNA
ttcagttgggacatggtct

SEQ ID NO: 601 - RNA, 19 nt, synthetic construct
  misc_feature 1..19: note = antisense sequence
  source 1..19: mol_type = other RNA
agaccatgtcccaactgaa

SEQ ID NO: 602 - RNA, 19 nt, synthetic construct
  misc_feature 1..19: note = sense sequence
  source 1..19: mol_type = other RNA
tcagttgggacatggtctt

SEQ ID NO: 603 - RNA, 19 nt, synthetic construct
  misc_feature 1..19: note = antisense sequence
  source 1..19: mol_type = other RNA
aagaccatgtcccaactga

SEQ ID NO: 604 - RNA, 19 nt, synthetic construct
  misc_feature 1..19: note = sense sequence
  source 1..19: mol_type = other RNA
ttgggacatggtcttaaag

SEQ ID NO: 605 - RNA, 19 nt, synthetic construct
  misc_feature 1..19: note = antisense sequence
  source 1..19: mol_type = other RNA
ctttaagaccatgtcccaa

SEQ ID NO: 606 - RNA, 19 nt, synthetic construct
  misc_feature 1..19: note = sense sequence
  source 1..19: mol_type = other RNA
tgggacatggtcttaaaga

SEQ ID NO: 607 - RNA, 19 nt, synthetic construct
  misc_feature 1..19: note = antisense sequence
  source 1..19: mol_type = other RNA
tctttaagaccatgtccca

SEQ ID NO: 608 - RNA, 19 nt, synthetic construct
  misc_feature 1..19: note = sense sequence
  source 1..19: mol_type = other RNA
tcttaaagactttgtccat

SEQ ID NO: 609 - RNA, 19 nt, synthetic construct
  misc_feature 1..19: note = antisense sequence
  source 1..19: mol_type = other RNA
atggacaaagtctttaaga

SEQ ID NO: 610 - RNA, 19 nt, synthetic construct
  misc_feature 1..19: note = sense sequence
  source 1..19: mol_type = other RNA
cttaaagactttgtccata

SEQ ID NO: 611 - RNA, 19 nt, synthetic construct
  misc_feature 1..19: note = antisense sequence
  source 1..19: mol_type = other RNA
tatggacaaagtctttaag

SEQ ID NO: 612 - RNA, 19 nt, synthetic construct
  misc_feature 1..19: note = sense sequence
  source 1..19: mol_type = other RNA
taaagactttgtccataag

SEQ ID NO: 613 - RNA, 19 nt, synthetic construct
  misc_feature 1..19: note = antisense sequence
  source 1..19: mol_type = other RNA
cttatggacaaagtcttta

SEQ ID NO: 614 - RNA, 19 nt, synthetic construct
  misc_feature 1..19: note = sense sequence
  source 1..19: mol_type = other RNA
aaagactttgtccataaga

SEQ ID NO: 615 - RNA, 19 nt, synthetic construct
  misc_feature 1..19: note = antisense sequence
  source 1..19: mol_type = other RNA
tcttatggacaaagtcttt

SEQ ID NO: 616 - RNA, 19 nt, synthetic construct
  misc_feature 1..19: note = sense sequence
  source 1..19: mol_type = other RNA
actttgtccataagacgaa

SEQ ID NO: 617 - RNA, 19 nt, synthetic construct
  misc_feature 1..19: note = antisense sequence
  source 1..19: mol_type = other RNA
ttcgtcttatggacaaagt

SEQ ID NO: 618 - RNA, 19 nt, synthetic construct
  misc_feature 1..19: note = sense sequence
  source 1..19: mol_type = other RNA
ctttgtccataagacgaag

SEQ ID NO: 619 - RNA, 19 nt, synthetic construct
  misc_feature 1..19: note = antisense sequence
  source 1..19: mol_type = other RNA
cttcgtcttatggacaaag

SEQ ID NO: 620 - RNA, 19 nt, synthetic construct
  misc_feature 1..19: note = sense sequence
  source 1..19: mol_type = other RNA
tttgtccataagacgaagg

SEQ ID NO: 621 - RNA, 19 nt, synthetic construct
  misc_feature 1..19: note = antisense sequence
  source 1..19: mol_type = other RNA
ccttcgtcttatggacaaa

SEQ ID NO: 622 - RNA, 19 nt, synthetic construct
  misc_feature 1..19: note = sense sequence
  source 1..19: mol_type = other RNA
ttgtccataagacgaaggg

SEQ ID NO: 623 - RNA, 19 nt, synthetic construct
  misc_feature 1..19: note = antisense sequence
  source 1..19: mol_type = other RNA
cccttcgtcttatggacaa

SEQ ID NO: 624 - RNA, 19 nt, synthetic construct
  misc_feature 1..19: note = sense sequence
  source 1..19: mol_type = other RNA
tgtccataagacgaagggc

SEQ ID NO: 625 - RNA, 19 nt, synthetic construct
  misc_feature 1..19: note = antisense sequence
  source 1..19: mol_type = other RNA
gcccttcgtcttatggaca

SEQ ID NO: 626 - RNA, 19 nt, synthetic construct
  misc_feature 1..19: note = sense sequence
  source 1..19: mol_type = other RNA
gtccataagacgaagggcc

SEQ ID NO: 627 - RNA, 19 nt, synthetic construct
  misc_feature 1..19: note = antisense sequence
  source 1..19: mol_type = other RNA
ggcccttcgtcttatggac

SEQ ID NO: 628 - RNA, 19 nt, synthetic construct
  misc_feature 1..19: note = sense sequence
  source 1..19: mol_type = other RNA
ataagacgaagggccaaat

SEQ ID NO: 629 - RNA, 19 nt, synthetic construct
  misc_feature 1..19: note = antisense sequence
  source 1..19: mol_type = other RNA
atttggcccttcgtcttat

SEQ ID NO: 630 - RNA, 19 nt, synthetic construct
  misc_feature 1..19: note = sense sequence
  source 1..19: mol_type = other RNA
taagacgaagggccaaatt

SEQ ID NO: 631 - RNA, 19 nt, synthetic construct
  misc_feature 1..19: note = antisense sequence
  source 1..19: mol_type = other RNA
aatttggcccttcgtctta

SEQ ID NO: 632 - RNA, 19 nt, synthetic construct
  misc_feature 1..19: note = sense sequence
  source 1..19: mol_type = other RNA
aagacgaagggccaaatta

SEQ ID NO: 633 - RNA, 19 nt, synthetic construct
  misc_feature 1..19: note = antisense sequence
  source 1..19: mol_type = other RNA
taatttggcccttcgtctt

SEQ ID NO: 634 - RNA, 19 nt, synthetic construct
  misc_feature 1..19: note = sense sequence
  source 1..19: mol_type = other RNA
agacgaagggccaaattaa

SEQ ID NO: 635 - RNA, 19 nt, synthetic construct
  misc_feature 1..19: note = antisense sequence
  source 1..19: mol_type = other RNA
ttaatttggcccttcgtct

SEQ ID NO: 636 - RNA, 19 nt, synthetic construct
  misc_feature 1..19: note = sense sequence
  source 1..19: mol_type = other RNA
gacgaagggccaaattaat

SEQ ID NO: 637 - RNA, 19 nt, synthetic construct
  misc_feature 1..19: note = antisense sequence
  source 1..19: mol_type = other RNA
attaatttggcccttcgtc

SEQ ID NO: 638 - RNA, 19 nt, synthetic construct
  misc_feature 1..19: note = sense sequence
  source 1..19: mol_type = other RNA
acgaagggccaaattaatg

SEQ ID NO: 639 - RNA, 19 nt, synthetic construct
  misc_feature 1..19: note = antisense sequence
  source 1..19: mol_type = other RNA
cattaatttggcccttcgt

SEQ ID NO: 640 - RNA, 19 nt, synthetic construct
  misc_feature 1..19: note = sense sequence
  source 1..19: mol_type = other RNA
cgaagggccaaattaatga

SEQ ID NO: 641 - RNA, 19 nt, synthetic construct
  misc_feature 1..19: note = antisense sequence
  source 1..19: mol_type = other RNA
tcattaatttggcccttcg

SEQ ID NO: 642 - RNA, 19 nt, synthetic construct
  misc_feature 1..19: note = sense sequence
  source 1..19: mol_type = other RNA
gaagggccaaattaatgac

SEQ ID NO: 643 - RNA, 19 nt, synthetic construct
  misc_feature 1..19: note = antisense sequence
  source 1..19: mol_type = other RNA
gtcattaatttggcccttc

SEQ ID NO: 644 - RNA, 19 nt, synthetic construct
  misc_feature 1..19: note = sense sequence
  source 1..19: mol_type = other RNA
gggccaaattaatgacata

SEQ ID NO: 645 - RNA, 19 nt, synthetic construct
  misc_feature 1..19: note = antisense sequence
  source 1..19: mol_type = other RNA
tatgtcattaatttggccc

SEQ ID NO: 646 - RNA, 19 nt, synthetic construct
  misc_feature 1..19: note = sense sequence
  source 1..19: mol_type = other RNA
ggccaaattaatgacatat

SEQ ID NO: 647 - RNA, 19 nt, synthetic construct
  misc_feature 1..19: note = antisense sequence
  source 1..19: mol_type = other RNA
atatgtcattaatttggcc

SEQ ID NO: 648 - RNA, 19 nt, synthetic construct
  misc_feature 1..19: note = sense sequence
  source 1..19: mol_type = other RNA
ttttatgatctatcgctgc

SEQ ID NO: 649 - RNA, 19 nt, synthetic construct
  misc_feature 1..19: note = antisense sequence
  source 1..19: mol_type = other RNA
gcagcgatagatcataaaa

SEQ ID NO: 650 - RNA, 19 nt, synthetic construct
  misc_feature 1..19: note = sense sequence
  source 1..19: mol_type = other RNA
ttatgatctatcgctgcaa

SEQ ID NO: 651 - RNA, 19 nt, synthetic construct
  misc_feature 1..19: note = antisense sequence
  source 1..19: mol_type = other RNA
ttgcagcgatagatcataa

SEQ ID NO: 652 - RNA, 19 nt, synthetic construct
  misc_feature 1..19: note = sense sequence
  source 1..19: mol_type = other RNA
tatgatctatcgctgcaaa

SEQ ID NO: 653 - RNA, 19 nt, synthetic construct
  misc_feature 1..19: note = antisense sequence
  source 1..19: mol_type = other RNA
tttgcagcgatagatcata

SEQ ID NO: 654 - RNA, 19 nt, synthetic construct
  misc_feature 1..19: note = sense sequence
  source 1..19: mol_type = other RNA
gaagaggtaaagaatatgt

SEQ ID NO: 655 - RNA, 19 nt, synthetic construct
  misc_feature 1..19: note = antisense sequence
  source 1..19: mol_type = other RNA
acatattctttacctcttc

SEQ ID NO: 656 - RNA, 19 nt, synthetic construct
  misc_feature 1..19: note = sense sequence
  source 1..19: mol_type = other RNA
aagaggtaaagaatatgtc

SEQ ID NO: 657 - RNA, 19 nt, synthetic construct
  misc_feature 1..19: note = antisense sequence
  source 1..19: mol_type = other RNA
gacatattctttacctctt

SEQ ID NO: 658 - RNA, 19 nt, synthetic construct
  misc_feature 1..19: note = sense sequence
  source 1..19: mol_type = other RNA
gaagagcaactaactaact

SEQ ID NO: 659 - RNA, 19 nt, synthetic construct
  misc_feature 1..19: note = antisense sequence
  source 1..19: mol_type = other RNA
agttagttagttgctcttc

SEQ ID NO: 660 - RNA, 19 nt, synthetic construct
  misc_feature 1..19: note = sense sequence
  source 1..19: mol_type = other RNA
aagagcaactaactaactt

SEQ ID NO: 661 - RNA, 19 nt, synthetic construct
  misc_feature 1..19: note = antisense sequence
  source 1..19: mol_type = other RNA
aagttagttagttgctctt

SEQ ID NO: 662 - RNA, 19 nt, synthetic construct
  misc_feature 1..19: note = sense sequence
  source 1..19: mol_type = other RNA
agagcaactaactaactta

SEQ ID NO: 663 - RNA, 19 nt, synthetic construct
  misc_feature 1..19: note = antisense sequence
  source 1..19: mol_type = other RNA
taagttagttagttgctct

SEQ ID NO: 664 - RNA, 19 nt, synthetic construct
  misc_feature 1..19: note = sense sequence
  source 1..19: mol_type = other RNA
gagcaactaactaacttaa

SEQ ID NO: 665 - RNA, 19 nt, synthetic construct
  misc_feature 1..19: note = antisense sequence
  source 1..19: mol_type = other RNA
ttaagttagttagttgctc

SEQ ID NO: 666 - RNA, 19 nt, synthetic construct
  misc_feature 1..19: note = sense sequence
  source 1..19: mol_type = other RNA
caagataatagcatcaaag

SEQ ID NO: 667 - RNA, 19 nt, synthetic construct
  misc_feature 1..19: note = antisense sequence
  source 1..19: mol_type = other RNA
ctttgatgctattatcttg

SEQ ID NO: 668 - RNA, 19 nt, synthetic construct
  misc_feature 1..19: note = sense sequence
  source 1..19: mol_type = other RNA
agataatagcatcaaagac

SEQ ID NO: 669 - RNA, 19 nt, synthetic construct
  misc_feature 1..19: note = antisense sequence
  source 1..19: mol_type = other RNA
gtctttgatgctattatct

SEQ ID NO: 670 - RNA, 19 nt, synthetic construct
  misc_feature 1..19: note = sense sequence
  source 1..19: mol_type = other RNA
gataatagcatcaaagacc

SEQ ID NO: 671 - RNA, 19 nt, synthetic construct
  misc_feature 1..19: note = antisense sequence
  source 1..19: mol_type = other RNA
ggtctttgatgctattatc

SEQ ID NO: 672 - RNA, 19 nt, synthetic construct
  misc_feature 1..19: note = sense sequence
  source 1..19: mol_type = other RNA
ataatagcatcaaagacct

SEQ ID NO: 673 - RNA, 19 nt, synthetic construct
  misc_feature 1..19: note = antisense sequence
  source 1..19: mol_type = other RNA
aggtctttgatgctattat

SEQ ID NO: 674 - RNA, 19 nt, synthetic construct
  misc_feature 1..19: note = sense sequence
  source 1..19: mol_type = other RNA
taatagcatcaaagacctt

SEQ ID NO: 675 - RNA, 19 nt, synthetic construct
  misc_feature 1..19: note = antisense sequence
  source 1..19: mol_type = other RNA
aaggtctttgatgctatta

SEQ ID NO: 676 - RNA, 19 nt, synthetic construct
  misc_feature 1..19: note = sense sequence
  source 1..19: mol_type = other RNA
agcatcaaagaccttctcc

SEQ ID NO: 677 - RNA, 19 nt, synthetic construct
  misc_feature 1..19: note = antisense sequence
  source 1..19: mol_type = other RNA
ggagaaggtctttgatgct

SEQ ID NO: 678 - RNA, 19 nt, synthetic construct
  misc_feature 1..19: note = sense sequence
  source 1..19: mol_type = other RNA
gcatcaaagaccttctcca

SEQ ID NO: 679 - RNA, 19 nt, synthetic construct
  misc_feature 1..19: note = antisense sequence
  source 1..19: mol_type = other RNA
tggagaaggtctttgatgc

SEQ ID NO: 680 - RNA, 19 nt, synthetic construct
  misc_feature 1..19: note = sense sequence
  source 1..19: mol_type = other RNA
catcaaagaccttctccag

SEQ ID NO: 681 - RNA, 19 nt, synthetic construct
  misc_feature 1..19: note = antisense sequence
  source 1..19: mol_type = other RNA
ctggagaaggtctttgatg

SEQ ID NO: 682 - RNA, 19 nt, synthetic construct
  misc_feature 1..19: note = sense sequence
  source 1..19: mol_type = other RNA
atcaaagaccttctccaga

SEQ ID NO: 683 - RNA, 19 nt, synthetic construct
  misc_feature 1..19: note = antisense sequence
  source 1..19: mol_type = other RNA
tctggagaaggtctttgat

SEQ ID NO: 684 - RNA, 19 nt, synthetic construct
  misc_feature 1..19: note = sense sequence
  source 1..19: mol_type = other RNA
tcaaagaccttctccagac

SEQ ID NO: 685 - RNA, 19 nt, synthetic construct
  misc_feature 1..19: note = antisense sequence
  source 1..19: mol_type = other RNA
gtctggagaaggtctttga

SEQ ID NO: 686 - RNA, 19 nt, synthetic construct
  misc_feature 1..19: note = sense sequence
  source 1..19: mol_type = other RNA
caaagaccttctccagacc

SEQ ID NO: 687 - RNA, 19 nt, synthetic construct
  misc_feature 1..19: note = antisense sequence
  source 1..19: mol_type = other RNA
ggtctggagaaggtctttg

SEQ ID NO: 688 - RNA, 19 nt, synthetic construct
  misc_feature 1..19: note = sense sequence
  source 1..19: mol_type = other RNA
aaagaccttctccagaccg

SEQ ID NO: 689 - RNA, 19 nt, synthetic construct
  misc_feature 1..19: note = antisense sequence
  source 1..19: mol_type = other RNA
cggtctggagaaggtcttt

SEQ ID NO: 690 - RNA, 19 nt, synthetic construct
  misc_feature 1..19: note = sense sequence
  source 1..19: mol_type = other RNA
aagaccttctccagaccgt

SEQ ID NO: 691 - RNA, 19 nt, synthetic construct
  misc_feature 1..19: note = antisense sequence
  source 1..19: mol_type = other RNA
acggtctggagaaggtctt

SEQ ID NO: 692 - RNA, 19 nt, synthetic construct
  misc_feature 1..19: note = sense sequence
  source 1..19: mol_type = other RNA
agaccttctccagaccgtg

SEQ ID NO: 693 - RNA, 19 nt, synthetic construct
  misc_feature 1..19: note = antisense sequence
  source 1..19: mol_type = other RNA
cacggtctggagaaggtct

SEQ ID NO: 694 - RNA, 19 nt, synthetic construct
  misc_feature 1..19: note = sense sequence
  source 1..19: mol_type = other RNA
gaccttctccagaccgtgg

SEQ ID NO: 695 - RNA, 19 nt, synthetic construct
  misc_feature 1..19: note = antisense sequence
  source 1..19: mol_type = other RNA
ccacggtctggagaaggtc

SEQ ID NO: 696 - RNA, 19 nt, synthetic construct
  misc_feature 1..19: note = sense sequence
  source 1..19: mol_type = other RNA
gaccgtggaagaccaatat

SEQ ID NO: 697 - RNA, 19 nt, synthetic construct
  misc_feature 1..19: note = antisense sequence
  source 1..19: mol_type = other RNA
atattggtcttccacggtc

SEQ ID NO: 698 - RNA, 19 nt, synthetic construct
  misc_feature 1..19: note = sense sequence
  source 1..19: mol_type = other RNA
ccgtggaagaccaatataa

SEQ ID NO: 699 - RNA, 19 nt, synthetic construct
  misc_feature 1..19: note = antisense sequence
  source 1..19: mol_type = other RNA
ttatattggtcttccacgg

SEQ ID NO: 700 - RNA, 19 nt, synthetic construct
  misc_feature 1..19: note = sense sequence
  source 1..19: mol_type = other RNA
cgtggaagaccaatataaa

SEQ ID NO: 701 - RNA, 19 nt, synthetic construct
  misc_feature 1..19: note = antisense sequence
  source 1..19: mol_type = other RNA
tttatattggtcttccacg

SEQ ID NO: 702 - RNA, 19 nt, synthetic construct
  misc_feature 1..19: note = sense sequence
  source 1..19: mol_type = other RNA
cagcatagtcaaataaaag

SEQ ID NO: 703 - RNA, 19 nt, synthetic construct
  misc_feature 1..19: note = antisense sequence
  source 1..19: mol_type = other RNA
cttttatttgactatgctg

SEQ ID NO: 704 - RNA, 19 nt, synthetic construct
  misc_feature 1..19: note = sense sequence
  source 1..19: mol_type = other RNA
aaatcagctcagaaggact

SEQ ID NO: 705 - RNA, 19 nt, synthetic construct
  misc_feature 1..19: note = antisense sequence
  source 1..19: mol_type = other RNA
agtccttctgagctgattt

SEQ ID NO: 706 - RNA, 19 nt, synthetic construct
  misc_feature 1..19: note = sense sequence
  source 1..19: mol_type = other RNA
aatcagctcagaaggacta

SEQ ID NO: 707 - RNA, 19 nt, synthetic construct
  misc_feature 1..19: note = antisense sequence
  source 1..19: mol_type = other RNA
tagtccttctgagctgatt

SEQ ID NO: 708 - RNA, 19 nt, synthetic construct
  misc_feature 1..19: note = sense sequence
  source 1..19: mol_type = other RNA
atcagctcagaaggactag

SEQ ID NO: 709 - RNA, 19 nt, synthetic construct
  misc_feature 1..19: note = antisense sequence
  source 1..19: mol_type = other RNA
ctagtccttctgagctgat

SEQ ID NO: 710 - RNA, 19 nt, synthetic construct
  misc_feature 1..19: note = sense sequence
  source 1..19: mol_type = other RNA
tcagctcagaaggactagt

SEQ ID NO: 711 - RNA, 19 nt, synthetic construct
  misc_feature 1..19: note = antisense sequence
  source 1..19: mol_type = other RNA
actagtccttctgagctga

SEQ ID NO: 712 - RNA, 19 nt, synthetic construct
  misc_feature 1..19: note = sense sequence
  source 1..19: mol_type = other RNA
agctcagaaggactagtat

SEQ ID NO: 713 - RNA, 19 nt, synthetic construct
  misc_feature 1..19: note = antisense sequence
  source 1..19: mol_type = other RNA
atactagtccttctgagct

SEQ ID NO: 714 - RNA, 19 nt, synthetic construct
  misc_feature 1..19: note = sense sequence
  source 1..19: mol_type = other RNA
cagaaggactagtattcaa

SEQ ID NO: 715 - RNA, 19 nt, synthetic construct
  misc_feature 1..19: note = antisense sequence
  source 1..19: mol_type = other RNA
ttgaatactagtccttctg

SEQ ID NO: 716 - RNA, 19 nt, synthetic construct
  misc_feature 1..19: note = sense sequence
  source 1..19: mol_type = other RNA
agaaggactagtattcaag

SEQ ID NO: 717 - RNA, 19 nt, synthetic construct
  misc_feature 1..19: note = antisense sequence
  source 1..19: mol_type = other RNA
cttgaatactagtccttct

SEQ ID NO: 718 - RNA, 19 nt, synthetic construct
  misc_feature 1..19: note = sense sequence
  source 1..19: mol_type = other RNA
gaaggactagtattcaaga

SEQ ID NO: 719 - RNA, 19 nt, synthetic construct
  misc_feature 1..19: note = antisense sequence
  source 1..19: mol_type = other RNA
tcttgaatactagtccttc

SEQ ID NO: 720 - RNA, 19 nt, synthetic construct
  misc_feature 1..19: note = sense sequence
  source 1..19: mol_type = other RNA
aaggactagtattcaagaa

SEQ ID NO: 721 - RNA, 19 nt, synthetic construct
  misc_feature 1..19: note = antisense sequence
  source 1..19: mol_type = other RNA
ttcttgaatactagtcctt

SEQ ID NO: 722 - RNA, 19 nt, synthetic construct
  misc_feature 1..19: note = sense sequence
  source 1..19: mol_type = other RNA
aatttctctatcttccaag

SEQ ID NO: 723 - RNA, 19 nt, synthetic construct
  misc_feature 1..19: note = antisense sequence
  source 1..19: mol_type = other RNA
cttggaagatagagaaatt

SEQ ID NO: 724 - RNA, 19 nt, synthetic construct
  misc_feature 1..19: note = sense sequence
  source 1..19: mol_type = other RNA
atttctctatcttccaagc

SEQ ID NO: 725 - RNA, 19 nt, synthetic construct
  misc_feature 1..19: note = antisense sequence
  source 1..19: mol_type = other RNA
gcttggaagatagagaaat

SEQ ID NO: 726 - RNA, 19 nt, synthetic construct
  misc_feature 1..19: note = sense sequence
  source 1..19: mol_type = other RNA
tttctctatcttccaagcc

SEQ ID NO: 727 - RNA, 19 nt, synthetic construct
  misc_feature 1..19: note = antisense sequence
  source 1..19: mol_type = other RNA
ggcttggaagatagagaaa

SEQ ID NO: 728 - RNA, 19 nt, synthetic construct
  misc_feature 1..19: note = sense sequence
  source 1..19: mol_type = other RNA
ttctctatcttccaagcca

SEQ ID NO: 729 - RNA, 19 nt, synthetic construct
  misc_feature 1..19: note = antisense sequence
  source 1..19: mol_type = other RNA
tggcttggaagatagagaa

SEQ ID NO: 730 - RNA, 19 nt, synthetic construct
  misc_feature 1..19: note = sense sequence
  source 1..19: mol_type = other RNA
tctctatcttccaagccaa

SEQ ID NO: 731 - RNA, 19 nt, synthetic construct
  misc_feature 1..19: note = antisense sequence
  source 1..19: mol_type = other RNA
ttggcttggaagatagaga

SEQ ID NO: 732 - RNA, 19 nt, synthetic construct
  misc_feature 1..19: note = sense sequence
  source 1..19: mol_type = other RNA
ctctatcttccaagccaag

SEQ ID NO: 733 - RNA, 19 nt, synthetic construct
  misc_feature 1..19: note = antisense sequence
  source 1..19: mol_type = other RNA
cttggcttggaagatagag

SEQ ID NO: 734 - RNA, 19 nt, synthetic construct
  misc_feature 1..19: note = sense sequence
  source 1..19: mol_type = other RNA
ccaagccaagagcaccaag

SEQ ID NO: 735 - RNA, 19 nt, synthetic construct
  misc_feature 1..19: note = antisense sequence
  source 1..19: mol_type = other RNA
cttggtgctcttggcttgg

SEQ ID NO: 736 - RNA, 19 nt, synthetic construct
  misc_feature 1..19: note = sense sequence
  source 1..19: mol_type = other RNA
caagccaagagcaccaaga

SEQ ID NO: 737 - RNA, 19 nt, synthetic construct
  misc_feature 1..19: note = antisense sequence
  source 1..19: mol_type = other RNA
tcttggtgctcttggcttg

SEQ ID NO: 738 - RNA, 19 nt, synthetic construct
  misc_feature 1..19: note = sense sequence
  source 1..19: mol_type = other RNA
aagccaagagcaccaagaa

SEQ ID NO: 739 - RNA, 19 nt, synthetic construct
  misc_feature 1..19: note = antisense sequence
  source 1..19: mol_type = other RNA
ttcttggtgctcttggctt

SEQ ID NO: 740 - RNA, 19 nt, synthetic construct
  misc_feature 1..19: note = sense sequence
  source 1..19: mol_type = other RNA
ctactccctttcttcagtt

SEQ ID NO: 741 - RNA, 19 nt, synthetic construct
  misc_feature 1..19: note = antisense sequence
  source 1..19: mol_type = other RNA
aactgaagaaagggagtag

SEQ ID NO: 742 - RNA, 19 nt, synthetic construct
  misc_feature 1..19: note = sense sequence
  source 1..19: mol_type = other RNA
tactccctttcttcagttg

SEQ ID NO: 743 - RNA, 19 nt, synthetic construct
  misc_feature 1..19: note = antisense sequence
  source 1..19: mol_type = other RNA
caactgaagaaagggagta

SEQ ID NO: 744 - RNA, 19 nt, synthetic construct
  misc_feature 1..19: note = sense sequence
  source 1..19: mol_type = other RNA
actccctttcttcagttga

SEQ ID NO: 745 - RNA, 19 nt, synthetic construct
  misc_feature 1..19: note = antisense sequence
  source 1..19: mol_type = other RNA
tcaactgaagaaagggagt

SEQ ID NO: 746 - RNA, 19 nt, synthetic construct
  misc_feature 1..19: note = sense sequence
  source 1..19: mol_type = other RNA
ctccctttcttcagttgaa

SEQ ID NO: 747 - RNA, 19 nt, synthetic construct
  misc_feature 1..19: note = antisense sequence
  source 1..19: mol_type = other RNA
ttcaactgaagaaagggag

SEQ ID NO: 748 - RNA, 19 nt, synthetic construct
  misc_feature 1..19: note = sense sequence
  source 1..19: mol_type = other RNA
tccctttcttcagttgaat

SEQ ID NO: 749 - RNA, 19 nt, synthetic construct
  misc_feature 1..19: note = antisense sequence
  source 1..19: mol_type = other RNA
attcaactgaagaaaggga

SEQ ID NO: 750 - RNA, 19 nt, synthetic construct
  misc_feature 1..19: note = sense sequence
  source 1..19: mol_type = other RNA
ccctttcttcagttgaatg

SEQ ID NO: 751 - RNA, 19 nt, synthetic construct
  misc_feature 1..19: note = antisense sequence
  source 1..19: mol_type = other RNA
cattcaactgaagaaaggg

SEQ ID NO: 752 - RNA, 19 nt, synthetic construct
  misc_feature 1..19: note = sense sequence
  source 1..19: mol_type = other RNA
gtatgccatcagacccagc

SEQ ID NO: 753 - RNA, 19 nt, synthetic construct
  misc_feature 1..19: note = antisense sequence
  source 1..19: mol_type = other RNA
gctgggtctgatggcatac

SEQ ID NO: 754 - RNA, 19 nt, synthetic construct
  misc_feature 1..19: note = sense sequence
  source 1..19: mol_type = other RNA
tatgccatcagacccagca

SEQ ID NO: 755 - RNA, 19 nt, synthetic construct
  misc_feature 1..19: note = antisense sequence
  source 1..19: mol_type = other RNA
tgctgggtctgatggcata

SEQ ID NO: 756 - RNA, 19 nt, synthetic construct
  misc_feature 1..19: note = sense sequence
  source 1..19: mol_type = other RNA
tgccatcagacccagcaac

SEQ ID NO: 757 - RNA, 19 nt, synthetic construct
  misc_feature 1..19: note = antisense sequence
  source 1..19: mol_type = other RNA
gttgctgggtctgatggca

SEQ ID NO: 758 - RNA, 19 nt, synthetic construct
  misc_feature 1..19: note = sense sequence
  source 1..19: mol_type = other RNA
gccatcagacccagcaact

SEQ ID NO: 759 - RNA, 19 nt, synthetic construct
  misc_feature 1..19: note = antisense sequence
  source 1..19: mol_type = other RNA
agttgctgggtctgatggc

SEQ ID NO: 760 - RNA, 19 nt, synthetic construct
  misc_feature 1..19: note = sense sequence
  source 1..19: mol_type = other RNA
tcagacccagcaactctca

SEQ ID NO: 761 - RNA, 19 nt, synthetic construct
  misc_feature 1..19: note = antisense sequence
  source 1..19: mol_type = other RNA
tgagagttgctgggtctga

SEQ ID NO: 762 - RNA, 19 nt, synthetic construct
  misc_feature 1..19: note = sense sequence
  source 1..19: mol_type = other RNA
cagacccagcaactctcaa

SEQ ID NO: 763 - RNA, 19 nt, synthetic construct
  misc_feature 1..19: note = antisense sequence
  source 1..19: mol_type = other RNA
ttgagagttgctgggtctg

SEQ ID NO: 764 - RNA, 19 nt, synthetic construct
  misc_feature 1..19: note = sense sequence
  source 1..19: mol_type = other RNA
gacccagcaactctcaagt

SEQ ID NO: 765 - RNA, 19 nt, synthetic construct
  misc_feature 1..19: note = antisense sequence
  source 1..19: mol_type = other RNA
acttgagagttgctgggtc

SEQ ID NO: 766 - RNA, 19 nt, synthetic construct
  misc_feature 1..19: note = sense sequence
  source 1..19: mol_type = other RNA
ttcatgtctactgtgatgt

SEQ ID NO: 767 - RNA, 19 nt, synthetic construct
  misc_feature 1..19: note = antisense sequence
  source 1..19: mol_type = other RNA
acatcacagtagacatgaa

SEQ ID NO: 768 - RNA, 19 nt, synthetic construct
  misc_feature 1..19: note = sense sequence
  source 1..19: mol_type = other RNA
tcatgtctactgtgatgtt

SEQ ID NO: 769 - RNA, 19 nt, synthetic construct
  misc_feature 1..19: note = antisense sequence
  source 1..19: mol_type = other RNA
aacatcacagtagacatga

SEQ ID NO: 770 - RNA, 19 nt, synthetic construct
  misc_feature 1..19: note = sense sequence
  source 1..19: mol_type = other RNA
catgtctactgtgatgtta

SEQ ID NO: 771 - RNA, 19 nt, synthetic construct
  misc_feature 1..19: note = antisense sequence
  source 1..19: mol_type = other RNA
taacatcacagtagacatg

SEQ ID NO: 772 - RNA, 19 nt, synthetic construct
  misc_feature 1..19: note = sense sequence
  source 1..19: mol_type = other RNA
atgtctactgtgatgttat

SEQ ID NO: 773 - RNA, 19 nt, synthetic construct
  misc_feature 1..19: note = antisense sequence
  source 1..19: mol_type = other RNA
ataacatcacagtagacat

SEQ ID NO: 774 - RNA, 19 nt, synthetic construct
  misc_feature 1..19: note = sense sequence
  source 1..19: mol_type = other RNA
tgtctactgtgatgttata

SEQ ID NO: 775 - RNA, 19 nt, synthetic construct
  misc_feature 1..19: note = antisense sequence
  source 1..19: mol_type = other RNA
tataacatcacagtagaca

SEQ ID NO: 776 - RNA, 19 nt, synthetic construct
  misc_feature 1..19: note = sense sequence
  source 1..19: mol_type = other RNA
gatgttatatcaggtagtc

SEQ ID NO: 777 - RNA, 19 nt, synthetic construct
  misc_feature 1..19: note = antisense sequence
  source 1..19: mol_type = other RNA
gactacctgatataacatc

SEQ ID NO: 778 - RNA, 19 nt, synthetic construct
  misc_feature 1..19: note = sense sequence
  source 1..19: mol_type = other RNA
atgttatatcaggtagtcc

SEQ ID NO: 779 - RNA, 19 nt, synthetic construct
  misc_feature 1..19: note = antisense sequence
  source 1..19: mol_type = other RNA
ggactacctgatataacat

SEQ ID NO: 780 - RNA, 19 nt, synthetic construct
  misc_feature 1..19: note = sense sequence
  source 1..19: mol_type = other RNA
tgttatatcaggtagtcca

SEQ ID NO: 781 - RNA, 19 nt, synthetic construct
  misc_feature 1..19: note = antisense sequence
  source 1..19: mol_type = other RNA
tggactacctgatataaca

SEQ ID NO: 782 - RNA, 19 nt, synthetic construct
  misc_feature 1..19: note = sense sequence
  source 1..19: mol_type = other RNA
gttatatcaggtagtccat

SEQ ID NO: 783 - RNA, 19 nt, synthetic construct
  misc_feature 1..19: note = antisense sequence
  source 1..19: mol_type = other RNA
atggactacctgatataac

SEQ ID NO: 784 - RNA, 19 nt, synthetic construct
  misc_feature 1..19: note = sense sequence
  source 1..19: mol_type = other RNA
tatcaggtagtccatggac

SEQ ID NO: 785 - RNA, 19 nt, synthetic construct
  misc_feature 1..19: note = antisense sequence
  source 1..19: mol_type = other RNA
gtccatggactacctgata

SEQ ID NO: 786 - RNA, 19 nt, synthetic construct
  misc_feature 1..19: note = sense sequence
  source 1..19: mol_type = other RNA
atcaggtagtccatggaca

SEQ ID NO: 787 - RNA, 19 nt, synthetic construct
  misc_feature 1..19: note = antisense sequence
  source 1..19: mol_type = other RNA
tgtccatggactacctgat

SEQ ID NO: 788 - RNA, 19 nt, synthetic construct
  misc_feature 1..19: note = sense sequence
  source 1..19: mol_type = other RNA
tcaggtagtccatggacat

SEQ ID NO: 789 - RNA, 19 nt, synthetic construct
  misc_feature 1..19: note = antisense sequence
  source 1..19: mol_type = other RNA
atgtccatggactacctga

SEQ ID NO: 790 - RNA, 19 nt, synthetic construct
  misc_feature 1..19: note = sense sequence
  source 1..19: mol_type = other RNA
caggtagtccatggacatt

SEQ ID NO: 791 - RNA, 19 nt, synthetic construct
  misc_feature 1..19: note = antisense sequence
  source 1..19: mol_type = other RNA
aatgtccatggactacctg

SEQ ID NO: 792 - RNA, 19 nt, synthetic construct
  misc_feature 1..19: note = sense sequence
  source 1..19: mol_type = other RNA
aggtagtccatggacatta

SEQ ID NO: 793 - RNA, 19 nt, synthetic construct
  misc_feature 1..19: note = antisense sequence
  source 1..19: mol_type = other RNA
taatgtccatggactacct

SEQ ID NO: 794 - RNA, 19 nt, synthetic construct
  misc_feature 1..19: note = sense sequence
  source 1..19: mol_type = other RNA
ggtagtccatggacattaa

SEQ ID NO: 795 - RNA, 19 nt, synthetic construct
  misc_feature 1..19: note = antisense sequence
  source 1..19: mol_type = other RNA
ttaatgtccatggactacc

SEQ ID NO: 796 - RNA, 19 nt, synthetic construct
  misc_feature 1..19: note = sense sequence
  source 1..19: mol_type = other RNA
gtagtccatggacattaat

SEQ ID NO: 797 - RNA, 19 nt, synthetic construct
  misc_feature 1..19: note = antisense sequence
  source 1..19: mol_type = other RNA
attaatgtccatggactac

SEQ ID NO: 798 - RNA, 19 nt, synthetic construct
  misc_feature 1..19: note = sense sequence
  source 1..19: mol_type = other RNA
tagtccatggacattaatt

SEQ ID NO: 799 - RNA, 19 nt, synthetic construct
  misc_feature 1..19: note = antisense sequence
  source 1..19: mol_type = other RNA
aattaatgtccatggacta

SEQ ID NO: 800 - RNA, 19 nt, synthetic construct
  misc_feature 1..19: note = sense sequence
  source 1..19: mol_type = other RNA
agtccatggacattaattc

SEQ ID NO: 801 - RNA, 19 nt, synthetic construct
  misc_feature 1..19: note = antisense sequence
  source 1..19: mol_type = other RNA
gaattaatgtccatggact

SEQ ID NO: 802 - RNA, 19 nt, synthetic construct
  misc_feature 1..19: note = sense sequence
  source 1..19: mol_type = other RNA
gtccatggacattaattca

SEQ ID NO: 803 - RNA, 19 nt, synthetic construct
  misc_feature 1..19: note = antisense sequence
  source 1..19: mol_type = other RNA
tgaattaatgtccatggac

SEQ ID NO: 804 - RNA, 19 nt, synthetic construct
  misc_feature 1..19: note = sense sequence
  source 1..19: mol_type = other RNA
tccatggacattaattcaa

SEQ ID NO: 805 - RNA, 19 nt, synthetic construct
  misc_feature 1..19: note = antisense sequence
  source 1..19: mol_type = other RNA
ttgaattaatgtccatgga

SEQ ID NO: 806 - RNA, 19 nt, synthetic construct
  misc_feature 1..19: note = sense sequence
  source 1..19: mol_type = other RNA
ccatggacattaattcaac

SEQ ID NO: 807 - RNA, 19 nt, synthetic construct
  misc_feature 1..19: note = antisense sequence
  source 1..19: mol_type = other RNA
gttgaattaatgtccatgg

SEQ ID NO: 808 - RNA, 19 nt, synthetic construct
  misc_feature 1..19: note = sense sequence
  source 1..19: mol_type = other RNA
atcgaatagatggatcaca

SEQ ID NO: 809 - RNA, 19 nt, synthetic construct
  misc_feature 1..19: note = antisense sequence
  source 1..19: mol_type = other RNA
tgtgatccatctattcgat

SEQ ID NO: 810 - RNA, 19 nt, synthetic construct
  misc_feature 1..19: note = sense sequence
  source 1..19: mol_type = other RNA
aatatggttttgggaggct

SEQ ID NO: 811 - RNA, 19 nt, synthetic construct
  misc_feature 1..19: note = antisense sequence
  source 1..19: mol_type = other RNA
agcctcccaaaaccatatt

SEQ ID NO: 812 - RNA, 19 nt, synthetic construct
  misc_feature 1..19: note = sense sequence
  source 1..19: mol_type = other RNA
atatggttttgggaggctt

SEQ ID NO: 813 - RNA, 19 nt, synthetic construct
  misc_feature 1..19: note = antisense sequence
  source 1..19: mol_type = other RNA
aagcctcccaaaaccatat

SEQ ID NO: 814 - RNA, 19 nt, synthetic construct
  misc_feature 1..19: note = sense sequence
  source 1..19: mol_type = other RNA
tatggttttgggaggcttg

SEQ ID NO: 815 - RNA, 19 nt, synthetic construct
  misc_feature 1..19: note = antisense sequence
  source 1..19: mol_type = other RNA
caagcctcccaaaaccata

SEQ ID NO: 816 - RNA, 19 nt, synthetic construct
  misc_feature 1..19: note = sense sequence
  source 1..19: mol_type = other RNA
atggttttgggaggcttga

SEQ ID NO: 817 - RNA, 19 nt, synthetic construct
  misc_feature 1..19: note = antisense sequence
  source 1..19: mol_type = other RNA
tcaagcctcccaaaaccat

SEQ ID NO: 818 - RNA, 19 nt, synthetic construct
  misc_feature 1..19: note = sense sequence
  source 1..19: mol_type = other RNA
tggttttgggaggcttgat

SEQ ID NO: 819 - RNA, 19 nt, synthetic construct
  misc_feature 1..19: note = antisense sequence
  source 1..19: mol_type = other RNA
atcaagcctcccaaaacca

SEQ ID NO: 820 - RNA, 19 nt, synthetic construct
  misc_feature 1..19: note = sense sequence
  source 1..19: mol_type = other RNA
ggttttgggaggcttgatg

SEQ ID NO: 821 - RNA, 19 nt, synthetic construct
  misc_feature 1..19: note = antisense sequence
  source 1..19: mol_type = other RNA
catcaagcctcccaaaacc

SEQ ID NO: 822 - RNA, 19 nt, synthetic construct
  misc_feature 1..19: note = sense sequence
  source 1..19: mol_type = other RNA
gttttgggaggcttgatgg

SEQ ID NO: 823 - RNA, 19 nt, synthetic construct
  misc_feature 1..19: note = antisense sequence
  source 1..19: mol_type = other RNA
ccatcaagcctcccaaaac

SEQ ID NO: 824 - RNA, 19 nt, synthetic construct
  misc_feature 1..19: note = sense sequence
  source 1..19: mol_type = other RNA
ttttgggaggcttgatgga

SEQ ID NO: 825 - RNA, 19 nt, synthetic construct
  misc_feature 1..19: note = antisense sequence
  source 1..19: mol_type = other RNA
tccatcaagcctcccaaaa

SEQ ID NO: 826 - RNA, 19 nt, synthetic construct
  misc_feature 1..19: note = sense sequence
  source 1..19: mol_type = other RNA
agaattttggttgggccta

SEQ ID NO: 827 - RNA, 19 nt, synthetic construct
  misc_feature 1..19: note = antisense sequence
  source 1..19: mol_type = other RNA
taggcccaaccaaaattct

SEQ ID NO: 828 - RNA, 19 nt, synthetic construct
  misc_feature 1..19: note = sense sequence
  source 1..19: mol_type = other RNA
gaattttggttgggcctag

SEQ ID NO: 829 - RNA, 19 nt, synthetic construct
  misc_feature 1..19: note = antisense sequence
  source 1..19: mol_type = other RNA
ctaggcccaaccaaaattc

SEQ ID NO: 830 - RNA, 19 nt, synthetic construct
  misc_feature 1..19: note = sense sequence
  source 1..19: mol_type = other RNA
aattttggttgggcctaga

SEQ ID NO: 831 - RNA, 19 nt, synthetic construct
  misc_feature 1..19: note = antisense sequence
  source 1..19: mol_type = other RNA
tctaggcccaaccaaaatt

SEQ ID NO: 832 - RNA, 19 nt, synthetic construct
  misc_feature 1..19: note = sense sequence
  source 1..19: mol_type = other RNA
attttggttgggcctagag

SEQ ID NO: 833 - RNA, 19 nt, synthetic construct
  misc_feature 1..19: note = antisense sequence
  source 1..19: mol_type = other RNA
ctctaggcccaaccaaaat

SEQ ID NO: 834 - RNA, 19 nt, synthetic construct
  misc_feature 1..19: note = sense sequence
  source 1..19: mol_type = other RNA
agaagatatactccatagt

SEQ ID NO: 835 - RNA, 19 nt, synthetic construct
  misc_feature 1..19: note = antisense sequence
  source 1..19: mol_type = other RNA
actatggagtatatcttct

SEQ ID NO: 836 - RNA, 19 nt, synthetic construct
  misc_feature 1..19: note = sense sequence
  source 1..19: mol_type = other RNA
gaagatatactccatagtg

SEQ ID NO: 837 - RNA, 19 nt, synthetic construct
  misc_feature 1..19: note = antisense sequence
  source 1..19: mol_type = other RNA
cactatggagtatatcttc

SEQ ID NO: 838 - RNA, 19 nt, synthetic construct
  misc_feature 1..19: note = sense sequence
  source 1..19: mol_type = other RNA
aagatatactccatagtga

SEQ ID NO: 839 - RNA, 19 nt, synthetic construct
  misc_feature 1..19: note = antisense sequence
  source 1..19: mol_type = other RNA
tcactatggagtatatctt

SEQ ID NO: 840 - RNA, 19 nt, synthetic construct
  misc_feature 1..19: note = sense sequence
  source 1..19: mol_type = other RNA
gatatactccatagtgaag

SEQ ID NO: 841 - RNA, 19 nt, synthetic construct
  misc_feature 1..19: note = antisense sequence
  source 1..19: mol_type = other RNA
cttcactatggagtatatc

SEQ ID NO: 842 - RNA, 19 nt, synthetic construct
  misc_feature 1..19: note = sense sequence
  source 1..19: mol_type = other RNA
atatactccatagtgaagc

SEQ ID NO: 843 - RNA, 19 nt, synthetic construct
  misc_feature 1..19: note = antisense sequence
  source 1..19: mol_type = other RNA
gcttcactatggagtatat

SEQ ID NO: 844 - RNA, 19 nt, synthetic construct
  misc_feature 1..19: note = sense sequence
  source 1..19: mol_type = other RNA
tatactccatagtgaagca

SEQ ID NO: 845 - RNA, 19 nt, synthetic construct
  misc_feature 1..19: note = antisense sequence
  source 1..19: mol_type = other RNA
tgcttcactatggagtata

SEQ ID NO: 846 - RNA, 19 nt, synthetic construct
  misc_feature 1..19: note = sense sequence
  source 1..19: mol_type = other RNA
atactccatagtgaagcaa

SEQ ID NO: 847 - RNA, 19 nt, synthetic construct
  misc_feature 1..19: note = antisense sequence
  source 1..19: mol_type = other RNA
ttgcttcactatggagtat

SEQ ID NO: 848 - RNA, 19 nt, synthetic construct
  misc_feature 1..19: note = sense sequence
  source 1..19: mol_type = other RNA
tactccatagtgaagcaat

SEQ ID NO: 849 - RNA, 19 nt, synthetic construct
  misc_feature 1..19: note = antisense sequence
  source 1..19: mol_type = other RNA
attgcttcactatggagta

SEQ ID NO: 850 - RNA, 19 nt, synthetic construct
  misc_feature 1..19: note = sense sequence
  source 1..19: mol_type = other RNA
actccatagtgaagcaatc

SEQ ID NO: 851 - RNA, 19 nt, synthetic construct
  misc_feature 1..19: note = antisense sequence
  source 1..19: mol_type = other RNA
gattgcttcactatggagt

SEQ ID NO: 852 - RNA, 19 nt, synthetic construct
  misc_feature 1..19: note = sense sequence
  source 1..19: mol_type = other RNA
ctccatagtgaagcaatct

SEQ ID NO: 853 - RNA, 19 nt, synthetic construct
  misc_feature 1..19: note = antisense sequence
  source 1..19: mol_type = other RNA
agattgcttcactatggag

SEQ ID NO: 854 - RNA, 19 nt, synthetic construct
  misc_feature 1..19: note = sense sequence
  source 1..19: mol_type = other RNA
tccatagtgaagcaatcta

SEQ ID NO: 855 - RNA, 19 nt, synthetic construct
  misc_feature 1..19: note = antisense sequence
  source 1..19: mol_type = other RNA
tagattgcttcactatgga

SEQ ID NO: 856 - RNA, 19 nt, synthetic construct
  misc_feature 1..19: note = sense sequence
  source 1..19: mol_type = other RNA
ccatagtgaagcaatctaa

SEQ ID NO: 857 - RNA, 19 nt, synthetic construct
  misc_feature 1..19: note = antisense sequence
  source 1..19: mol_type = other RNA
ttagattgcttcactatgg

SEQ ID NO: 858 - RNA, 19 nt, synthetic construct
  misc_feature 1..19: note = sense sequence
  source 1..19: mol_type = other RNA
catagtgaagcaatctaat

SEQ ID NO: 859 - RNA, 19 nt, synthetic construct
  misc_feature 1..19: note = antisense sequence
  source 1..19: mol_type = other RNA
attagattgcttcactatg

SEQ ID NO: 860 - RNA, 19 nt, synthetic construct
  misc_feature 1..19: note = sense sequence
  source 1..19: mol_type = other RNA
gtgaagcaatctaattatg

SEQ ID NO: 861 - RNA, 19 nt, synthetic construct
  misc_feature 1..19: note = antisense sequence
  source 1..19: mol_type = other RNA
cataattagattgcttcac

SEQ ID NO: 862 - RNA, 19 nt, synthetic construct
  misc_feature 1..19: note = sense sequence
  source 1..19: mol_type = other RNA
caactatacgctacatcta

SEQ ID NO: 863 - RNA, 19 nt, synthetic construct
  misc_feature 1..19: note = antisense sequence
  source 1..19: mol_type = other RNA
tagatgtagcgtatagttg

SEQ ID NO: 864 - RNA, 19 nt, synthetic construct
  misc_feature 1..19: note = sense sequence
  source 1..19: mol_type = other RNA
aactatacgctacatctag

SEQ ID NO: 865 - RNA, 19 nt, synthetic construct
  misc_feature 1..19: note = antisense sequence
  source 1..19: mol_type = other RNA
ctagatgtagcgtatagtt

SEQ ID NO: 866 - RNA, 19 nt, synthetic construct
  misc_feature 1..19: note = sense sequence
  source 1..19: mol_type = other RNA
tacgctacatctagttgcg

SEQ ID NO: 867 - RNA, 19 nt, synthetic construct
  misc_feature 1..19: note = antisense sequence
  source 1..19: mol_type = other RNA
cgcaactagatgtagcgta

SEQ ID NO: 868 - RNA, 19 nt, synthetic construct
  misc_feature 1..19: note = sense sequence
  source 1..19: mol_type = other RNA
acgctacatctagttgcga

SEQ ID NO: 869 - RNA, 19 nt, synthetic construct
  misc_feature 1..19: note = antisense sequence
  source 1..19: mol_type = other RNA
tcgcaactagatgtagcgt

SEQ ID NO: 870 - RNA, 19 nt, synthetic construct
  misc_feature 1..19: note = sense sequence
  source 1..19: mol_type = other RNA
cgctacatctagttgcgat

SEQ ID NO: 871 - RNA, 19 nt, synthetic construct
  misc_feature 1..19: note = antisense sequence
  source 1..19: mol_type = other RNA
atcgcaactagatgtagcg

SEQ ID NO: 872 - RNA, 19 nt, synthetic construct
  misc_feature 1..19: note = sense sequence
  source 1..19: mol_type = other RNA
gctacatctagttgcgatt

SEQ ID NO: 873 - RNA, 19 nt, synthetic construct
  misc_feature 1..19: note = antisense sequence
  source 1..19: mol_type = other RNA
aatcgcaactagatgtagc

SEQ ID NO: 874 - RNA, 19 nt, synthetic construct
  misc_feature 1..19: note = sense sequence
  source 1..19: mol_type = other RNA
ctacatctagttgcgatta

SEQ ID NO: 875 - RNA, 19 nt, synthetic construct
  misc_feature 1..19: note = antisense sequence
  source 1..19: mol_type = other RNA
taatcgcaactagatgtag

SEQ ID NO: 876 - RNA, 19 nt, synthetic construct
  misc_feature 1..19: note = sense sequence
  source 1..19: mol_type = other RNA
tacatctagttgcgattac

SEQ ID NO: 877 - RNA, 19 nt, synthetic construct
  misc_feature 1..19: note = antisense sequence
  source 1..19: mol_type = other RNA
gtaatcgcaactagatgta

SEQ ID NO: 878 - RNA, 19 nt, synthetic construct
  misc_feature 1..19: note = sense sequence
  source 1..19: mol_type = other RNA
acatctagttgcgattact

SEQ ID NO: 879 - RNA, 19 nt, synthetic construct
  misc_feature 1..19: note = antisense sequence
  source 1..19: mol_type = other RNA
agtaatcgcaactagatgt

SEQ ID NO: 880 - RNA, 19 nt, synthetic construct
  misc_feature 1..19: note = sense sequence
  source 1..19: mol_type = other RNA
tgcgattactggcaatgtc

SEQ ID NO: 881 - RNA, 19 nt, synthetic construct
  misc_feature 1..19: note = antisense sequence
  source 1..19: mol_type = other RNA
gacattgccagtaatcgca

SEQ ID NO: 882 - RNA, 19 nt, synthetic construct
  misc_feature 1..19: note = sense sequence
  source 1..19: mol_type = other RNA
gcgattactggcaatgtcc

SEQ ID NO: 883 - RNA, 19 nt, synthetic construct
  misc_feature 1..19: note = antisense sequence
  source 1..19: mol_type = other RNA
ggacattgccagtaatcgc

SEQ ID NO: 884 - RNA, 19 nt, synthetic construct
  misc_feature 1..19: note = sense sequence
  source 1..19: mol_type = other RNA
cgattactggcaatgtccc

SEQ ID NO: 885 - RNA, 19 nt, synthetic construct
  misc_feature 1..19: note = antisense sequence
  source 1..19: mol_type = other RNA
gggacattgccagtaatcg

SEQ ID NO: 886 - RNA, 19 nt, synthetic construct
  misc_feature 1..19: note = sense sequence
  source 1..19: mol_type = other RNA
caaagatttggtgttttct

SEQ ID NO: 887 - RNA, 19 nt, synthetic construct
  misc_feature 1..19: note = antisense sequence
  source 1..19: mol_type = other RNA
agaaaacaccaaatctttg

SEQ ID NO: 888 - RNA, 19 nt, synthetic construct
  misc_feature 1..19: note = sense sequence
  source 1..19: mol_type = other RNA
tttggtgttttctacttgg

SEQ ID NO: 889 - RNA, 19 nt, synthetic construct
  misc_feature 1..19: note = antisense sequence
  source 1..19: mol_type = other RNA
ccaagtagaaaacaccaaa

SEQ ID NO: 890 - RNA, 19 nt, synthetic construct
  misc_feature 1..19: note = sense sequence
  source 1..19: mol_type = other RNA
ttggtgttttctacttggg

SEQ ID NO: 891 - RNA, 19 nt, synthetic construct
  misc_feature 1..19: note = antisense sequence
  source 1..19: mol_type = other RNA
cccaagtagaaaacaccaa

SEQ ID NO: 892 - RNA, 19 nt, synthetic construct
  misc_feature 1..19: note = sense sequence
  source 1..19: mol_type = other RNA
gttttctacttgggatcac

SEQ ID NO: 893 - RNA, 19 nt, synthetic construct
  misc_feature 1..19: note = antisense sequence
  source 1..19: mol_type = other RNA
gtgatcccaagtagaaaac

SEQ ID NO: 894 - RNA, 19 nt, synthetic construct
  misc_feature 1..19: note = sense sequence
  source 1..19: mol_type = other RNA
ttttctacttgggatcaca

SEQ ID NO: 895 - RNA, 19 nt, synthetic construct
  misc_feature 1..19: note = antisense sequence
  source 1..19: mol_type = other RNA
tgtgatcccaagtagaaaa

SEQ ID NO: 896 - RNA, 19 nt, synthetic construct
  misc_feature 1..19: note = sense sequence
  source 1..19: mol_type = other RNA
aagcaaaaggacacttcaa

SEQ ID NO: 897 - RNA, 19 nt, synthetic construct
  misc_feature 1..19: note = antisense sequence
  source 1..19: mol_type = other RNA
ttgaagtgtccttttgctt

SEQ ID NO: 898 - RNA, 19 nt, synthetic construct
  misc_feature 1..19: note = sense sequence
  source 1..19: mol_type = other RNA
agcaaaaggacacttcaac

SEQ ID NO: 899 - RNA, 19 nt, synthetic construct
  misc_feature 1..19: note = antisense sequence
  source 1..19: mol_type = other RNA
gttgaagtgtccttttgct

SEQ ID NO: 900 - RNA, 19 nt, synthetic construct
  misc_feature 1..19: note = sense sequence
  source 1..19: mol_type = other RNA
gcaaaaggacacttcaact

SEQ ID NO: 901 - RNA, 19 nt, synthetic construct
  misc_feature 1..19: note = antisense sequence
  source 1..19: mol_type = other RNA
agttgaagtgtccttttgc

SEQ ID NO: 902 - RNA, 19 nt, synthetic construct
  misc_feature 1..19: note = sense sequence
  source 1..19: mol_type = other RNA
caaaaggacacttcaactg

SEQ ID NO: 903 - RNA, 19 nt, synthetic construct
  misc_feature 1..19: note = antisense sequence
  source 1..19: mol_type = other RNA
cagttgaagtgtccttttg

SEQ ID NO: 904 - RNA, 19 nt, synthetic construct
  misc_feature 1..19: note = sense sequence
  source 1..19: mol_type = other RNA
aaaaggacacttcaactgt

SEQ ID NO: 905 - RNA, 19 nt, synthetic construct
  misc_feature 1..19: note = antisense sequence
  source 1..19: mol_type = other RNA
acagttgaagtgtcctttt

SEQ ID NO: 906 - RNA, 19 nt, synthetic construct
  misc_feature 1..19: note = sense sequence
  source 1..19: mol_type = other RNA
cttcaactgtccagagggt

SEQ ID NO: 907 - RNA, 19 nt, synthetic construct
  misc_feature 1..19: note = antisense sequence
  source 1..19: mol_type = other RNA
accctctggacagttgaag

SEQ ID NO: 908 - RNA, 19 nt, synthetic construct
  misc_feature 1..19: note = sense sequence
  source 1..19: mol_type = other RNA
ttcaactgtccagagggtt

SEQ ID NO: 909 - RNA, 19 nt, synthetic construct
  misc_feature 1..19: note = antisense sequence
  source 1..19: mol_type = other RNA
aaccctctggacagttgaa

SEQ ID NO: 910 - RNA, 19 nt, synthetic construct
  misc_feature 1..19: note = sense sequence
  source 1..19: mol_type = other RNA
tcaactgtccagagggtta

SEQ ID NO: 911 - RNA, 19 nt, synthetic construct
  misc_feature 1..19: note = antisense sequence
  source 1..19: mol_type = other RNA
taaccctctggacagttga

SEQ ID NO: 912 - RNA, 19 nt, synthetic construct
  misc_feature 1..19: note = sense sequence
  source 1..19: mol_type = other RNA
caactgtccagagggttat

SEQ ID NO: 913 - RNA, 19 nt, synthetic construct
  misc_feature 1..19: note = antisense sequence
  source 1..19: mol_type = other RNA
ataaccctctggacagttg

SEQ ID NO: 914 - RNA, 19 nt, synthetic construct
  misc_feature 1..19: note = sense sequence
  source 1..19: mol_type = other RNA
aactgtccagagggttatt

SEQ ID NO: 915 - RNA, 19 nt, synthetic construct
  misc_feature 1..19: note = antisense sequence
  source 1..19: mol_type = other RNA
aataaccctctggacagtt

SEQ ID NO: 916 - RNA, 19 nt, synthetic construct
  misc_feature 1..19: note = sense sequence
  source 1..19: mol_type = other RNA
actgtccagagggttattc

SEQ ID NO: 917 - RNA, 19 nt, synthetic construct
  misc_feature 1..19: note = antisense sequence
  source 1..19: mol_type = other RNA
gaataaccctctggacagt

SEQ ID NO: 918 - RNA, 19 nt, synthetic construct
  misc_feature 1..19: note = sense sequence
  source 1..19: mol_type = other RNA
ctgtccagagggttattca

SEQ ID NO: 919 - RNA, 19 nt, synthetic construct
  misc_feature 1..19: note = antisense sequence
  source 1..19: mol_type = other RNA
tgaataaccctctggacag

SEQ ID NO: 920 - RNA, 19 nt, synthetic construct
  misc_feature 1..19: note = sense sequence
  source 1..19: mol_type = other RNA
tgtccagagggttattcag

SEQ ID NO: 921 - RNA, 19 nt, synthetic construct
  misc_feature 1..19: note = antisense sequence
  source 1..19: mol_type = other RNA
ctgaataaccctctggaca

SEQ ID NO: 922 - RNA, 19 nt, synthetic construct
  misc_feature 1..19: note = sense sequence
  source 1..19: mol_type = other RNA
gtccagagggttattcagg

SEQ ID NO: 923 - RNA, 19 nt, synthetic construct
  misc_feature 1..19: note = antisense sequence
  source 1..19: mol_type = other RNA
cctgaataaccctctggac

SEQ ID NO: 924 - RNA, 19 nt, synthetic construct
  misc_feature 1..19: note = sense sequence
  source 1..19: mol_type = other RNA
agagggttattcaggaggc

SEQ ID NO: 925 - RNA, 19 nt, synthetic construct
  misc_feature 1..19: note = antisense sequence
  source 1..19: mol_type = other RNA
gcctcctgaataaccctct

SEQ ID NO: 926 - RNA, 19 nt, synthetic construct
  misc_feature 1..19: note = sense sequence
  source 1..19: mol_type = other RNA
gagggttattcaggaggct

SEQ ID NO: 927 - RNA, 19 nt, synthetic construct
  misc_feature 1..19: note = antisense sequence
  source 1..19: mol_type = other RNA
agcctcctgaataaccctc

SEQ ID NO: 928 - RNA, 19 nt, synthetic construct
  misc_feature 1..19: note = sense sequence
  source 1..19: mol_type = other RNA
agggttattcaggaggctg

SEQ ID NO: 929 - RNA, 19 nt, synthetic construct
  misc_feature 1..19: note = antisense sequence
  source 1..19: mol_type = other RNA
cagcctcctgaataaccct

SEQ ID NO: 930 - RNA, 19 nt, synthetic construct
  misc_feature 1..19: note = sense sequence
  source 1..19: mol_type = other RNA
gggttattcaggaggctgg

SEQ ID NO: 931 - RNA, 19 nt, synthetic construct
  misc_feature 1..19: note = antisense sequence
  source 1..19: mol_type = other RNA
ccagcctcctgaataaccc

SEQ ID NO: 932 - RNA, 19 nt, synthetic construct
  misc_feature 1..19: note = sense sequence
  source 1..19: mol_type = other RNA
ggttattcaggaggctggt

SEQ ID NO: 933 - RNA, 19 nt, synthetic construct
  misc_feature 1..19: note = antisense sequence
  source 1..19: mol_type = other RNA
accagcctcctgaataacc

SEQ ID NO: 934 - RNA, 19 nt, synthetic construct
  misc_feature 1..19: note = sense sequence
  source 1..19: mol_type = other RNA
gttattcaggaggctggtg

SEQ ID NO: 935 - RNA, 19 nt, synthetic construct
  misc_feature 1..19: note = antisense sequence
  source 1..19: mol_type = other RNA
caccagcctcctgaataac

SEQ ID NO: 936 - RNA, 19 nt, synthetic construct
  misc_feature 1..19: note = sense sequence
  source 1..19: mol_type = other RNA
ttattcaggaggctggtgg

SEQ ID NO: 937 - RNA, 19 nt, synthetic construct
  misc_feature 1..19: note = antisense sequence
  source 1..19: mol_type = other RNA
ccaccagcctcctgaataa

SEQ ID NO: 938 - RNA, 19 nt, synthetic construct
  misc_feature 1..19: note = sense sequence
  source 1..19: mol_type = other RNA
tattcaggaggctggtggt

SEQ ID NO: 939 - RNA, 19 nt, synthetic construct
  misc_feature 1..19: note = antisense sequence
  source 1..19: mol_type = other RNA
accaccagcctcctgaata

SEQ ID NO: 940 - RNA, 19 nt, synthetic construct
  misc_feature 1..19: note = sense sequence
  source 1..19: mol_type = other RNA
attcaggaggctggtggtg

SEQ ID NO: 941 - RNA, 19 nt, synthetic construct
  misc_feature 1..19: note = antisense sequence
  source 1..19: mol_type = other RNA
caccaccagcctcctgaat

SEQ ID NO: 942 - RNA, 19 nt, synthetic construct
  misc_feature 1..19: note = sense sequence
  source 1..19: mol_type = other RNA
ttcaggaggctggtggtgg

SEQ ID NO: 943 - RNA, 19 nt, synthetic construct
  misc_feature 1..19: note = antisense sequence
  source 1..19: mol_type = other RNA
ccaccaccagcctcctgaa

SEQ ID NO: 944 - RNA, 19 nt, synthetic construct
  misc_feature 1..19: note = sense sequence
  source 1..19: mol_type = other RNA
gaggctggtggtggcatga

SEQ ID NO: 945 - RNA, 19 nt, synthetic construct
  misc_feature 1..19: note = antisense sequence
  source 1..19: mol_type = other RNA
tcatgccaccaccagcctc

SEQ ID NO: 946 - RNA, 19 nt, synthetic construct
  misc_feature 1..19: note = sense sequence
  source 1..19: mol_type = other RNA
aggctggtggtggcatgat

SEQ ID NO: 947 - RNA, 19 nt, synthetic construct
  misc_feature 1..19: note = antisense sequence
  source 1..19: mol_type = other RNA
atcatgccaccaccagcct

SEQ ID NO: 948 - RNA, 19 nt, synthetic construct
  misc_feature 1..19: note = sense sequence
  source 1..19: mol_type = other RNA
ctggtggtggcatgatgag

SEQ ID NO: 949 - RNA, 19 nt, synthetic construct
  misc_feature 1..19: note = antisense sequence
  source 1..19: mol_type = other RNA
ctcatcatgccaccaccag

SEQ ID NO: 950 - RNA, 19 nt, synthetic construct
  misc_feature 1..19: note = sense sequence
  source 1..19: mol_type = other RNA
tggtggtggcatgatgagt

SEQ ID NO: 951 - RNA, 19 nt, synthetic construct
  misc_feature 1..19: note = antisense sequence
  source 1..19: mol_type = other RNA
actcatcatgccaccacca

SEQ ID NO: 952 - RNA, 19 nt, synthetic construct
  misc_feature 1..19: note = sense sequence
  source 1..19: mol_type = other RNA
ggtggtggcatgatgagtg

SEQ ID NO: 953 - RNA, 19 nt, synthetic construct
  misc_feature 1..19: note = antisense sequence
  source 1..19: mol_type = other RNA
cactcatcatgccaccacc

SEQ ID NO: 954 - RNA, 19 nt, synthetic construct
  misc_feature 1..19: note = sense sequence
  source 1..19: mol_type = other RNA
gtggtggcatgatgagtgt

SEQ ID NO: 955 - RNA, 19 nt, synthetic construct
  misc_feature 1..19: note = antisense sequence
  source 1..19: mol_type = other RNA
acactcatcatgccaccac

SEQ ID NO: 956 - RNA, 19 nt, synthetic construct
  misc_feature 1..19: note = sense sequence
  source 1..19: mol_type = other RNA
tggtggcatgatgagtgtg

SEQ ID NO: 957 - RNA, 19 nt, synthetic construct
  misc_feature 1..19: note = antisense sequence
  source 1..19: mol_type = other RNA
cacactcatcatgccacca

SEQ ID NO: 958 - RNA, 19 nt, synthetic construct
  misc_feature 1..19: note = sense sequence
  source 1..19: mol_type = other RNA
ggtggcatgatgagtgtgg

SEQ ID NO: 959 - RNA, 19 nt, synthetic construct
  misc_feature 1..19: note = antisense sequence
  source 1..19: mol_type = other RNA
ccacactcatcatgccacc

SEQ ID NO: 960 - RNA, 19 nt, synthetic construct
  misc_feature 1..19: note = sense sequence
  source 1..19: mol_type = other RNA
gtggcatgatgagtgtgga

SEQ ID NO: 961 - RNA, 19 nt, synthetic construct
  misc_feature 1..19: note = antisense sequence
  source 1..19: mol_type = other RNA
tccacactcatcatgccac

SEQ ID NO: 962 - RNA, 19 nt, synthetic construct
  misc_feature 1..19: note = sense sequence
  source 1..19: mol_type = other RNA
tggcatgatgagtgtggag

SEQ ID NO: 963 - RNA, 19 nt, synthetic construct
  misc_feature 1..19: note = antisense sequence
  source 1..19: mol_type = other RNA
ctccacactcatcatgcca

SEQ ID NO: 964 - RNA, 19 nt, synthetic construct
  misc_feature 1..19: note = sense sequence
  source 1..19: mol_type = other RNA
accaagagcaaaatctaag

SEQ ID NO: 965 - RNA, 19 nt, synthetic construct
  misc_feature 1..19: note = antisense sequence
  source 1..19: mol_type = other RNA
cttagattttgctcttggt

SEQ ID NO: 966 - RNA, 19 nt, synthetic construct
  misc_feature 1..19: note = sense sequence
  source 1..19: mol_type = other RNA
ccaagagcaaaatctaagc

SEQ ID NO: 967 - RNA, 19 nt, synthetic construct
  misc_feature 1..19: note = antisense sequence
  source 1..19: mol_type = other RNA
gcttagattttgctcttgg

SEQ ID NO: 968 - RNA, 19 nt, synthetic construct
  misc_feature 1..19: note = sense sequence
  source 1..19: mol_type = other RNA
caagagcaaaatctaagcc

SEQ ID NO: 969 - RNA, 19 nt, synthetic construct
  misc_feature 1..19: note = antisense sequence
  source 1..19: mol_type = other RNA
ggcttagattttgctcttg

SEQ ID NO: 970 - RNA, 19 nt, synthetic construct
  misc_feature 1..19: note = sense sequence
  source 1..19: mol_type = other RNA
agaggattatcttggaagt

SEQ ID NO: 971 - RNA, 19 nt, synthetic construct
  misc_feature 1..19: note = antisense sequence
  source 1..19: mol_type = other RNA
acttccaagataatcctct

SEQ ID NO: 972 - RNA, 19 nt, synthetic construct
  misc_feature 1..19: note = sense sequence
  source 1..19: mol_type = other RNA
gaggattatcttggaagtc

SEQ ID NO: 973 - RNA, 19 nt, synthetic construct
  misc_feature 1..19: note = antisense sequence
  source 1..19: mol_type = other RNA
gacttccaagataatcctc

SEQ ID NO: 974 - RNA, 19 nt, synthetic construct
  misc_feature 1..19: note = sense sequence
  source 1..19: mol_type = other RNA
aggattatcttggaagtct

SEQ ID NO: 975 - RNA, 19 nt, synthetic construct
  misc_feature 1..19: note = antisense sequence
  source 1..19: mol_type = other RNA
agacttccaagataatcct

SEQ ID NO: 976 - RNA, 19 nt, synthetic construct
  misc_feature 1..19: note = sense sequence
  source 1..19: mol_type = other RNA
ggattatcttggaagtctc

SEQ ID NO: 977 - RNA, 19 nt, synthetic construct
  misc_feature 1..19: note = antisense sequence
  source 1..19: mol_type = other RNA
gagacttccaagataatcc

SEQ ID NO: 978 - RNA, 19 nt, synthetic construct
  misc_feature 1..19: note = sense sequence
  source 1..19: mol_type = other RNA
gattatcttggaagtctca

SEQ ID NO: 979 - RNA, 19 nt, synthetic construct
  misc_feature 1..19: note = antisense sequence
  source 1..19: mol_type = other RNA
tgagacttccaagataatc

SEQ ID NO: 980 - RNA, 19 nt, synthetic construct
  misc_feature 1..19: note = sense sequence
  source 1..19: mol_type = other RNA
attatcttggaagtctcaa

SEQ ID NO: 981 - RNA, 19 nt, synthetic construct
  misc_feature 1..19: note = antisense sequence
  source 1..19: mol_type = other RNA
ttgagacttccaagataat

SEQ ID NO: 982 - RNA, 19 nt, synthetic construct
  misc_feature 1..19: note = sense sequence
  source 1..19: mol_type = other RNA
ttatcttggaagtctcaaa

SEQ ID NO: 983 - RNA, 19 nt, synthetic construct
  misc_feature 1..19: note = antisense sequence
  source 1..19: mol_type = other RNA
tttgagacttccaagataa

SEQ ID NO: 984 - RNA, 19 nt, synthetic construct
  misc_feature 1..19: note = sense sequence
  source 1..19: mol_type = other RNA
tatcttggaagtctcaaaa

SEQ ID NO: 985 - RNA, 19 nt, synthetic construct
  misc_feature 1..19: note = antisense sequence
  source 1..19: mol_type = other RNA
ttttgagacttccaagata

SEQ ID NO: 986 - RNA, 19 nt, synthetic construct
  misc_feature 1..19: note = sense sequence
  source 1..19: mol_type = other RNA
atcttggaagtctcaaaat

SEQ ID NO: 987 - RNA, 19 nt, synthetic construct
  misc_feature 1..19: note = antisense sequence
  source 1..19: mol_type = other RNA
attttgagacttccaagat

SEQ ID NO: 988 - RNA, 19 nt, synthetic construct
  misc_feature 1..19: note = sense sequence
  source 1..19: mol_type = other RNA
tcttggaagtctcaaaatg

SEQ ID NO: 989 - RNA, 19 nt, synthetic construct
  misc_feature 1..19: note = antisense sequence
  source 1..19: mol_type = other RNA
cattttgagacttccaaga

SEQ ID NO: 990 - RNA, 19 nt, synthetic construct
  misc_feature 1..19: note = sense sequence
  source 1..19: mol_type = other RNA
cttggaagtctcaaaatgg

SEQ ID NO: 991 - RNA, 19 nt, synthetic construct
  misc_feature 1..19: note = antisense sequence
  source 1..19: mol_type = other RNA
ccattttgagacttccaag

SEQ ID NO: 992 - RNA, 19 nt, synthetic construct
  misc_feature 1..19: note = sense sequence
  source 1..19: mol_type = other RNA
ttggaagtctcaaaatgga

SEQ ID NO: 993 - RNA, 19 nt, synthetic construct
  misc_feature 1..19: note = antisense sequence
  source 1..19: mol_type = other RNA
tccattttgagacttccaa

SEQ ID NO: 994 - RNA, 19 nt, synthetic construct
  misc_feature 1..19: note = sense sequence
  source 1..19: mol_type = other RNA
tggaagtctcaaaatggaa

SEQ ID NO: 995 - RNA, 19 nt, synthetic construct
  misc_feature 1..19: note = antisense sequence
  source 1..19: mol_type = other RNA
ttccattttgagacttcca

SEQ ID NO: 996 - RNA, 19 nt, synthetic construct
  misc_feature 1..19: note = sense sequence
  source 1..19: mol_type = other RNA
ggaagtctcaaaatggaag

SEQ ID NO: 997 - RNA, 19 nt, synthetic construct
  misc_feature 1..19: note = antisense sequence
  source 1..19: mol_type = other RNA
cttccattttgagacttcc

SEQ ID NO: 998 - RNA, 19 nt, synthetic construct
  misc_feature 1..19: note = sense sequence
  source 1..19: mol_type = other RNA
aagtctcaaaatggaaggt

SEQ ID NO: 999 - RNA, 19 nt, synthetic construct
  misc_feature 1..19: note = antisense sequence
  source 1..19: mol_type = other RNA
accttccattttgagactt

SEQ ID NO: 1000 - RNA, 19 nt, synthetic construct
  misc_feature 1..19: note = sense sequence
  source 1..19: mol_type = other RNA
agtctcaaaatggaaggtt

SEQ ID NO: 1001 - RNA, 19 nt, synthetic construct
  misc_feature 1..19: note = antisense sequence
  source 1..19: mol_type = other RNA
aaccttccattttgagact

SEQ ID NO: 1002 - RNA, 19 nt, synthetic construct
  misc_feature 1..19: note = sense sequence
  source 1..19: mol_type = other RNA
gtctcaaaatggaaggtta

SEQ ID NO: 1003 - RNA, 19 nt, synthetic construct
  misc_feature 1..19: note = antisense sequence
  source 1..19: mol_type = other RNA
taaccttccattttgagac

SEQ ID NO: 1004 - RNA, 19 nt, synthetic construct
  misc_feature 1..19: note = sense sequence
  source 1..19: mol_type = other RNA
tctcaaaatggaaggttat

SEQ ID NO: 1005 - RNA, 19 nt, synthetic construct
  misc_feature 1..19: note = antisense sequence
  source 1..19: mol_type = other RNA
ataaccttccattttgaga

SEQ ID NO: 1006 - RNA, 19 nt, synthetic construct
  misc_feature 1..19: note = sense sequence
  source 1..19: mol_type = other RNA
ctcaaaatggaaggttata

SEQ ID NO: 1007 - RNA, 19 nt, synthetic construct
  misc_feature 1..19: note = antisense sequence
  source 1..19: mol_type = other RNA
tataaccttccattttgag

SEQ ID NO: 1008 - RNA, 19 nt, synthetic construct
  misc_feature 1..19: note = sense sequence
  source 1..19: mol_type = other RNA
tcaaaatggaaggttatac

SEQ ID NO: 1009 - RNA, 19 nt, synthetic construct
  misc_feature 1..19: note = antisense sequence
  source 1..19: mol_type = other RNA
gtataaccttccattttga

SEQ ID NO: 1010 - RNA, 19 nt, synthetic construct
  misc_feature 1..19: note = sense sequence
  source 1..19: mol_type = other RNA
caaaatggaaggttatact

SEQ ID NO: 1011 - RNA, 19 nt, synthetic construct
  misc_feature 1..19: note = antisense sequence
  source 1..19: mol_type = other RNA
agtataaccttccattttg

SEQ ID NO: 1012 - RNA, 19 nt, synthetic construct
  misc_feature 1..19: note = sense sequence
  source 1..19: mol_type = other RNA
atggaaggttatactctat

SEQ ID NO: 1013 - RNA, 19 nt, synthetic construct
  misc_feature 1..19: note = antisense sequence
  source 1..19: mol_type = other RNA
atagagtataaccttccat

SEQ ID NO: 1014 - RNA, 19 nt, synthetic construct
  misc_feature 1..19: note = sense sequence
  source 1..19: mol_type = other RNA
aaccaaaatgttgatccat

SEQ ID NO: 1015 - RNA, 19 nt, synthetic construct
  misc_feature 1..19: note = antisense sequence
  source 1..19: mol_type = other RNA
atggatcaacattttggtt

SEQ ID NO: 1016 - RNA, 19 nt, synthetic construct
  misc_feature 1..19: note = sense sequence
  source 1..19: mol_type = other RNA
ccaaaatgttgatccatcc

SEQ ID NO: 1017 - RNA, 19 nt, synthetic construct
  misc_feature 1..19: note = antisense sequence
  source 1..19: mol_type = other RNA
ggatggatcaacattttgg

SEQ ID NO: 1018 - RNA, 19 nt, synthetic construct
  misc_feature 1..19: note = sense sequence
  source 1..19: mol_type = other RNA
caaaatgttgatccatcca

SEQ ID NO: 1019 - RNA, 19 nt, synthetic construct
  misc_feature 1..19: note = antisense sequence
  source 1..19: mol_type = other RNA
tggatggatcaacattttg

SEQ ID NO: 1020 - RNA, 19 nt, synthetic construct
  misc_feature 1..19: note = sense sequence
  source 1..19: mol_type = other RNA
actgaggcaaatttaaaag

SEQ ID NO: 1021 - RNA, 19 nt, synthetic construct
  misc_feature 1..19: note = antisense sequence
  source 1..19: mol_type = other RNA
cttttaaatttgcctcagt

SEQ ID NO: 1022 - RNA, 19 nt, synthetic construct
  misc_feature 1..19: note = sense sequence
  source 1..19: mol_type = other RNA
ctgaggcaaatttaaaagg

SEQ ID NO: 1023 - RNA, 19 nt, synthetic construct
  misc_feature 1..19: note = antisense sequence
  source 1..19: mol_type = other RNA
ccttttaaatttgcctcag

SEQ ID NO: 1024 - RNA, 19 nt, synthetic construct
  misc_feature 1..19: note = sense sequence
  source 1..19: mol_type = other RNA
gaggcaaatttaaaaggca

SEQ ID NO: 1025 - RNA, 19 nt, synthetic construct
  misc_feature 1..19: note = antisense sequence
  source 1..19: mol_type = other RNA
tgccttttaaatttgcctc

SEQ ID NO: 1026 - RNA, 19 nt, synthetic construct
  misc_feature 1..19: note = sense sequence
  source 1..19: mol_type = other RNA
aggcaaatttaaaaggcaa

SEQ ID NO: 1027 - RNA, 19 nt, synthetic construct
  misc_feature 1..19: note = antisense sequence
  source 1..19: mol_type = other RNA
ttgccttttaaatttgcct

SEQ ID NO: 1028 - RNA, 19 nt, synthetic construct
  misc_feature 1..19: note = sense sequence
  source 1..19: mol_type = other RNA
acattaacctcattccaag

SEQ ID NO: 1029 - RNA, 19 nt, synthetic construct
  misc_feature 1..19: note = antisense sequence
  source 1..19: mol_type = other RNA
cttggaatgaggttaatgt

SEQ ID NO: 1030 - RNA, 19 nt, synthetic construct
  misc_feature 1..19: note = sense sequence
  source 1..19: mol_type = other RNA
cattaacctcattccaagt

SEQ ID NO: 1031 - RNA, 19 nt, synthetic construct
  misc_feature 1..19: note = antisense sequence
  source 1..19: mol_type = other RNA
acttggaatgaggttaatg

SEQ ID NO: 1032 - RNA, 19 nt, synthetic construct
  misc_feature 1..19: note = sense sequence
  source 1..19: mol_type = other RNA
attaacctcattccaagtt

SEQ ID NO: 1033 - RNA, 19 nt, synthetic construct
  misc_feature 1..19: note = antisense sequence
  source 1..19: mol_type = other RNA
aacttggaatgaggttaat

SEQ ID NO: 1034 - RNA, 19 nt, synthetic construct
  misc_feature 1..19: note = sense sequence
  source 1..19: mol_type = other RNA
aacctcattccaagttaat

SEQ ID NO: 1035 - RNA, 19 nt, synthetic construct
  misc_feature 1..19: note = antisense sequence
  source 1..19: mol_type = other RNA
attaacttggaatgaggtt

SEQ ID NO: 1036 - RNA, 19 nt, synthetic construct
  misc_feature 1..19: note = sense sequence
  source 1..19: mol_type = other RNA
tccaagttaatgtggtcta

SEQ ID NO: 1037 - RNA, 19 nt, synthetic construct
  misc_feature 1..19: note = antisense sequence
  source 1..19: mol_type = other RNA
tagaccacattaacttgga

SEQ ID NO: 1038 - RNA, 19 nt, synthetic construct
  misc_feature 1..19: note = sense sequence
  source 1..19: mol_type = other RNA
ccaagttaatgtggtctaa

SEQ ID NO: 1039 - RNA, 19 nt, synthetic construct
  misc_feature 1..19: note = antisense sequence
  source 1..19: mol_type = other RNA
ttagaccacattaacttgg

SEQ ID NO: 1040 - RNA, 19 nt, synthetic construct
  misc_feature 1..19: note = sense sequence
  source 1..19: mol_type = other RNA
caagttaatgtggtctaat

SEQ ID NO: 1041 - RNA, 19 nt, synthetic construct
  misc_feature 1..19: note = antisense sequence
  source 1..19: mol_type = other RNA
attagaccacattaacttg

SEQ ID NO: 1042 - RNA, 19 nt, synthetic construct
  misc_feature 1..19: note = sense sequence
  source 1..19: mol_type = other RNA
aatgtggtctaataatctg

SEQ ID NO: 1043 - RNA, 19 nt, synthetic construct
  misc_feature 1..19: note = antisense sequence
  source 1..19: mol_type = other RNA
cagattattagaccacatt

SEQ ID NO: 1044 - RNA, 19 nt, synthetic construct
  misc_feature 1..19: note = sense sequence
  source 1..19: mol_type = other RNA
tggtctaataatctggtat

SEQ ID NO: 1045 - RNA, 19 nt, synthetic construct
  misc_feature 1..19: note = antisense sequence
  source 1..19: mol_type = other RNA
ataccagattattagacca

SEQ ID NO: 1046 - RNA, 19 nt, synthetic construct
  misc_feature 1..19: note = sense sequence
  source 1..19: mol_type = other RNA
ttatcttaaagtcactgtc

SEQ ID NO: 1047 - RNA, 19 nt, synthetic construct
  misc_feature 1..19: note = antisense sequence
  source 1..19: mol_type = other RNA
gacagtgactttaagataa

SEQ ID NO: 1048 - RNA, 19 nt, synthetic construct
  misc_feature 1..19: note = sense sequence
  source 1..19: mol_type = other RNA
tatcttaaagtcactgtct

SEQ ID NO: 1049 - RNA, 19 nt, synthetic construct
  misc_feature 1..19: note = antisense sequence
  source 1..19: mol_type = other RNA
agacagtgactttaagata

SEQ ID NO: 1050 - RNA, 19 nt, synthetic construct
  misc_feature 1..19: note = sense sequence
  source 1..19: mol_type = other RNA
atcttaaagtcactgtcta

SEQ ID NO: 1051 - RNA, 19 nt, synthetic construct
  misc_feature 1..19: note = antisense sequence
  source 1..19: mol_type = other RNA
tagacagtgactttaagat

SEQ ID NO: 1052 - RNA, 19 nt, synthetic construct
  misc_feature 1..19: note = sense sequence
  source 1..19: mol_type = other RNA
tcttaaagtcactgtctat

SEQ ID NO: 1053 - RNA, 19 nt, synthetic construct
  misc_feature 1..19: note = antisense sequence
  source 1..19: mol_type = other RNA
atagacagtgactttaaga

SEQ ID NO: 1054 - RNA, 19 nt, synthetic construct
  misc_feature 1..19: note = sense sequence
  source 1..19: mol_type = other RNA
cttaaagtcactgtctatt

SEQ ID NO: 1055 - RNA, 19 nt, synthetic construct
  misc_feature 1..19: note = antisense sequence
  source 1..19: mol_type = other RNA
aatagacagtgactttaag

SEQ ID NO: 1056 - RNA, 19 nt, synthetic construct
  misc_feature 1..19: note = sense sequence
  source 1..19: mol_type = other RNA
aagtcactgtctatttaag

SEQ ID NO: 1057 - RNA, 19 nt, synthetic construct
  misc_feature 1..19: note = antisense sequence
  source 1..19: mol_type = other RNA
cttaaatagacagtgactt

SEQ ID NO: 1058 - RNA, 19 nt, synthetic construct
  misc_feature 1..19: note = sense sequence
  source 1..19: mol_type = other RNA
agtcactgtctatttaaga

SEQ ID NO: 1059 - RNA, 19 nt, synthetic construct
  misc_feature 1..19: note = antisense sequence
  source 1..19: mol_type = other RNA
tcttaaatagacagtgact

SEQ ID NO: 1060 - RNA, 19 nt, synthetic construct
  misc_feature 1..19: note = sense sequence
  source 1..19: mol_type = other RNA
caatcacataaccttaaag

SEQ ID NO: 1061 - RNA, 19 nt, synthetic construct
  misc_feature 1..19: note = antisense sequence
  source 1..19: mol_type = other RNA
ctttaaggttatgtgattg

SEQ ID NO: 1062 - RNA, 19 nt, synthetic construct
  misc_feature 1..19: note = sense sequence
  source 1..19: mol_type = other RNA
ataaccttaaagaataccg

SEQ ID NO: 1063 - RNA, 19 nt, synthetic construct
  misc_feature 1..19: note = antisense sequence
  source 1..19: mol_type = other RNA
cggtattctttaaggttat

SEQ ID NO: 1064 - RNA, 19 nt, synthetic construct
  misc_feature 1..19: note = sense sequence
  source 1..19: mol_type = other RNA
taaccttaaagaataccgt

SEQ ID NO: 1065 - RNA, 19 nt, synthetic construct
  misc_feature 1..19: note = antisense sequence
  source 1..19: mol_type = other RNA
acggtattctttaaggtta

SEQ ID NO: 1066 - RNA, 19 nt, synthetic construct
  misc_feature 1..19: note = sense sequence
  source 1..19: mol_type = other RNA
aaccttaaagaataccgtt

SEQ ID NO: 1067 - RNA, 19 nt, synthetic construct
  misc_feature 1..19: note = antisense sequence
  source 1..19: mol_type = other RNA
aacggtattctttaaggtt

SEQ ID NO: 1068 - RNA, 19 nt, synthetic construct
  misc_feature 1..19: note = sense sequence
  source 1..19: mol_type = other RNA
accttaaagaataccgttt

SEQ ID NO: 1069 - RNA, 19 nt, synthetic construct
  misc_feature 1..19: note = antisense sequence
  source 1..19: mol_type = other RNA
aaacggtattctttaaggt

SEQ ID NO: 1070 - RNA, 19 nt, synthetic construct
  misc_feature 1..19: note = sense sequence
  source 1..19: mol_type = other RNA
agaataccgtttacatttc

SEQ ID NO: 1071 - RNA, 19 nt, synthetic construct
  misc_feature 1..19: note = antisense sequence
  source 1..19: mol_type = other RNA
gaaatgtaaacggtattct

SEQ ID NO: 1072 - RNA, 19 nt, synthetic construct
  misc_feature 1..19: note = sense sequence
  source 1..19: mol_type = other RNA
gaataccgtttacatttct

SEQ ID NO: 1073 - RNA, 19 nt, synthetic construct
  misc_feature 1..19: note = antisense sequence
  source 1..19: mol_type = other RNA
agaaatgtaaacggtattc

SEQ ID NO: 1074 - RNA, 19 nt, synthetic construct
  misc_feature 1..19: note = sense sequence
  source 1..19: mol_type = other RNA
accgtttacatttctcaat

SEQ ID NO: 1075 - RNA, 19 nt, synthetic construct
  misc_feature 1..19: note = antisense sequence
  source 1..19: mol_type = other RNA
attgagaaatgtaaacggt

SEQ ID NO: 1076 - RNA, 19 nt, synthetic construct
  misc_feature 1..19: note = sense sequence
  source 1..19: mol_type = other RNA
ccgtttacatttctcaatc

SEQ ID NO: 1077 - RNA, 19 nt, synthetic construct
  misc_feature 1..19: note = antisense sequence
  source 1..19: mol_type = other RNA
gattgagaaatgtaaacgg

SEQ ID NO: 1078 - RNA, 19 nt, synthetic construct
  misc_feature 1..19: note = sense sequence
  source 1..19: mol_type = other RNA
cgtttacatttctcaatca

SEQ ID NO: 1079 - RNA, 19 nt, synthetic construct
  misc_feature 1..19: note = antisense sequence
  source 1..19: mol_type = other RNA
tgattgagaaatgtaaacg

SEQ ID NO: 1080 - RNA, 19 nt, synthetic construct
  misc_feature 1..19: note = sense sequence
  source 1..19: mol_type = other RNA
ttaaattttgtgatgtggg

SEQ ID NO: 1081 - RNA, 19 nt, synthetic construct
  misc_feature 1..19: note = antisense sequence
  source 1..19: mol_type = other RNA
cccacatcacaaaatttaa

SEQ ID NO: 1082 - RNA, 19 nt, synthetic construct
  misc_feature 1..19: note = sense sequence
  source 1..19: mol_type = other RNA
attttgtgatgtgggaatc

SEQ ID NO: 1083 - RNA, 19 nt, synthetic construct
  misc_feature 1..19: note = antisense sequence
  source 1..19: mol_type = other RNA
gattcccacatcacaaaat

SEQ ID NO: 1084 - RNA, 19 nt, synthetic construct
  misc_feature 1..19: note = sense sequence
  source 1..19: mol_type = other RNA
gatgtgggaatcaatttta

SEQ ID NO: 1085 - RNA, 19 nt, synthetic construct
  misc_feature 1..19: note = antisense sequence
  source 1..19: mol_type = other RNA
taaaattgattcccacatc

SEQ ID NO: 1086 - RNA, 19 nt, synthetic construct
  misc_feature 1..19: note = sense sequence
  source 1..19: mol_type = other RNA
atgtgggaatcaattttag

SEQ ID NO: 1087 - RNA, 19 nt, synthetic construct
  misc_feature 1..19: note = antisense sequence
  source 1..19: mol_type = other RNA
ctaaaattgattcccacat

SEQ ID NO: 1088 - RNA, 19 nt, synthetic construct
  misc_feature 1..19: note = sense sequence
  source 1..19: mol_type = other RNA
tgtgggaatcaattttaga

SEQ ID NO: 1089 - RNA, 19 nt, synthetic construct
  misc_feature 1..19: note = antisense sequence
  source 1..19: mol_type = other RNA
tctaaaattgattcccaca

SEQ ID NO: 1090 - RNA, 19 nt, synthetic construct
  misc_feature 1..19: note = sense sequence
  source 1..19: mol_type = other RNA
gtgggaatcaattttagat

SEQ ID NO: 1091 - RNA, 19 nt, synthetic construct
  misc_feature 1..19: note = antisense sequence
  source 1..19: mol_type = other RNA
atctaaaattgattcccac

SEQ ID NO: 1092 - RNA, 19 nt, synthetic construct
  misc_feature 1..19: note = sense sequence
  source 1..19: mol_type = other RNA
tgggaatcaattttagatg

SEQ ID NO: 1093 - RNA, 19 nt, synthetic construct
  misc_feature 1..19: note = antisense sequence
  source 1..19: mol_type = other RNA
catctaaaattgattccca

SEQ ID NO: 1094 - RNA, 19 nt, synthetic construct
  misc_feature 1..19: note = sense sequence
  source 1..19: mol_type = other RNA
gggaatcaattttagatgg

SEQ ID NO: 1095 - RNA, 19 nt, synthetic construct
  misc_feature 1..19: note = antisense sequence
  source 1..19: mol_type = other RNA
ccatctaaaattgattccc

SEQ ID NO: 1096 - RNA, 19 nt, synthetic construct
  misc_feature 1..19: note = sense sequence
  source 1..19: mol_type = other RNA
ggaatcaattttagatggt

SEQ ID NO: 1097 - RNA, 19 nt, synthetic construct
  misc_feature 1..19: note = antisense sequence
  source 1..19: mol_type = other RNA
accatctaaaattgattcc

SEQ ID NO: 1098 - RNA, 19 nt, synthetic construct
  misc_feature 1..19: note = sense sequence
  source 1..19: mol_type = other RNA
gaatcaattttagatggtc

SEQ ID NO: 1099 - RNA, 19 nt, synthetic construct
  misc_feature 1..19: note = antisense sequence
  source 1..19: mol_type = other RNA
gaccatctaaaattgattc

SEQ ID NO: 1100 - RNA, 19 nt, synthetic construct
  misc_feature 1..19: note = sense sequence
  source 1..19: mol_type = other RNA
tcaattttagatggtcaca

SEQ ID NO: 1101 - RNA, 19 nt, synthetic construct
  misc_feature 1..19: note = antisense sequence
  source 1..19: mol_type = other RNA
tgtgaccatctaaaattga

SEQ ID NO: 1102 - RNA, 19 nt, synthetic construct
  misc_feature 1..19: note = sense sequence
  source 1..19: mol_type = other RNA
ttaaaaggcatcatatgag

SEQ ID NO: 1103 - RNA, 19 nt, synthetic construct
  misc_feature 1..19: note = antisense sequence
  source 1..19: mol_type = other RNA
ctcatatgatgccttttaa

SEQ ID NO: 1104 - RNA, 19 nt, synthetic construct
  misc_feature 1..19: note = sense sequence
  source 1..19: mol_type = other RNA
aaggcatcatatgagctaa

SEQ ID NO: 1105 - RNA, 19 nt, synthetic construct
  misc_feature 1..19: note = antisense sequence
  source 1..19: mol_type = other RNA
ttagctcatatgatgcctt

SEQ ID NO: 1106 - RNA, 19 nt, synthetic construct
  misc_feature 1..19: note = sense sequence
  source 1..19: mol_type = other RNA
aggcatcatatgagctaat

SEQ ID NO: 1107 - RNA, 19 nt, synthetic construct
  misc_feature 1..19: note = antisense sequence
  source 1..19: mol_type = other RNA
attagctcatatgatgcct

SEQ ID NO: 1108 - RNA, 19 nt, synthetic construct
  misc_feature 1..19: note = sense sequence
  source 1..19: mol_type = other RNA
ggcatcatatgagctaata

SEQ ID NO: 1109 - RNA, 19 nt, synthetic construct
  misc_feature 1..19: note = antisense sequence
  source 1..19: mol_type = other RNA
tattagctcatatgatgcc

SEQ ID NO: 1110 - RNA, 19 nt, synthetic construct
  misc_feature 1..19: note = sense sequence
  source 1..19: mol_type = other RNA
aacttgactaaatacagag

SEQ ID NO: 1111 - RNA, 19 nt, synthetic construct
  misc_feature 1..19: note = antisense sequence
  source 1..19: mol_type = other RNA
ctctgtatttagtcaagtt

SEQ ID NO: 1112 - RNA, 19 nt, synthetic construct
  misc_feature 1..19: note = sense sequence
  source 1..19: mol_type = other RNA
acttgactaaatacagagg

SEQ ID NO: 1113 - RNA, 19 nt, synthetic construct
  misc_feature 1..19: note = antisense sequence
  source 1..19: mol_type = other RNA
cctctgtatttagtcaagt

SEQ ID NO: 1114 - RNA, 19 nt, synthetic construct
  misc_feature 1..19: note = sense sequence
  source 1..19: mol_type = other RNA
cttgactaaatacagagga

SEQ ID NO: 1115 - RNA, 19 nt, synthetic construct
  misc_feature 1..19: note = antisense sequence
  source 1..19: mol_type = other RNA
tcctctgtatttagtcaag

SEQ ID NO: 1116 - RNA, 19 nt, synthetic construct
  misc_feature 1..19: note = sense sequence
  source 1..19: mol_type = other RNA
ttgactaaatacagaggac

SEQ ID NO: 1117 - RNA, 19 nt, synthetic construct
  misc_feature 1..19: note = antisense sequence
  source 1..19: mol_type = other RNA
gtcctctgtatttagtcaa

SEQ ID NO: 1118 - RNA, 19 nt, synthetic construct
  misc_feature 1..19: note = sense sequence
  source 1..19: mol_type = other RNA
tgactaaatacagaggact

SEQ ID NO: 1119 - RNA, 19 nt, synthetic construct
  misc_feature 1..19: note = antisense sequence
  source 1..19: mol_type = other RNA
agtcctctgtatttagtca

SEQ ID NO: 1120 - RNA, 19 nt, synthetic construct
  misc_feature 1..19: note = sense sequence
  source 1..19: mol_type = other RNA
taaatacagaggactggta

SEQ ID NO: 1121 - RNA, 19 nt, synthetic construct
  misc_feature 1..19: note = antisense sequence
  source 1..19: mol_type = other RNA
taccagtcctctgtattta

SEQ ID NO: 1122 - RNA, 19 nt, synthetic construct
  misc_feature 1..19: note = sense sequence
  source 1..19: mol_type = other RNA
aaatacagaggactggtaa

SEQ ID NO: 1123 - RNA, 19 nt, synthetic construct
  misc_feature 1..19: note = antisense sequence
  source 1..19: mol_type = other RNA
ttaccagtcctctgtattt

SEQ ID NO: 1124 - RNA, 19 nt, synthetic construct
  misc_feature 1..19: note = sense sequence
  source 1..19: mol_type = other RNA
aatacagaggactggtaat

SEQ ID NO: 1125 - RNA, 19 nt, synthetic construct
  misc_feature 1..19: note = antisense sequence
  source 1..19: mol_type = other RNA
attaccagtcctctgtatt

SEQ ID NO: 1126 - RNA, 19 nt, synthetic construct
  misc_feature 1..19: note = sense sequence
  source 1..19: mol_type = other RNA
tacagaggactggtaattg

SEQ ID NO: 1127 - RNA, 19 nt, synthetic construct
  misc_feature 1..19: note = antisense sequence
  source 1..19: mol_type = other RNA
caattaccagtcctctgta

SEQ ID NO: 1128 - RNA, 19 nt, synthetic construct
  misc_feature 1..19: note = sense sequence
  source 1..19: mol_type = other RNA
acagaggactggtaattgt

SEQ ID NO: 1129 - RNA, 19 nt, synthetic construct
  misc_feature 1..19: note = antisense sequence
  source 1..19: mol_type = other RNA
acaattaccagtcctctgt

SEQ ID NO: 1130 - RNA, 19 nt, synthetic construct
  misc_feature 1..19: note = sense sequence
  source 1..19: mol_type = other RNA
cagaggactggtaattgta

SEQ ID NO: 1131 - RNA, 19 nt, synthetic construct
  misc_feature 1..19: note = antisense sequence
  source 1..19: mol_type = other RNA
tacaattaccagtcctctg

SEQ ID NO: 1132 - RNA, 19 nt, synthetic construct
  misc_feature 1..19: note = sense sequence
  source 1..19: mol_type = other RNA
agaggactggtaattgtac

SEQ ID NO: 1133 - RNA, 19 nt, synthetic construct
  misc_feature 1..19: note = antisense sequence
  source 1..19: mol_type = other RNA
gtacaattaccagtcctct

SEQ ID NO: 1134 - RNA, 19 nt, synthetic construct
  misc_feature 1..19: note = sense sequence
  source 1..19: mol_type = other RNA
ctggtaattgtacagttct

SEQ ID NO: 1135 - RNA, 19 nt, synthetic construct
  misc_feature 1..19: note = antisense sequence
  source 1..19: mol_type = other RNA
agaactgtacaattaccag

SEQ ID NO: 1136 - RNA, 19 nt, synthetic construct
  misc_feature 1..19: note = sense sequence
  source 1..19: mol_type = other RNA
tggtaattgtacagttctt

SEQ ID NO: 1137 - RNA, 19 nt, synthetic construct
  misc_feature 1..19: note = antisense sequence
  source 1..19: mol_type = other RNA
aagaactgtacaattacca

SEQ ID NO: 1138 - RNA, 19 nt, synthetic construct
  misc_feature 1..19: note = sense sequence
  source 1..19: mol_type = other RNA
ggtaattgtacagttctta

SEQ ID NO: 1139 - RNA, 19 nt, synthetic construct
  misc_feature 1..19: note = antisense sequence
  source 1..19: mol_type = other RNA
taagaactgtacaattacc

SEQ ID NO: 1140 - RNA, 19 nt, synthetic construct
  misc_feature 1..19: note = sense sequence
  source 1..19: mol_type = other RNA
cagttcttaaatgttgtag

SEQ ID NO: 1141 - RNA, 19 nt, synthetic construct
  misc_feature 1..19: note = antisense sequence
  source 1..19: mol_type = other RNA
ctacaacatttaagaactg

SEQ ID NO: 1142 - RNA, 19 nt, synthetic construct
  misc_feature 1..19: note = sense sequence
  source 1..19: mol_type = other RNA
aaaatcgtcagcacagagt

SEQ ID NO: 1143 - RNA, 19 nt, synthetic construct
  misc_feature 1..19: note = antisense sequence
  source 1..19: mol_type = other RNA
actctgtgctgacgatttt

SEQ ID NO: 1144 - RNA, 19 nt, synthetic construct
  misc_feature 1..19: note = sense sequence
  source 1..19: mol_type = other RNA
aaatcgtcagcacagagta

SEQ ID NO: 1145 - RNA, 19 nt, synthetic construct
  misc_feature 1..19: note = antisense sequence
  source 1..19: mol_type = other RNA
tactctgtgctgacgattt

SEQ ID NO: 1146 - RNA, 19 nt, synthetic construct
  misc_feature 1..19: note = sense sequence
  source 1..19: mol_type = other RNA
aatcgtcagcacagagtat

SEQ ID NO: 1147 - RNA, 19 nt, synthetic construct
  misc_feature 1..19: note = antisense sequence
  source 1..19: mol_type = other RNA
atactctgtgctgacgatt

SEQ ID NO: 1148 - RNA, 19 nt, synthetic construct
  misc_feature 1..19: note = sense sequence
  source 1..19: mol_type = other RNA
atcgtcagcacagagtatg

SEQ ID NO: 1149 - RNA, 19 nt, synthetic construct
  misc_feature 1..19: note = antisense sequence
  source 1..19: mol_type = other RNA
catactctgtgctgacgat

SEQ ID NO: 1150 - RNA, 19 nt, synthetic construct
  misc_feature 1..19: note = sense sequence
  source 1..19: mol_type = other RNA
aactgatgcttcattttgc

SEQ ID NO: 1151 - RNA, 19 nt, synthetic construct
  misc_feature 1..19: note = antisense sequence
  source 1..19: mol_type = other RNA
gcaaaatgaagcatcagtt

SEQ ID NO: 1152 - RNA, 19 nt, synthetic construct
  misc_feature 1..19: note = sense sequence
  source 1..19: mol_type = other RNA
actgatgcttcattttgct

SEQ ID NO: 1153 - RNA, 19 nt, synthetic construct
  misc_feature 1..19: note = antisense sequence
  source 1..19: mol_type = other RNA
agcaaaatgaagcatcagt

SEQ ID NO: 1154 - RNA, 19 nt, synthetic construct
  misc_feature 1..19: note = sense sequence
  source 1..19: mol_type = other RNA
ctgatgcttcattttgcta

SEQ ID NO: 1155 - RNA, 19 nt, synthetic construct
  misc_feature 1..19: note = antisense sequence
  source 1..19: mol_type = other RNA
tagcaaaatgaagcatcag

SEQ ID NO: 1156 - RNA, 19 nt, synthetic construct
  misc_feature 1..19: note = sense sequence
  source 1..19: mol_type = other RNA
gatgcttcattttgctaca

SEQ ID NO: 1157 - RNA, 19 nt, synthetic construct
  misc_feature 1..19: note = antisense sequence
  source 1..19: mol_type = other RNA
tgtagcaaaatgaagcatc

SEQ ID NO: 1158 - RNA, 19 nt, synthetic construct
  misc_feature 1..19: note = sense sequence
  source 1..19: mol_type = other RNA
gaagcagaattaaatactg

SEQ ID NO: 1159 - RNA, 19 nt, synthetic construct
  misc_feature 1..19: note = antisense sequence
  source 1..19: mol_type = other RNA
cagtatttaattctgcttc

SEQ ID NO: 1160 - RNA, 19 nt, synthetic construct
  misc_feature 1..19: note = sense sequence
  source 1..19: mol_type = other RNA
ttaaaataagttcgctgtc

SEQ ID NO: 1161 - RNA, 19 nt, synthetic construct
  misc_feature 1..19: note = antisense sequence
  source 1..19: mol_type = other RNA
gacagcgaacttattttaa

SEQ ID NO: 1162 - RNA, 19 nt, synthetic construct
  misc_feature 1..19: note = sense sequence
  source 1..19: mol_type = other RNA
taaaataagttcgctgtct

SEQ ID NO: 1163 - RNA, 19 nt, synthetic construct
  misc_feature 1..19: note = antisense sequence
  source 1..19: mol_type = other RNA
agacagcgaacttatttta

SEQ ID NO: 1164 - RNA, 19 nt, synthetic construct
  misc_feature 1..19: note = sense sequence
  source 1..19: mol_type = other RNA
aaaataagttcgctgtctt

SEQ ID NO: 1165 - RNA, 19 nt, synthetic construct
  misc_feature 1..19: note = antisense sequence
  source 1..19: mol_type = other RNA
aagacagcgaacttatttt

SEQ ID NO: 1166 - RNA, 19 nt, synthetic construct
  misc_feature 1..19: note = sense sequence
  source 1..19: mol_type = other RNA
taagttcgctgtctttaaa

SEQ ID NO: 1167 - RNA, 19 nt, synthetic construct
  misc_feature 1..19: note = antisense sequence
  source 1..19: mol_type = other RNA
tttaaagacagcgaactta

SEQ ID NO: 1168 - RNA, 19 nt, synthetic construct
  misc_feature 1..19: note = sense sequence
  source 1..19: mol_type = other RNA
attgtaaaggaatcttgtc

SEQ ID NO: 1169 - RNA, 19 nt, synthetic construct
  misc_feature 1..19: note = antisense sequence
  source 1..19: mol_type = other RNA
gacaagattcctttacaat

SEQ ID NO: 1170 - RNA, 19 nt, synthetic construct
  misc_feature 1..19: note = sense sequence
  source 1..19: mol_type = other RNA
ttgtaaaggaatcttgtca

SEQ ID NO: 1171 - RNA, 19 nt, synthetic construct
  misc_feature 1..19: note = antisense sequence
  source 1..19: mol_type = other RNA
tgacaagattcctttacaa

SEQ ID NO: 1172 - RNA, 19 nt, synthetic construct
  misc_feature 1..19: note = sense sequence
  source 1..19: mol_type = other RNA
tgtaaaggaatcttgtcag

SEQ ID NO: 1173 - RNA, 19 nt, synthetic construct
  misc_feature 1..19: note = antisense sequence
  source 1..19: mol_type = other RNA
ctgacaagattcctttaca

SEQ ID NO: 1174 - RNA, 19 nt, synthetic construct
  misc_feature 1..19: note = sense sequence
  source 1..19: mol_type = other RNA
gtaaaggaatcttgtcaga

SEQ ID NO: 1175 - RNA, 19 nt, synthetic construct
  misc_feature 1..19: note = antisense sequence
  source 1..19: mol_type = other RNA
tctgacaagattcctttac

SEQ ID NO: 1176 - RNA, 19 nt, synthetic construct
  misc_feature 1..19: note = sense sequence
  source 1..19: mol_type = other RNA
cttgtcagattacagtaag

SEQ ID NO: 1177 - RNA, 19 nt, synthetic construct
  misc_feature 1..19: note = antisense sequence
  source 1..19: mol_type = other RNA
cttactgtaatctgacaag

SEQ ID NO: 1178 - RNA, 19 nt, synthetic construct
  misc_feature 1..19: note = sense sequence
  source 1..19: mol_type = other RNA
ttgtcagattacagtaaga

SEQ ID NO: 1179 - RNA, 19 nt, synthetic construct
  misc_feature 1..19: note = antisense sequence
  source 1..19: mol_type = other RNA
tcttactgtaatctgacaa

SEQ ID NO: 1180 - RNA, 19 nt, synthetic construct
  misc_feature 1..19: note = sense sequence
  source 1..19: mol_type = other RNA
tgtcagattacagtaagaa

SEQ ID NO: 1181 - RNA, 19 nt, synthetic construct
  misc_feature 1..19: note = antisense sequence
  source 1..19: mol_type = other RNA
ttcttactgtaatctgaca

SEQ ID NO: 1182 - RNA, 19 nt, synthetic construct
  misc_feature 1..19: note = sense sequence
  source 1..19: mol_type = other RNA
gtcagattacagtaagaat

SEQ ID NO: 1183 - RNA, 19 nt, synthetic construct
  misc_feature 1..19: note = antisense sequence
  source 1..19: mol_type = other RNA
attcttactgtaatctgac

SEQ ID NO: 1184 - RNA, 19 nt, synthetic construct
  misc_feature 1..19: note = sense sequence
  source 1..19: mol_type = other RNA
tcagattacagtaagaatg

SEQ ID NO: 1185 - RNA, 19 nt, synthetic construct
  misc_feature 1..19: note = antisense sequence
  source 1..19: mol_type = other RNA
cattcttactgtaatctga

SEQ ID NO: 1186 - RNA, 19 nt, synthetic construct
  misc_feature 1..19: note = sense sequence
  source 1..19: mol_type = other RNA
tatgctgtgattctaatac

SEQ ID NO: 1187 - RNA, 19 nt, synthetic construct
  misc_feature 1..19: note = antisense sequence
  source 1..19: mol_type = other RNA
gtattagaatcacagcata

SEQ ID NO: 1188 - RNA, 19 nt, synthetic construct
  misc_feature 1..19: note = sense sequence
  source 1..19: mol_type = other RNA
atgctgtgattctaataca

SEQ ID NO: 1189 - RNA, 19 nt, synthetic construct
  misc_feature 1..19: note = antisense sequence
  source 1..19: mol_type = other RNA
tgtattagaatcacagcat

SEQ ID NO: 1190 - RNA, 19 nt, synthetic construct
  misc_feature 1..19: note = sense sequence
  source 1..19: mol_type = other RNA
gctgtgattctaatacatt

SEQ ID NO: 1191 - RNA, 19 nt, synthetic construct
  misc_feature 1..19: note = antisense sequence
  source 1..19: mol_type = other RNA
aatgtattagaatcacagc

SEQ ID NO: 1192 - RNA, 19 nt, synthetic construct
  misc_feature 1..19: note = sense sequence
  source 1..19: mol_type = other RNA
ctgtgattctaatacattc

SEQ ID NO: 1193 - RNA, 19 nt, synthetic construct
  misc_feature 1..19: note = antisense sequence
  source 1..19: mol_type = other RNA
gaatgtattagaatcacag

SEQ ID NO: 1194 - RNA, 19 nt, synthetic construct
  misc_feature 1..19: note = sense sequence
  source 1..19: mol_type = other RNA
tgtgattctaatacattcg

SEQ ID NO: 1195 - RNA, 19 nt, synthetic construct
  misc_feature 1..19: note = antisense sequence
  source 1..19: mol_type = other RNA
cgaatgtattagaatcaca

SEQ ID NO: 1196 - RNA, 19 nt, synthetic construct
  misc_feature 1..19: note = sense sequence
  source 1..19: mol_type = other RNA
gtgattctaatacattcgt

SEQ ID NO: 1197 - RNA, 19 nt, synthetic construct
  misc_feature 1..19: note = antisense sequence
  source 1..19: mol_type = other RNA
acgaatgtattagaatcac

SEQ ID NO: 1198 - RNA, 19 nt, synthetic construct
  misc_feature 1..19: note = sense sequence
  source 1..19: mol_type = other RNA
tgattctaatacattcgtg

SEQ ID NO: 1199 - RNA, 19 nt, synthetic construct
  misc_feature 1..19: note = antisense sequence
  source 1..19: mol_type = other RNA
cacgaatgtattagaatca

SEQ ID NO: 1200 - RNA, 19 nt, synthetic construct
  misc_feature 1..19: note = sense sequence
  source 1..19: mol_type = other RNA
gattctaatacattcgtgt

SEQ ID NO: 1201 - RNA, 19 nt, synthetic construct
  misc_feature 1..19: note = antisense sequence
  source 1..19: mol_type = other RNA
acacgaatgtattagaatc

SEQ ID NO: 1202 - RNA, 19 nt, synthetic construct
  misc_feature 1..19: note = sense sequence
  source 1..19: mol_type = other RNA
acattcgtgtaggttttca

SEQ ID NO: 1203 - RNA, 19 nt, synthetic construct
  misc_feature 1..19: note = antisense sequence
  source 1..19: mol_type = other RNA
tgaaaacctacacgaatgt

SEQ ID NO: 1204 - RNA, 19 nt, synthetic construct
  misc_feature 1..19: note = sense sequence
  source 1..19: mol_type = other RNA
cattcgtgtaggttttcaa

SEQ ID NO: 1205 - RNA, 19 nt, synthetic construct
  misc_feature 1..19: note = antisense sequence
  source 1..19: mol_type = other RNA
ttgaaaacctacacgaatg

SEQ ID NO: 1206 - RNA, 19 nt, synthetic construct
  misc_feature 1..19: note = sense sequence
  source 1..19: mol_type = other RNA
attcgtgtaggttttcaag

SEQ ID NO: 1207 - RNA, 19 nt, synthetic construct
  misc_feature 1..19: note = antisense sequence
  source 1..19: mol_type = other RNA
cttgaaaacctacacgaat

SEQ ID NO: 1208 - RNA, 19 nt, synthetic construct
  misc_feature 1..19: note = sense sequence
  source 1..19: mol_type = other RNA
ttcgtgtaggttttcaagt

SEQ ID NO: 1209 - RNA, 19 nt, synthetic construct
  misc_feature 1..19: note = antisense sequence
  source 1..19: mol_type = other RNA
acttgaaaacctacacgaa

SEQ ID NO: 1210 - RNA, 19 nt, synthetic construct
  misc_feature 1..19: note = sense sequence
  source 1..19: mol_type = other RNA
tcgtgtaggttttcaagta

SEQ ID NO: 1211 - RNA, 19 nt, synthetic construct
  misc_feature 1..19: note = antisense sequence
  source 1..19: mol_type = other RNA
tacttgaaaacctacacga

SEQ ID NO: 1212 - RNA, 19 nt, synthetic construct
  misc_feature 1..19: note = sense sequence
  source 1..19: mol_type = other RNA
cgtgtaggttttcaagtag

SEQ ID NO: 1213 - RNA, 19 nt, synthetic construct
  misc_feature 1..19: note = antisense sequence
  source 1..19: mol_type = other RNA
ctacttgaaaacctacacg

SEQ ID NO: 1214 - RNA, 19 nt, synthetic construct
  misc_feature 1..19: note = sense sequence
  source 1..19: mol_type = other RNA
gtgtaggttttcaagtaga

SEQ ID NO: 1215 - RNA, 19 nt, synthetic construct
  misc_feature 1..19: note = antisense sequence
  source 1..19: mol_type = other RNA
tctacttgaaaacctacac

SEQ ID NO: 1216 - RNA, 19 nt, synthetic construct
  misc_feature 1..19: note = sense sequence
  source 1..19: mol_type = other RNA
caaagacccagtccctaaa

SEQ ID NO: 1217 - RNA, 19 nt, synthetic construct
  misc_feature 1..19: note = antisense sequence
  source 1..19: mol_type = other RNA
tttagggactgggtctttg

SEQ ID NO: 1218 - RNA, 19 nt, synthetic construct
  misc_feature 1..19: note = sense sequence
  source 1..19: mol_type = other RNA
aaagacccagtccctaaat

SEQ ID NO: 1219 - RNA, 19 nt, synthetic construct
  misc_feature 1..19: note = antisense sequence
  source 1..19: mol_type = other RNA
atttagggactgggtcttt

SEQ ID NO: 1220 - RNA, 19 nt, synthetic construct
  misc_feature 1..19: note = sense sequence
  source 1..19: mol_type = other RNA
aagacccagtccctaaatt

SEQ ID NO: 1221 - RNA, 19 nt, synthetic construct
  misc_feature 1..19: note = antisense sequence
  source 1..19: mol_type = other RNA
aatttagggactgggtctt

SEQ ID NO: 1222 - RNA, 19 nt, synthetic construct
  misc_feature 1..19: note = sense sequence
  source 1..19: mol_type = other RNA
gacccagtccctaaattat

SEQ ID NO: 1223 - RNA, 19 nt, synthetic construct
  misc_feature 1..19: note = antisense sequence
  source 1..19: mol_type = other RNA
ataatttagggactgggtc

SEQ ID NO: 1224 - RNA, 19 nt, synthetic construct
  misc_feature 1..19: note = sense sequence
  source 1..19: mol_type = other RNA
tattcttgcatgtttatcg

SEQ ID NO: 1225 - RNA, 19 nt, synthetic construct
  misc_feature 1..19: note = antisense sequence
  source 1..19: mol_type = other RNA
cgataaacatgcaagaata

SEQ ID NO: 1226 - RNA, 19 nt, synthetic construct
  misc_feature 1..19: note = sense sequence
  source 1..19: mol_type = other RNA
attcttgcatgtttatcga

SEQ ID NO: 1227 - RNA, 19 nt, synthetic construct
  misc_feature 1..19: note = antisense sequence
  source 1..19: mol_type = other RNA
tcgataaacatgcaagaat

SEQ ID NO: 1228 - RNA, 19 nt, synthetic construct
  misc_feature 1..19: note = sense sequence
  source 1..19: mol_type = other RNA
ttcttgcatgtttatcgac

SEQ ID NO: 1229 - RNA, 19 nt, synthetic construct
  misc_feature 1..19: note = antisense sequence
  source 1..19: mol_type = other RNA
gtcgataaacatgcaagaa

SEQ ID NO: 1230 - RNA, 19 nt, synthetic construct
  misc_feature 1..19: note = sense sequence
  source 1..19: mol_type = other RNA
tcttgcatgtttatcgaca

SEQ ID NO: 1231 - RNA, 19 nt, synthetic construct
  misc_feature 1..19: note = antisense sequence
  source 1..19: mol_type = other RNA
tgtcgataaacatgcaaga

SEQ ID NO: 1232 - RNA, 19 nt, synthetic construct
  misc_feature 1..19: note = sense sequence
  source 1..19: mol_type = other RNA
aatccctaaatccctaaag

SEQ ID NO: 1233 - RNA, 19 nt, synthetic construct
  misc_feature 1..19: note = antisense sequence
  source 1..19: mol_type = other RNA
ctttagggatttagggatt

SEQ ID NO: 1234 - RNA, 19 nt, synthetic construct
  misc_feature 1..19: note = sense sequence
  source 1..19: mol_type = other RNA
tccctaaatccctaaagat

SEQ ID NO: 1235 - RNA, 19 nt, synthetic construct
  misc_feature 1..19: note = antisense sequence
  source 1..19: mol_type = other RNA
atctttagggatttaggga

SEQ ID NO: 1236 - RNA, 19 nt, synthetic construct
  misc_feature 1..19: note = sense sequence
  source 1..19: mol_type = other RNA
ttttaccacagtatcactt

SEQ ID NO: 1237 - RNA, 19 nt, synthetic construct
  misc_feature 1..19: note = antisense sequence
  source 1..19: mol_type = other RNA
aagtgatactgtggtaaaa

SEQ ID NO: 1238 - RNA, 19 nt, synthetic construct
  misc_feature 1..19: note = sense sequence
  source 1..19: mol_type = other RNA
tttaccacagtatcacttg

SEQ ID NO: 1239 - RNA, 19 nt, synthetic construct
  misc_feature 1..19: note = antisense sequence
  source 1..19: mol_type = other RNA
caagtgatactgtggtaaa

SEQ ID NO: 1240 - RNA, 19 nt, synthetic construct
  misc_feature 1..19: note = sense sequence
  source 1..19: mol_type = other RNA
ttaccacagtatcacttgt

SEQ ID NO: 1241 - RNA, 19 nt, synthetic construct
  misc_feature 1..19: note = antisense sequence
  source 1..19: mol_type = other RNA
acaagtgatactgtggtaa

SEQ ID NO: 1242 - RNA, 19 nt, synthetic construct
  misc_feature 1..19: note = sense sequence
  source 1..19: mol_type = other RNA
taccacagtatcacttgtc

SEQ ID NO: 1243 - RNA, 19 nt, synthetic construct
  misc_feature 1..19: note = antisense sequence
  source 1..19: mol_type = other RNA
gacaagtgatactgtggta

SEQ ID NO: 1244 - RNA, 19 nt, synthetic construct
  misc_feature 1..19: note = sense sequence
  source 1..19: mol_type = other RNA
accacagtatcacttgtca

SEQ ID NO: 1245 - RNA, 19 nt, synthetic construct
  misc_feature 1..19: note = antisense sequence
  source 1..19: mol_type = other RNA
tgacaagtgatactgtggt

SEQ ID NO: 1246 - RNA, 19 nt, synthetic construct
  misc_feature 1..19: note = sense sequence
  source 1..19: mol_type = other RNA
ccacagtatcacttgtcag

SEQ ID NO: 1247 - RNA, 19 nt, synthetic construct
  misc_feature 1..19: note = antisense sequence
  source 1..19: mol_type = other RNA
ctgacaagtgatactgtgg

SEQ ID NO: 1248 - RNA, 19 nt, synthetic construct
  misc_feature 1..19: note = sense sequence
  source 1..19: mol_type = other RNA
cacagtatcacttgtcaga

SEQ ID NO: 1249 - RNA, 19 nt, synthetic construct
  misc_feature 1..19: note = antisense sequence
  source 1..19: mol_type = other RNA
tctgacaagtgatactgtg

SEQ ID NO: 1250 - RNA, 19 nt, synthetic construct
  misc_feature 1..19: note = sense sequence
  source 1..19: mol_type = other RNA
acagtatcacttgtcagaa

SEQ ID NO: 1251 - RNA, 19 nt, synthetic construct
  misc_feature 1..19: note = antisense sequence
  source 1..19: mol_type = other RNA
ttctgacaagtgatactgt

SEQ ID NO: 1252 - RNA, 19 nt, synthetic construct
  misc_feature 1..19: note = sense sequence
  source 1..19: mol_type = other RNA
cagtatcacttgtcagaat

SEQ ID NO: 1253 - RNA, 19 nt, synthetic construct
  misc_feature 1..19: note = antisense sequence
  source 1..19: mol_type = other RNA
attctgacaagtgatactg

SEQ ID NO: 1254 - RNA, 19 nt, synthetic construct
  misc_feature 1..19: note = sense sequence
  source 1..19: mol_type = other RNA
agtatcacttgtcagaatt

SEQ ID NO: 1255 - RNA, 19 nt, synthetic construct
  misc_feature 1..19: note = antisense sequence
  source 1..19: mol_type = other RNA
aattctgacaagtgatact

SEQ ID NO: 1256 - RNA, 19 nt, synthetic construct
  misc_feature 1..19: note = sense sequence
  source 1..19: mol_type = other RNA
ttgttaaaggatatagtgc

SEQ ID NO: 1257 - RNA, 19 nt, synthetic construct
  misc_feature 1..19: note = antisense sequence
  source 1..19: mol_type = other RNA
gcactatatcctttaacaa

SEQ ID NO: 1258 - RNA, 19 nt, synthetic construct
  misc_feature 1..19: note = sense sequence
  source 1..19: mol_type = other RNA
tgttaaaggatatagtgcc

SEQ ID NO: 1259 - RNA, 19 nt, synthetic construct
  misc_feature 1..19: note = antisense sequence
  source 1..19: mol_type = other RNA
ggcactatatcctttaaca

SEQ ID NO: 1260 - RNA, 19 nt, synthetic construct
  misc_feature 1..19: note = sense sequence
  source 1..19: mol_type = other RNA
gttaaaggatatagtgccc

SEQ ID NO: 1261 - RNA, 19 nt, synthetic construct
  misc_feature 1..19: note = antisense sequence
  source 1..19: mol_type = other RNA
gggcactatatcctttaac

SEQ ID NO: 1262 - RNA, 19 nt, synthetic construct
  misc_feature 1..19: note = sense sequence
  source 1..19: mol_type = other RNA
ttaaaggatatagtgccca

SEQ ID NO: 1263 - RNA, 19 nt, synthetic construct
  misc_feature 1..19: note = antisense sequence
  source 1..19: mol_type = other RNA
tgggcactatatcctttaa

SEQ ID NO: 1264 - RNA, 19 nt, synthetic construct
  misc_feature 1..19: note = sense sequence
  source 1..19: mol_type = other RNA
taaaggatatagtgcccaa

SEQ ID NO: 1265 - RNA, 19 nt, synthetic construct
  misc_feature 1..19: note = antisense sequence
  source 1..19: mol_type = other RNA
ttgggcactatatccttta

SEQ ID NO: 1266 - RNA, 19 nt, synthetic construct
  misc_feature 1..19: note = sense sequence
  source 1..19: mol_type = other RNA
aaaggatatagtgcccaag

SEQ ID NO: 1267 - RNA, 19 nt, synthetic construct
  misc_feature 1..19: note = antisense sequence
  source 1..19: mol_type = other RNA
cttgggcactatatccttt

SEQ ID NO: 1268 - RNA, 19 nt, synthetic construct
  misc_feature 1..19: note = sense sequence
  source 1..19: mol_type = other RNA
aaggatatagtgcccaagt

SEQ ID NO: 1269 - RNA, 19 nt, synthetic construct
  misc_feature 1..19: note = antisense sequence
  source 1..19: mol_type = other RNA
acttgggcactatatcctt

SEQ ID NO: 1270 - RNA, 19 nt, synthetic construct
  misc_feature 1..19: note = sense sequence
  source 1..19: mol_type = other RNA
aggatatagtgcccaagtt

SEQ ID NO: 1271 - RNA, 19 nt, synthetic construct
  misc_feature 1..19: note = antisense sequence
  source 1..19: mol_type = other RNA
aacttgggcactatatcct

SEQ ID NO: 1272 - RNA, 19 nt, synthetic construct
  misc_feature 1..19: note = sense sequence
  source 1..19: mol_type = other RNA
ggatatagtgcccaagtta

SEQ ID NO: 1273 - RNA, 19 nt, synthetic construct
  misc_feature 1..19: note = antisense sequence
  source 1..19: mol_type = other RNA
taacttgggcactatatcc

SEQ ID NO: 1274 - RNA, 19 nt, synthetic construct
  misc_feature 1..19: note = sense sequence
  source 1..19: mol_type = other RNA
agtgcccaagttatatggt

SEQ ID NO: 1275 - RNA, 19 nt, synthetic construct
  misc_feature 1..19: note = antisense sequence
  source 1..19: mol_type = other RNA
accatataacttgggcact

SEQ ID NO: 1276 - RNA, 19 nt, synthetic construct
  misc_feature 1..19: note = sense sequence
  source 1..19: mol_type = other RNA
gtgcccaagttatatggtg

SEQ ID NO: 1277 - RNA, 19 nt, synthetic construct
  misc_feature 1..19: note = antisense sequence
  source 1..19: mol_type = other RNA
caccatataacttgggcac

SEQ ID NO: 1278 - RNA, 19 nt, synthetic construct
  misc_feature 1..19: note = sense sequence
  source 1..19: mol_type = other RNA
tgcccaagttatatggtga

SEQ ID NO: 1279 - RNA, 19 nt, synthetic construct
  misc_feature 1..19: note = antisense sequence
  source 1..19: mol_type = other RNA
tcaccatataacttgggca

SEQ ID NO: 1280 - RNA, 19 nt, synthetic construct
  misc_feature 1..19: note = sense sequence
  source 1..19: mol_type = other RNA
gcccaagttatatggtgac

SEQ ID NO: 1281 - RNA, 19 nt, synthetic construct
  misc_feature 1..19: note = antisense sequence
  source 1..19: mol_type = other RNA
gtcaccatataacttgggc

SEQ ID NO: 1282 - RNA, 19 nt, synthetic construct
  misc_feature 1..19: note = sense sequence
  source 1..19: mol_type = other RNA
cccaagttatatggtgacc

SEQ ID NO: 1283 - RNA, 19 nt, synthetic construct
  misc_feature 1..19: note = antisense sequence
  source 1..19: mol_type = other RNA
ggtcaccatataacttggg

SEQ ID NO: 1284 - RNA, 19 nt, synthetic construct
  misc_feature 1..19: note = sense sequence
  source 1..19: mol_type = other RNA
agttatatggtgacctacc

SEQ ID NO: 1285 - RNA, 19 nt, synthetic construct
  misc_feature 1..19: note = antisense sequence
  source 1..19: mol_type = other RNA
ggtaggtcaccatataact

SEQ ID NO: 1286 - RNA, 19 nt, synthetic construct
  misc_feature 1..19: note = sense sequence
  source 1..19: mol_type = other RNA
atatggtgacctacctttg

SEQ ID NO: 1287 - RNA, 19 nt, synthetic construct
  misc_feature 1..19: note = antisense sequence
  source 1..19: mol_type = other RNA
caaaggtaggtcaccatat

SEQ ID NO: 1288 - RNA, 19 nt, synthetic construct
  misc_feature 1..19: note = sense sequence
  source 1..19: mol_type = other RNA
tatggtgacctacctttgt

SEQ ID NO: 1289 - RNA, 19 nt, synthetic construct
  misc_feature 1..19: note = antisense sequence
  source 1..19: mol_type = other RNA
acaaaggtaggtcaccata

SEQ ID NO: 1290 - RNA, 19 nt, synthetic construct
  misc_feature 1..19: note = sense sequence
  source 1..19: mol_type = other RNA
atggtgacctacctttgtc

SEQ ID NO: 1291 - RNA, 19 nt, synthetic construct
  misc_feature 1..19: note = antisense sequence
  source 1..19: mol_type = other RNA
gacaaaggtaggtcaccat

SEQ ID NO: 1292 - RNA, 19 nt, synthetic construct
  misc_feature 1..19: note = sense sequence
  source 1..19: mol_type = other RNA
gacctacctttgtcaatac

SEQ ID NO: 1293 - RNA, 19 nt, synthetic construct
  misc_feature 1..19: note = antisense sequence
  source 1..19: mol_type = other RNA
gtattgacaaaggtaggtc

SEQ ID NO: 1294 - RNA, 19 nt, synthetic construct
  misc_feature 1..19: note = sense sequence
  source 1..19: mol_type = other RNA
acctacctttgtcaatact

SEQ ID NO: 1295 - RNA, 19 nt, synthetic construct
  misc_feature 1..19: note = antisense sequence
  source 1..19: mol_type = other RNA
agtattgacaaaggtaggt

SEQ ID NO: 1296 - RNA, 19 nt, synthetic construct
  misc_feature 1..19: note = sense sequence
  source 1..19: mol_type = other RNA
ctacctttgtcaatactta

SEQ ID NO: 1297 - RNA, 19 nt, synthetic construct
  misc_feature 1..19: note = antisense sequence
  source 1..19: mol_type = other RNA
taagtattgacaaaggtag

SEQ ID NO: 1298 - RNA, 19 nt, synthetic construct
  misc_feature 1..19: note = sense sequence
  source 1..19: mol_type = other RNA
tacctttgtcaatacttag

SEQ ID NO: 1299 - RNA, 19 nt, synthetic construct
  misc_feature 1..19: note = antisense sequence
  source 1..19: mol_type = other RNA
ctaagtattgacaaaggta

SEQ ID NO: 1300 - RNA, 19 nt, synthetic construct
  misc_feature 1..19: note = sense sequence
  source 1..19: mol_type = other RNA
acctttgtcaatacttagc

SEQ ID NO: 1301 - RNA, 19 nt, synthetic construct
  misc_feature 1..19: note = antisense sequence
  source 1..19: mol_type = other RNA
gctaagtattgacaaaggt

SEQ ID NO: 1302 - RNA, 19 nt, synthetic construct
  misc_feature 1..19: note = sense sequence
  source 1..19: mol_type = other RNA
cctttgtcaatacttagca

SEQ ID NO: 1303 - RNA, 19 nt, synthetic construct
  misc_feature 1..19: note = antisense sequence
  source 1..19: mol_type = other RNA
tgctaagtattgacaaagg

SEQ ID NO: 1304 - RNA, 19 nt, synthetic construct
  misc_feature 1..19: note = sense sequence
  source 1..19: mol_type = other RNA
ctttgtcaatacttagcat

SEQ ID NO: 1305 - RNA, 19 nt, synthetic construct
  misc_feature 1..19: note = antisense sequence
  source 1..19: mol_type = other RNA
atgctaagtattgacaaag

SEQ ID NO: 1306 - RNA, 19 nt, synthetic construct
  misc_feature 1..19: note = sense sequence
  source 1..19: mol_type = other RNA
tgtatgatctatgtgaatc

SEQ ID NO: 1307 - RNA, 19 nt, synthetic construct
  misc_feature 1..19: note = antisense sequence
  source 1..19: mol_type = other RNA
gattcacatagatcataca

SEQ ID NO: 1308 - RNA, 19 nt, synthetic construct
  misc_feature 1..19: note = sense sequence
  source 1..19: mol_type = other RNA
tatgatctatgtgaatcct

SEQ ID NO: 1309 - RNA, 19 nt, synthetic construct
  misc_feature 1..19: note = antisense sequence
  source 1..19: mol_type = other RNA
aggattcacatagatcata

SEQ ID NO: 1310 - RNA, 19 nt, synthetic construct
  misc_feature 1..19: note = sense sequence
  source 1..19: mol_type = other RNA
atgatctatgtgaatccta

SEQ ID NO: 1311 - RNA, 19 nt, synthetic construct
  misc_feature 1..19: note = antisense sequence
  source 1..19: mol_type = other RNA
taggattcacatagatcat

SEQ ID NO: 1312 - RNA, 19 nt, synthetic construct
  misc_feature 1..19: note = sense sequence
  source 1..19: mol_type = other RNA
tgatctatgtgaatcctaa

SEQ ID NO: 1313 - RNA, 19 nt, synthetic construct
  misc_feature 1..19: note = antisense sequence
  source 1..19: mol_type = other RNA
ttaggattcacatagatca

SEQ ID NO: 1314 - RNA, 19 nt, synthetic construct
  misc_feature 1..19: note = sense sequence
  source 1..19: mol_type = other RNA
gatctatgtgaatcctaag

SEQ ID NO: 1315 - RNA, 19 nt, synthetic construct
  misc_feature 1..19: note = antisense sequence
  source 1..19: mol_type = other RNA
cttaggattcacatagatc

SEQ ID NO: 1316 - RNA, 19 nt, synthetic construct
  misc_feature 1..19: note = sense sequence
  source 1..19: mol_type = other RNA
atctatgtgaatcctaagt

SEQ ID NO: 1317 - RNA, 19 nt, synthetic construct
  misc_feature 1..19: note = antisense sequence
  source 1..19: mol_type = other RNA
acttaggattcacatagat

SEQ ID NO: 1318 - RNA, 19 nt, synthetic construct
  misc_feature 1..19: note = sense sequence
  source 1..19: mol_type = other RNA
tctatgtgaatcctaagta

SEQ ID NO: 1319 - RNA, 19 nt, synthetic construct
  misc_feature 1..19: note = antisense sequence
  source 1..19: mol_type = other RNA
tacttaggattcacataga

SEQ ID NO: 1320 - RNA, 19 nt, synthetic construct
  misc_feature 1..19: note = sense sequence
  source 1..19: mol_type = other RNA
tctataattttcaggacca

SEQ ID NO: 1321 - RNA, 19 nt, synthetic construct
  misc_feature 1..19: note = antisense sequence
  source 1..19: mol_type = other RNA
tggtcctgaaaattataga

SEQ ID NO: 1322 - RNA, 19 nt, synthetic construct
  misc_feature 1..19: note = sense sequence
  source 1..19: mol_type = other RNA
ctataattttcaggaccac

SEQ ID NO: 1323 - RNA, 19 nt, synthetic construct
  misc_feature 1..19: note = antisense sequence
  source 1..19: mol_type = other RNA
gtggtcctgaaaattatag

SEQ ID NO: 1324 - RNA, 19 nt, synthetic construct
  misc_feature 1..19: note = sense sequence
  source 1..19: mol_type = other RNA
attttcaggaccacagact

SEQ ID NO: 1325 - RNA, 19 nt, synthetic construct
  misc_feature 1..19: note = antisense sequence
  source 1..19: mol_type = other RNA
agtctgtggtcctgaaaat

SEQ ID NO: 1326 - RNA, 19 nt, synthetic construct
  misc_feature 1..19: note = sense sequence
  source 1..19: mol_type = other RNA
ttcaggaccacagactaag

SEQ ID NO: 1327 - RNA, 19 nt, synthetic construct
  misc_feature 1..19: note = antisense sequence
  source 1..19: mol_type = other RNA
cttagtctgtggtcctgaa

SEQ ID NO: 1328 - RNA, 19 nt, synthetic construct
  misc_feature 1..19: note = sense sequence
  source 1..19: mol_type = other RNA
tcaggaccacagactaagc

SEQ ID NO: 1329 - RNA, 19 nt, synthetic construct
  misc_feature 1..19: note = antisense sequence
  source 1..19: mol_type = other RNA
gcttagtctgtggtcctga

SEQ ID NO: 1330 - RNA, 19 nt, synthetic construct
  misc_feature 1..19: note = sense sequence
  source 1..19: mol_type = other RNA
ttttagggccagaatacca

SEQ ID NO: 1331 - RNA, 19 nt, synthetic construct
  misc_feature 1..19: note = antisense sequence
  source 1..19: mol_type = other RNA
tggtattctggccctaaaa

SEQ ID NO: 1332 - RNA, 19 nt, synthetic construct
  misc_feature 1..19: note = sense sequence
  source 1..19: mol_type = other RNA
tttagggccagaataccaa

SEQ ID NO: 1333 - RNA, 19 nt, synthetic construct
  misc_feature 1..19: note = antisense sequence
  source 1..19: mol_type = other RNA
ttggtattctggccctaaa

SEQ ID NO: 1334 - RNA, 19 nt, synthetic construct
  misc_feature 1..19: note = sense sequence
  source 1..19: mol_type = other RNA
ttagggccagaataccaaa

SEQ ID NO: 1335 - RNA, 19 nt, synthetic construct
  misc_feature 1..19: note = antisense sequence
  source 1..19: mol_type = other RNA
tttggtattctggccctaa

SEQ ID NO: 1336 - RNA, 19 nt, synthetic construct
  misc_feature 1..19: note = sense sequence
  source 1..19: mol_type = other RNA
tagggccagaataccaaaa

SEQ ID NO: 1337 - RNA, 19 nt, synthetic construct
  misc_feature 1..19: note = antisense sequence
  source 1..19: mol_type = other RNA
ttttggtattctggcccta

SEQ ID NO: 1338 - RNA, 19 nt, synthetic construct
  misc_feature 1..19: note = sense sequence
  source 1..19: mol_type = other RNA
agggccagaataccaaaat

SEQ ID NO: 1339 - RNA, 19 nt, synthetic construct
  misc_feature 1..19: note = antisense sequence
  source 1..19: mol_type = other RNA
attttggtattctggccct

SEQ ID NO: 1340 - RNA, 19 nt, synthetic construct
  misc_feature 1..19: note = sense sequence
  source 1..19: mol_type = other RNA
gggccagaataccaaaatg

SEQ ID NO: 1341 - RNA, 19 nt, synthetic construct
  misc_feature 1..19: note = antisense sequence
  source 1..19: mol_type = other RNA
cattttggtattctggccc

SEQ ID NO: 1342 - RNA, 19 nt, synthetic construct
  misc_feature 1..19: note = sense sequence
  source 1..19: mol_type = other RNA
ggccagaataccaaaatgg

SEQ ID NO: 1343 - RNA, 19 nt, synthetic construct
  misc_feature 1..19: note = antisense sequence
  source 1..19: mol_type = other RNA
ccattttggtattctggcc

SEQ ID NO: 1344 - RNA, 19 nt, synthetic construct
  misc_feature 1..19: note = sense sequence
  source 1..19: mol_type = other RNA
ccagaataccaaaatggct

SEQ ID NO: 1345 - RNA, 19 nt, synthetic construct
  misc_feature 1..19: note = antisense sequence
  source 1..19: mol_type = other RNA
agccattttggtattctgg

SEQ ID NO: 1346 - RNA, 19 nt, synthetic construct
  misc_feature 1..19: note = sense sequence
  source 1..19: mol_type = other RNA
cagaataccaaaatggctc

SEQ ID NO: 1347 - RNA, 19 nt, synthetic construct
  misc_feature 1..19: note = antisense sequence
  source 1..19: mol_type = other RNA
gagccattttggtattctg

SEQ ID NO: 1348 - RNA, 19 nt, synthetic construct
  misc_feature 1..19: note = sense sequence
  source 1..19: mol_type = other RNA
taccaaaatggctcctctc

SEQ ID NO: 1349 - RNA, 19 nt, synthetic construct
  misc_feature 1..19: note = antisense sequence
  source 1..19: mol_type = other RNA
gagaggagccattttggta

SEQ ID NO: 1350 - RNA, 19 nt, synthetic construct
  misc_feature 1..19: note = sense sequence
  source 1..19: mol_type = other RNA
accaaaatggctcctctct

SEQ ID NO: 1351 - RNA, 19 nt, synthetic construct
  misc_feature 1..19: note = antisense sequence
  source 1..19: mol_type = other RNA
agagaggagccattttggt

SEQ ID NO: 1352 - RNA, 19 nt, synthetic construct
  misc_feature 1..19: note = sense sequence
  source 1..19: mol_type = other RNA
taatatatgagttgcttcc

SEQ ID NO: 1353 - RNA, 19 nt, synthetic construct
  misc_feature 1..19: note = antisense sequence
  source 1..19: mol_type = other RNA
ggaagcaactcatatatta

SEQ ID NO: 1354 - RNA, 19 nt, synthetic construct
  misc_feature 1..19: note = sense sequence
  source 1..19: mol_type = other RNA
aatatatgagttgcttcct

SEQ ID NO: 1355 - RNA, 19 nt, synthetic construct
  misc_feature 1..19: note = antisense sequence
  source 1..19: mol_type = other RNA
aggaagcaactcatatatt

SEQ ID NO: 1356 - RNA, 19 nt, synthetic construct
  misc_feature 1..19: note = sense sequence
  source 1..19: mol_type = other RNA
atatatgagttgcttcctc

SEQ ID NO: 1357 - RNA, 19 nt, synthetic construct
  misc_feature 1..19: note = antisense sequence
  source 1..19: mol_type = other RNA
gaggaagcaactcatatat

SEQ ID NO: 1358 - RNA, 19 nt, synthetic construct
  misc_feature 1..19: note = sense sequence
  source 1..19: mol_type = other RNA
tgagttgcttcctctattt

SEQ ID NO: 1359 - RNA, 19 nt, synthetic construct
  misc_feature 1..19: note = antisense sequence
  source 1..19: mol_type = other RNA
aaatagaggaagcaactca

SEQ ID NO: 1360 - RNA, 19 nt, synthetic construct
  misc_feature 1..19: note = sense sequence
  source 1..19: mol_type = other RNA
gagttgcttcctctatttg

SEQ ID NO: 1361 - RNA, 19 nt, synthetic construct
  misc_feature 1..19: note = antisense sequence
  source 1..19: mol_type = other RNA
caaatagaggaagcaactc

SEQ ID NO: 1362 - RNA, 19 nt, synthetic construct
  misc_feature 1..19: note = sense sequence
  source 1..19: mol_type = other RNA
agttgcttcctctatttgg

SEQ ID NO: 1363 - RNA, 19 nt, synthetic construct
  misc_feature 1..19: note = antisense sequence
  source 1..19: mol_type = other RNA
ccaaatagaggaagcaact

SEQ ID NO: 1364 - RNA, 19 nt, synthetic construct
  misc_feature 1..19: note = sense sequence
  source 1..19: mol_type = other RNA
gttgcttcctctatttggt

SEQ ID NO: 1365 - RNA, 19 nt, synthetic construct
  misc_feature 1..19: note = antisense sequence
  source 1..19: mol_type = other RNA
accaaatagaggaagcaac

SEQ ID NO: 1366 - RNA, 19 nt, synthetic construct
  misc_feature 1..19: note = sense sequence
  source 1..19: mol_type = other RNA
ttgcttcctctatttggtt

SEQ ID NO: 1367 - RNA, 19 nt, synthetic construct
  misc_feature 1..19: note = antisense sequence
  source 1..19: mol_type = other RNA
aaccaaatagaggaagcaa